(19) United States
(12) Patent Application Publication
(10) Pub. No.: US 2016/0128944 A1
(43) Pub. Date: May 12, 2016

(54) COATED PARTICLES AND COMPOSITIONS COMPRISING SAME

(71) Applicant: VYOME BIOSCIENCES PVT. LTD., New Delhi (IN)

(72) Inventors: Suresh CHAWRAI, New Delhi (IN); Sudhanand PRASAD, New Delhi (IN); Kirti BAJAJ, New Delhi (IN)

(21) Appl. No.: 14/895,738

(22) PCT Filed: Jun. 3, 2014
(86) PCT No.: PCT/IB2014/061925

§ 371 Date: Dec. 3, 2015

(30) Foreign Application Priority Data
Jun. 4, 2013 (IN) 1684/DEL/2013

(51) Int. Cl. A61K 9/48; A61K 31/496; A61K 31/60; A61K 31/122; A61K 31/12; A61K 9/16; A61K 31/055; A61K 31/375; A61K 8/11; A61Q 5/02; A61Q 5/12; A61Q 11/00; A61K 31/55; A61K 31/4545; A61K 31/439; A61K 31/192; A61K 31/555

(52) U.S. Cl. CPC A61K 9/4816; A61K 31/555; A61K 31/496; A61K 31/60; A61K 31/122; A61K 31/12; A61K 9/1676; A61K 31/055; A61K 31/375; A61K 8/11; A61Q 5/02; A61Q 5/12; A61Q 11/00; A61K 31/55; A61K 31/4545; A61K 31/439; A61K 31/192; A61K 9/4825

(57) ABSTRACT

The invention provides species of lipid, protein, and/or cationic molecule coated active agents and compositions comprising the coated particles. The compositions can be personal care compositions such as anti-dandruff shampoo or conditioner, skin care compositions, creams, gels, toothpastes, mouth washes, and chewing gums.

### RELATED APPLICATIONS

This application claims benefit under one or more of 35 U.S.C. §119(a)-119(d) of Indian Provisional Patent Application No. 1684/DEL/2013, filed on Jun. 4, 2013, the content of which is incorporated herein by reference in its entirety.

### TECHNICAL FIELD

The present disclosure relates generally to particles comprising an active agent and one or more lipids and/or proteins and/or carbohydrates and/or cationic molecules, compositions comprising the same and methods of use and manufacturing thereof.

### BACKGROUND

Current antifungal, antibacterial, antioxidant, antiaging, anti-wrinkle, wound healing and similar therapies suffer from a number of limitations. U.S. patent publication no. 2005/0118276 describes micron sized zinc pyrithione particles coated with lipids and their use in shampoo. U.S. patent publication no. 2002/0106461 describes methods and apparatus for coating particles. WO 2010/038066 describes hair care compositions comprising porous silicons. Hot solution of zinc pyrithione (ZPT) in ethanolamine was poured over porous silicon powder and allowed to dry. The dried cake was ground into powder which was blended in shampoo. WO 2011/009083 describes antimicrobial agents adsorbed or embedded into/onto silica particles. US 2012/0171272 describes stabilized biocidal dispersion prepared via submicronized carrier particles and process for making the same. ZPT solution was adsorbed onto nanocarriers, such as ZnO, TiO₂, etc. . . . . Limitations of the current therapy include: limited exposure time; high application frequency; high relapse rates; and limited efficacy.

Therefore, there remains a need in the art for compositions comprising active agents and having improved efficacy and/or duration of effect.

### SUMMARY

In one aspect, the present disclosure provides particle comprising a core comprising one or more active agents and one or more coating layers, each coating layer comprising one or more lipids on the core. While the coating layers are described as comprising one or more lipids, the lipids can be replaced by other materials, such as, but not limited to, carbohydrates, proteins, polymers, and mixtures thereof. Thus any reference to a lipid coating is meant to include a coating comprising a material other than a lipid. The present disclosure further provides particle comprising a core coated with an active agent, i.e., the coating layer comprises the active agent. The present disclosure also provides particles comprising a core comprising an active agent and a coating layer comprising an active agent. The active agent in the core and the coating layer can be the same or different.

Without limitations, the particle can comprise two or more (e.g., two, three, four, five, six, seven, eight, nine, ten or more) different active agents and one or more (e.g., one, two, three, four, five, six, seven, eight, nine, ten or more) coating layers. The different active agents can be active against the same indication, different indications, or any combinations of same and different indications. Further, the different coating layers can comprise the same components or different components or any combinations of same and different components. In some embodiments, the particle comprises at least two different active agents and one coating layer. In some other embodiments, the particle comprises at least two different active agents and two coating layers.

In some embodiments, the particle comprises alternating layers of lipid coating and layer comprising an active agent, i.e., active agent layer. The active agents in the different layers can be the same or different or any combinations of same and different. Again, the different active agents can be active against the same indication, different indications, or any combinations of same and different indications. Further, components of the different lipid coating layers can be the same, different, or any combinations of same and different. In one non-limiting example, the particle comprises a first active agent in the core, a first coating layer on the core, a layer comprising a second active agent on the first coating layer, and a second coating layer on the layer comprising the second active agent. Without limitations, the first and the second active agent can be the same or they can be different. The first and second active agents can be active against the same indication. Alternatively, the first and second active agents can be active against different indications. Alternatively, the first and second active agents can be active against different indications.

In some embodiments, the lipid can be a lipid comprising 11 or fewer (e.g., 6, 8, or 10) carbons.

In some embodiments, the lipid is ethylene glycol distearate (EGDS), caprylic acid, capric acid, lauric acid, myristic acid, or palmitic acid, or their derivatives.

In some embodiments, the lipid is a fatty acid salt. In one embodiment, the fatty acid salt is zinc recinoleate.

In some embodiments, the coating layer comprises coenzyme-Q10 (CoQ10).

In some embodiments, the coating layer comprises two different lipids. In one embodiment, the coating layer comprises EGDS and myristic acid. In another embodiment, the coating layer comprises EGDS and lauric acid.

In some embodiments, the coating layer comprises a lipid and a protein. In one embodiment, the coating layer comprises EGDS and egg albumin.

In some embodiments, the coating layer comprises paraffin and a lipid. In one embodiment, the coating layer comprises paraffin and a lipid selected from the group consisting of caprylic acid, capric acid, lauric acid, myristic acid, and palmitic acid.

In another aspect, the disclosure provides a particle comprising a core comprising an active agent and a coating layer comprising a protein on the core. In one embodiment, the protein is egg albumin.

In some embodiments, the particle comprises a core comprising an active agent and a coating layer comprising a carbohydrate on the core.

In one aspect, the disclosure provides a particle comprising a core comprising an active agent and a coating layer comprising a cationic molecule on the core. In one embodiment, the cationic molecule is a polyamine.

In another aspect, the disclosure provides a composition comprising one or more of the particles disclosed herein. In some embodiments, the composition is a cream, ointment, oil, lotion, serum, gel, shampoo, conditioner, tooth paste, mouth wash, chewing gum, nail varnish, ointment, foam, spray, or aerosol. In some embodiments, the composition is an anti-dandruff hair care composition, a skin care composition, or an oral care composition.

In yet another aspect, the disclosure provides an antifungal composition comprising the particles disclosed herein. In one embodiment, the antifungal composition is a personal care composition, such as shampoo, e.g., an anti-dandruff shampoo.

In some embodiments, the composition is an anti-acne composition.

In some embodiments, the active agent is zinc pyrithione, ketoconazole, salicylic acid, curcumin or a derivative thereof (e.g., curcuminoids or tetrahydro curcuminoids), titanium oxide (TiO₂), zinc oxide (ZnO), chloroxylenol, or ascorbic acid.

### BRIEF DESCRIPTION OF THE DRAWINGS

FIG. 1 shows size distribution data using ZetaSizer (left panel) and Scanning Electron Microscopy (SEM) image (right panel) of EGDS coated zinc pyrithione nanoparticles (Composition D1).

FIG. 2 shows size distribution data using ZetaSizer (left panel) and Scanning Electron Microscopy (SEM) image (right panel) of caprylic acid (blend with paraffin) coated zinc pyrithione nanoparticles (Composition D8).

FIGS. 3A and 3B show size distribution data using ZetaSizer of EGDS coated ketoconazole nanoparticles (FIG. 3A, composition D12) and polyglyceryl-6 distearate coated ketoconazole nanoparticles (FIG. 3B, composition D13).

FIG. 4 shows comparative bar graphs for 1/MIC values of different dispersion compositions of ZPT particles (uncoated and lipid coated nanoparticles, and uncoated non-nanoparticles). Higher the value of I/MIC greater is the efficacy.

FIG. 5 shows comparative bar graphs for 1/MIC values of different shampoo compositions of ZPT particles (uncoated and lipid coated nanoparticles, and uncoated non-nanoparticles). Higher the value of I/MIC greater is the efficacy.

FIG. 6 shows comparative bar graphs for ZPT retained (%) on skin from applied dispersion compositions of ZPT particles (EGDS coated nanoparticles with two different ratios of EGDS-to-ZPT, and commercially available uncoated particles). EGDS coated ZPT particle dispersions D1 and D11 show 2.5-fold and 1.4-fold higher skin retention/deposition compared to ZPT FPS (uncoated non-nanoparticles). While dispersion D1 shows 1.8-fold higher retention/deposition compared to dispersion D11, which indicates larger lipid:ZPT ratio facilitates skin retention/deposition.

FIG. 7 shows Dose Response Curves (Log Trend lines) of in-house shampoos containing different ZPT APIs, plotted using Zones of Inhibition (ZOI) data. From the data, it is apparent that other factors being constant, shampoo composition S1 is slightly better than standard of care.

FIG. 8: Dose Response Curves (Log Trend lines) of shampoo compositions according to embodiments of the invention containing different ZPT APIs, plotted using ZOI data. Considering 2-fold higher retention for API from the shampoo formulation disclosed herein, there would be 42% efficacy improvement for the shampoo formulation disclosed herein relative to the standard of care at 20 and 10 μg/ml concentrations respectively.

FIG. 9 shows polynomial curves for CFU counts per ml for composition D1 versus standard ZPT dispersion at 0.5 and 1.0 μg/ml concentrations, recorded at various time points (experiment done in triplicates). Considering 2-fold higher retention for composition D1, composition D1 would be expected to perform 58% better in efficacy and would act 30% faster than standard ZPT at 1.0 and 0.5 μg/ml concentrations respectively.

FIG. 10 shows polynomial curves for CFU counts per ml for shampoo composition S1, control shampoos (using uncoated and non-nano ZPT particles) and standard of care (Head&Shoulders® shampoo) at ZPT concentration of 100 μg/ml, recorded at various time points for 2 hours (experiment done in triplicates). The shampoo composition S1 shows greater efficacy compared to any other shampoo composition in the study.

FIG. 11 shows kinetics of commercial ZPT powder (10 μg/ml) with different concentrations of Capmul 908-P (0%, 3%, 5% & 9%) on M. furfur.

FIG. 12 shows kinetics of commercial ZPT powder (50 μg/ml) with different concentrations of Capmul 908-P (0%, 3%, 5% & 9%) on M. furfur.

FIG. 13 shows the effect of Propylene Glycol Monocaprylate on ZPT Retention on Ex-Vivo Skin Model.

FIG. 14 shows the particle size distribution of stearic acid coated besifloxacin particles measured using Zeta-Sizer.

FIG. 15 shows the surface morphology of ethylene glycol distearate coated zinc pyrithione particles visualized under scanning electron microscope.

FIG. 16 shows the Size Distribution Curve of ethylene glycol distearate coated zinc pyrithione particles as analyzed by Mastersizer.

FIG. 17 shows the Minimum Inhibitory Concentration of In-house gels against P. acnes.

FIG. 18 shows the Dose Response Curves (Log Trend lines) of In-house shampoos and marketed shampoos, plotted using data of Zones of Inhibition on M. furfur.

### DETAILED DESCRIPTION

Aspects of the invention are based on inventors' discovery that skin, e.g., scalp micro-cracks, sweat or secretion pores, and hair follicles can act as reservoirs for microparticles of particular sizes. Efficacy of active agents, e.g., antifungal and antibacterial formulations can be enhanced using infundibular (intrafollicular space) delivery. Without wishing to be bound by a theory, it is believed that forming particles of the active agent by coating and/or associating with lipids can enhance the delivery of particles on sebum filled hair follicles and also exhibit fusogenecity of such particles to lipophilic fungal cell walls. This allows retention of particles comprising the pyrithione salt into hair follicles, followed by slow and continuous release of the active agent from the particles. Various aspects disclosed herein relate to an outcome of synergy. For example, synergy between two or more of indications (such as, an anti-propionibacterium lipid coated onto anti-inflammatory core for acne), actives (such as, anti-inflammatory agent and anti-Malassezia agent for dandruff; or keratolytic agent with anti-propionibacterium agent for acne), type of lipid (such as, EGDS or EGDP act as food for Malassezia and thus act as chemoattractants; a zinc pyrithione particle coated with EGDS or EGDP thus presents greater advantages for fungal kill by increased internalization and lipophilic interactions or fusogenicity with fungus), type of carbohydrate (such as anti-acne agent coated with chitosan finds easy entry into the biofilm enveloped P. acnes.), delivery (e.g., delivery of particles to the sebaceous glands can be enhanced by coating with a lipophilic material), and retention (e.g., intrafollicular and epidermal deposition of an active can be enhanced by coating with a lipophilic material).

Accordingly, in one aspect, the disclosure provides a particle comprising an active agent and a lipid component. The active agent can be present in the core of the particle and the lipid component can form at least one coating layer (e.g., one, two, three, four, five, six, seven, eight, nine, ten or more coating layers) over the core. The present disclosure further provides particles comprising a core coated with an active agent, i.e., the coating layer comprises the active agent. The present disclosure also provides particles comprising a core comprising an active agent and a coating layer comprising an active agent. The active agent in the core and the coating layer can be the same or different. While the lipid component is described as forming the coating layer, the lipid component can be replaced by other materials. Exemplary materials include, but are not limited to, carbohydrates, proteins, polymers, and the like.

The inventors have discovered inter alia that having lipid coated onto the particles (core) is essential for enhancing the overall activity as well as achieving the desired effects. For example, effective partitioning of the active from the formulation to the skin, lipophilicity enhancement of the unmodified actives, fusogenicity with the microorganism, delivery and retention at the site of action. This is in contrast to just having the two components (lipid and active) together in a vehicle, which would not be expected to give provide the desired effects since lipid alone may not be able to travel at the site of action and the lipophilicity of the active per se remain unchanged. Thus, having a having lipid coated onto the core makes the particles disclosed herein novel and provides the desired effects.

It is noted that art only describes lipohilic attraction of coated API with scalp and fungus. However, the art does not teach or suggest selecting or choosing coating material for different activities, such as, but not limited to, increasing or enhancing the activity of the active agent, having activity complementary to the active agent, or provide a synergistic effect with the active agent. For example, the particles disclosed herein comprise coating materials with different activities (sometimes augmentation of the parent activity, sometimes a complementary activity, etc. . . . ). Thus, the particles disclosed herein have properties beyond the simple lipophilic interactions of what is known in the art.

In some embodiments, the particle comprises a core comprising the active agent and one or more coating layers comprising a mixture comprising a lipid selected from the group consisting of caprylic acid, capric acid, lauric acid, myristic acid, and palmitic acid, and paraffin. In some embodiments, the particle comprises a core and a coating layer comprising a mixture comprising a lipid selected from the group consisting of caprylic acid, capric acid, lauric acid, myristic acid, and palmitic acid, and paraffin, and wherein the coating layer comprises an active agent. In some embodiments, the particle comprises a core comprising the active agent and a coating layer comprising a mixture comprising a lipid selected from the group consisting of caprylic acid, capric acid, lauric acid, myristic acid, and palmitic acid, and paraffin, and wherein the coating layer comprises an active agent. The active agent in the core and the coating layer can be the same or different.

Without limitations, the core can be partially of fully coated with the coating layer.

As used herein, the term “active agent” means a compound or composition that has a particular desired activity. For example, an active agent can be a therapeutic compound. Without limitations the active agent can be selected from the group consisting of small organic or inorganic molecules, saccharines, oligosaccharides, polysaccharides, peptides; proteins, peptide analogs and derivatives, peptidomimetics, nucleic acids, nucleic acid analogs and derivatives, antibodies, antigen binding fragments of antibodies, lipids, extracts made from biological materials, naturally occurring or synthetic compositions, and any combinations thereof.

In some embodiments, the active agent can be selected from the group consisting of antifungal agents, antibacterial agents, antimicrobial agents, antioxidant agents, cooling agents, soothing agents, wound healing agents, anti-inflammatory-agents, anti-aging agents, anti-wrinkle agents, skin whitening or bleaching agents, ultraviolet (UV) light absorbing or scattering agents, skin depigmentation agents, dyes or coloring agents, deodorizing agents, fragrances, and any combinations thereof.

In some embodiments, the active agent includes herbal active agent. Without limitations, the herbal active agent can be selected from the group consisting of bioactive herbs, herbal extracts, tinctures, essential oils, and mixtures thereof.

In some embodiments, the active agent is an antifungal agent. As used herein, the term “antifungal agent” is intended to mean a substance capable of inhibiting or preventing the growth, viability and/or reproduction of a fungal cell. Preferable antifungal agents are those capable of preventing or treating a fungal infection in an animal or plant. A preferable antifungal agent is a broad spectrum antifungal agent. However, an antifungal agent can also be specific to one or more particular species of fungus.

Examples of antifungal agents include, but are not limited to, azoles (e.g., Fluconazole, Isavuconazole, Itraconazole, Ketoconazole, Miconazole, Clortrimazole, Voriconazole, Posaconazole, Ravuconazole, Ciclopirox, etc.), polyenes (e.g., natamycin, lucensomycin, nystatin, amphotericin B, etc.), echinocandins (e.g., Cancidas), pradimicins (e.g., beanomicins, nikkomycins, sordarins, allylamines, etc.), Triclosan, Piroctone, fenpropimorph, terbinafine, and derivatives and analogs thereof. Additional antifungal agents include those described, for example, in Int. Pat. Pub. No. WO2001/066551, No. WO2002/090354, No. WO2000/043390, No. WO2010/032652, No. WO2003/008391, No. WO2004/018485, No. WO2005/006860, No. WO2003/086271, No. WO2002/067880; in U.S. Pat. App. Pub. No. 2008/0194661, No. 2008/0287440, No. 2005/0130940, No. 2010/0063285, No. 2008/0032994, No. 2006/0047135, No. 2008/0182885; and in U.S. Pat. No. 6,812,238; No. 4,588,525; No. 6,235,728; No. 6,265,584; No. 4,942,162; and No. 6,362,172, content of all of which is incorporated herein by reference.

In some embodiments, the antifungal agent is an antifungal peptide. Antifungal peptides are well known in the art (see for example, De Lucca et al., Rev. Iberoam. Micol. 17:116-120 (2000)). The antifungal peptide can be a naturally occurring peptide or an analog thereof, or it can be a synthetic peptide. As used herein, the term “analog” refers to a naturally occurring antifungal peptide that has been chemically modified to improve its effectiveness and/or reduce its toxic/side effects. Exemplary antifungal peptides can include, but are not limited to, syringomycins, syringostatins, syringotoxins, nikkomycins, echinocandins, pneumocadins, aculeacins, mulundocadins, cecropins, alpha-defensins, beta-defensins, novispirins, and combinations thereof. Other antifungal peptides include those described, for example, in U.S. Pat. No. 6,255,279 and U.S. Pat. App. Pub. No. 2005/0239709; No. 2005/0187151; No. 2005/0282755, and No. 2005/0245452, content all of which is incorporated herein by reference.

As used herein, the terms “fungus” or “fungi” include a variety of nucleated, spore-bearing organisms which are devoid of chlorophyll. Examples include yeasts, mildews, molds, rusts, and mushrooms. Examples of fungi include, but are not limited to Aspergillus fumigates, Aspergillus flavus, Aspergillus nidulans, Candida albicans, Candida glabrata, Candida guilliermondii, Candida krusei, Candida lusitaniae, Candida parapsilosis, Candida tropicalis, Cryptococcus neoformans, Issatchenkia orientalis, Coccidioides, Paracoccidioides, Histoplasma, Blastomyces, Trichophyton rubrum, and Neurospora crassa. In some embodiments, fungus is of the genus Malassezia (e.g., M. furfur, M. pachydermatis, M. globosa, M. restricta, M. slooffiae, M. sympodialis, M. nana, M. yamatoensis, M. dermatis, and M. obtuse). In one embodiments, the fungus is Trichophyton rubrum.

Without wishing to be bound by a theory, the Malassezia species causing most skin disease in humans, including the most common cause of dandruff and seborrhoeic dermatitis, is M. globosa (though M. restricta and M. furfur are also involved). The skin rash of tinea versicolor (pityriasis versicolor) is also due to infection by this fungus. As the fungus requires fat to grow, it is most common in areas with many sebaceous glands: on the scalp, face, and upper part of the body. When the fungus grows too rapidly, the natural renewal of cells is disturbed and dandruff appears with itching (a similar process may also occur with other fungi or bacteria).

In some embodiments, the antifungal agent is an antifungal agent effective against the fungus of genus Malassezia. In some further embodiments of this, the antifungal agent is an antifungal agent that is effective against the fungus M. globosa. In some embodiments, the antifungal agent is an antifungal agent effective against Trichophyton rubrum.

In some embodiments, the antifungal agent is Ketoconazole or a pyrithione salt. Examples of useful pyrithione salts include, but are not limited to, zinc pyrithione, sodium pyrithione, potassium pyrithione, lithium pyrithione, ammonium pyrithione, copper pyrithione, calcium pyrithione, magnesium pyrithione, strontium pyrithione, silver pyrithione, gold pyrithione, manganese pyrithione, and combinations thereof. Non-metal pyrithione salts such as the ethanolamine salt, chitosan salt, and the disulfide salt of pyrithione (which is commercially available as OMADINE MDS or OMDS), can also be used. The pyrithione salt can be used in any particulate form, including, but not limited to, crystalline form such as platelets, rods, needles, blocks, round and amorphous, regularly or irregularly shaped particles.

In some embodiments, the pyrithione salt is zinc pyrithione. Zinc pyrithione is best known for its use in treating dandruff and seborrhoeic dermatitis. It also has antibacterial properties and is effective against many pathogens from the Streptococcus and Staphylococcus genera. Its other medical applications include treatments of psoriasis, eczema, ringworm, fungus, athlete's foot, dry skin, atopic dermatitis, tinea, and vitiligo.

In some embodiments, the active agent is an anti-dandruff, anti-seborrheic dermatitis, or anti-psoriasis agent. Examples of suitable anti-dandruff agents, anti-seborrheic dermatitis agents, and anti-psoriasis agents include, but are not limited to, zinc pyrithione, selenium sulfide, sulfur; sulfonated shale oil; salicylic acid; coal tar; povidone-iodine, imidazoles such as ketoconazole, dichlorophenyl imidazolodioxalan, clotrimazole, itraconazoie, miconazole, climbazole, tioconazole, sulconazole, butoconazole, fluconazole, miconazolenitrite and any possible stereo isomers and derivatives thereof such as anthralin; piroctone olamine (Octopirox); selenium sulfide; ciclopirox olamine; anti-psoriasis agents; vitamin A analogs; corticosteroids and mixtures thereof.

In some embodiments, the active agent is an antibacterial agent. As used herein, the term “antibacterial agent” is defined as a compound having either a bactericidal or bacteriostatic effect upon bacteria contacted by the compound. As used herein, the term “bactericidal” is defined to mean having a destructive killing action upon bacteria. As used herein, the term “bacteriostatic” is defined to mean having an inhibiting action upon the growth of bacteria. Examples of antibacterial agents include, but are not limited to, macrolides or ketolides such as erythromycin, azithromycin, clarithromycin, and telithromycin; beta-lactams including penicillin, cephalosporin, and carbapenems such as carbapenem, imipenem, and meropenem; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, and astreonam; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, besifloxacin, besifloxaxin, clintafloxacin, ganefloxacin, gemifloxacin and pazufloxacin; antibacterial sulfonamides and antibacterial sulphanilamides, including para-aminobenzoic acid, sulfadiazine, sulfisoxazole, sulfamethoxazole and sulfathalidine; aminoglycosides such as streptomycin, neomycin, kanamycin, paromycin, gentamicin, tobramycin, amikacin, netilmicin, spectinomycin, sisomicin, dibekalin and isepamicin; tetracyclines such as tetracycline, chlortetracycline, demeclocycline, minocycline, oxytetracycline, methacycline, doxycycline; rifamycins such as rifampicin (also called rifampin), rifapentine, rifabutin, bezoxazinorifamycin and rifaximin; lincosamides such as lincomycin and clindamycin; glycopeptides such as vancomycin and teicoplanin; streptogramins such as quinupristin and daflopristin; oxazolidinones such as linezolid; polymyxin, colistin and colymycin; trimethoprim, bacitracin, and phosphonomycin.

In some embodiments, the antibacterial agent is an anti-acne agent. As used herein, the term “anti-acne agent” refers to any chemical that is effective in the treatment of acne and/or the symptoms associated therewith. Anti-acne agents are well known in the art such as U.S. Pat. App. Pub. No. 2006/0008538 and U.S. Pat. No. 5,607,980, content of both of which is incorporated herein by reference. Examples of useful anti-acne agents include, but are not limited to keratolytics, such as salicylic acid, derivatives of salicylic acid, and resorcinol; retinoids, such as retinoic acid, tretinoin, adapalene, tazarotene; sulfur-containing D- and L-amino acids and their derivatives and salts; lipoic acid; antibiotics and antimicrobials, such as benzoyl peroxide, triclosan, chlorhexidine gluconate, octopirox, tetracycline, 2,4,4′-trichloro-2′-hydroxy diphenyl ether, 3,4,4′-trichlorobanilide, nicotinamide, tea tree oil, rofecoxib, azelaic acid and its derivatives, phenoxyethanol, phenoxypropanol, phenoxisopropanol, ethyl acetate, clindamycin, erythromycin, and meclocycline; sebostats, such as flavonoids; and bile salts, such as scymnol sulfate and its derivatives, deoxycholate, and cholate; and combinations thereof. These agents are well known and commonly used in the field of personal care.

In some embodiments, the anti-acne agent can be an antimicrobial peptide having activity against P. acnes. Antimicrobial peptides are ubiquitous in nature and play an important role in the innate immune system of many species (Zasloff, Nature 415:389-395 (2002) and Epand et al., Biochim Biophys Acta 1462:11-28 (1999)). The antimicrobial peptide can be a naturally occurring peptide or an analog thereof, or it can be a synthetic peptide. As used herein an “analog” refers to a naturally-occurring antimicrobial peptide that has been chemically modified to improve its effectiveness and/or reduce its toxic side effects. The antimicrobial peptide can be a peptide known to be effective against Gram positive bacteria. Non-limiting examples include lantibiotics, such as nisin, subtilin, epidermin and gallidermin; defensins; attacins, such as sarcotoxin; cecropins, such as cecropin A, bactericidin, and lepidopteran; magainins; melittins; histatins; brevinins; and combinations thereof. Additionally, antimicrobial peptides having activity against P. acnes have been reported, for example, in U.S. Pat. App. Pub. No. 2005/0282755; No. 2005/02455452; and No. 2005/0209157, and U.S. Pat. No. 6,255,279, content of all of which is incorporated herein by reference. Suitable examples of antimicrobial peptides having reported activity against P. acnes include, but are not limited to, novispirins (Hogenhaug, supra), and those described in U.S. Pat. App. Pub. No. 2007/0265431, content of which is incorporated herein by reference.

In some embodiments, the active agent is an anti-inflammatory agent. As used herein the term “anti-inflammatory agent” refers to a compound (including its analogs, derivatives, prodrugs and pharmaceutically salts) which can be used to treat inflammation or inflammation related disease or disorder. Exemplary anti-inflammatory agents include, but are not limited to, the known steroidal anti-inflammatory and non-steroidal antiinflammatory drugs (NSAIDs). Exemplary steroidal anti-inflammatory agents include but are not limited to 21-acetoxypregnenolone, alclometasone, algestone, amcinonide, beclomethasone, betamethasone, budesonide, chloroprednisone, clobetasol, clobetansone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furcate, paramethosone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, derivatives thereof and mixtures thereof. Exemplary nonsteroidal anti-inflammatory agents include but are not limited to COX inhibitors (COX-1 or COX nonspecific inhibitors) and selective COX-2 inhibitors. Exemplary COX inhibitors include but are not limited to salicylic acid derivatives such as aspirin, sodium salicylate, choline magnesium trisalicylate, salicylate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam); alkanones such as nabumetone; derivatives thereof and mixtures thereof. Exemplary COX-2 inhibitors include but are not limited to diarylsubstituted furanones such as refecoxib; diaryl-substituted pyrazoles such as celecoxib; indole acetic acids such as etodolac and sulfonanilides such as nimesulide; derivatives thereof and mixtures thereof.

In some embodiments, the active agent is an anti-aging agent. As used herein, the term “anti-aging agent” means a compound or composition that inhibits or reduces signs of aging, such as wrinkles, fine lines, and other manifestations of photodamage. Examples of anti-aging agents include, but are not limited to, flavonoids such as quercetin, hesperidin, quercitrin, rutin, tangeritin, and epicatechin; CoQ10; inorganic sunscreens such as titanium dioxide and zinc oxide; organic sunscreens such as octyl-methyl cinnamates and derivatives thereof; retinoids; vitamins such as vitamin E, vitamin A, vitamin C (ascorbic acid), vitamin B, and derivatives thereof such as vitamin E acetate, vitamin C palmitate, and the like; antioxidants including alpha hydroxy acid such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucopehtonic acid, glucopheptono 1,4-lactone, gluconic acid, gluconolactone, glucuronic acid, glucurronolactone, glycolic acid, isopropyl pyruvate, methyl pyruvate, mucic acid, pyruvia acid, saccharic acid, saccaric acid 1,4-lactone, tartaric acid, and tartronic acid; beta hydroxy acids such as beta-hydroxybutyric acid, beta-phenyl-lactic acid, beta-phenylpyruvic acid; botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, rice, safflower, and mixtures thereof.

In some embodiments, the active agent is an ultraviolet (UV) light absorbing or scattering agent. Ultraviolet light absorbing agents include, for example, ultraviolet absorber of benzoic acid system such as para-aminobenzoic acid (hereinafter, abbreviated as PABA), PABA monoglycerin ester, N,N-dipropoxy PABA ethyl ester, N,N-diethoxy PABA ethyl ester, N,N-dimethyl PABA ethyl ester, N,N-dimethyl PABA butyl ester, and N,N-dimethyl PABA methyl ester and the like; ultraviolet absorber of anthranilic acid system such as homomenthyl-N-acetyl anthranilate and the like; ultraviolet absorber of salicylic acid system such as amyl salicylate, menthyl salicylate, homomenthyl salicylate, octyl salicylate, phenyl salicylate, benzyl salicylate, p-isopropanol phenyl salicylate and the like; ultraviolet absorber of cinnamic acid system such as octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxy cinnamate, isopropyl-p-methoxy cinnamate, isoamyl-p-methoxy cinnamate, octyl-p-methoxy cinnamate(2-ethylhexyl-p-methoxy cinnamate), 2-ethoxyethyl-p-methoxy cinnamate, cyclohexyl-p-methoxy cinnamate, ethyl-α-cyano-β-phenyl cinnamate, 2-ethylhexyl-α-cyano-β-phenyl cinnamate, glyceryl mono-2-ethylhexanoyl-dipara-methoxy cinnamate, methyl-bis(trimethylsiloxane)silylisopentyl trimethoxy cinnamate and the like; 3-(4′-methylbenzylidene)-d,l-camphor; 3-benzylidene-d,l-camphor; urocanic acid, urocanic acid ethyl ester; 2-phenyl-5-methylbenzoxazole; 2,2′-hydroxy-5-methylphenylbenzotriazole; 2-(2′-hydroxy-5′-t-octylphenyl)benzotriazole; 2-(2′-hydroxy-5′-methylphenylbenzotriazole; dibenzaladine; dianisoylmethane, 4-methoxy-4′-t-butyldibenzoylmethane; 5-(3,3-dimethyl-2-norbornylidene)-3-pentane-2-one; dimorpholinopyridazinone; and combinations thereof. Ultraviolet light scattering agents include, for example, powders such as titanium oxide, particulate titanium oxide, zinc oxide, particulate zinc oxide, ferric oxide, particulate ferric oxide, ceric oxide and the like.

In some embodiments, the active agent is an anti-wrinkle agent, e.g., a dermatological anti-wrinkle agent. Anti-wrinkle agents include, without limitations, flavonoids such as quercetin, hesperidin, quercitrin, rutin, tangeritin, and epicatechin; CoQ10; vitamin C; hydroxy acids including C₂-C₃₀ alpha-hydroxy acids such as glycolic acid, lactic acid, 2-hydroxy butanoic acid, malic acid, citric acid tartaric acid, alpha-hydroxyethanoic acid, hydroxycaprylic acid and the like; beta hydroxy acids including salicylic acid and polyhydroxy acids including gluconolactone (G4); and mixtures of these acids. Further anti-wrinkle agents include retinoic acid and gamma-linolenic acid.

In some embodiments, the active agent is a skin whitening or bleaching agent. Skin whitening and bleaching agents include hydrogen peroxide, zinc peroxide, sodium peroxide, hydroquinone, 4-isopropylcatechol, hydroquinone monobenzyl ether, kojic acid; lactic acid; ascorbyl acid and derivatives such as magnesium ascorbyl phosphate; arbutin; and licorice root. Sunless tanning actives include dihydroxyacetone (DHA); glyceryl aldehyde; tyrosine and tyrosine derivatives such as malyltyrosine, tyrosine glucosinate, and ethyl tyrosine; phospho-DOPA, indoles and derivatives; and mixtures thereof. Other skin whitening agents include sugar amines, such as glucosamine, N-acetyl glucosamine, glucosamine sulfate, mannosamine, N-acetyl mannosamine, galactosamine, N-acetyl galactosamine, their isomers (e.g., stereoisomers), and their salts (e.g., HCl salt); and N-acyl amino acid compounds, such as N-acyl phenylalanine, N-acyl tyrosine, their isomers, including their D and L isomers, salts, derivatives, and mixtures thereof. An example of a suitable N-acyl amino acid is N-undecylenoyl-L-phenylalanine is commercially available under the trade name SEPIWHITE™ from Seppic (France).

In some embodiments, the active agent is a skin depigmentation agent. Examples of suitable depigmentation agents include, but are not limited to: soy extract; soy isoflavones; retinoids such as retinol; kojic acid; kojic dipalmitate; hydroquinone; arbutin; transexamic acid; vitamins such as niacin and vitamin C; azelaic acid; linolenic acid and linoleic acid; placertia; licorice; and extracts such as chamomile and green tea; and salts and prodrugs thereof.

In some embodiments, the active agent is an antioxidant agent. As used herein, the term “antioxidant agent” refers to any molecule capable of slowing, reducing, inhibiting, or preventing the oxidation of other molecules. Examples of antioxidants include, but are not limited to, hydrophilic antioxidants, lipophilic antioxidants, and mixtures thereof. Non-limiting examples of hydrophilic antioxidants include chelating agents (e.g., metal chelators) such as ethylenediaminetetraacetic acid (EDTA), citrate, ethylene glycol tetraacetic acid (EGTA), 1,2-bis(o-aminophenoxy)ethane-N,N,N′,N′-tetraacetic acid (BAPTA), diethylene triamine pentaacetic acid (DTPA), 2,3-dimercapto-1-propanesulfonic acid (DMPS), dimercaptosuccinic acid (DMSA), α-lipoic acid, salicylaldehyde isonicotinoyl hydrazone (SIH), hexyl thioethylamine hydrochloride (HTA), desferrioxamine, salts thereof, and mixtures thereof. Additional hydrophilic antioxidants include ascorbic acid (vitamin C), cysteine, glutathione, dihydrolipoic acid, 2-mercaptoethane sulfonic acid, 2-mercaptobenzimidazole sulfonic acid, 6-hydroxy-2,5,7,8-tetramethylchroman-2-carboxylic acid, sodium metabisulfite, salts thereof, and mixtures thereof. Non-limiting examples of lipophilic antioxidants include vitamin E isomers such as α-, β-, γ-, and δ-tocopherols and α-, β-, γ-, and δ-tocotrienols; polyphenols such as 2-tert-butyl-4-methyl phenol, 2-tert-butyl-5-methyl phenol, and 2-tert-butyl-6-methyl phenol; butylated hydroxyanisole (BHA) (e.g., 2-tert-butyl-4-hydroxyanisole and 3-tert-butyl-4-hydroxyanisole); butylhydroxytoluene (BHT); tert-butylhydroquinone (TBHQ); ascorbyl palmitate; n-propyl gallate; salts thereof; and mixtures thereof. One of skill in the art will appreciate that antioxidants can be classified as primary antioxidants, secondary antioxidants, or metal chelators based upon the mechanisms in which they act. Primary antioxidants quench free radicals which are often the source of oxidative pathways, whereas secondary antioxidants function by decomposing the peroxides that are reactive intermediates of the pathways. Metal chelators function by sequestering the trace metals that promote free radical development.

In some embodiments, the active agent is a wound healing agent. As used herein, the term “wound healing agent” means active agents that are effective for promoting natural wound healing processes over days, weeks, or months. Exemplary wound healing agents include, but are not limited to, proteinaceous growth factors, vascular endothelial growth factors, anti-proliferant agent, antimicrobials, and anti-inflammatory agents.

In some embodiments, the active agent is a soothing agent. As used herein, the term “soothing agent” means a molecule which helps in reducing the discomfort of the skin and/or scalp, for example by soothing the feelings of itching. Exemplary soothing agents include, but are not limited to, aloe, avocado oil, green tea extract, hops extract, chamomile extract, colloidal oatmeal, calamine, cucumber extract, sodium palmate, sodium palm kernelate, butyrospermum parkii (i.e., shea butter), menthe piperita (i.e., peppermint) leaf oil, sericin, pyridoxine (a form of vitamin B6), retinyl palmitate and/or other forms of vitamin A, tocopheryl acetate and/or other forms of vitamin E, lauryl laurate, hyaluronic acid, aloe barbadensis leaf juice powder, euterpe oleracea (i.e., acai berry) fruit extract, riboflavin (i.e., vitamin B2), thiamin HCl and/or other forms of vitamin B1, and/any combinations thereof.

In some embodiments, the active agent is a cooling agent. As used herein, the term “cooling agent” refers to molecules which provide a sensation of cooling on application. Some exemplary cooling agents include, but are not limited to, WS-3; WS-23; menthol; 3-substituted-P-menthanes; N-substituted-P-menthane-3-carboxamides; isopulegol; 3-(1-menthoxy)propane-1,2-diol; 3-(1-menthoxy)-2-methylpropane-1,2-diol; p-menthane-2,3-diol; p-menthane-3,8-diol; 6-isopropyl-9-methyl-1,4-dioxaspiro[4,5]decane-2-methanol; menthyl succinate and its alkaline earth metal salts; trimethylcyclohexanol; N-ethyl-2-isopropyl-5-methylcyclohexanecarboxamide; Japanese mint oil; peppermint oil; menthone; menthone glycerol ketal; menthyl lactate; 3-(1-menthoxy)ethan-1-ol; 3-(1-menthoxy)propan-1-ol; 3-(1-menthoxy)butan-1-ol; 1-menthylacetic acid N-ethyl amide; 1-menthyl-4-hydroxypentanoate; 1-menthyl-3-hydroxybutyrate; N,2,3-trimethyl-2-(1-methylethyl)-butanamide; n-ethyl-t-2-c-6 nonadienamide; N,N-dimethyl menthyl succinamide; menthyl pyrrolidone carboxylate; and the like.

In some embodiments, the active agent is a coloring agent. As used herein, the term “coloring agent” means any substance that can be employed to produce a desired color. Gen. Such coloring agents are approved for human consumption pursuant an appropriate governmental agency and/or act, such as the Food and Drug Administration (FDA)/Federal Food Drug and Cosmetic Act (FD&C) in the US or an analogous agency of the European Union. For example, the coloring agent can be a food-grade dye or a lake. A “dye” is a water soluble compound, which is available as a powder, granule, liquid or other special purpose form. A “lake” is a water insoluble form of a dye. Exemplary coloring agents include, but are not limited to, FD&C Blue No. 1 (Brilliant Blue), FD&C Blue No. 2 (Indigotine), FD&C Green No. 3 (Fast Green), FD&C Red No. 3 (Erythrosine), FD&C Red No. 40 (Allura Red), FD&C Yellow No. 5 (Tartrazine), FD&C Yellow No. 6 (Sunset Yellow), annatto extract, anthocyanis, aronia/redfruit, beet juice, beet powder, beta-carotene, beta-apo-8-carotenal, black currant, burnt sugar, canthaxanthin, caramel, carbo medicinalis, carmine, carmine/beta-carotene, carmine blue, carminic acid, carrot, carrot oils, chlorophyll, chlorophyllin, cochineal extract, copper-chlorophyll, copper-chlorophyllin, curcumin, curcumin/Cu-chlorophyllin, elderberry, grape, grape skin extracts, hibiscus, lutein, mixed carotenoids, paprika, paprika extract, paprika oleoresin, riboflavin, saffron, spinach, stinging nettle, titanium dioxide, turmeric, and combinations thereof. Preferred coloring agents according to the present invention are FD&C Blue No. 1 (Brilliant Blue), FD&C Blue No. 2 (Indigotine), FD&C Green No. 3 (Fast Green), FD&C Red No. 3 (Erythrosine), FD&C Red No. 40 (Allura Red), FD&C Yellow No. 5 (Tartrazine), FD&C Yellow No. 6 (Sunset Yellow), and any combinations thereof.

In some embodiments; the active agent is a fragrance. Exemplary fragrances include; but are not limited to, 2,4-dimethyl-3-cyclohexene-1-carbaldehyde; isocyclocitral; menthone; isomenthone; ROMASCONE® (methyl 2,2-dimethyl-6-methylene-1-cyclohexanecarboxylate); nerone; terpineol; dihydroterpineol; terpenyl acetate; dihydroterpenyl acetate; dipentene; eucalyptol; hexylate; rose oxide; PERYCOROLLE® ((S)-1,8-p-menthadiene-7-ol); 1-p-menthene-4-ol; (1RS,3RS,4SR)-3-p-mentanyl acetate; (1R,2S,4R)-4,6,6-trimethyl-bicyclo[3,1,1]heptan-2-ol; DOREMOX® (tetrahydro-4-methyl-2-phenyl-2H-pyran); cyclohexyl acetate; cyclanol acetate; Fructalate (1,4-cyclohexane diethyldicarboxylate); KOUMALACTONE® ((3ARS,6SR,7ASR)-perhydro-3,6-dimethyl-benzo[B]furan-2-one); Natactone ((6R)-perhydro-3,6-dimethyl-benzo[B]furan-2-one); 2,4,6-trimethyl-4-phenyl-1,3-dioxane; 2,4,6-trimethyl-3-cyclohexene-1-carbaldehyde; (E)-3-methyl-5-(2,2,3-trimethyl-3-cyclopenten-1-yl)-4-penten-2-ol; (1′R,E)-2-ethyl-4-(2′,2′,3′-trimethyl-3′-cyclopenten-1′-yl)-2-buten-1-ol; POLYSANTOL® ((1′R,E)-3,3-dimethyl-5-(2′,2′,3′-trimethyl-3′-cyclopenten-1′-yl)-4-penten-2-ol); fleuramone; PARADISONE® (methyl-(1R)-cis-3-oxo-2-pentyl-1-cyclopentane acetate); Veloutone (2,2,5-Trimethyl-5-pentyl-1-cyclopentanone); NIRVANOL® (3,3-dimethyl-5-(2,2,3-trimethyl-3-cyclopenten-1-yl)-4-penten-2-ol); 3-methyl-5-(2,2,3-trimethyl-3-cyclopenten-1-yl)-2-pentanol; damascones; NEOBUTENONE® (1-(5,5-dimethyl-1-cyclohexen-1-yl)-4-penten-1-one); nectalactone ((1′R)-2-[2-(4′-methyl-3′-cyclohexen-1′-yl)propyl]cyclopentanone); alpha-ionone; beta-ionone; damascenone; DYNASCONE® (mixture of 1-(5,5-dimethyl-1-cyclohexen-1-yl)-4-penten-1-one and 1-(3,3-dimethyl-1-cyclohexen-1-yl)-4-penten-1-one); DORINONE® beta (1-(2,6,6-trimethyl-1-cyclohexen-1-yl)-2-buten-1-one); ROMANDOLIDE® ((1S,1′R)-[1-(3′,3′-Dimethyl-1′-cyclohexyl)ethoxycarbonyl]methyl propanoate); 2-tert-butyl-1-cyclohexyl acetate; LIMBANOL® (1-(2,2,3,6-tetramethyl-cyclohexyl)-3-hexanol); trans-1-(2,2,6-trimethyl-1-cyclohexyl)-3-hexanol; (E)-3-methyl-4-(2,6,6-trimethyl-2-cyclohexen-1-yl)-3-buten-2-one; terpenyl isobutyrate; LORYSIA® (4-(1,1-dimethylethyl)-1-cyclohexyl acetate); 8-methoxy-1-p-menthene; HELVETOLIDE® ((1S,1′R)-2-[1-(3′,3′-dimethyl-1′-cyclohexyl)ethoxy]-2-methylpropyl propanoate); para tert-butylcyclohexanone; menthenethiol; 1-methyl-4-(4-methyl-3-pentenyl)-3-cyclohexene-1-carbaldehyde; allyl cyclohexylpropionate; cyclohexyl salicylate; Methyl cedryl ketone; Verdylate; vetyverol; vetyverone; 1-(octahydro-2,3,8,8-tetramethyl-2-naphtalenyl)-1-ethanone; (5RS,9RS,10SR)-2,6,9,10-tetramethyl-1-oxaspiro[4.5]deca-3,6-diene and the (5RS,9SR,10RS) isomer; 6-ethyl-2,10,10-trimethyl-1-oxaspiro[4.5]deca-3,6-diene; 1,2,3,5,6,7-hexahydro-1,1,2,3,3-pentamethyl-4-indenone; HIVERNAL® (a mixture of 3-(3,3-dimethyl-5-indanyl)propanal and 3-(1,1-dimethyl-5-indanyl)propanal); Rhubofix® (3′,4-dimethyl-tricyclo[6.2.1.0(2,7)]undec-4-ene-9-spiro-2′-oxirane); 9/10-ethyldiene-3-oxatricyclo[6.2.1.0(2,7)]undecane; POLYWOOD® (perhydro-5,5,8A-trimethyl-2-naphthalenyl acetate); octalynol; CETALOX® (dodecahydro-3a,6,6,9a-tetramethyl-naphtho[2,1-b]furan); tricyclo[5.2.1.0(2,6)]dec-3-en-8-yl acetate and tricyclo[5.2.1.0(2,6)]dec-4-en-8-yl acetate as well as tricyclo[5.2.1.0(2,6)]dec-3-en-8-yl propanoate and tricyclo[5.2.1.0(2,6)]dec-4-en-8-yl propanoate; camphor; borneol; isobornyl acetate; 8-isopropyl-6-methyl-bicyclo[2.2.2]oct-5-ene-2-carbaldehyde; camphopinene; cedramber (8-methoxy-2,6,6,8-tetramethyl-tricyclo[5.3.1.0(1,5)]undecane); cedrene; cedrenol; cedrol; FLOREX® (mixture of 9-ethylidene-3-oxatricyclo[6.2.1.0(2,7)]undecan-4-one and 10-ethylidene-3-oxatricyclo[6.2.1.0(2,7)]undecan-4-one); 3-methoxy-7,7-dimethyl-10-methylene-bicyclo[4.3.1]decane; CEDROXYDE® (trimethyl-13-oxabicyclo-[10.1.0]-trideca-4,8-diene); Ambrettolide LG ((E)-9-hexadecen-16-olide); HABANOLIDE® (pentadecenolide); muscenone (3-methyl-(4/5)-cyclopentadecenone); muscone; EXALTOLIDE® (pentadecanolide); EXALTONE® (cyclopentadecanone); (1-ethoxyethoxy)cyclododecane; Astrotone; LILIAL®; rosinol; and the like.

In some embodiments, the active agent is zinc pyrithione; ketoconazole; salicylic acid; curcumin or a derivative of curcumin (e.g., curcuminoids or tetrahydro curcuminoids); titanium dioxide (TiO₂); zinc oxide (ZnO); chloroxylenol; querciptin; CoQ10; vitamin C; herbal extracts; alkaloids; flavonoids; 13-cis retinoic acid; 3,4-methylenedioxymethamphetamine; 5-fluorouracil; 6,8-dimercaptooctanoic acid (dihydrolipoic acid); abacavir; acebutolol; acetaminophen; acetaminosalol; acetazolamide; acetohydroxamic acid; acetylsalicylic acid; acitretin; aclovate; acrivastine; actiq; acyclovir; adapalene; adefovir dipivoxil; adenosine; Albaconazole; albuterol; alfuzosin; Allicin; allopurinol; alloxanthine; allylamines; almotriptan; alpha-hydroxy acids; alprazolam; alprenolol; aluminum acetate; aluminum chloride; aluminum chlorohydroxide; aluminum hydroxide; amantadine; amiloride; aminacrine; aminobenzoic acid (PABA); aminocaproic acid; aminoglycosides such as streptomycin, neomycin, kanamycin, paromycin, gentamicin, tobramycin, amikacin, netilmicin, spectinomycin, sisomicin, dibekalin and isepamicin; aminosalicylic acid; amiodarone; amitriptyline; amlodipine; amocarzine; amodiaquin; Amorolfin; amoxapine; amphetamine; amphotericin B; ampicillin; anagrelide; anastrozole; Anidulafungin; anthralin; antibacterial sulfonamides and antibacterial sulphanilamides, including para-aminobenzoic acid, sulfadiazine, sulfisoxazole, sulfamethoxazole and sulfathalidine; antifungal peptide and derivatives and analogs thereof; apomorphine; aprepitant; arbutin; aripiprazole; ascorbic acid; ascorbyl palmitate; atazanavir; atenolol; atomoxetine; atropine; azathioprine; azelaic acid; azelaic acid; azelastine; azithromycin; bacitracin; bacitracin; beanomicins; beclomefhasone dipropionate; bemegride; benazepril; bendroflumethiazide; benzocaine; Benzoic acid with a keratolytic agent; benzonatate; benzophenone; benztropine; bepridil; beta-hydroxy acids; beta-lactams including penicillin, cephalosporin, and carbapenems such as carbapenem, imipenem, and meropenem; betamethasone dipropionate; betamethasone valerate; brimonidine; brompheniramine; bupivacaine; buprenorphine; bupropion; burimamide; butenafine; Butenafine; butoconazole; Butoconazole; cabergoline; caffeic acid; caffeine; calcipotriene; camphor; Cancidas; candesartan cilexetil; capsaicin; carbamazepine; Caspofungin; cefditoren pivoxil; cefepime; cefpodoxime proxetil; celecoxib; cetirizine; cevimeline; chitosan; chlordiazepoxide; chlorhexidine; chloroquine; chlorothiazide; chloroxylenol; chlorpheniramine; chlorpromazine; chlorpropamide; ciclopirox; Ciclopirox (ciclopirox olamine); cilostazol; cimetidine; cinacalcet; ciprofloxacin; citalopram; citric acid; Citronella oil; cladribine; clarithromycin; clemastine; clindamycin; clioquinol; clobetasol propionate; clomiphene; clonidine; clopidogrel; Clortrimazole; clotrimazole; Clotrimazole; clozapine; cocaine; Coconut oil; codeine; colistin; colymycin; cromolyn; crotamiton; Crystal violet; cyclizine; cyclobenzaprine; cycloserine; cytarabine; dacarbazine; dalfopristin; dapsone; daptomycin; daunorubicin; deferoxamine; dehydroepiandrosterone; delavirdine; desipramine; desloratadine; desmopressin; desoximetasone; dexamethasone; dexmedetomidine; dexmethylphenidate; dexrazoxane; dextroamphetamine; diazepam; dicyclomine; didanosine; dihydrocodeine; dihydromorphine; diltiazem; diphenhydramine; diphenoxylate; dipyridamole; disopyramide; dobutamine; dofetilide; dolasetron; donepezil; dopa esters; dopamine; dopamnide; dorzolamide; doxepin; doxorubicin; doxycycline; doxylamine; doxypin; duloxetine; dyclonine; echinocandins; econazole; Econazole; eflormthine; eletriptan; emtricitabine; enalapril; ephedrine; epinephrine; epinine; epirubicin; eptifibatide; ergotarnine; erythromycin; escitalopram; esmolol; esomeprazole; estazolam; estradiol; ethacrynic acid; ethinyl estradiol; etidocaine; etomidate; famciclovir; famotidine; felodipine; fentanyl; Fenticonazole; ferulic acid; fexofenadine; flecainide; fluconazole; Fluconazole; flucytosiine; Flucytosine or 5-fluorocytosine; fluocinolone acetonide; fluocinonide; fluoxetine; fluphenazine; flurazepam; fluvoxamine; formoterol; furosemide; galactarolactone; galactonic acid; galactonolactone; galactose; galantamine; gatifloxacin; gefitinib; gemcitabine; gemifloxacin; gluconic acid; glycolic acid; glycolic acid; glycopeptides such as vancomycin and teicoplanin; griseofulvin; Griseofulvin; guaifenesin; guanethidine; haloperidol; haloprogin; Haloprogin; herbal extract, an alkaloid, a flvanoid, Abafungin; hexylresorcinol; homatropine; homosalate; hydralazine; hydrochlorothiazide; hydrocortisone; hydrocortisone 17-butyrate; hydrocortisone 17-valerate; hydrocortisone 21-acetate; hydromorphone; hydroquinone; hydroquinone monoether; hydroxyzine; hyoscyamine; hypoxanthine; ibuprofen; ichthammol; idarubicin; imatinib; imipramine; imiquimod; indinavir; indomethacin; Iodine; irbesartan; irinotecan; Isavuconazole; Isoconazole; isoetharine; isoproterenol; itraconazole; Itraconazole; kanamycin; ketamine; ketanserin; ketoconazole; ketoprofen; ketotifen; kojic acid; labetalol; lactic acid; lactobionic acid; lactobionic acid; lamivudine; lamotrigine; lansoprazole; lemon myrtle; letrozole; leuprolide; levalbuterol; levofloxacin; lidocaine; lincosamides such as lincomycin and clindamycin; linezolid; lobeline; loperamide; losartan; loxapine; lucensomycin; lysergic diethylamide; macrolides or ketolides such as erythromycin, azithromycin, clarithromycin, and telithromycin; mafenide; malic acid; maltobionic acid; mandelic acid; mandelic acid; maprotiline; mebendazole; mecamylamine; meclizine; meclocycline; memantine; menthol; meperidine; mepivacaine; mercaptopurine; mescaline; metanephrine; metaproterenol; metaraminol; metformin; methadone; methamphetamine; methotrexate; methoxamine; methyl nicotinate; methyl salicylate; methyldopa esters; methyldopamide; methyllactic acid; methylphenidate; metiamide; metolazone; metoprolol; metronidazole; mexiletine; Micafungin; miconazole; Miconazole; midazolam; midodrine; miglustat; minocycline; minoxidil; mirtazapine; mitoxantrone; moexiprilat; molindone; monobenzone; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, and astreonam; morphine; moxifloxacin; moxonidine; mupirocin; nadolol; naftifine; Naftifine; nalbuphine; nalmefene; naloxone; naproxen; natamycin; Neem Seed Oil; nefazodone; nelfinavir; neomycin; nevirapine; N-guanylhistamine; nicardipine; nicotine; nifedipine; nikkomycins; nimodipine; nisoldipine; nizatidine; norepinephrine; nystatin; nystatin; octopamine; octreotide; octyl methoxycinnamate; octyl salicylate; ofloxacin; olanzapine; Olive leaf extract; olmesartan medoxomil; olopatadine; omeprazole; Omoconazole; ondansetron; Orange oil; oxazolidinones such as linezolid; oxiconazole; Oxiconazole; oxotremorine; oxybenzone; oxybutynin; oxycodone; oxymetazoline; padimate O; palmarosa oil; palonosetron; pantothenic acid; pantoyl lactone; paroxetine; patchouli; pemoline; penciclovir; penicillamine; penicillins; pentazocine; pentobarbital; pentostatin; pentoxifylline; pergolide; perindopril; permethrin; phencyclidine; phenelzine; pheniramine; phenmetrazine; phenobarbital; phenol; phenoxybenzamine; phenpropimorph; phentolamine; phenylephrine; phenylpropanolamine; phenytoin; phosphonomycin; physostigmine; pilocarpine; pimozide; pindolol; pioglitazone; pipamazine; piperonyl butoxide; pirenzepine; Piroctone; piroctone olamine; podofilox; podophyllin; Polygodial; polyhydroxy acids; polymyxin; Posaconazole; pradimicins; pramoxine; pratipexole; prazosin; prednisone; prenalterol; prilocaine; procainamide; procaine; procarbazine; promazine; promethazine; promethazine propionate; propafenone; propoxyphene; propranolol; propylthiouracil; protriptyline; pseudoephedrine; pyrethrin; pyrilamine; pyrimethamine; quetiapine; quinapril; quinethazone; quinidine; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, besifloxacin, besifloxaxin, clintafloxacin, ganefloxacin, gemifloxacin and pazufloxacin; quinupristin; rabeprazole; Ravuconazole; reserpine; resorcinol; retinal; retinoic acid; retinol; retinyl acetate; retinyl palmitate; ribavirin; ribonic acid; ribonolactone; rifampin; rifamycins such as rifampicin (also called rifampin), rifapentine, rifabutin, bezoxazinorifamycin and rifaximin; rifapentine; rifaximin; riluzole; rimantadine; risedronic acid; risperidone; ritodrine; rivasfigmine; rizatriptan; ropinirole; ropivacaine; salicylamide; salicylic acid; salicylic acid; salmeterol; scopolamine; selegiline; Selenium; selenium sulfide; serotonin; Sertaconazole; sertindole; sertraline; sibutramine; sildenafil; sordarins; sotalol; streptogramins such as quinupristin and daflopristin; streptomycin; strychnine; sulconazole; Sulconazole; sulfabenz; sulfabenzamide; sulfabromomethazine; sulfacetamide; sulfachlorpyridazine; sulfacytine; sulfadiazine; sulfadimethoxine; sulfadoxine; sulfaguanole; sulfalene; sulfamethizole; sulfamethoxazole; sulfanilamide; sulfapyrazine; sulfapyridine; sulfasalazine; sulfasomizole; sulfathiazole; sulfisoxazole; tadalafil; tamsulosin; tartaric acid; tazarotene; Tea tree oil—ISO 4730 (“Oil of Melaleuca, Terpinen-4-ol type”); tegaserol; telithromycin; telmisartan; temozolomide; tenofovir disoproxil; terazosin; terbinafine; Terbinafine; terbutaline; terconazole; Terconazole; terfenadine; tetracaine; tetracycline; tetracyclines such as tetracycline, chlortetracycline, demeclocycline, minocycline, oxytetracycline, methacycline, doxycycline; tetrahydrozoline; theobromine; theophylline; thiabendazole; thioridazine; thiothixene; thymol; tiagabine; timolol; tinidazole; tioconazole; Tioconazole; tirofiban; tizanidine; tobramycin; tocainide; tolazoline; tolbutamide; tolnaftate; Tolnaftate; tolterodine; tramadol; tranylcypromine; trazodone; triamcinolone acetonide; triamcinolone diacetate; triamcinolone hexacetonide; triamterene; triazolam; triclosan; triclosan; Triclosan; triflupromazine; trimethoprim; trimethoprim; trimipramine; tripelennamine; triprolidine; tromethamine; tropic acid; tyramine; undecylenic acid; Undecylenic acid; urea; urocanic acid; ursodiol; vardenafil; venlafaxine; verapamil; vitamin C; vitamin E acetate; voriconazole; Voriconazole; warfarin; xanthine; zafirlukast; zaleplon; zinc pyrithione; Zinc Selenium sulfide; ziprasidone; zolmitriptan; Zolpidem; WS-3; WS-23; menthol; 3-substituted-P-menthanes; N-substituted-P-menthane-3-carboxamides; isopulegol; 3-(1-menthoxy)propane-1,2-diol; 3-(1-menthoxy)-2-methylpropane-1,2-diol; p-menthane-2,3-diol; p-menthane-3,8-diol; 6-isopropyl-9-methyl-1,4-dioxaspiro[4,5]decane-2-methanol; menthyl succinate and its alkaline earth metal salts; trimethylcyclohexanol; N-ethyl-2-isopropyl-5-methylcyclohexanecarboxamide; Japanese mint oil; peppermint oil; menthone; menthone glycerol ketal; menthyl lactate; 3-(1-menthoxy)ethan-1-ol; 3-(1-menthoxy)propan-1-ol; 3-(1-menthoxy)butan-1-ol; 1-menthylacetic acid N-ethylamide; 1-menthyl-4-hydroxypentanoate; 1-menthyl-3-hydroxybutyrate; N,2,3-trimethyl-2-(1-methylethyl)-butanamide; n-ethyl-t-2-c-6 nonadienamide; N,N-dimethyl menthyl succinamide; menthyl pyrrolidone carboxylate; aloe; avocado oil; green tea extract; hops extract; chamomile extract; colloidal oatmeal; calamine; cucumber extract; sodium palmate; sodium palm kernelate; butyrospermum parkii (i.e., shea butter); menthe piperita (i.e.; peppermint) leaf oil; sericin; pyridoxine (a form of vitamin B6); retinyl palmitate and/or other forms of vitamin A; tocopheryl acetate and/or other forms of vitamin E; lauryl laurate; hyaluronic acid; aloe barbadensis leaf juice powder; euterpe oleracea (i.e., acai berry) fruit extract; riboflavin (i.e., vitamin B2); thiamin HCl and/or other forms of vitamin B1; ethylenediaminetetraacetic acid (EDTA); citrate; ethylene glycol tetraacetic acid (EGTA); 1,2-bis(o-aminophenoxy)ethane-N,N,N′,N′-tetraacetic acid (BAPTA); diethylene triamine pentaacetic acid (DTPA); 2,3-dimercapto-1-propanesulfonic acid (DMPS); dimercaptosuccinic acid (DMSA); α-lipoic acid; salicylaldehyde isonicotinoyl hydrazone (SIH); hexyl thioethylamine hydrochloride (HTA); desferrioxamine; ascorbic acid (vitamin C); cysteine; glutathione; dihydrolipoic acid; 2-mercaptoethane sulfonic acid; 2-mercaptobenzimidazole sulfonic acid; 6-hydroxy-2,5,7,8-tetramethylchroman-2-carboxylic acid; sodium metabisulfite; vitamin E isomers such as α-, β-, γ-, and δ-tocopherols and α-, β-, γ-, and δ-tocotrienols; polyphenols such as 2-tert-butyl-4-methyl phenol, 2-tert-butyl-5-methyl phenol, and 2-tert-butyl-6-methyl phenol; butylated hydroxyanisole (BHA) such as 2-tert-butyl-4-hydroxyanisole and 3-tert-butyl-4-hydroxyanisole; butylhydroxytoluene (BHT); tert-butylhydroquinone (TBHQ); ascorbyl palmitate; n-propyl gallate; soy extract; soy isoflavones; retinoids such as retinol; kojic acid; kojic dipalmitate; hydroquinone; arbutin; transexamic acid; vitamins such as niacin and vitamin C; azelaic acid; linolenic acid and linoleic acid; placertia; licorice; and extracts such as chamomile and green tea; hydrogen peroxide; zinc peroxide; sodium peroxide; hydroquinone; 4-isopropylcatechol; hydroquinone monobenzyl ether; kojic acid; lactic acid; ascorbyl acid and derivatives such as magnesium ascorbyl phosphate; arbutin; licorice root; dihydroxyacetone (DHA); glyceryl aldehyde; tyrosine and tyrosine derivatives such as malyltyrosine, tyrosine glucosinate, and ethyl tyrosine; phospho-DOPA; indoles and derivatives; glucosamine; N-acetyl glucosamine; glucosamine sulfate; mannosamine; N-acetyl mannosamine; galactosamine; N-acetyl galactosamine; N-acyl amino acid compounds (e.g., N-undecylenoyl-L-phenylalanine); flavonoids such as quercetin, hesperidin, quercitrin, rutin, tangeritin, and epicatechin; CoQ10; vitamin C; hydroxy acids including C₂-C₃₀ alpha-hydroxy acids such as glycolic acid, lactic acid, 2-hydroxy butanoic acid, malic acid, citric acid tartaric acid, alpha-hydroxyethanoic acid, hydroxycaprylic acid and the like; beta hydroxy acids including salicylic acid and polyhydroxy acids including gluconolactone (G4); retinoic acid; gamma-linolenic acid; ultraviolet absorber of benzoic acid system such as para-aminobenzoic acid (hereinafter, abbreviated as PABA), PABA monoglycerin ester, N,N-dipropoxy PABA ethyl ester, N,N-diethoxy PABA ethyl ester, N,N-dimethyl PABA ethyl ester, N,N-dimethyl PABA butyl ester, and N,N-dimethyl PABA methyl ester and the like; ultraviolet absorber of anthranilic acid system such as homomenthyl-N-acetyl anthranilate and the like; ultraviolet absorber of salicylic acid system such as amyl salicylate, menthyl salicylate, homomenthyl salicylate, octyl salicylate, phenyl salicylate, benzyl salicylate, p-isopropanol phenyl salicylate and the like; ultraviolet absorber of cinnamic acid system such as octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxy cinnamate, isopropyl-p-methoxy cinnamate, isoamyl-p-methoxy cinnamate, octyl-p-methoxy cinnamate(2-ethylhexyl-p-methoxy cinnamate), 2-ethoxyethyl-p-methoxy cinnamate, cyclohexyl-p-methoxy cinnamate, ethyl-α-cyano-β-phenyl cinnamate, 2-ethylhexyl-α-cyano-β-phenyl cinnamate, glyceryl mono-2-ethylhexanoyl-dipara-methoxy cinnamate, methyl bis(trimethylsiloxane)silylisopentyl trimethoxy cinnamate and the like; 3-(4′-methylbenzylidene)-d,l-camphor; 3-benzylidene-d,l-camphor; urocanic acid, urocanic acid ethyl ester; 2-phenyl-5-methylbenzoxazole; 2,2′-hydroxy-5-methylphenylbenzotriazole; 2-(2′-hydroxy-5′-t-octylphenyl)benzotriazole; 2-(2′-hydroxy-5′-methylphenylbenzotriazole; dibenzaladine; dianisoylmethane; 4-methoxy-4′-t-butyldibenzoylmethane; 5-(3,3-dimethyl-2-norbornylidene)-3-pentane-2-one; dimorpholinopyridazinone; titanium oxide; particulate titanium oxide; zinc oxide; particulate zinc oxide; ferric oxide; particulate ferric oxide; ceric oxide; inorganic sunscreens such as titanium dioxide and zinc oxide; organic sunscreens such as octyl-methyl cinnamates and derivatives thereof; retinoids; vitamins such as vitamin E, vitamin A, vitamin C (ascorbic acid), vitamin B, and derivatives thereof such as vitamin E acetate, vitamin C palmitate, and the like; antioxidants including alpha hydroxy acid such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucopehtonic acid, glucopheptono 1,4-lactone, gluconic acid, gluconolactone, glucuronic acid, glucurronolactone, glycolic acid, isopropyl pyruvate, methyl pyruvate, mucic acid, pyruvia acid, saccharic acid, saccaric acid 1,4-lactone, tartaric acid, and tartronic acid; beta hydroxy acids such as beta-hydroxybutyric acid, beta-phenyl-lactic acid, beta-phenylpyruvic acid; botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, rice, and safflower; 21-acetoxypregnenolone; alclometasone; algestone; amcinonide; beclomethasone; betamethasone; budesonide; chloroprednisone; clobetasol; clobetansone; clocortolone; cloprednol; corticosterone; cortisone; cortivazol; deflazacort; desonide; desoximetasone; dexamethasone; diflorasone; diflucortolone; difluprednate; enoxolone; fluazacort; flucloronide; flumethasone flunisolide; fluocinolone acetonide; fluocinonide; fluocortin butyl; fluocortolone; fluorometholone; fluperolone acetate; fluprednidene acetate; fluprednisolone; flurandrenolide; fluticasone propionate; formocortal; halcinonide; halobetasol propionate; halometasone; halopredone acetate; hydrocortamate; hydrocortisone; loteprednol etabonate; mazipredone; medrysone; meprednisone; methylprednisolone; mometasone furcate; paramethosone; prednicarbate; prednisolone; prednisolone 25-diethylamino-acetate; prednisolone sodium phosphate; prednisone; prednival; prednylidene; rimexolone; tixocortol; triamcinolone; triamcinolone acetonide; triamcinolone benetonide; triamcinolone hexacetonide; COX inhibitors such as salicylic acid derivatives (e.g., aspirin, sodium salicylate, choline magnesium trisalicylate, salicylate, diflunisal, sulfasalazine and olsalazine); para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam); alkanones such as nabumetone; diaryl substituted furanones such as refecoxib; diaryl-substituted pyrazoles such as celecoxib; indole acetic acids such as etodolac; sulfonanilides such as nimesulide; selenium sulfide; sulfur; sulfonated shale oil; salicylic acid; coal tar; povidone-iodine, imidazoles such as ketoconazole, dichlorophenyl imidazolodioxalan, clotrimazole, itraconazoie, miconazole, climbazole, tioconazole, sulconazole, butoconazole, fluconazole, miconazolenitrite; anthralin; piroctone olamine (Octopirox); ciclopirox olamine; anti-psoriasis agents; vitamin A analogs; corticosteroids; and any combinations thereof.

The particle disclosed herein can comprise any amount of the active agent. For example, the particle can comprise between about 0.01% to about 99% (w/w) of the active agent. For example, the particle can comprise between about 0.01% to about 99% (w/w) of the active agent. In some embodiments, the active agent comprises greater than 1% (w/w), greater than 5% (w/w), greater than 10% (w/w), greater than 15% (w/w), greater than 20% (w/w), greater than 25% (w/w), greater than 30% (w/w), greater than 35% (w/w), greater than 40% (w/w), greater than 45% (w/w), greater than 50% (w/w), greater than 55% (w/w), greater than 60% (w/w), greater than 65% (w/w), greater than 70% (w/w), greater than 75% (w/w), greater than 80% (w/w), greater than 85% (w/w), greater than 90% (w/w), or greater than 95% (w/w) of the total weight of the particles. In some embodiments, the content of active agent in the particles can range from about 75% to about 97% (w/w). In some other embodiments, the content of active agent in the particles can range from about 3% to about 25% (w/w).

A lipid for use in the particles disclosed herein can be selected from the group consisting of fatty acids, fatty alcohols, glycerolipids (e.g., monoglycerides, diglycerides, and triglycerides), phospholipids, glycerophospholipids, sphingolipids, sterol lipids, prenol lipids, saccharolipids, polyketides, and any combination thereof. In some embodiments, the lipid can be selected from the group consisting of 1,3-Propanediol Dicaprylate/Dicaprate; 10-undecenoic acid; 1-dotriacontanol; 1-heptacosanol; 1-nonacosanol; 2-ethyl hexanol; Androstanes; Arachidic acid; Arachidonic acid; arachidyl alcohol; Behenic acid; behenyl alcohol; Capmul MCM C10; Capric acid; capric alcohol; capryl alcohol; Caprylic acid; Caprylic/Capric Acid Ester of Saturated Fatty Alcohol C12-C18; Caprylic/Capric Triglyceride; Caprylic/Capric Triglyceride; Ceramide phosphorylcholine (Sphingomyelin, SPH); Ceramide phosphorylethanolamine (Sphingomyelin, Cer-PE); Ceramide phosphorylglycerol; Ceroplastic acid; Cerotic acid; Cerotic acid; ceryl alcohol; Cetearyl alcohol; Ceteth-10; cetyl alcohol; Cholanes; Cholestanes; cholesterol; cis-11-eicosenoic acid; cis-11-octadecenoic acid; cis-13-docosenoic acid; cluytyl alcohol; coenzyme Q10 (CoQ10); Dihomo-γ-linolenic; Docosahexaenoic acid; egg lecithin; Eicosapentaenoic acid; Eicosenoic acid; Elaidic acid; elaidolinolenyl alcohol; elaidolinoleyl alcohol; elaidyl alcohol; Erucic acid; erucyl alcohol; Estranes; Ethylene glycol distearate (EGDS); Geddic acid; geddyl alcohol; glycerol distearate (type I) EP (Precirol ATO 5); Glycerol Tricaprylate/Caprate; Glycerol Tricaprylate/Caprate (CAPTEX® 355 EP/NF); glyceryl monocaprylate (Capmul MCM C8 EP); Glyceryl Triacetate; Glyceryl Tricaprylate; Glyceryl Tricaprylate/Caprate/Laurate; Glyceryl Tricaprylate/Tricaprate; glyceryl tripalmitate (Tripalmitin); Henatriacontylic acid; Heneicosyl alcohol; Heneicosylic acid; Heptacosylic acid; Heptadecanoic acid; Heptadecyl alcohol; Henatriacontylic acid; isostearic acid; isostearyl alcohol; Lacceroic acid; Lauric acid; Lauryl alcohol; Lignoceric acid; lignoceryl alcohol; Linoelaidic acid; Linoleic acid; linolenyl alcohol; linoleyl alcohol; Margaric acid; Mead; Melissic acid; melissyl alcohol; Montanic acid; montanyl alcohol; myricyl alcohol; Myristic acid; Myristoleic acid; Myristyl alcohol; neodecanoic acid; neoheptanoic acid; neononanoic acid; Nervonic; Nonacosylic acid; Nonadecyl alcohol; Nonadecylic acid; Nonadecylic acid; Oleic acid; oleyl alcohol; Palmitic acid; Palmitoleic acid; palmitoleyl alcohol; Pelargonic acid; pelargonic alcohol; Pentacosylic acid; Pentadecyl alcohol; Pentadecylic acid; Phosphatidic acid (phosphatidate, PA); Phosphatidylcholine (lecithin, PC); Phosphatidylethanolamine (cephalin, PE); Phosphatidylinositol (PI); Phosphatidylinositol bisphosphate (PIP2); Phosphatidylinositol phosphate (PIP); Phosphatidylinositol triphosphate (PIP3); Phosphatidylserine (PS); polyglyceryl-6-distearate; Pregnanes; Propylene Glycol Dicaprate; Propylene Glycol Dicaprylocaprate; Propylene Glycol Dicaprylocaprate; Psyllic acid; recinoleaic acid; recinoleyl alcohol; Sapienic acid; soy lecithin; Stearic acid; Stearidonic; stearyl alcohol; Tricosylic acid; Tridecyl alcohol; Tridecylic acid; Triolein; Undecyl alcohol; undecylenic acid; Undecylic acid; Vaccenic acid; α-Linolenic acid; γ-Linolenic acid; a fatty acid salt of 10-undecenoic acid, adapalene, arachidic acid, arachidonic acid, behenic acid, butyric acid, capric acid, caprylic acid, cerotic acid, cis-11-eicosenoic acid, cis-11-octadecenoic acid, cis-13-docosenoic acid, docosahexaenoic acid, eicosapentaenoic acid, elaidic acid, erucic acid, heneicosylic acid, heptacosylic acid, heptadecanoic acid, isostearic acid, lauric acid, lignoceric acid, linoelaidic acid, linoleic acid, montanic acid, myristic acid, myristoleic acid, neodecanoic acid, neoheptanoic acid, neononanoic acid, nonadecylic acid, oleic acid, palmitic acid, palmitoleic acid, pelargonic acid, pentacosylic acid, pentadecylic acid, recinoleaic acid (e.g. zinc recinoleate), sapienic acid, stearic acid, tricosylic acid, tridecylic acid, undecylenic acid, undecylic acid, vaccenic acid, valeric acid, α-linolenic acid, or γ-linolenic acid; paraffin; and any combinations thereof. In some embodiments, the lipid can be a fatty acid comprising 11 or fewer carbons. For example the fatty acid can comprise 6, 7, 8, 9, 10, or 11 carbons.

Without wishing to be bound by a theory, it is believed that fatty acid salts can be used in the particles comprising pyrithione salts to potentiate antifungal activity of pyrithione salts and provide stability in compositions comprising said particles. Accordingly, in some embodiments, the lipid is a fatty acid salt. Without limitations, the fatty acid salt can be selected from the group consisting of zinc, sodium, potassium, lithium, ammonium, copper, calcium, magnesium, strontium, manganese, and combinations thereof. In some embodiments, the fatty acid salt is a salt comprising zinc. In some embodiments, the fatty acid salt is zinc recinoleate.

Without limitations, the particle can comprise any amount of the lipid component. For example, the particle can comprise between about 0.01% to about 99% (w/w) of the lipid component. In some embodiments, the lipid component comprises greater than 0.1% (w/w), greater than 0.5% (w/w), greater than 1% (w/w), greater than 2% (w/w), greater than 3% (w/w), greater than 4% (w/w), greater than 5% (w/w), greater than 6% (w/w), greater than 7% (w/w), greater than 8% (w/w), greater than 9% (w/w), greater than 10% (w/w), greater than 11% (w/w), greater than 12% (w/w), greater than 13% (w/w), greater than 14% (w/w), greater than 15% (w/w), greater than 16% (w/w), greater than 17% (w/w), greater than 18% (w/w), greater than 19% (w/w), greater than 20% (w/w), greater than 25% (w/w), greater than 30% (w/w), greater than 35% (w/w), greater than 40% (w/w), greater than 45% (w/w), or greater than 50% (w/w) of the total weight of the particles. Typically, the content of the lipid component in the particles are in the range of about 2-25% (w/w).

Ratio of the active agent to the total lipid component of the coating layer can be any desired ratio. For example, ratio of the active agent to the total lipid component can range from about 100:1 to about 1:100. In some embodiments, the ratio of the active agent to the total lipid component can range from about 75:1 to about 1:75, from about 50:1 to about 1:50, from about 25:1 to about 1:25, from about 20:1 to about 1:20, from about 15:1 to about 1:15, from about 5:1 to about 1:5, or from about 25:1 to about 1:5. In some embodiments, the ratio of the active agent to the total lipid component is about 30:1, about 25:1, about 20:1, about 15:1, about 10:1, about 5:1, or about 1:1. The ratio can be based on weight, mass, or moles.

Thickness of the coating layer can range from nanometers to millimeters. For example, the coating layer thickness can range from about 1 nm to about 5000 nm, from about 5 nm to about 2500 nm, from about 10 nm to about 2000 nm, from about 50 nm to about 1500 nm, from about 20 nm to about 1000 nm, from about 1 nm to about 1000 nm, from about 1 nm to about 500 nm, from about 1 nm to about 250 nm, from about 1 nm to about 200 nm, from about 1 nm to about 150 nm, from about 1 nm to about 100 nm, from about 2 nm to about 50 nm, or from about 5 nm to about 25 nm.

In some embodiments, the particle can comprise two or more (e.g., 1, 2, 3, 4, 5, 6, 7, 8, 9, 10, or more) lipids, i.e., the particle can comprise a first lipid and a second lipid. For example, the coating layer can comprise a second lipid that is different from the first lipid. Accordingly, the particle can comprise a core comprising the active agent and a coating layer comprising a first lipid and a second lipid. Without wishing to be bound by a theory, it is believed that the combination of the lipids in the particle can provide a synergistic effect. For example, the presence of a second lipid can provide a synergistic antifungal effect. In another example, the second lipid can be chosen to provide solubility in components of a personal care composition. Accordingly, without limitations, the second lipid can be selected from the group consisting of fatty acids, fatty alcohols, glycerolipids (e.g., monoglycerides, diglycerides, and triglycerides), phospholipids, glycerophospholipids, sphingolipids, sterol lipids, prenol lipids, saccharolipids, polyketides, and any combination thereof. In some embodiments, the second lipid can be a fatty acid or an ester or salt thereof. In some embodiments, the second lipid is myristic acid or EGDS.

In some embodiments, the particle comprises a first lipid selected from the group consisting of ethylene glycol distearate (EGDS), caprylic acid, capric acid, lauric acid, myristic acid, undecalinic acid, and palmitic acid, and a second lipid. In some embodiments, the second lipid is selected from the group consisting of EGDS, caprylic acid, capric acid, lauric acid, myristic acid, undecalinic acid, and palmitic acid. In one embodiment, the second lipid is myristic acid, lauric acid, or undecalinic acid.

In one embodiment, the particle comprises a coating layer comprising EGDS and a second lipid. In a further embodiment of this, the particle comprises a coating layer comprising EGDS and myristic acid. In some other embodiments, the particle comprises a coating layer comprising EGDS and lauric acid. In some other embodiments, the particle comprises a coating layer comprising EGDS and undecalinic acid.

When a second lipid is present in addition to the first lipid, the particle can comprise between about 0.01% to about 99% (w/w) of the second lipid. In some embodiments, the second lipid component comprises greater than 0.1% (w/w), greater than 0.5% (w/w), greater than 1% (w/w), greater than 2% (w/w), greater than 3% (w/w), greater than 4% (w/w), greater than 5% (w/w), greater than 6% (w/w), greater than 7% (w/w), greater than 8% (w/w), greater than 9% (w/w), greater than 10% (w/w), greater than 11% (w/w), greater than 12% (w/w), greater than 13% (w/w), greater than 14% (w/w), greater than 15% (w/w), greater than 16% (w/w), greater than 17% (w/w), greater than 18% (w/w), greater than 19% (w/w), greater than 20% (w/w), greater than 25% (w/w), greater than 30% (w/w), greater than 35% (w/w), greater than 40% (w/w), greater than 45% (w/w), or greater than 50% (w/w) of the total weight of the particles. Typically, the content of the second lipid component in the particles are in the range of about 1-25% (w/w).

Ratio of the first lipid component to the second lipid component can be any desired ratio. For example, ratio of the first lipid component to the second lipid component can range from about 100:1 to about 1:100. In some embodiments, the ratio of the first lipid component to the second lipid component can range from about 75:1 to about 1:75, from about 50:1 to about 1:50, from about 25:1 to about 1:25, from about 20:1 to about 1:20, from about 15:1 to about 1:15, from about 5:1 to about 1:5, from about 2.5:1 to about 1:2.5, from about 2:1 to about 1:2, or from about 1.5:1 to 1:1.5. In some embodiments, the ratio of the first lipid component to the second lipid component is about 1:1. The ratio can be based on weight, mass, or moles.

In some embodiments, the particle can further comprise paraffin, i.e., the particle can comprise a lipid and paraffin. For example, the coating layer can comprise a lipid and paraffin. Accordingly, the particle can comprise a core comprising the active agent and a coating layer comprising a lipid and paraffin. In some embodiments, the lipid is a fatty acid or an ester or a salt thereof.

In some embodiments, the particle comprises a core comprising the active agent and a coating layer comprising a mixture comprising a lipid selected from the group consisting of caprylic acid, capric acid, lauric acid, myristic acid, and palmitic acid, and paraffin.

When paraffin is present in addition to the lipid component, the particle can comprise between about 0.01% to about 99% (w/w) of the paraffin. In some embodiments, the paraffin comprises greater than 0.1% (w/w), greater than 0.5% (w/w), greater than 1% (w/w), greater than 2% (w/w), greater than 3% (w/w), greater than 4% (w/w), greater than 5% (w/w), greater than 6% (w/w), greater than 7% (w/w), greater than 8% (w/w), greater than 9% (w/w), greater than 10% (w/w), greater than 11% (w/w), greater than 12% (w/w), greater than 13% (w/w), greater than 14% (w/w), greater than 15% (w/w), greater than 16% (w/w), greater than 17% (w/w), greater than 18% (w/w), greater than 19% (w/w), greater than 20% (w/w), greater than 25% (w/w), greater than 30% (w/w), greater than 35% (w/w), greater than 40% (w/w), greater than 45% (w/w), or greater than 50% (w/w) of the total weight of the particles. Typically, the content of the paraffin in the particles are in the range of about 2-25% (w/w).

Ratio of the lipid component to the paraffin can be any desired ratio. For example, ratio of the lipid component to the paraffin can range from about 100:1 to about 1:100. In some embodiments, the ratio of the lipid component to the paraffin component can range from about 75:1 to about 1:75, from about 50:1 to about 1:50, from about 25:1 to about 1:25, from about 20:1 to about 1:20, from about 15:1 to about 1:15, from about 5:1 to about 1:5, from about 2.5:1 to about 1:2.5, from about 2:1 to about 1:2, from about 1.5:1 to about 1:1.5. In some embodiments, the ratio of the lipid component to the paraffin is about 1:1. The ratio can be based on weight, mass, or moles.

In some embodiments, the coating layer can comprise a mixture of a lipid and a protein. For example, the particle can comprise a core comprising the active agent and a coating layer comprising a lipid and a protein.

Exemplary proteins include, but are not limited to, Actin, Albumin, Amaranth Protein, Ammonium Hydrolyzed Animal Protein, Animal protein, Barley Protein, Brazil Nut Protein, Casein, Collagen, Collagen protein hydrolyzed, Conchiolin Protein, corn protein, Cottonseed Protein, Elastin, Extensin, Fibroin, Fibronectin, Fish Protein, Gadidae Protein, Gelatin, Glutein, Glycoproteins, Hazelnut Protein, Hemoglobin, Hemp Seed Protein, Honey Protein, Hydrolyzed Actin, Hydrolyzed Amaranth Protein, Hydrolyzed animal protein, Hydrolyzed Barley Protein, Hydrolyzed Brazil Nut Protein, Hydrolyzed Conchiolin Protein, Hydrolyzed corn protein, Hydrolyzed Cottonseed Protein, Hydrolyzed Elastin, Hydrolyzed Extensin, Hydrolyzed Fibroin, Hydrolyzed Fibronectin, Hydrolyzed Fish Protein, Hydrolyzed Gadidae Protein, Hydrolyzed Gadidae Protein, Hydrolyzed Gelatin, Hydrolyzed Hair Keratin, Hydrolyzed Hazelnut, Hydrolyzed Hazelnut Protein, Hydrolyzed Hemoglobin, Hydrolyzed Hemp Seed Protein, Hydrolyzed Honey Protein, Hydrolyzed Keratin, Hydrolyzed Lupine Protein, Hydrolyzed Maple Sycamore Protein, Hydrolyzed Milk Protein, Hydrolyzed Oat Protein, Hydrolyzed Pea Protein, Hydrolyzed Potato Protein, Hydrolyzed Reticulin, Hydrolyzed Royal Jelly Protein, Hydrolyzed Sericin, Hydrolyzed Serum Protein, Hydrolyzed Sesame Protein, Hydrolyzed Soy Protein, Hydrolyzed Soymilk Protein, Hydrolyzed Spinal Protein, Hydrolyzed Spongin, Hydrolyzed Sweet Almond Protein, Hydrolyzed Vegetable Protein, Hydrolyzed Wheat Gluten, Hydrolyzed Wheat Protein, Hydrolyzed Whey Protein, Hydrolyzed Yeast Protein, Hydrolyzed Yogurt Protein, Hydrolyzed Zein, Integrin, Jojoba protein HP, Hydrolyzed, keratin, Lupine Protein, Maple Sycamore Protein, MEA-Hydrolyzed Collagen, MEA-Hydrolyzed Silk, Milk Protein, Myosin, Oat Protein, Pea Protein, polylysine, Potato Protein, Reticulin, Rice Quat, Royal Jelly Protein, Sericin, Serum Protein, Sesame Protein, Silk powder, Sodium Hydrolyzed Casein, Soy Protein, Soy Rice Peptides, Soymilk Protein, Spinal Protein, Spongin, Sweet Almond Protein, Vegetable Protein, Wheat Gluten, Whey Protein, Yeast Protein, Yogurt Protein, Zein, and Zinc Hydrolyzed Collagen.

In some embodiments, the protein is an albumin. The albumin can be a naturally occurring albumin, an albumin related protein or a variant thereof such as a natural or engineered variant. Variants include polymorphisms, fragments such as domains and subdomains, fragments and/or fusion proteins. An albumin can comprise the sequence of an albumin protein obtained from any source. A number of proteins are known to exist within the albumin family. Accordingly, the albumin can comprise the sequence of an albumin derived from one of serum albumin from African clawed frog (e.g., see Swissprot accession number P08759-1), bovine (e.g., see Swissprot accession number P02769-1), cat (e.g., see Swissprot accession number P49064-1), chicken (e.g., see Swissprot accession number P19121-1), chicken ovalbumin (e.g., see Swissprot accession number P01012-1), cobra ALB (e.g., see Swissprot accession number Q91134-1), dog (e.g., see Swissprot accession number P49822-1), donkey (e.g., see Swissprot accession number QSXLE4-1), European water frog (e.g., see Swissprot accession number Q9YGH6-1), blood fluke (e.g., see Swissprot accession number AAL08579 and Q95VB7-1), Mongolian gerbil (e.g., see Swissprot accession number O35090-1 and JC5838), goat (e.g., see Swissprot accession number B3VHM9-1 and as available from Sigma as product no. A2514 or A4164), guinea pig (e.g., see Swissprot accession number Q6WDN9-1), hamster (see DeMarco et al. (2007). International Journal for Parasitology 37(11): 1201-1208), horse (e.g., see Swissprot accession number P35747-1), human (e.g., see Swissprot accession number P02768-1), Australian Lung-fish (e.g., see Swissprot accession number P83517), macaque (Rhesus monkey) (e.g., see Swissprot accession number Q28522-), mouse (e.g., see Swissprot accession number P07724-1), North American bull frog (e.g., see Swissprot accession number P21847-1), pig (e.g., see Swissprot accession number P08835-1), pigeon (e.g. as defined by Khan et al, 2002, 1112. J. Biol. Macromol, 30(3-4), 171-8), rabbit (e.g., see Swissprot accession number P490 65-1), rat (e.g., see Swissprot accession number P02770-1), salamander (e.g., see Swissprot accession number Q8UW05-1), salmon ALB1 (e.g., see Swissprot accession number P21848-1), salmon ALB2 (e.g., see Swissprot accession number Q03156-1), sea lamprey (e.g., see Swissprot accession number Q91274-1 and O42279-1) sheep (e.g., see Swissprot accession number P14639-1), Sumatran orangutan (e.g., see Swissprot accession number Q5NVH5-1), tuatara (e.g., see Swissprot accession number Q8JIA9-1), turkey ovalbumin (e.g., see Swissprot accession number O73860-1), Western clawed frog (e.g., see Swissprot accession number Q6D.I95-1), and includes variants and fragments thereof as defined herein. Many naturally occurring mutant forms of albumin are known. Many are described in Peters, (1996, All About Albumin: Biochemistry, Genetics and Medical Applications, Academic Press, Inc., San Diego, Calif., p. 170-181), content of which is incorporated herein by reference. The term albumin also encompasses albumin variants, such as genetically engineered forms, mutated forms, and fragments etc. having one or more binding sites that are analogous to a binding site unique for one or more albumins as defined above. By analogous binding sites in the context of the invention are contemplated structures that are able to compete with each other for binding to one and the same ligand structure.

In one embodiment, albumin is bovine serum albumin, egg albumin, hydrolyzed lactalbumin, or lactalbumin, including variants and fragments thereof. In one embodiment, the protein is egg albumin.

In some embodiments, the coating layer comprises ethylene glycol distearate (EGDS) and a protein. In one embodiment, the coating layer comprises EGDS and albumin. In one embodiment, the coating layer comprises EGDS and egg albumin.

The protein can comprise between about 0.01% to about 99% (w/w) of the particle. In some embodiments, the protein component comprises greater than 0.1% (w/w), greater than 0.5% (w/w), greater than 1% (w/w), greater than 2% (w/w), greater than 3% (w/w), greater than 4% (w/w), greater than 5% (w/w), greater than 6% (w/w), greater than 7% (w/w), greater than 8% (w/w), greater than 9% (w/w), greater than 10% (w/w), greater than 11% (w/w), greater than 12% (w/w), greater than 13% (w/w), greater than 14% (w/w), greater than 15% (w/w), greater than 16% (w/w), greater than 17% (w/w), greater than 18% (w/w), greater than 19% (w/w), greater than 20% (w/w), greater than 25% (w/w), greater than 30% (w/w), greater than 35% (w/w), greater than 40% (w/w), greater than 45% (w/w), or greater than 50% (w/w) of the total weight of the particles. Typically, the content of the protein component in the particles are in the range of about 1-25% (w/w), about 0.1-10% (w/w), about 0.5-5% (w/w), or about 1-1.5% (w/w).

Ratio of the active agent to the protein component can be any desired ratio. For example, ratio of the active agent to the protein component can range from about 100:1 to about 1:100. In some embodiments, the ratio the active agent to the protein can range from about 100:1 to about 1:1, from about 90:1 to about 10:1, from about 85:1 to about 15:1, from about 80:1 to about 25:1, or from 75:1 to about 50:1. In some embodiments, the ratio of the active agent to the protein component is about 75:1. The ratio can be based on weight, mass, or moles.

Ratio of the lipid component to the protein component can be any desired ratio. For example, ratio of the lipid component to the protein component can range from about 100:1 to about 1:100. In some embodiments, the ratio of the lipid component to the protein can range from about 75:1 to about 1:75, from about 50:1 to about 1:50, from about 25:1 to about 1:25, from about 20:1 to about 1:20, from about 15:1 to about 1:15, from about 10:1 to about 1:10, from about 5:1 to about 1:5, or from about 2:1 to about 1:1. In some embodiments, the ratio of the lipid component to the protein component is about 1.5:1. The ratio can be based on weight, mass, or moles.

Ratio of the core to the total of the lipid and protein components can be any desired ratio. For example, ratio of the core to the total of the lipid and protein components can range from about 100:1 to about 1:100. In some embodiments, the ratio of the core to the total of the lipid and protein components can range from about 75:1 to about 1:75, from about 50:1 to about 1:50, from about 25:1 to about 1:25, from about 20:1 to about 1:20, from about 15:1 to about 1:15, from about 5:1 to about 1:5, or from about 25:1 to about 1:5. In some embodiments, the ratio of the core to the total of the lipid and protein components is about 30:1, about 25:1, about 20:1, about 15:1, about 10:1, about 5:1, or about 1:1. The ratio can be based on weight, mass, or moles.

Ratio of the active agent to the total of the lipid and protein components can be any desired ratio. For example, ratio of the active agent to the total of the lipid and protein components can range from about 100:1 to about 1:100. In some embodiments, the ratio of the active agent to the total of the lipid and protein components can range from about 75:1 to about 1:75, from about 50:1 to about 1:50, from about 25:1 to about 1:25, from about 20:1 to about 1:20, from about 15:1 to about 1:15, from about 5:1 to about 1:5, or from about 25:1 to about 1:5. In some embodiments, the ratio of the active agent to the total of the lipid and protein components is about 30:1, about 25:1, about 20:1, about 15:1, about 10:1, about 5:1, or about 1:1. The ratio can be based on weight, mass, or moles.

In some embodiments, the coating layer can comprise a mixture of a lipid and a cationic molecule. For example, the particle can comprise a core comprising the active agent and a coating layer comprising a lipid and a cationic molecule.

In some embodiments, the cationic molecule is a polyamine. Exemplary cationic molecules include, but are not limited to, Putrescine (Butane-1,4-diamine), Cadaverine (Pentane-1,5-diamine), Spermidine, Spermine, Cyclen (1,4,7,10-tetrazacyclododecane), Cyclam (1,4,8,11-Tetraazacyclotetradecane), Linear Polyethyleneimine (Poly(iminoethylene)), Norspermidine, p-Phenylenediamine (1,4-diaminobenzene), Diethylenetriamine (N-(2-aminoethyl)-1,2-ethanediamine), thermospermine, Tris(2-aminoethyl)amine, Hexamethylenediamine, Beta-lysine (3,6-diaminohexanoic acid), m-Phenylenediamine (1,3-diaminobenzene), Diaminopropane (1,2-Diaminopropane), Ethylenediamine dihydroiodide, and polyamine D 400 (Polyoxyalkyleneamine D 400).

In some embodiments, the protein is an albumin. The albumin can be a naturally occurring albumin, an albumin related protein or a variant thereof such as a natural or engineered variant. Variants include polymorphisms, fragments such as domains and subdomains, fragments and/or fusion proteins. An albumin can comprise the sequence of an albumin protein obtained from any source. A number of proteins are known to exist within the albumin family. Accordingly, the albumin can comprise the sequence of an albumin derived from one of serum albumin from African clawed frog (e.g., see Swissprot accession number P08759-1), bovine (e.g., see Swissprot accession number P02769-1), cat (e.g., see Swissprot accession number P49064-1), chicken (e.g., see Swissprot accession number P19121-1), chicken ovalbumin (e.g., see Swissprot accession number P01012-1), cobra ALB (e.g., see Swissprot accession number Q91134-1), dog (e.g., see Swissprot accession number P49822-1), donkey (e.g., see Swissprot accession number QSXLE4-1), European water frog (e.g., see Swissprot accession number Q9YGH6-1), blood fluke (e.g., see Swissprot accession number AAL08579 and Q95VB7-1), Mongolian gerbil (e.g., see Swissprot accession number O35090-1 and JC5838), goat (e.g., see Swissprot accession number B3VHM9-1 and as available from Sigma as product no. A2514 or A4164), guinea pig (e.g., see Swissprot accession number Q6WDN9-1), hamster (see DeMarco et al. (2007). International Journal for Parasitology 37(11): 1201-1208), horse (e.g., see Swissprot accession number P35747-1), human (e.g., see Swissprot accession number P02768-1), Australian Lung-fish (e.g., see Swissprot accession number P83517), macaque (Rhesus monkey) (e.g., see Swissprot accession number Q28522-), mouse (e.g., see Swissprot accession number P07724-1), North American bull frog (e.g., see Swissprot accession number P21847-1), pig (e.g., see Swissprot accession number P08835-1), pigeon (e.g. as defined by Khan et al, 2002, 1112. J. Biol. Macromol, 30(3-4), 171-8), rabbit (e.g., see Swissprot accession number P490 65-1), rat (e.g., see Swissprot accession number P02770-1), salamander (e.g., see Swissprot accession number Q8UW05-1), salmon ALB1 (e.g., see Swissprot accession number P21848-1), salmon ALB2 (e.g., see Swissprot accession number Q03156-1), sea lamprey (e.g., see Swissprot accession number Q91274-1 and O42279-1) sheep (e.g., see Swissprot accession number P14639-1), Sumatran orangutan (e.g., see Swissprot accession number Q5NVH5-1), tuatara (e.g., see Swissprot accession number Q8JIA9-1), turkey ovalbumin (e.g., see Swissprot accession number O73860-1), Western clawed frog (e.g., see Swissprot accession number Q6D.I95-1), and includes variants and fragments thereof as defined herein. Many naturally occurring mutant forms of albumin are known. Many are described in Peters, (1996, All About Albumin: Biochemistry, Genetics and Medical Applications, Academic Press, Inc., San Diego, Calif., p. 170-181), content of which is incorporated herein by reference. The term albumin also encompasses albumin variants, such as genetically engineered forms, mutated forms, and fragments etc. having one or more binding sites that are analogous to a binding site unique for one or more albumins as defined above. By analogous binding sites in the context of the invention are contemplated structures that are able to compete with each other for binding to one and the same ligand structure.

The cationic molecule can comprise between about 0.01% to about 99% (w/w) of the particle. In some embodiments, the cationic molecule comprises greater than 0.1% (w/w), greater than 0.5% (w/w), greater than 1% (w/w), greater than 2% (w/w), greater than 3% (w/w), greater than 4% (w/w), greater than 5% (w/w), greater than 6% (w/w), greater than 7% (w/w), greater than 8% (w/w), greater than 9% (w/w), greater than 10% (w/w), greater than 11% (w/w), greater than 12% (w/w), greater than 13% (w/w), greater than 14% (w/w), greater than 15% (w/w), greater than 16% (w/w), greater than 17% (w/w), greater than 18% (w/w), greater than 19% (w/w), greater than 20% (w/w), greater than 25% (w/w), greater than 30% (w/w), greater than 35% (w/w), greater than 40% (w/w), greater than 45% (w/w), or greater than 50% (w/w) of the total weight of the particles. Typically, the content of the cationic molecule in the particles are in the range of about 1-25% (w/w), about 0.1-10% (w/w), about 0.5-5% (w/w), or about 1-1.5% (w/w).

Ratio of the active agent to the cationic molecule can be any desired ratio. For example, ratio of the active agent to the cationic molecule can range from about 100:1 to about 1:100. In some embodiments, the ratio the active agent to the cationic molecule can range from about 100:1 to about 1:1, from about 90:1 to about 10:1, from about 85:1 to about 15:1, from about 80:1 to about 25:1, or from 75:1 to about 50:1. In some embodiments, the ratio of the active agent to the cationic molecule is about 75:1. The ratio can be based on weight, mass, or moles.

Ratio of the lipid component to the cationic molecule can be any desired ratio. For example, ratio of the lipid component to the cationic molecule can range from about 100:1 to about 1:100. In some embodiments, the ratio of the lipid component to the cationic molecule can range from about 75:1 to about 1:75, from about 50:1 to about 1:50, from about 25:1 to about 1:25, from about 20:1 to about 1:20, from about 15:1 to about 1:15, from about 10:1 to about 1:10, from about 5:1 to about 1:5, or from about 2:1 to about 1:1. In some embodiments, the ratio of the lipid component to the cationic molecule is about 1.5:1. The ratio can be based on weight, mass, or moles.

In some embodiments, the polymer in the coating layer is a biocompatible polymer. As used herein, the term “biocompatible” means exhibition of essentially no cytotoxicity or immunogenicity while in contact with body fluids or tissues. As used herein, the term “polymer” refers to oligomers, co-oligomers, polymers and co-polymers, e.g., random block, multiblock, star, grafted, gradient copolymers and combination thereof.

The term “biocompatible polymer” refers to polymers which are non-toxic, chemically inert, and substantially non-immunogenic when used internally in a subject and which are substantially insoluble in blood. The biocompatible polymer can be either non-biodegradable or preferably biodegradable. Preferably, the biocompatible polymer is also noninflammatory when employed in situ.

Biodegradable polymers are disclosed in the art. Examples of suitable biodegradable polymers include, but are not limited to, linear-chain polymers such as polylactides, polyglycolides, polycaprolactones, copolymers of polylactic acid and polyglycolic acid, polyanhydrides, polyepsilon caprolactone, polyamides, polyurethanes, polyesteramides, polyorthoesters, polydioxanones, polyacetals, polyketals, polycarbonates, polyorthocarbonates, polydihydropyrans, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, polymethyl methacrylate, chitin, chitosan, copolymers of polylactic acid and polyglycolic acid, poly(glycerol sebacate) (PGS), and copolymers, terpolymers, and copolymers including one or more of the foregoing. Other biodegradable polymers include, for example, gelatin, collagen, silk, chitosan, alginate, cellulose, poly-nucleic acids, etc.

Suitable non-biodegradable biocompatible polymers include, by way of example, cellulose acetates (including cellulose diacetate), polyethylene, polypropylene, polybutylene, polyethylene terphthalate (PET), polyvinyl chloride, polystyrene, polyamides, nylon, polycarbonates, polysulfides, polysulfones, hydrogels (e.g., acrylics), polyacrylonitrile, polyvinylacetate, cellulose acetate butyrate, nitrocellulose, copolymers of urethane/carbonate, copolymers of styrene/maleic acid, poly(ethylenimine), Pluronic (Poloxamers 407, 188), Hyaluron, heparin, agarose, Pullulan, and copolymers including one or more of the foregoing, such as ethylene/vinyl alcohol copolymers (EVOH).

In some embodiments, the biocompatible polymer is a copolymer of polylactic acid and polyglycolic acid, poly(glycerol sebacate) (PGS), poly(ethylenimine), Pluronic (Poloxamers 407, 188), Hyaluron, heparin, agarose, or Pullulan.

In some embodiments, the coating layer comprises a carbohydrate or a carbohydrate based polymer. As used herein, the term “carbohydrate based polymer,” includes, but is not limited to, oligomers or polymers that contain monomers having the formula Cₘ(H₂O)ₙ wherein m and n are ≧3 and where in m and n can be same or different. Preferably m and n are independently 3, 4, 5, 6, or 7. Carbohydrate based polymers include, but are not limited to, compounds such as oligosaccharides, polysaccharides, glycoproteins, glycolipids and the like.

In some embodiments of this and other aspects of the invention, the carbohydrate polymer comprises at least 5, at least 6, at least 7, at least 8, at least 9, or at least 10 or sugar monomers.

Without limitation, the carbohydrate polymer comprises sugar monomers independently selected from the group consisting of erythrose, threose, ribose, arabinose, xylose, lyxose, ribulose, xylulose, allose, altrose, glucose, mannose, gulose, idose, galactose, galactosamine, N-acetylgalactose, glucosamine, N-acetylglucosamine, sialic acid, talose, psicose, fructose, sorbose, tagatose, fucose, fuculose, rhamonse, sedoheptulose, octose, sulfoquinovose and nonose (neuraminic acid), wherein the sugar may be optionally substituted. Without limitation each sugar can independently have the L- or the D-conformation.

The linkage between two sugar monomers can independently have the α- or β-configuration. Furthermore, the linkage between the two sugar can be 1->3, 1->4, 1->5, or 1->6.

In some embodiments, at least one (e.g., 1, 2, 3, or 4) hydroxyl of the sugar monomer is replaced by an amino group. In some embodiments, the hydroxyl at position 2 of the sugar monomer is replaced by an amino group. The amino group can be optionally substituted with an C₁-C₆ alkyl or an acyl group. Preferred C₁-C₆ alkyl groups include methyl, ethyl, propyl, butyl, and t-butyl. One preferred acyl group is acetyl.

In some embodiments of this and other aspects of the invention, the carbohydrate polymer comprises one or more (e.g., 1, 2, 3, 4, 5, 6, 7, 8, 9, or 10 or more) disaccharide, trisaccharide or tetrasaccharide monomers independently selected from the group consisting of sucrose, lactulose, lactose, maltose, trehalose, cellobiose, kojibiose, nigerose, isomaltose, β,β-Trehalose, α,β-Trehalose, sophorose, laminaribiose, gentibiose, turanose, maltulose, palatinose, gentibiulose, mannobiose, melibiose, rutinose, rutinulose, xylobiose, raffinose, melezitose, acarbose and stachyose.

As used herein, the term “oligosaccharide” refers without limitation to several (e.g., five to ten) covalently linked monosaccharide units. As used herein, the term “polysaccharide” refers without limitation to many (e.g., eleven or more) covalently linked sugar units. Polysaccharides can have molecular masses ranging well into millions of daltons. Exemplary oligosaccharides and polysaccharides include, but are not limited to, fructooligosaccharide, galactooligosaccharides, mannanoligosaccharides, glycogen, starch (amylase, amylopectin), glycosaminoglycans (e.g., hyaluronic acid, chondroitin-4-sulfate, chondroitin-6-sulfate, dermatan sulfate, keratin sulfate, heparin and the like), cellulose, beta-glucan (zymosan, lentinan, sizofiran), maltodextrin, inulin, levan beta (2->6), chitin, and chitosan.

In some embodiments of this and other aspects of the invention, the carbohydrate polymer is chitin or a derivative thereof. One preferred chitin derivative is chitosan (α-(1-4) 2-amino-2-deoxy-β-D-glucan) and derivatives thereof. Exemplary derivatives of chitosan include, but are not limited to, N-(aminoalkyl) chitosans, succinyl chitosans, quteraminated chiotosans, N-acylated chitosans (e.g., caproyl chitosan, octanoyl chitosan, myristoyl chitosan, and palmitoyl chitosan), N-methylene phosphonic chitosans, N-lauryl-N-methylene phosphonic chitosans, N-lauryl-carboxymethyl chitosans, N-alkyl-O-sulfated chitosans, thiolated chitosans (e.g., chitosan-2-iminthiolane, chitosan-4-thiobutylamidine, and chitosan-thioglycolic acid), and phosphorylated chitosans).

One of skill in the art is well aware of synthetic methods which can be used for the synthesis of carbohydrate polymers. See for example, Stick, R. V., Carbohydrates: The Sweet Molecules of Life.; Academic Press, pp 113-177 (2002); Crich, D. & Dudkin V., J. Am. Chem. Soc., 123:6819-6825 (2001); Garegg, P. J., Chemtracts-Org. Chem., 5:389 (1992); Mayer, T. G., Kratzer, B. & Schmidt, R. R. Synthesis of a GPI anchor of the yeast Saccharomyces cerevisiae. Angew. Chem. Int. Ed. Engl. 33, 2177-2181 (1994); Seifert, J., Lergenmuller, M. & Ito, Y. Synthesis of an α-(2,3)-sialylated complex-type undecasaccharide. Angew. Chem. Int. Ed. 39, 531-534 (2000); Wang, Z.-G. et al. Toward fully synthetic homogeneous glycoproteins: a high mannose core containing glycopeptide containing carrying full H-type 2 human blood group specifity. Angew. Chem. Int. Ed. 40, 1728-1732 (2001); Caruthers, M. H. Gene synthesis machines: DNA chemistry and its uses. Science 230, 281-285 (1985); Sears, P. & Wong, C.-H. Toward automated synthesis of oligosaccharides and glycoproteins. Science 291, 2344-2350 (2001); Zhang, Z. et al. Programmable one-pot oligosaccharide synthesis. J. Am. Chem. Soc. 121, 734-753 (1999; Nishimura, S. Automated glycosynthesizer ‘Golgi’ by mimicking biosynthetic process. Tanpakushitsu Kakusan Koso 48, 1220-1225 (2003); Plante, O. J., Palmacci, E. R. & Seeberger, P. H. Automated solid-phase synthesis of oligosaccharides. Science 291, 1523-1527 (2001); Andrade, R. B., Plante, O. J., Melean, L. G. & Seeberger, P. H. Solid-phase oligosaccharide synthesis: preparation of complex structures using a novel linker and different glycosylating agents. Org. Lett. 1, 1811-1814 (1999); Love, K. R. & Seeberger, P. H. Automated solid-phase synthesis of protected tumor-associated antigen and blood group determinant oligosaccharides. Angew. Chem. Int. Ed. 43, 602-605 (2004); and Seeberger, P. H. & Werz, D. B. Synthesis and medical applications of oligosaccharides. Nature 446, 1046-1051 (2007), content of all of which is herein incorporated by reference.

In some embodiments, the particle further comprises an excipient. In some embodiments, the excipient is a wetting agent. Without limitations, the wetting agent can be selected from alkyl sulfates, e.g. sodium lauryl sulfate, sodium stearyl sulfate, sodium oleyl sulfate and sodium cetyl sulfate, alkyl aryl sulfonates, e.g. sodium dodecylbenzene sulfonate and dialkyl sodium sulfosuccinates, e.g. sodium bis-(2-ethylhexyl)sulfosuccinate, and most preferably sodium lauryl sulfate. Further examples of the pharmaceutically acceptable wetting agent include benzethonium chloride, cetylpyridinium chloride, docusate sodium, poloxamer, polysorbate and sorbitan esters.

In some embodiments, the excipient is a stabilizer, e.g., a surface stabilizer. Suitable surface stabilizers can preferably be selected from known organic and inorganic pharmaceutical excipients. Such excipients include various polymers, low molecular weight oligomers, natural products, and surfactants. Preferred surface stabilizers include nonionic and ionic surfactants. Two or more surface stabilizers can be used in combination. Representative examples of surface stabilizers include sodium docusate, cetyl pyridinium chloride, gelatin, casein, lecithin (phosphatides), dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80®(ICI Specialty Chemicals)); polyethylene glycols (e.g., Carbowaxs 3350® and 1450®, and Carbopol 934® (Union Carbide)), dodecyl trimethyl ammonium bromide, polyoxyethylene stearates, colloidal silicon dioxide, phosphates, sodium dodecyl sulfate, carboxymethylcellulose calcium, hydroxypropyl celluloses (e.g., HPC, HPC-SL, and HPC-L), hydroxypropyl methylcellulose (HPMC), carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), polyvinylpyrrolidone (PVP), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); a charged phospholipid such as dimyristoyl phophatidyl glycerol, dioctylsulfosuccinate (DOSS); Tetronic 1508® (T-1508) (BASF Wyandotte Corporation), dialkylesters of sodium sulfosuccinic acid (e.g., Aerosol OT®, which is a dioctyl ester of sodium sulfosuccinic acid (American Cyanamid)); Duponol P®, which is a sodium lauryl sulfate (DuPont); Tritons X-200®, which is an alkyl aryl polyether sulfonate (Rohm and Haas); Crodestas F-1 10®, which is a mixture of sucrose stearate and sucrose distearate (Croda Inc.); p-isononylphenoxypoly-(glycidol), also known as Olin-IOG® or Surfactant 10-G® (Olin Chemicals, Stamford, Conn.); Crodestas SL-40® (Croda, Inc.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; and the like. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 1986), content of which is incorporated herein by reference in its entirety. In one embodiment, the excipient is sodium docusate.

Generally, the particles have an average diameter of from about 5 nm to about 5000 nm. In some embodiments, the particles have an average diameter of from about 50 nm to about 2500 nm. In some embodiments, the particles have an average diameter of from about 100 nm to about 2000 nm. In some embodiments, the particles have an average diameter of from about 150 nm to about 1700 nm. In some embodiments, the particles have an average diameter of from about 200 nm to about 1500 nm. In some embodiment, the particles have an average diameter of about 260 nm. In one embodiment, the particles have an average diameter of about 30 nm to about 150 nm. In some embodiments, the particle have an average diameter of about 100 nm to about 1000 nm, from about 200 nm to about 800 nm, from about 200 nm to about 700 nm, or from about 300 nm to about 700 nm.

Generally, the particle disclosed herein can be of any shape or form, e.g., spherical, rod, elliptical, cylindrical, capsule, or disc; and these particles can be part of a network or an aggregate. Without limitations, the particle can have any size from nm to millimeters. In some embodiments, the particles can have a size ranging from about 5 nm to about 5000 nm.

In some embodiments, the particle is a microparticle or a nanoparticle. As used herein, the term “microparticle” refers to a particle having a particle size of about 1 μm to about 1000 μm. As used herein, the term “nanoparticle” refers to particle having a particle size of about 0.1 nm to about 1000 nm.

Without wishing to be bound by a theory, particle size can affect the desired properties. Thus, the particle size or size range can be selected for a desired indication or a desired site of action.

It will be understood by one of ordinary skill in the art that particles usually exhibit a distribution of particle sizes around the indicated “size.” Unless otherwise stated, the term “particle size” as used herein refers to the mode of a size distribution of particles, i.e., the value that occurs most frequently in the size distribution. Methods for measuring the particle size are known to a skilled artisan, e.g., by dynamic light scattering (such as photocorrelation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

In some embodiments, the particles can be substantially spherical. What is meant by “substantially spherical” is that the ratio of the lengths of the longest to the shortest perpendicular axes of the particle cross section is less than or equal to about 1.5. Substantially spherical does not require a line of symmetry. Further, the particles can have surface texturing, such as lines or indentations or protuberances that are small in scale when compared to the overall size of the particle and still be substantially spherical. In some embodiments, the ratio of lengths between the longest and shortest axes of the particle is less than or equal to about 1.5, less than or equal to about 1.45, less than or equal to about 1.4, less than or equal to about 1.35, less than or equal to about 1.30, less than or equal to about 1.25, less than or equal to about 1.20, less than or equal to about 1.15 less than or equal to about 1.1. Without wishing to be bound by a theory, surface contact is minimized in particles that are substantially spherical, which minimizes the undesirable agglomeration of the particles upon storage. Many crystals or flakes have flat surfaces that can allow large surface contact areas where agglomeration can occur by ionic or non-ionic interactions. A sphere permits contact over a much smaller area.

In some embodiments, the particles have substantially the same particle size. Particles having a broad size distribution where there are both relatively big and small particles allow for the smaller particles to fill in the gaps between the larger particles, thereby creating new contact surfaces. A broad size distribution can result in larger spheres by creating many contact opportunities for binding agglomeration. The particles described herein are within a narrow size distribution, thereby minimizing opportunities for contact agglomeration. What is meant by a “narrow size distribution” is a particle size distribution that has a ratio of the volume diameter of the 90th percentile of the small spherical particles to the volume diameter of the 10th percentile less than or equal to 5. In some embodiments, the volume diameter of the 90th percentile of the small spherical particles to the volume diameter of the 10th percentile is less than or equal to 4.5, less than or equal to 4, less than or equal to 3.5, less than or equal to 3, less than or equal to 2.5, less than or equal to 2, less than or equal to 1.5, less than or equal to 1.45, less than or equal to 1.40, less than or equal to 1.35, less than or equal to 1.3, less than or equal to 1.25, less than or equal to 1.20, less than or equal to 1.15, or less than or equal to 1.1.

Geometric Standard Deviation (GSD) can also be used to indicate the narrow size distribution. GSD calculations involved determining the effective cutoff diameter (ECD) at the cumulative less than percentages of 15.9% and 84.1%. GSD is equal to the square root of the ratio of the ECD less than 84.17% to ECD less than 15.9%. The GSD has a narrow size distribution when GSD<2.5. In some embodiments, GSD is less than 2, less than 1.75, or less than 1.5. In one embodiment, GSD is less than 1.8.

While, the particles are discussed in terms of coated particles, there are at least eight types of particles that can be formulated with the active agent and the lipid component: (1) particles comprising a core formed by the active agent to which the lipid component absorbs/adsorbs or the lipid component forms one or more coating layers on the particle core; (2) particles comprising a generally homogeneous mixture of the active agent and the lipid component; (3) particles comprising a core comprising a generally homogeneous mixture of the active agent and the lipid, and the lipid component forms one or more coating layers on the particle core; (4) particles comprising a core formed by the lipid component and the active agent forms one or more coating layers on the particle core; (5) particles comprising a core comprising a generally homogeneous mixture of the active agent and the lipid, and the active agent forms one or more coating over the particle core; (6) particle comprising a core of material other than the active agent and the lipid component, and a mixture of the active agent and the lipid forms one or more coating layers on the particle core; (7) particles comprising a core comprising a generally homogeneous mixture of the active agent and the lipid, and a material other than the active agent or the lipid component forms one or more coating layers on the particle core; and (8) particle comprising any of the particles of (1)-(7) and further comprising one or more layers of a material other than the active agent or the lipid component, e.g., a protein or a polymer. In particles of (8), the further layer can be the outermost layer, a first layer on the core, interspersed between the layers described in (1)-(7), or any combinations thereof. Without limitations, the coating layer can comprise components other than indicated above. For example, the above indicated coating component can be mixed with other molecules or compositions to form the coating layer. This can be useful in instances wherein the specified component may not be able to form a coating layer by itself. For example, the active agent can be mixed with a second lipid, a protein, or a polymer to form the coating layer. While, the above is discussed with reference to a single active agent, it is to be understood that the single active agent can be replaced by two or more (e.g., two, three, four, five, six, seven, eight, nine, ten or more) different active agents. The different active agents can be active against same indication, different indications, or any combinations of same and different indications. Further, while the above is discussed with reference to a single coating layer, it is to be understood that two or more (e.g., 1, 2, 3, 4, 5, 6, 7, 8, 9, 10 or more) coating layers can be present. The different coating layers can comprise the same components, different components or any combinations of same and different components. While above is discussed in term of lipid components, same also applies to particles in which the lipid component of a coating layer is replaced by a protein, carbohydrate, polymer etc. . . . , e.g., particles comprising active agent in the core and the protein, carbohydrate or polymer forming a coating layer on the core.

In some embodiments, the particle comprises a core comprising the active agent and the lipid component forms one or more (e.g., 1, 2, 3, 4, 5, 6, 7, 8, 9, 10 or more) coating layers on the core. In some embodiments, the particle comprises a core comprising the active agent and alternating layers (e.g., 1, 2, 3, 4, 5, 6, 7, 8, 9, 10 or more layers) of lipid and active agent on the core. The lipid layer components can be the same or different for the different layers and the active agent in the alternating layers can be the same or different. Further, the active agent in the layers and the core can be the same or different. By way of example, the particle can comprise a core comprising the active agent and coating layer comprising an active agent layer sandwiched between two lipid layers. In some embodiments, the outer most layer is an active agent layer. In some other embodiments, the outer most layer is a lipid coating layer.

The particle can be fabricated using methods and instruments well known in the art. For example, the particles can be made using microprecipitation, encapsulation, deaggregation, hybrid of deaggregation and encapsulation, homogenization, hybrid of deaggregation and hot homogenization, or any combinations thereof. In some embodiments, the particles can be made using the method described in the Examples section.

In some embodiments, the process of making the particles comprises the step of selecting particles of a desired size.

The disclosure also provides a particle comprising an active agent and a protein coating layer without having a lipid in the coating layer. Without wishing to be bound by a theory, it is believed that protein, e.g., pan-binding protein, can be used in place of the lipid component to provide the same benefit as lipid component. For example, the protein can be used to bind or associate the particle to keratin, sebum, and/or hair to provide adhesion or retention on scalp. Accordingly, in some embodiments, the particle can comprises a core comprising the active agent and a coating layer comprising a protein, wherein the coating layer does not comprise a lipid.

Generally, any protein can be used for the coating. Exemplary proteins include, but are not limited to, Actin, Albumin, Amaranth Protein, Ammonium Hydrolyzed Animal Protein, Animal protein, Barley Protein, Bovine serum albumin, Brazil Nut Protein, Casein, Collagen, Collagen protein hydrolyzed, Conchiolin Protein, corn protein, Cottonseed Protein, Egg albumin, Elastin, Extensin, Fibroin, Fibronectin, Fish Protein, Gadidae Protein, Gelatin, Glutein, Glycoproteins, Hazelnut Protein, Hemoglobin, Hemp Seed Protein, Honey Protein, Hydrolyzed Actin, Hydrolyzed Amaranth Protein, Hydrolyzed animal protein, Hydrolyzed Barley Protein, Hydrolyzed Brazil Nut Protein, Hydrolyzed Conchiolin Protein, Hydrolyzed corn protein, Hydrolyzed Cottonseed Protein, Hydrolyzed Elastin, Hydrolyzed Extensin, Hydrolyzed Fibroin, Hydrolyzed Fibronectin, Hydrolyzed Fish Protein, Hydrolyzed Gadidae Protein, Hydrolyzed Gadidae Protein, Hydrolyzed Gelatin, Hydrolyzed Hair Keratin, Hydrolyzed Hazelnut, Hydrolyzed Hazelnut Protein, Hydrolyzed Hemoglobin, Hydrolyzed Hemp Seed Protein, Hydrolyzed Honey Protein, Hydrolyzed Keratin, Hydrolyzed Lactalbumin, Hydrolyzed Lupine Protein, Hydrolyzed Maple Sycamore Protein, Hydrolyzed Milk Protein, Hydrolyzed Oat Protein, Hydrolyzed Pea Protein, Hydrolyzed Potato Protein, Hydrolyzed Reticulin, Hydrolyzed Royal Jelly Protein, Hydrolyzed Sericin, Hydrolyzed Serum Protein, Hydrolyzed Sesame Protein, Hydrolyzed Soy Protein, Hydrolyzed Soymilk Protein, Hydrolyzed Spinal Protein, Hydrolyzed Spongin, Hydrolyzed Sweet Almond Protein, Hydrolyzed Vegetable Protein, Hydrolyzed Wheat Gluten, Hydrolyzed Wheat Protein, Hydrolyzed Whey Protein, Hydrolyzed Yeast Protein, Hydrolyzed Yogurt Protein, Hydrolyzed Zein, Integrin, Jojoba protein HP, Hydrolyzed, keratin, Lactalbumin, Lupine Protein, Maple Sycamore Protein, MEA-Hydrolyzed Collagen, MEA-Hydrolyzed Silk, Milk Protein, Myosin, Oat Protein, Pea Protein, polylysine, Potato Protein, Reticulin, Rice Quat, Royal Jelly Protein, Sericin, Serum Protein, Sesame Protein, Silk powder, Sodium Hydrolyzed Casein, Soy Protein, Soy Rice Peptides, Soymilk Protein, Spinal Protein, Spongin, Sweet Almond Protein, Vegetable Protein, Wheat Gluten, Whey Protein, Yeast Protein, Yogurt Protein, Zein, and Zinc Hydrolyzed Collagen.

In some embodiments, the protein is an albumin, Zein, or another pan-binding protein. In one embodiment, the protein is egg albumin.

In some embodiments, the particle comprises a core comprising the active agent and a coating layer comprising egg albumin. In one embodiment, the particle comprises a core comprising zinc pyrithione and a coating layer comprising egg albumin.

The protein can comprise between about 0.01% to about 99% (w/w) of the particle. In some embodiments, the protein comprises greater than 0.1% (w/w), greater than 0.5% (w/w), greater than 1% (w/w), greater than 2% (w/w), greater than 3% (w/w), greater than 4% (w/w), greater than 5% (w/w), greater than 6% (w/w), greater than 7% (w/w), greater than 8% (w/w), greater than 9% (w/w), greater than 10% (w/w), greater than 11% (w/w), greater than 12% (w/w), greater than 13% (w/w), greater than 14% (w/w), greater than 15% (w/w), greater than 16% (w/w), greater than 17% (w/w), greater than 18% (w/w), greater than 19% (w/w), greater than 20% (w/w), greater than 25% (w/w), greater than 30% (w/w), greater than 35% (w/w), greater than 40% (w/w), greater than 45% (w/w), or greater than 50% (w/w) of the total weight of the particles. Typically, the content of the protein in the particles are in the range of about 2-25% (w/w).

Ratio of the active agent to the protein can be any desired ratio. For example, ratio of the active agent to the protein can range from about 100:1 to about 1:100. In some embodiments, the ratio of the active agent to the protein can range from about 75:1 to about 1:75, from about 50:1 to about 1:50, from about 25:1 to about 1:25, from about 20:1 to about 1:20, from about 15:1 to about 1:15, from about 5:1 to about 1:5, or from about 25:1 to about 1:5. In some embodiments, the ratio of active agent to the protein is about 25:1, about 5:1, or about 1:1. The ratio can be based on weight, mass, or moles.

In some embodiments, the protein coating layer comprises an active agent. The active agent in the coating layer can be same or different from the active agent in the core.

The disclosure also provides a particle comprising an active agent and a cationic molecule coating layer without having a lipid in the coating layer. Without wishing to be bound by a theory, it is believed that the cationic molecules can be used in place of the lipid component to provide the same benefit as lipid component. For example, the cationic molecule can be used to bind or associate the particle to keratin, sebum, and/or hair to provide adhesion or retention on scalp. Accordingly, in some embodiments, the particle can comprises a core comprising the active agent and a coating layer comprising a cationic molecule, wherein the coating layer does not comprise a lipid.

Generally, any cationic molecule can be used for the coating. In some embodiments, the cationic molecule is a polyamine. Exemplary cationic molecules include, but are not limited to, Putrescine (Butane-1,4-diamine), Cadaverine (Pentane-1,5-diamine), Spermidine, Spermine, Cyclen (1,4,7,10-tetrazacyclododecane), Cyclam (1,4,8,11-Tetraazacyclotetradecane), Linear Polyethyleneimine (Poly(iminoethylene)), Norspermidine, p-Phenylenediamine (1,4-diaminobenzene), Diethylenetriamine (N-(2-aminoethyl)-1,2-ethanediamine), thermospermine, Tris(2-aminoethyl)amine, Hexamethylenediamine, Beta-lysine (3,6-diaminohexanoic acid), m-Phenylenediamine (1,3-diaminobenzene), Diaminopropane (1,2-Diaminopropane), Ethylenediamine dihydroiodide, and polyamine D 400 (Polyoxyalkyleneamine D 400).

In some embodiments, the particle comprises a core comprising the active agent and a coating layer comprising a polyamine. In one embodiment, the particle comprises a core comprising zinc pyrithione and a coating layer comprising a polyamine.

The cationic molecule can comprise between about 0.01% to about 99% (w/w) of the particle. In some embodiments, the cationic comprises greater than 0.1% (w/w), greater than 0.5% (w/w), greater than 1% (w/w), greater than 2% (w/w), greater than 3% (w/w), greater than 4% (w/w), greater than 5% (w/w), greater than 6% (w/w), greater than 7% (w/w), greater than 8% (w/w), greater than 9% (w/w), greater than 10% (w/w), greater than 11% (w/w), greater than 12% (w/w), greater than 13% (w/w), greater than 14% (w/w), greater than 15% (w/w), greater than 16% (w/w), greater than 17% (w/w), greater than 18% (w/w), greater than 19% (w/w), greater than 20% (w/w), greater than 25% (w/w), greater than 30% (w/w), greater than 35% (w/w), greater than 40% (w/w), greater than 45% (w/w), or greater than 50% (w/w) of the total weight of the particles. Typically, the content of the cationic molecule in the particles are in the range of about 2-25% (w/w).

Ratio of the active agent to the cationic molecule can be any desired ratio. For example, ratio of active agent to the cationic molecule can range from about 100:1 to about 1:100. In some embodiments, the ratio of the active agent to the cationic molecule can range from about 75:1 to about 1:75, from about 50:1 to about 1:50, from about 25:1 to about 1:25, from about 20:1 to about 1:20, from about 15:1 to about 1:15, from about 5:1 to about 1:5, or from about 25:1 to about 1:5. In some embodiments, the ratio of the active agent to the cationic molecule is about 25:1, about 5:1, or about 1:1. The ratio can be based on weight, mass, or moles.

In some embodiments, the cationic molecule coating layer comprises an active agent. The active agent in the coating layer can be same or different from the active agent in the core.

### Some Exemplary Particles

In some embodiments, the particle comprises a core comprising zinc pyrithione as the active agent and a coating layer comprising ethylene glycol distearate, caprylic acid, capric acid, lauric acid, myristic acid, palmitic acid, zinc recinoleate, CoQ10, or paraffin.

In some embodiments, the particle comprises a core comprising zinc pyrithione as the active agent and a coating layer comprising ethylene glycol distearate, wherein the zinc pyrithione and ethylene glycol distearate are in about 5:1 ratio.

In some embodiments, the particle comprises a core comprising zinc pyrithione as the active agent and a coating layer comprising palmitic acid, wherein the zinc pyrithione and palmitic acid are in about 5:1 ratio.

In some embodiments, the particle comprises a core comprising zinc pyrithione as the active agent and a coating layer comprising zinc recinoleate, wherein the zinc pyrithione and zinc recinoleate are in about 5:1 ratio.

In some embodiments, the particle comprises a core comprising zinc pyrithione as the active agent and a coating layer comprising coenzyme Q10, wherein the zinc pyrithione and coenzyme Q10 are in about 5:1 ratio.

In some embodiments, the particle comprises a core comprising zinc pyrithione as the active agent and a coating layer comprising zinc recinoleate, wherein the zinc pyrithione and zinc recinoleate are in about 5:1 ratio.

In some embodiments, the particle comprises a core comprising zinc pyrithione as the active agent and a coating layer comprising ethylene glycol distearate and egg albumin, caprylic acid and paraffin, capric acid and paraffin, lauric acid and paraffin, or myristic acid and paraffin.

In some embodiments, the particle comprises a core comprising zinc pyrithione as the active agent and a coating layer comprising caprylic acid and paraffin, capric acid and paraffin, lauric acid and paraffin, or myristic acid and paraffin, wherein the lipid and paraffin are in about 1:1 ratio.

In some embodiments, the particle comprises a core comprising zinc pyrithione as the active agent and a coating layer comprising caprylic acid and paraffin, capric acid and paraffin, lauric acid and paraffin, or myristic acid and paraffin, wherein zinc pyrithione and total lipid plus paraffin is in about 25:1 ratio.

In some embodiments, the particle comprises a core comprising zinc pyrithione as the active agent and a coating layer comprising caprylic acid and paraffin, capric acid and paraffin, lauric acid and paraffin, or myristic acid and paraffin, wherein the lipid and paraffin are in about 1:1 ratio and zinc pyrithione and total lipid plus paraffin is in about 25:1 ratio.

In some embodiments, the particle comprises a core comprising zinc pyrithione as the active agent and a coating layer comprising caprylic acid and paraffin, wherein zinc pyrithione and total lipid plus paraffin is in about 5:1 ratio.

In some embodiments, the particle comprises a core comprising zinc pyrithione as the active agent and a coating layer comprising caprylic acid and paraffin, wherein caprylic acid and paraffin are in about 1:1 ratio and zinc pyrithione and total lipid plus paraffin is in about 5:1 ratio.

In some embodiments, the particle comprises a core comprising zinc pyrithione as the active agent and a coating layer comprising ethylene glycol distearate and egg albumin, wherein the EGDS and egg albumin are in about 3:2 ratio.

In some embodiments, the particle comprises a core comprising zinc pyrithione as the active agent and a coating layer comprising ethylene glycol distearate and egg albumin, wherein the EGDS and egg albumin are in about 3:2 ratio and the zinc pyrithione and total of EGDS and egg albumin is in about 30:1 ratio.

In some embodiments, the particle comprises a core comprising ketoconazole as the active agent and a coating layer comprising ethylene glycol distearate, triplamitin, or polyglyceryl-6-distearate.

In some embodiments, the particle comprises a core comprising ketoconazole as the active agent and a coating layer comprising ethylene glycol distearate, triplamitin, or polyglyceryl-6-distearate, wherein the ketoconazole and the lipid are in about 5:1 ratio.

In some embodiments, the particle comprises a core comprising salicylic acid as the active agent.

In some embodiments, the particle comprises a core comprising curcuminoid or a tetrahydro curcuminoid as the active agent and a coating layer comprising coenzyme Q10.

In some embodiments, the particle comprises a core comprising titanium dioxide as the active agent and a coating layer comprising coenzyme Q10.

In some embodiments, the particle comprises a core comprising zinc oxide as the active agent and a coating layer comprising coenzyme Q10.

In some embodiments, the particle comprises a core comprising chloroxylenol as the active agent.

In some embodiments, the particle comprises a core comprising ascorbic acid as the active agent and a coating layer comprising coenzyme Q10.

### Compositions Comprising the Particles

In another aspect, the disclosure provides a composition comprising a particle disclosed herein. The composition comprising the particles can be administered to a subject. As used herein, the terms “administer”, “administering,” and “introducing” are used interchangeably herein and refer to the placement of a composition into a subject by a method or route which results in at least partial localization of the composition at a desired site such that desired effect is produced. The compound or composition can be administered by any appropriate route known in the art which results in an effective treatment in the subject, including, but not limited to, oral or parenteral routes, including intravenous, intramuscular, intradermal, subcutaneous, transdermal, airway (aerosol), pulmonary, nasal, opthalamic, rectal, topical (including buccal and sublingual), and superficial administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrases “parenteral administration” and “administered parenterally” as used herein mean modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration therapeutic compositions other than directly into a tumor such that it enters the animal's system and, thus, is subject to metabolism and other like processes.

The terms “composition” or “pharmaceutical composition” used interchangeably herein refer to compositions or formulations that usually comprise an excipient, such as a pharmaceutically acceptable carrier that is conventional in the art and that is suitable for administration to mammals, and preferably humans or human cells. Such compositions can be specifically formulated for administration via one or more of a number of routes, including but not limited to, oral, ocular parenteral, intravenous, intraarterial, subcutaneous, intranasal, sublingual, intraspinal, intracerebroventricular, and the like. In addition, compositions for topical (e.g., oral mucosa, respiratory mucosa) and/or oral administration can form solutions, suspensions, tablets, pills, capsules, sustained-release formulations, oral rinses, or powders, as known in the art are described herein. The compositions also can include stabilizers and preservatives. For examples of carriers, stabilizers and adjuvants, University of the Sciences in Philadelphia (2005) Remington: The Science and Practice of Pharmacy with Facts and Comparisons, 21st Ed.

In some embodiments, the administration is topical or superficial.

Without limitations the composition can be a cream, oil, lotion, serum, gel, soap, face wash, shampoo, conditioner, toothpaste, mouth wash, chewing gum, sunscreen, nail varnish, ointment, foam, spray, or aerosol. In some embodiments, the composition comprising the particles disclosed herein is an antifungal, antibacterial, anti-inflammatory, anti-aging, anti-wrinkle, or skin whitening or skin bleaching composition. In one embodiment, the composition comprising the particles disclosed herein is an anti-acne composition.

In some embodiments, the composition comprising the particles disclosed herein is an antifungal composition. The composition can be a personal care composition. In some embodiments, the composition is a cream, oil, lotion, serum, shampoo, nail varnish, ointment, foam, spray or aerosol. The composition comprises an effective amount of the particles. As used here, the term “effective amount” is that amount of the pyrithione salt containing particles necessary to achieve the desired improvement.

In some embodiments, the composition is an anti-dandruff hair care composition. The hair care composition can be selected from the group consisting of a shampoo, a conditioner, a rinse, a lotion, an aerosol, a gel, a mousse, and a hair dye. In one embodiment, the hair care composition is a shampoo.

In some embodiments, the composition is an anti-acne composition.

In some embodiments, the composition is a skin care composition. As defined herein, the term “skin care composition” refers to materials applied topically to the skin that benefit, improve, or enhance the condition of the skin, or treat skin suffering from an infectious or diseased condition. Such skin care compositions include bases such as soap bases, cosmetic bases, medicament bases, cream bases, emollient bases, and combinations thereof, as well as other bases known in the art. Exemplary skin care compositions include, but are not limited to, lotions, creams, gels, sticks, sprays, ointments, cleansing liquid washes, cleansing solid bars, pastes, foams, powders, shaving creams, wipes, and the like.

In some embodiments, the composition is an oral care composition. Oral care composition can be selected from the group consisting of toothpastes, mouthwashes, chewing gums, and the like.

Without limitations, the composition can comprise any desired amount of the particles disclosed herein. For example, the composition can comprise from about 0.01% to about 99% (w/w or w/v) of the particles. In some embodiments, the composition can comprise from about 0.1% to about 75% (w/w or w/v), from about 1% to about 50% (w/w or w/v), from about 1.5% to about 40% (w/w or w/v), from about 2% to about 25% (w/w or w/v), or from about 2.5% to about 25% (w/w or w/v) of the particles. In some embodiments, the composition can comprise from about 2.5%, 3%, 3.5%, 4%, 4.5%, 5%, 7.5%, 10%, 12.5%, 15%, 17.5%, 20%, 22.5%, or 25% (w/w or w/v) of the particles.

In some embodiments, the composition can further comprise one or more excipients. The excipient can be a solvent or an additive. The additive can be selected from the group consisting of surfactants, stabilizers, rheology modifiers, conditioning agents, fragrances, potentiating agents, preservatives, opacifiers, pH modifiers, and any combinations thereof. Amount of the excipients in the composition can range from a bout 5% to 99.99% (w/w or w/v). Generally, the pH of intended use of the composition will generally range from about pH 2 to about pH10, from about pH 3 to about pH 9, from about pH 4 and about pH 8, or from about pH 5.5 to about pH 7.5.

The compositions disclosed herein can further comprise one or more optional components known for use in hair care or personal care products, provided that the optional components are physically and chemically compatible with the essential components described herein, or do not otherwise unduly impair product stability, aesthetics or performance. Individual concentrations of such optional components can range from about 0.001% to about 10% by weight of the compositions.

Non-limiting examples of optional components for use in the composition include a deposition aid, cationic polymers, nonionic polymers, dispersed particles, conditioning agents (silicones and organic conditioning oils), humectant, suspending agent, additional anti-dandruff actives, viscosity modifiers, dyes, nonvolatile solvents or diluents (water soluble and insoluble), pearlescent aids, foam boosters, additional surfactants or nonionic cosurfactants, pediculocides, pH adjusting agents, perfumes, preservatives, chelants, proteins, skin active agents, sunscreens, UV absorbers, vitamins, antioxidants, preserving agents, fillers, surfactants, UVA and/or UVB sunscreens, fragrances, viscosifying agents, wetting agents, anionic polymers, nonionic polymers, amphoteric polymers, viscosity/foam stabilizers, opacifying/pearlizing agents, sequestering agents, stabilizing agents, hair conditioning agents, humectants, anti-static agents, antifreezing agents, buffering agents, dyes, and pigments. These adjuvants are well known in the field of cosmetics and are described in many publications, for example see Harry's Book of Cosmeticology, 8th edition, Martin Rieger, ed., Chemical Publishing, New York (2000).

The compositions disclosed herein can also include a deposition aid. The deposition aid is included to effectively enhance deposition of the composition components. The deposition aid can comprise any material that enhances the deposition of the composition components onto the hair, scalp, or skin. In some embodiments, the deposition aids are cationic polymers. The concentration of the deposition aid in the composition should be sufficient to effectively enhance the deposition of the components and typically range from about 0.05% to about 5%, preferably from about 0.075% to about 2.5%, more preferably from about 0.1% to about 1.0%, by weight of the composition.

The compositions disclosed herein can comprise a cationic polymer. Concentrations of the cationic polymer in the composition typically range from about 0.05% to about 3%, preferably from about 0.075% to about 2.0%, more preferably from about 0.1% to about 1.0%, by weight of the composition. Preferred cationic polymers will have cationic charge densities of at least about 0.9 meq/gm, preferably at least about 1.2 meq/gm, more preferably at least about 1.5 meq/gm, but also preferably less than about 7 meq/gm, more preferably less than about 5 meq/gin. The average molecular weight of such suitable cationic polymers will generally be between about 10,000 and 10 million, preferably between about 50,000 and about 5 million, more preferably between about 100,000 and about 3 million.

Suitable cationic polymers for use in the compositions contain cationic nitrogen containing moieties such as quaternary ammonium or cationic protonated amino moieties. The cationic protonated amines can be primary, secondary, or tertiary amines (preferably secondary or tertiary), depending upon the particular species and the selected pH of the composition. Any anionic counterions can be used in association with the cationic polymers so long as the polymers remain soluble in water, in the composition, or in a coacervate phase of the composition, and so long as the counterions are physically and chemically compatible with the essential components of the composition or do not otherwise unduly impair product performance, stability or aesthetics. Non limiting examples of such counterions include halides (e.g., chloride, fluoride, bromide, iodide), sulfate and methylsulfate.

Non limiting examples of cationic polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)).

Non limiting examples of suitable cationic polymers include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone or vinyl pyrrolidone.

Suitable cationic protonated amino and quaternary ammonium monomers, for inclusion in the cationic polymers of the composition herein, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts.

Other suitable cationic polymers for use in the compositions include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16); copolymers of 1-vinyl-2-pyrrolidone and dimethylaminoethyl methacrylate (referred to in the industry by CTFA as Polyquaternium-11); cationic diallyl quaternary ammonium containing polymers, including, for example, dimethyldiallylammonium chloride homopolymer, copolymers of acrylamide and dimethyldiallylammonium chloride (referred to in the industry by CTFA as Polyquaternium 6 and Polyquaternium 7, respectively); amphoteric copolymers of acrylic acid including copolymers of acrylic acid and dimethyldiallylammonium chloride (referred to in the industry by CTFA as Polyquaternium 22), terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride and methylacrylate (referred to in the industry by CTFA as Polyquaternium 47).

Other suitable cationic polymers for use in the composition include polysaccharide polymers, such as cationic cellulose derivatives and cationic starch derivatives. Preferred cationic cellulose polymers are salts of hydroxyethyl cellulose reacted with trimethyl ammonium substituted epoxide, referred to in the industry (CTFA) as Polyquaternium 10 and available from Amerchol Corp. (Edison, N.J., USA) in their Polymer LR, JR, and KG series of polymers. Other suitable types of cationic cellulose include the polymeric quaternary ammonium salts of hydroxyethyl cellulose reacted with lauryl dimethyl ammonium-substituted epoxide referred to in the industry (CTFA) as Polyquaternium 24. These materials are available from Amerchol Corp. under the tradename Polymer LM-200.

Other suitable cationic polymers include cationic guar gum and derivatives thereof, such as guar hydroxypropyltrimonium chloride, specific examples of which include the Jaguar series commercially available from Rhone-Poulenc Incorporated and the N-Hance series commercially available from Aqualon Division of Hercules, Inc. Other suitable cationic polymers include quaternary nitrogen-containing cellulose ethers, some examples of which are described in U.S. Pat. No. 3,962,418. Other suitable cationic polymers include copolymers of etherified cellulose, guar and starch, some examples of which are described in U.S. Pat. No. 3,958,581. When used, the cationic polymers herein are either soluble in the composition or are soluble in a complex coacervate phase in the composition formed by the cationic polymer and the anionic, amphoteric and/or zwitterionic detersive surfactant component described hereinbefore. Complex coacervates of the cationic polymer can also be formed with other charged materials in the composition.

Polyalkylene glycols having a molecular weight of more than about 1000 are useful herein. Polyethylene glycol polymers useful herein are PEG-2M (also known as Polyox WSR® N-10, which is available from Union Carbide and as PEG-2,000); PEG-5M (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

The composition can also include dispersed particles. The can include at least 0.025% by weight of the dispersed particles, more preferably at least 0.05%, still more preferably at least 0.1%, even more preferably at least 0.25%, and yet more preferably at least 0.5% by weight of the dispersed particles. In some embodiments, it is preferable to incorporate no more than about 20% by weight of the dispersed particles, more preferably no more than about 10%, still more preferably no more than 5%, even more preferably no more than 3%, and yet more preferably no more than 2% by weight of the dispersed particles.

Conditioning agents include any material which is used to give a particular conditioning benefit to hair and/or skin. The conditioning agents useful in the compositions of the present invention typically comprise a water insoluble, water dispersible, non-volatile, liquid that forms emulsified, liquid particles or are solubilized by the surfactant micelles, in the anionic detersive surfactant component (described above). Suitable conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein.

The conditioning agent of the compositions can be an insoluble silicone conditioning agent. The silicone conditioning agent particles can comprise volatile silicone, non-volatile silicone, or combinations thereof. Preferred are non-volatile silicone conditioning agents. If volatile silicones are present, they will typically be incidental to their use as a solvent or carrier for commercially available forms of non-volatile silicone material ingredients, such as silicone gums and resins. The silicone conditioning agent particles can comprise a silicone fluid conditioning agent and can also comprise other ingredients, such as a silicone resin to improve silicone fluid deposition efficiency or enhance glossiness of the hair.

The concentration of the silicone conditioning agent typically ranges from about 0.01% to about 10%, by weight of the composition, preferably from about 0.1% to about 8%, more preferably from about 0.1% to about 5%, more preferably from about 0.2% to about 3%. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609. The silicone conditioning agents for use in the compositions of the present invention preferably have a viscosity, as measured at 25° C., from about 20 to about 2,000,000 centistokes (“csk”), more preferably from about 1,000 to about 1,800,000 csk, even more preferably from about 50,000 to about 1,500,000 csk, more preferably from about 100,000 to about 1,500,000 csk.

The dispersed silicone conditioning agent particles typically have a volume average particle diameter ranging from about 0.01 m to about 50 μm. For small particle application to hair, the volume average particle diameters typically range from about 0.01 μm to about 41 μm, preferably from about 0.01 μm to about 2 μm, more preferably from about 0.01 μm to about 0.51 μm. For larger particle application to hair, the volume average particle diameters typically range from about 5 μm to about 125 μm, preferably from about 10 μm to about 90 μm, more preferably from about 15 μm to about 70 μm, more preferably from about 20 μm to about 50 μm.

Background material on silicones including sections discussing silicone fluids, gums, and resins, as well as manufacture of silicones, are found in Encyclopedia of Polymer Science and Engineering, vol. 15, 2d ed., pp 204-308, John Wiley & Sons, Inc. (1989).

Silicone fluids include silicone oils, which are flowable silicone materials having a viscosity, as measured at 25° C., less than 1,000,000 csk, preferably from about 5 csk to about 1,000,000 csk, more preferably from about 100 csk to about 600,000 csk. Suitable silicone oils for use in the compositions of the present invention include polyalkyl siloxanes, polyaryl siloxanes, polyalkylaryl siloxanes, polyether siloxane copolymers, and mixtures thereof. Other insoluble, non-volatile silicone fluids having hair conditioning properties can also be used.

Other silicone fluids suitable for use in the compositions are the insoluble silicone gums. These gums are polyorganosiloxane materials having a viscosity, as measured at 25° C., of greater than or equal to 1,000,000 csk. Silicone gums are described in U.S. Pat. No. 4,152,416; Noll and Walter, Chemistry and Technology of Silicones, New York: Academic Press (1968); and in General Electric Silicone Rubber Product Data Sheets SE 30, SE 33, SE 54 and SE 76. Specific non-limiting examples of silicone gums for use in the compositions of the present invention include polydimethylsiloxane, (polydimefhylsiloxane) (methylvinylsiloxane) copolymer, polydimethylsiloxane) (diphenyl siloxane)(mefhylvinylsiloxane) copolymer and mixtures thereof.

Other non-volatile, insoluble silicone fluid conditioning agents that are suitable for use in the compositions of the present invention are those known as “high refractive index silicones,” having a refractive index of at least about 1.46, preferably at least about 1.48, more preferably at least about 1.52, more preferably at least about 1.55. The refractive index of the polysiloxane fluid will generally be less than about 1.70, typically less than about 1.60. In this context, polysiloxane “fluid” includes oils as well as gums.

Silicone fluids suitable for use in the compositions of the present invention are disclosed in U.S. Pat. No. 2,826,551, U.S. Pat. No. 3,964,500, U.S. Pat. No. 4,364,837, British Pat. No. 849,433, and Silicon Compounds, Petrarch Systems, Inc. (1984).

Silicone resins can be included in the silicone conditioning agent of the compositions of the present invention. These resins are highly cross-linked polymeric siloxane systems. The cross-linking is introduced through the incorporation of trifunctional and tetrafunctional silanes with monofunctional or difunctional, or both, silanes during manufacture of the silicone resin.

Silicone materials and silicone resins in particular, can conveniently be identified according to a shorthand nomenclature system known to those of ordinary skill in the art as “MDTQ” nomenclature. Under this system, the silicone is described according to presence of various siloxane monomer units which make up the silicone. Briefly, the symbol M denotes the monofunctional unit (CH3)3SiO05; D denotes the difunctional unit (CH3)2SiO; T denotes the trifunctional unit (CH3)Si015; and Q denotes the quadra- or tetra-functional unit Si02. Primes of the unit symbols (e.g. M′, D′, T, and Q′) denote substituents other than methyl, and must be specifically defined for each occurrence.

Preferred silicone resins for use in the compositions of the present invention include, but are not limited to MQ, MT, MTQ, MDT and MDTQ resins. Methyl is a preferred silicone substituent. Especially preferred silicone resins are MQ resins, wherein the M:Q ratio is from about 0.5:1.0 to about 1.5:1.0 and the average molecular weight of the silicone resin is from about 1000 to about 10,000.

The conditioning component of the compositions of the present invention can also comprise from about 0.05% to about 3%, by weight of the composition, preferably from about 0.08% to about 1.5%, more preferably from about 0.1% to about 1%, of at least one organic conditioning oil as the conditioning agent, either alone or in combination with other conditioning agents, such as the silicones (described above).

Suitable organic conditioning oils for use as conditioning agents in the compositions of the present invention include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils preferably are from about C to about C19. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2, 2, 4, 4, 6, 6, 8, 8-dimethyl-10-methylundecane and 2, 2, 4, 4, 6, 6-dimethyl-8-methylnonane, available from Permethyl Corporation. Hydrocarbon polymers such as polybutene and polydecene are preferred. A preferred hydrocarbon polymer is polybutene, such as the copolymer of isobutylene and butene. A commercially available material of this type is L-14 polybutene from Amoco Chemical Corporation.

Organic conditioning oils for use in the compositions of the present invention can also include liquid polyolefins, more preferably liquid poly-a-olefins, more preferably hydrogenated liquid poly-a-olefins. Polyolefins for use herein are prepared by polymerization of C4 to about C14 olefenic monomers, preferably from about C6 to about C12.

Non-limiting examples of olefenic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin containing refinery feedstocks or effluents. Preferred hydrogenated a-olefin monomers include, but are not limited to: 1-hexene to 1-hexadecenes, 1-octene to 1-tetradecene, and mixtures thereof.

Other suitable organic conditioning oils for use as the conditioning agent in the compositions of the present invention include, but are not limited to, fatty esters having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof can include or have covalently bonded thereto other compatible functionalities, such as amides and alkoxy moieties (e.g., ethoxy or ether linkages, etc.).

Specific examples of preferred fatty esters include, but are not limited to: isopropyl isostearate, hexyl laurate, isohexyl laurate, isohexyl palmitate, isopropyl palmitate, decyl oleate, isodecyl oleate, hexadecyl stearate, decyl stearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the compositions of the present invention are mono-carboxylic acid esters of the general formula R′COOR, wherein R′ and R are alkyl or alkenyl radicals, and the sum of carbon atoms in R′ and R is at least 10, preferably at least 22.

Still other fatty esters suitable for use in the compositions of the present invention are di- and tri-alkyl and alkenyl esters of carboxylic acids, such as esters of C4 to C8 dicarboxylic acids (e.g. C1 to C22 esters, preferably C1 to C6, of succinic acid, glutaric acid, and adipic acid). Specific non-limiting examples of di- and tri-alkyl and alkenyl esters of carboxylic acids include isocetyl stearyol stearate, diisopropyl adipate, and tristearyl citrate.

Other fatty esters suitable for use in the compositions of the present invention are those known as polyhydric alcohol esters. Such polyhydric alcohol esters include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Still other fatty esters suitable for use in the compositions of the present invention are glycerides, including, but not limited to, mono-, di-, and tri-glycerides, preferably di- and tri-glycerides, more preferably triglycerides. For use in the compositions described herein, the glycerides are preferably the mono-, di-, and tri-esters of glycerol and long chain carboxylic acids, such as C10 to C22 carboxylic acids. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil. Synthetic oils include, but are not limited to, triolein and tristearin glyceryl dilaurate.

Other fatty esters suitable for use in the compositions of the present invention are water insoluble synthetic fatty esters.

Specific non-limiting examples of suitable synthetic fatty esters for use in the compositions of the present invention include: P-43 (C8-C10 triester of trimefhylolpropane), MCP-684 (tetraester of 3,3 diethanol-1,5 pentadiol), MCP 121 (C8-C10 diester of adipic acid), all of which are available from Mobil Chemical Company.

Also suitable for use in the compositions herein are the conditioning agents described by the Procter & Gamble Company in U.S. Pat. Nos. 5,674,478, and 5,750,122. Also suitable for use herein are those conditioning agents described in U.S. Pat. No. 4,529,586 (Clairol), U.S. Pat. No. 4,507,280 (Clairol), U.S. Pat. No. 4,663,158 (Clairol), U.S. Pat. No. 4,197,865 (L'Oreal), U.S. Pat. No. 4,217,914 (L'Oreal), U.S. Pat. No. 4,381,919 (L'Oreal), and U.S. Pat. No. 4,422,853 (L'Oreal).

The compositions of the present invention can contain a humectant. The humectants herein are selected from the group consisting of polyhydric alcohols, water soluble alkoxylated nonionic polymers, and mixtures thereof. The humectants, when used herein, are preferably used at levels by weight of the composition of from about 0.1% to about 20%, more preferably from about 0.5% to about 5%.

Polyhydric alcohols useful herein include glycerin, sorbitol, propylene glycol, butylene glycol, hexylene glycol, ethoxylated glucose, 1,2-hexane diol, hexanetriol, dipropylene glycol, erythritol, trehalose, diglycerin, xylitol, maltitol, maltose, glucose, fructose, sodium chondroitin sulfate, sodium hyaluronate, sodium adenosine phosphate, sodium lactate, pyrrolidone carbonate, glucosamine, cyclodextrin, and mixtures thereof.

Water soluble alkoxylated nonionic polymers useful herein include polyethylene glycols and polypropylene glycols having a molecular weight of up to about 1000 such as those with CTFA names PEG-200, PEG-400, PEG-600, PEG-1000, and mixtures thereof.

The compositions of the present invention can further comprise a suspending agent at concentrations effective for suspending water-insoluble material in dispersed form in the compositions or for modifying the viscosity of the composition. Such concentrations range from about 0.1% to about 10%, preferably from about 0.3% to about 5.0%, by weight of the compositions.

Suitable suspending agents include crystalline suspending agents that can be categorized as acyl derivatives, long chain amine oxides, or combinations thereof. These suspending agents are described in U.S. Pat. No. 4,741,855.

The compositions of the present invention can contain also vitamins and amino acids such as: water soluble vitamins such as vitamin B1, B2, B6, B12, C, pantothenic acid, pantothenyl ethyl ether, panthenol, biotin, and their derivatives, water soluble amino acids such as asparagine, alanin, indole, glutamic acid and their salts, water insoluble vitamins such as vitamin A, D, E, and their derivatives, water insoluble amino acids such as tyrosine, tryptamine, and their salts.

The compositions of the present invention can also contain pigment materials such as nitroso, monoazo, diazo, carotenoid, triphenyl methanes, triaryl methanes, xanthenes, quinolines, oxazines, azines, anthraquinones, indigoids, thionindigoids, quinacridones, phthalocyianines, botanicals, and natural colors including water soluble dye components. The compositions of the present invention can also contain chelating agents.

Personal care compositions are well known in the art. See for example, U.S. Pat. No. 6,274,150; No. 6,599,513; No. 6,0969,169; No. 4,735,742; No. 6,451,300; No. 4,942,161; No. 5,456,851; No. 5,854,246; No. 6,099,870; No. 7,094,422; No. 7,732,450; No. 6,663,875; No. 6,812,238; No. 7,732,450; No. 5,654,293; No. 6,099,870; No. 6,375,939; No. 6,451,300; No. 6,616,941; No. 6,649,155; No. 6,974,569; No. 6,491,902; No. 6,524,594; No. 6,419,913, No. 6,284,234; No. 6,908,889; No. 6,495,498; and No. 6,514,490, U.S. Pat. App. Pub No. US2010/0183539; No. US2009/0317502 No. US2006/0269501; No. US2003/0003070; No. US2008/0107749; No. US2008/0200539; No. US2003/0206958; No. US2002/0176894; US2006/0110415; No. US2010/0104646; No. US2010/0040697; No. US2010/0215775; No. US2009/0214628; No. US2007/0110700; and No. US20080152611, and Int. Pat. Pub. No. WO2001051014; No. WO2001066551; No. WO2002090354; No. WO2003006009; No. WO2000043390; No. WO2001032652; No. WO2001066551; No. WO2002090354; No. WO2003008391; No. WO2004028502; No. WO2004018485; No. WO2005006860; No. WO2010138674; No. WO2003086271; No. WO2002067880; No. WO2010/051918; No. WO2006109642; No. WO2009006212; No. WO2007021789; No. WO2008006712; No. WO2010149424; No. WO2010127924; No. WO2009071408; No. WO2009053431; No. WO2008006712; No. WO2008003677; No. WO2004035015; and No. WO2002067880, content of all of which is incorporated herein by reference.

In some embodiments, the personal care composition is a hair care composition. A hair care composition can be used to or prevent dandruff. Hair care compositions are herein defined as compositions for the treatment of hair including, but not limited to, shampoos, conditioners, rinses, lotions, aerosols, gels, mousses, and hair dyes. The hair care compositions of the present invention comprise an effective amount of the particles disclosed herein, ranging from about 0.001% to about 90%, preferably from about 0.1% to about 5%, and more preferably from about 0.5% to about 3% by weight relative to the total weight of the composition. As used here, the term “effective amount” is that amount of the particles in the hair care composition necessary to achieve the desired improvement.

In addition to the particles, the hair care composition can comprise a cosmetically acceptable medium for hair care compositions, examples of which are described for example in U.S. Pat. No. 6,280,747; No. 6,139,851; and No. 6,013,250, all of which are incorporated herein by reference. For example, these hair care compositions can be aqueous, alcoholic or aqueous-alcoholic solutions, the alcohol preferably being ethanol or isopropanol, in a proportion of from about 1 to about 75% by weight relative to the total weight, for the aqueous-alcoholic solutions. Additionally, the hair care compositions can contain one or more conventional cosmetic or dermatological additives or adjuvants including, but not limited to, antioxidants, preserving agents, fillers, surfactants, UVA and/or UVB sunscreens, fragrances, viscosifying agents, wetting agents, anionic polymers, nonionic polymers, amphoteric polymers, viscosity/foam stabilizers, opacifying/pearlizing agents, sequestering agents, stabilizing agents, hair conditioning agents, humectants, anti-static agents, antifreezing agents, buffering agents, dyes, and pigments. These adjuvants are well known in the field of cosmetics and are described in many publications, for example see Harry's Book of Cosmeticology, 8th edition, Martin Rieger, ed., Chemical Publishing, New York (2000).

The particles disclosed herein can be used in a shampoo. Suitable shampoo compositions are well known in the art. For example, components of shampoo compositions are described by Wells et al. in U.S. Pat. No. 6,930,078, by Patel et al. in U.S. Pat. No. 5,747,436 and by Niemiec et al. in U.S. Pat. No. 6,908,889. The hair shampoo composition can be an aqueous solution, aqueous-alcoholic solution or an oil-in-water (O/W) or water in oil in water (W/O/W) emulsion. The shampoo composition of the invention contains an effective amount of the particles from about 0.001% to about 10%, preferably from about 0.1% to about 5%, and more preferably from about 0.5% to about 3% by weight relative to the total weight of the composition. The balance of the shampoo composition is comprised of the fluid vehicle, surfactant, and other additives. Typically, the fluid vehicle comprises water and other solvents which can include, without limitation, mineral oils and fatty alcohols.

Surfactants are the primary components in shampoo compositions. The amount of primary surfactant is generally in the range of between about 10% and 20% as based on the final weight of the composition, more typically from about 8 to about 18%. A secondary surfactant can also be present, generally in the range of about 0 to about 6%. The surfactants in the shampoo composition according to the invention can include one or more, or a combination thereof of anionic, nonionic, amphoteric or cationic surfactants. Examples of anionic surfactants include, but are not limited to, soaps, alkyl and alkyl ether sulfates, and alpha-olefin sulfonates. The preferred anionic surfactants are lauryl (ammonium, sodium, triethanolamine and diethanolamine and laureth (sodium and ammonium)) sulfates. Secondary anionic surfactants include, but are not limited to, sulfosuccinates, linear alkylbenzene sulfonates, N-acyl methyltaurates, N-acyl sarcosinates, acyl isothionates, N-acyl polypeptide condensates, polyalkoxylated ether glycolates, monoglyceride sulfates, fatty glycerol ether sulfonates. Examples of nonionic surfactants include, but are not limited to, fatty alkanolamides, amine oxides, polymeric ethers, polysorbate 20, PEG-80 sorbitan, and nonoxynols. Examples of amphoteric surfactants include, but are not limited to, betaines, alkyl-substituted amino acids (sodium lauraminopropionate and sodium lauriminopropionate).

The shampoo composition according to the invention can also comprise viscosity and foam stabilizers, the amount of, generally in the range of about 1.5 to about 5% based on the final weight of the composition. Specific examples of viscosity/foam stabilizers include, but are not limited to, alkanolamides (such as Cocamide MEA).

Additionally, the shampoo composition can contain minor proportions of one or more conventional cosmetic or dermatological additives or adjuvants, provided that they do not interfere with the mildness, performance or aesthetic characteristics desired in the final products. The total concentration of added ingredients usually is less than 5%, preferably less than 3%, by weight of the total composition. Such minor components include but are not limited to, opacifying/pearlizing agents, such as stearic acid derivatives (e.g., ethylene glycol monostearate or ethylene glycol distearate); solvents; sequestering agents, such as disodium ethylene diaminetetraacetic acid (EDTA) and its salts, citric acid, or polyphosphates; stabilizing agents; viscosifying agents, such as salts (e.g, sodium chloride or ammonium chloride) for anionic formulations; PEG-120 methyl glucose dioleate and PEG-150 pentaerythrityl tetrastearate for anionic/nonionic formulations; hair conditioning agents, such as the cationic polymers polyquaternium 10 (Ucare Polymers), cationic guar (Jacquar C-261N), polyquaternium-7 (Merquat Polymers) and silicones such as dimethicone and aminodimethicone; humectants; anti-static agents; anti-freezing agents, buffering agents; antioxidants, such as BHT, BHA and tocopherol; UV absorbers, such as benzophenone; preservatives, such as parabens; fragrances; and dyes or pigments. These adjuvants are well known in the field of cosmetics and are described in many publications, for example see Harry's Book of Cosmeticology, supra.

The final essential component in the shampoo composition is water, which provides an aqueous medium that constitutes the balance of the shampoo composition. Generally, the proportion of water ranges from about 53% to about 95%, preferably, 68% to about 92%, and most preferably about 80% to about 87%, by weight of the resultant shampoo composition.

The shampoo compositions of the present invention can be prepared using conventional formulation and mixing techniques. Where melting or dissolution of solid surfactants or wax components is required these can be added to a premix of the surfactants, or some portion of the surfactants, mixed and heated to melt the solid components, e.g., about 50° C. to about 95° C. This mixture can then optionally be processed through a high shear mill and cooled, and then the remaining components mixed in. The compositions typically have a final viscosity of from about 2,000 to about 20,000 cps (centipoise). The viscosity of the composition can be adjusted by conventional techniques including addition of sodium chloride or ammonium xylenesulfonate as needed.

A hair care composition can also include one or more antidandruff agents. As used herein, the term “antidandruff agent” refers to any chemical that is effective in the treatment of dandruff and/or the symptoms associated therewith. Antidandruff agents are well known in the art. See for example, U.S. Pat. App. Pub. No. 2004/0202636 and No. 2003/0003070, and U.S. Pat. No. 6,284,234, content of all of which is incorporated herein by reference. Typically, the antidandruff agent is an antifungal agent effective against the fungus Malassezia. Suitable antidandruff agents include, but are not limited to pyridinethione salts, such as calcium, magnesium, barium, strontium, zinc, and zirconium pyridinethione salts; azoles, such as climbazole, ketoconazole, and itraconazole, piroctone olamine (octopirox); undecylenic acid, undecylenamidopropylbetaine (AMPHORAM U®), coal tar (NeutrogenaT/gel, CAS No. 8030-31-7; salisylic acid (Ionil T); selenium sulfide (Selsun Blue) and Tea tree, and mixtures thereof. One pyridinethione salt is the zinc salt of 1-hydroxy-2-pyridinethione (also known as zinc pyridinethione). These antifungal agents are generally available from commercial sources. For example, zinc pyridinethione is available from Olin Corporation (Norwalk, Conn.); octopirox is available from Hoechst AG (Frankfurt, Germany); AMPHORAM U® is available from CECA Arkema Group (France); and ketoconazole is available from Alfa Chem (Kings Point, N.Y.).

In some embodiments, the personal care composition is a skin care composition. A skin care composition can be used to or prevent acne. Skin care compositions are herein defined as compositions for the treatment of skin including, but not limited to, skin conditioners, moisturizers, foundations, anti-wrinkle products, skin cleansers, and body washes. The skin care compositions of the present invention include any composition that can be topically applied to the skin, including but not limited to, lotions, creams, gels, sticks, sprays, ointments, cleansing liquid washes, cleansing solid bars, pastes, foams, powders, shaving creams, and wipes.

The skin care compositions of the invention can comprise several types of cosmetically-acceptable topical carriers including, but not limited to solutions, colloidal suspensions, dispersions, emulsions (microemulsions, nanoemulsions, multiple and non-aqueous emulsions), hydrogels, and vesicles (liposomes, niosomes, novasomes). Components and formulation methods of suitable cosmetically-acceptable topical carriers are well known in the art and are described, for example, in U.S. Pat. No. 6,797,697 and U.S. Pat. App. Pub. No. 2005/0142094 and No. 2005/0008604, Int. Pat. App. Pub. No. 2006/029818 and No. 2000/062743, content of all of which is incorporated herein by reference. Those skilled in the art will appreciate the various methods for producing these various product forms.

Typically, the cosmetically acceptable medium for skin care compositions comprises water and other solvents which include, but are not limited to, mineral oils and fatty alcohols. The cosmetically-acceptable medium is from about 10% to about 99.99% by weight of the composition, preferably from about 50% to about 99% by weight of the composition, and can, in the absence of other additives, form the balance of the composition.

As used herein the term “cosmetically acceptable medium” refers to formulations that are used to treat skin, hair and/or nails and contain one or more ingredients used by those skilled in the art to formulate products used to treat skin, hair and/or nails. The cosmetically acceptable medium can be in any suitable form, i.e., a liquid, cream, emulsion, gel, thickening lotion or powder and will typically contain water, and can contain a cosmetically acceptable solvent and/or one or more surfactants.

The skin care composition can further comprise the following basic cosmetic raw materials, including, but not limited to hydrocarbons, esters, fatty alcohols, fatty acids, emulsifying agents, humectants, viscosity modifiers, and silicone-based materials. The compositions of the present invention can contain a wide range of these basic components. The total concentration of added ingredients usually is less than 50%, preferably less than 20%, and most preferably less than 10% by weight of the total composition. Those skilled in the art will appreciate the various concentrations and combinations for employing these basic components to achieve the desired product form.

Suitable hydrocarbons which can be used in the compositions of the invention include, but are not limited to mineral oil, isohexadecane, squalane, hydrogenated polyisobutene, petrolatum, paraffin, microcrystalline wax, and polyethylene.

Suitable esters which can be used in the compositions of the invention include, but are not limited to isopropyl palmitate, octyl stearate, caprylic/capric triglyceride, plant waxes (Canelilla, Caranauba), vegetable oils (natural glycerides) and plant oils (Jojoba).

Suitable fatty alcohols which can be used in the compositions of the invention include, but are not limited to myristyl, cety, stearyl, isostearyl, and behenyl.

Suitable emulsifying agents which can be used in the compositions of the invention include, but are not limited to anionic (TEA/K stearate (triethanolamine/potassium stearate), sodium lauryl stearate, sodium cetearyl sulfate, and beeswax/Borax), nonionic (glycerol di-stearate, PEG (polyethyleneglycol)-100 Stearate, Polysorbate 20, steareth 2 and steareth 20), and cationic (distearyldimethylammonium chloride, behenalkonium chloride and steapyrium chloride), polymeric (acrylates/C 10-30 alkyl acrylate crosspolymer, polyacrylamide, polyquaternium-37, propylene glycol, dicaprylate/dicaparate and PPG-1 Trideceth-6), and silicone based materials (alkyl modified dimethicone copolyols), and polyglyceryl esters, and ethoxylated di-fatty esters.

Exemplary humectants for use in the compositions of the invention include, but are not limited to propylene glycol, sorbitol, butylene glycol, hexylene glycol, acetamide MEA (acetylethanolamine), honey, and sodium PCA (sodium-2-pyrrolidone carboxylate).

Viscosity modifiers, which can be used in the compositions of the invention include, but are not limited to xanthum gum, magnesium aluminum silicate, cellulose gum, and hydrogenated castor oil.

Further, the skin care compositions can comprise one or more conventional functional cosmetic or dermatological additives or adjuvants, providing that they do not interfere with the mildness, performance or aesthetic characteristics desired in the final products. The CTFA (The Cosmetic, Toiletry, and Fragrance Association; now known as the Personal Care Products Council) International Cosmetic Ingredient Dictionary and Handbook, Eleventh Edition (2006), and McCutcheon's Functional Materials, North America and Internationals Editions, MC Publishing Co. (2007) describe a wide variety of cosmetic and pharmaceutical ingredients commonly used in skin care compositions, which are suitable for use in the compositions of the present invention. The compositions of the present invention can contain a wide range of these additional, optional components. The total concentration of added ingredients usually is less than about 20%, preferably less than about 5%, and most preferably less than about 3% by weight of the total composition. Such components include, but are not limited to surfactants, emollients, moisturizers, stabilizers, film-forming substances, fragrances, colorants, chelating agents, preservatives, antioxidants, pH adjusting agents, antimicrobial agents, water-proofing agents, dry feel modifiers, vitamins, plant extracts, hydroxy acids (such as alpha-hydroxy acids and beta-hydroxy acids), and sunless tanning agents.

In some embodiments, the composition is a cream and further comprises: (i) Stearic acid, Mineral oil, Glyceryl monostearate, Glyceryl monohydroxystearate, Cetearyl alcohol, Cetyl octanoate, Emulsifying wax, Cabopol, Triethanolamine, Water, Glycerin, Propylene glycol, Fragrance, and Preservative; (ii) Stearic acid, Mineral oil, Glyceryl monostearate, Glyceryl monohydroxystearate, Cetearyl alcohol, Cetyl octanoate, Cabopol, Triethanolamine, Glycerin, Salicylic acid, Citrus Aurantium Dulcis (Orange) Fruit Extract, Fragrance, and Preservative; (iii) Emulsifying wax, Behentrimonium methosulfate and Cetearyl alcohol, PPG-3 myristyl ether, Cetearyl isononanoate, Dimethicone PEG-7 Isostearate, Ethylhexyl methoxycinnamate, Cabopol, Acrylates/Acrylamide Copolymer (and) Mineral Oil, (and) Polysorbate 85, Triethanolamine, Glycerin, Hyaluronic acid, Wheat amino acids, Lactamide MEA and acetamide MEA, Hydrolyzed silk protein, Salicylic acid, Propylene glycol, Pyrus Malus (Apple) Fruit Extract, Citrus Aurantium Dulcis (Orange) Fruit Extract, Tocopheryl acetate, Fragrance, and Preservative; or (iv) Glyceryl monohydroxystearate, Limnanthes Alba (Meadowfoam) Seed Oil, Prunus Armeniaca (Apricot) Kernel Oil, Phenyl trimethicone, Diisopropyl sebacate, Ethylhexyl methoxycinnamate, Cabopol, Acrylates/Acrylamide Copolymer (and) Mineral Oil, (and) Polysorbate 85, AMP-Acrylates/Allyl Methacrylate Copolymer, Triethanolamine, PEG-20 Almond Glycerides, Titanium dioxide, Propylene glycol, Linoleamidopropyl PG-dimonium chloride phosphate, Cocodimonium hydroxypropyl hydrolyzed wheat protein, Tocopheryl acetate, Fragrance, and Preservative.

In some embodiments, the composition can further comprise one or more ingredients to provide additional benefits, such as enhancing the antifungal properties of the composition. For example, the composition can comprise salicyclic acid, curcumin, and analogues, derivatives and salts thereof.

In some embodiments the composition is toothpaste. Toothpaste is generally a paste or gel used to clean and improve the health and aesthetic appearance of teeth. Used in conjunction with a toothbrush, toothpaste promotes oral hygiene by aiding the removal of dental plaque and food from the teeth, and often includes fluoride for prevention of tooth and gum disease. In addition to the particles disclosed herein, the tooth paste can comprise, konjac gum, agar, alginates, gelatin, pectin, xanthan, tara gum, gum arabic, carrageenan, celluloses, gellan gum, guar gum, inulin, konjac, locust bean gum, pectin, tragacanth, xanthan, polyethylene glycol-3350, xylitol, calcium carbonate, stevia, quillaja, liquid bioflavonoid extract, or any combinations thereof.

Some exemplary additives for including in a composition disclosed herein include 45° Be'glucose syrup, Acrylates/10-30 alkyl acrylate crosspolymer, Acrylates/Acrylamide Copolymer, agar, Allantoin, Aminomethyl propanol, Ammonium lauryl sulfate (ALS), Amodimethicone emulsion, AMP-Acrylates/Allyl Methacrylate Copolymer, Behentrimonium methosulfate, Benzophenone-4, Butylene glycol, Carbapol Aqua SF-1, Carbapol-934, Carbapol-940, Carbopol, Carboxymethyl cellulose, Cassia hydroxy propyltrimoniumchloride, Cetearyl alcohol, Cetearyl alcohol, Cetearyl isononanoate, Cetrimoniumchloride (CTC), Cetyl Alcohol, Cetyl octanoate, Chloromethyl/Methylisothiazolinone, Chloromethyl/Methylisothiazolinone, Citric acid, Citrus Aurantium Dulcis (Orange) Fruit Extract, Cocamidopropyl betain (CAPB), Cocamidopropyl betain (CAPB), Coco monoethanolamide (CMEA), Cocodimonium hydroxypropyl hydrolyzed wheat protein, Coloring agent, CPB, Diisopropyl sebacate, Dimethicone PEG-7 Isostearate, Disodium EDTA, DMDM hydantoin, Dove AD shine, Emulsifying wax, Ethanol, Ethylene glycol distearate (EGDS), Ethylhexyl methoxycinnamate, eucalyptol, Flavors, Forte therapy, Fragrance, Glycerin, Glycerine, Glyceryl monohydroxystearate, Glyceryl monostearate, Glycolic acid, Guar gum, Gum base, Hyaluronic acid, Hydrated silica, Hydrolyzed silk protein, Intense repair, Lactamide MEA acetamide MEA, Lactic acid, Limnanthes Alba (Meadowfoam) Seed Oil, Linalool, Linoleamidopropyl PG-dimonium chloride phosphate, Macrogolcetostearyl ether 20, magnese chloride, Magnesium sulfate, Menthol, Methyl gluceth-20, methyl salicylate, Mineral oil, Mint-type flavor, PEG/PPG-8/3 laurate, PEG-12 dimethicone, PEG-20 Almond Glycerides, PEG-40 hydrogenated castor oil, PEG-80 glyceryl cocoate, peppermint oil, Phenyl trimethicone, Polyacrylate-1 crosspolymer, Polyethylene glycol 1450, Polyoxyethylene/polyoxypropylene block polymer (Poloxamer 407), Polyquaternium-22, Polyquaternium-39, Polysorbate 85, polysorbate-20, PPG-3 myristyl ether, Preservative, Propylene glycol, Propylene glycol monocaprylate, Prunus Armeniaca (Apricot) Kernel Oil, Pyrus Malus (Apple) Fruit Extract, retinyl palmitate, Saccharin sodium, Salicylic acid, Sensomer CT-250, Silica Xerogel, SLES, SLS, sodium chloride, sodium docusate, Sodium hydroxide, Sodium lauryl ether sulfate (SLES), sodium lauryl sulphate, Sodium monofluorophosphate, Sodium saccharin, Sorbitol, Stearamidopropyldimethylamine, Steareth-2, Steareth-21, Stearic acid, Sugar powder, sunflower seed oil, Tea tree oil, Titanium dioxide, Tocopheryl acetate, Triclosan, Triethanolamine, Wheat amino acids, Xanthan gum, zinc carbonate, zinc chloride, zinc recinoleate, zinc stearate, and any combinations thereof.

### Exemplary Compositions Comprising the Particles

In some embodiments, the composition is a shampoo. Some exemplary shampoo compositions comprise particles comprising zinc pyrithinone or ketoconazole. In some embodiments, the shampoo composition further comprises:
(i) one or more of Carbopol, Ammonium lauryl sulfate (ALS), Sodium lauryl ether sulfate (SLES), Sodium hydroxide, Coco monoethanolamide (CMEA), Ethylene glycol distearate (EGDS), Propylene glycol monocaprylate, Menthol, Magnesium sulfate, Amodimethicone emulsion, Propylene glycol, Zinc carbonate, Cocamidopropyl betaine (CAPB), Cassia hydroxyl propyltrimoniumchloride, Chloromethyl/Methylisothiazolinone, Linalool, Fragrance, Citric acid, and Sodium Chloride; or
(ii) one or more of Carbopol, Ammonium lauryl sulfate (ALS), Sodium lauryl ether sulfate (SLES), Sodium hydroxide, Ethylene glycol distearate (EGDS), Propylene glycol monocaprylate, Menthol, Magnesium sulfate, Amodimethicone emulsion, Propylene glycol, Zinc carbonate, Cocamidopropyl betaine (CAPB), Cassia hydroxyl propyltrimoniumchloride, Chloromethyl/Methylisothiazolinone, Linalool, Fragrance, Citric acid, and Sodium Chloride.

In some embodiments, the composition is a conditioner. Some exemplary conditioner compositions comprise zinc pyrithione or ketoconazole as the active agent. In some embodiments, the conditioner composition further comprises:
(i) one or more of Carbopol, Sodium lauryl ether sulphate (SLES), Sodium hydroxide, Steareth-2, Steareth-21, Propylene glycol monocaprylate, Macrogolcetostearyl ether 20, Coco monoethanolamide (CMEA), Cetyl Alcohol, Stearamidopropyldimethylamine, Lactic acid, Cocamidopropyl betain (CAPB), Cetrimoniumchloride (CTC), Polyquaternium-22, Amodimethicone emulsion, Cassia hydroxy propyltrimoniumchloride, Propylene glycol, Glycerine, Zinc carbonate, Titanium dioxide, Linalool, Fragrance, and Chloromethyl/Methylisothiazolinone; or
(ii) one or more of Carbopol, Sodium lauryl ether sulphate (SLES), Sodium hydroxide, Steareth-2, Steareth-21, Propylene glycol monocaprylate, Macrogolcetostearyl ether 20, Coco monoethanolamide (CMEA), Cetyl Alcohol, Stearamidopropyldimethylamine, Lactic acid, Cocamidopropyl betain (CAPB), Cetrimoniumchloride (CTC), Polyquaternium-22, Amodimethicone emulsion, Cassia hydroxy propyltrimoniumchloride, Propylene glycol, Glycerine, Titanium dioxide, Linalool, Fragrance, and Chloromethyl/Methylisothiazolinone.

In some embodiments, the composition is a cream. Some exemplary cream compositions comprise ketoconazole, salicylic acid, curcuminoids or tetrahydro cucuminioids as the active agent. In some embodiments, the cream composition further comprises:
(i) one or more of Stearic acid, Mineral oil, Glyceryl monostearate, Glyceryl monohydroxystearate, Cetearyl alcohol, Cetyl octanoate, Emulsifying wax, Cabopol, Triethanolamine, Water, Glycerin, Propylene glycol, Fragrance, and Preservative;
(ii) one or more of Stearic acid, Mineral oil, Glyceryl monostearate, Glyceryl monohydroxystearate, Cetearyl alcohol, Cetyl octanoate, Cabopol, Triethanolamine, Glycerin, Salicylic acid, Citrus Aurantium Dulcis (Orange) Fruit Extract, Fragrance, and Preservative;
(iii) one or more of Emulsifying wax, Behentrimonium methosulfate and Cetearyl alcohol, PPG-3 myristyl ether, Cetearyl isononanoate, Dimethicone PEG-7 Isostearate, Ethylhexyl methoxycinnamate, Cabopol, Acrylates/Acrylamide Copolymer (and) Mineral Oil, (and) Polysorbate 85, Triethanolamine, Glycerin, Hyaluronic acid, Wheat amino acids, Lactamide MEA and acetamide MEA, Hydrolyzed silk protein, Salicylic acid, Propylene glycol, Pyrus Malus (Apple) Fruit Extract, Citrus Aurantium Dulcis (Orange) Fruit Extract, Tocopheryl acetate, Fragrance, and Preservative; or
(iv) one or more of Glyceryl monohydroxystearate, Limnanthes Alba (Meadowfoam) Seed Oil, Prunus Armeniaca (Apricot) Kernel Oil, Phenyl trimethicone, Diisopropyl sebacate, Ethylhexyl methoxycinnamate, Cabopol, Acrylates/Acrylamide Copolymer (and) Mineral Oil, (and) Polysorbate 85, AMP-Acrylates/Allyl Methacrylate Copolymer, Triethanolamine, PEG-20 Almond Glycerides, Titanium dioxide, Propylene glycol, Linoleamidopropyl PG-dimonium chloride phosphate, Cocodimonium hydroxypropyl hydrolyzed wheat protein, Tocopheryl acetate, Fragrance, and Preservative.

In some embodiments, the composition is a gel. Some exemplary gel compositions comprise salicylic acids, curcuminoids or tetrahydro cucuminioids, titanium dioxide, or chloroxylenol as the active agent. In some embodiments, the gel composition further comprises:
(i) one or more of Glycerin, Methyl gluceth-20, Benzophenone-4, Acrylates/10-30 alkyl acrylate crosspolymer, PEG/PPG-8/3 laurate, Aminomethyl propanol, Polyquaternium-39, PEG-80 glyceryl cocoate, Coloring agent, Preservative, and Fragrance;
(ii) one or more of Disodium EDTA, Propylene glycol, Carbopol, Aminomethyl propanol, Tea tree oil, PEG-40 hydrogenated castor oil, polysorbate-20, Coloring agent, Preservative, and Fragrance;
(iii) one or more of Butylene glycol, Glycerin, Methyl gluceth-20, Allantoin, Disodium EDTA, PEG-12 dimethicone, Polyacrylate-1 crosspolymer, Glycolic acid, Triethanolamine, Tocopheryl acetate, retinyl palmitate, sunflower seed oil, agar, Coloring agent, Preservative, and Fragrance; or
(iv) one or more of Glycerin, Methyl gluceth-20, Carbopol, Triethanolamine, Ethanol, Triclosan, Coloring agent, Preservative, and Fragrance.

In some embodiments, the composition is a toothpaste. An exemplary toothpaste composition comprises particles comprising titanium oxide as the active agent. In some embodiments, the tooth paste composition further comprises Carboxymethyl cellulose, Polyethylene glycol 1450, Sorbitol, Glycerin, Sodium monofluorophosphate, Sodium saccharin, Preservative, Coloring agent, Silica Xerogel, Hydrated silica, Mint-type flavor, and Sodium lauryl sulfate.

In some embodiments, the composition is mouth wash. An exemplary mouth wash composition comprises chloroxylenol as the active agent. In some embodiments, the mouth wash composition further comprises Ethyl alcohol, menthol, methyl salicylate, peppermint oil, eucalyptol, Glycerin, Polyoxyethylene/polyoxypropylene block polymer (Poloxamer 407), and Saccharin sodium.

### Some Exemplary Embodiments

Exemplary embodiments of the invention can be described by any one of the following numbered paragraphs.
1. A particle comprising: (i) a core comprising a first active agent; and (ii) a first coating layer comprising a lipid at least partially covering the core.
2. A particle comprising: (i) a core comprising a first active agent; and (ii) a first coating layer comprising a carbohydrate at least partially covering the core.
3. A particle comprising: (i) a core comprising a first active agent; and (ii) a first coating layer comprising a protein at least partially covering the core.
4. A particle comprising: (i) a core comprising a first active agent; and (ii) a first coating layer comprising a cationic molecule at least partially covering the core.
5. A particle comprising: (i) a core comprising a first active agent; and (ii) a first coating layer comprising at least two molecules selected from lipids, carbohydrates, proteins, and cationic molecules and the coating layer at least partially covering the core.
6. The particle of any of paragraphs 1-5, wherein the core further comprises a lipid, a carbohydrate, a protein, a cationic molecule, or any combinations thereof.
7. A particle comprising: (i) a core comprising a lipid, a carbohydrate, a protein, a cationic molecule, or any combinations thereof; (ii) and a first coating comprising a first active agent at least partially covering the core.
8. The particle of any of paragraphs 1-7, wherein the coating acts a food for a pathogen.
9. The particle of any of paragraphs 1-7, wherein the coating enhances targeting, binding, or retention of the active agent to a desired site of action.
10. The particle of any of paragraphs 1-9, wherein the coating has a synergistic effect on activity of the active agent.
11. The particle of any of paragraphs 1-10, wherein the coating acts as an active agent.
12. The particle of any of paragraphs 1-11, wherein the particle further comprises a second active agent.
13. The particle of any of paragraphs 1-12, wherein the second active agent is present in the core.
14. The particle of any of paragraphs 1-13, wherein the second active agent is in the coating layer.
15. The particle of any of paragraphs 1-14, wherein the second active agent forms a second coating layer on the first coating layer.
16. The particle of any of paragraphs 1-15, wherein the particle further comprises a third coating layer on the second coating layer.
17. The particle of any of paragraphs 1-16, wherein the third layer comprises a lipid, a protein, a polymer, a carbohydrate, or any combinations thereof.
18. The particle of any of paragraphs 1-17, wherein the particle further comprises at least one second layer on the first coating layer.
19. The particle of paragraph 1-18, wherein the second layer comprises a lipid, a protein, a polymer, a carbohydrate, or any combinations thereof.
20. The particle of any of paragraphs 1-19, wherein the particle further comprises at least two alternating coating layers, wherein one layer in the alternating coating layers comprises an active agent and another layer in the alternating coating layers comprises a lipid, a protein, a polymer, a carbohydrate, or any combinations thereof.
21. The particle of any of paragraphs 1-20, wherein the outermost layer comprises the active agent.
22. The particle of any of paragraphs 1-21, wherein the outermost layer comprises the lipid, the protein, the polymer, the carbohydrate, or any combinations thereof.
23. The particle of any of paragraphs 1-22, wherein the active agent is selected from the group consisting of small organic or inorganic molecules, saccharines, oligosaccharides, polysaccharides, peptides; proteins, peptide analogs and derivatives, peptidomimetics, nucleic acids, nucleic acid analogs and derivatives, antibodies, antigen binding fragments of antibodies, lipids, extracts made from biological materials, naturally occurring or synthetic compositions, and any combinations thereof.
24. The particle of any of paragraphs 1-23, wherein the active agent is selected from the group consisting of antifungal agents, antibacterial agents, antimicrobial agents, antioxidant agents, cooling agents, soothing agents, wound healing agents, anti-inflammatory-agents, anti-aging agents, anti-wrinkle agents, skin whitening or bleaching agents, ultraviolet (UV) light absorbing or scattering agents, skin depigmentation agents, dyes or coloring agents, deodorizing agents, fragrances, and any combinations thereof.
25. The particle of any of paragraphs 1-24, wherein the active agent is selected from the group consisting of pyrithione salts; ketoconazole; salicylic acid; curcumin or a derivative of curcumin, curcuminoids; tetrahydro curcuminoids; titanium dioxide (TiO₂); zinc oxide (ZnO); chloroxylenol; flvanoids; CoQ10; vitamin C; herbal extracts; alkaloids; 13-cis retinoic acid; 3,4-methylenedioxymethamphetamine; 5-fluorouracil; 6,8-dimercaptooctanoic acid (dihydrolipoic acid); abacavir; acebutolol; acetaminophen; acetaminosalol; acetazolamide; acetohydroxamic acid; acetylsalicylic acid; acitretin; aclovate; acrivastine; actiq; acyclovir; adapalene; adefovir dipivoxil; adenosine; Albaconazole; albuterol; alfuzosin; Allicin; allopurinol; alloxanthine; allylamines; almotriptan; alpha-hydroxy acids; alprazolam; alprenolol; aluminum acetate; aluminum chloride; aluminum chlorohydroxide; aluminum hydroxide; amantadine; amiloride; aminacrine; aminobenzoic acid (PABA); aminocaproic acid; aminoglycosides such as streptomycin, neomycin, kanamycin, paromycin, gentamicin, tobramycin, amikacin, netilmicin, spectinomycin, sisomicin, dibekalin and isepamicin; aminosalicylic acid; amiodarone; amitriptyline; amlodipine; amocarzine; amodiaquin; Amorolfin; amoxapine; amphetamine; amphotericin B; ampicillin; anagrelide; anastrozole; Anidulafungin; anthralin; antibacterial sulfonamides and antibacterial sulphanilamides, including para-aminobenzoic acid, sulfadiazine, sulfisoxazole, sulfamethoxazole and sulfathalidine; antifungal peptide and derivatives and analogs thereof; apomorphine; aprepitant; arbutin; aripiprazole; ascorbic acid; ascorbyl palmitate; atazanavir; atenolol; atomoxetine; atropine; azathioprine; azelaic acid; azelaic acid; azelastine; azithromycin; bacitracin; bacitracin; beanomicins; beclomefhasone dipropionate; bemegride; benazepril; bendroflumethiazide; benzocaine; Benzoic acid with a keratolytic agent; benzonatate; benzophenone; benztropine; bepridil; beta-hydroxy acids; beta-lactams including penicillin, cephalosporin, and carbapenems such as carbapenem, imipenem, and meropenem; betamethasone dipropionate; betamethasone valerate; brimonidine; brompheniramine; bupivacaine; buprenorphine; bupropion; burimamide; butenafine; Butenafine; butoconazole; Butoconazole; cabergoline; caffeic acid; caffeine; calcipotriene; camphor; Cancidas; candesartan cilexetil; capsaicin; carbamazepine; Caspofungin; cefditoren pivoxil; cefepime; cefpodoxime proxetil; celecoxib; cetirizine; cevimeline; chitosan; chlordiazepoxide; chlorhexidine; chloroquine; chlorothiazide; chloroxylenol; chlorpheniramine; chlorpromazine; chlorpropamide; ciclopirox; Ciclopirox (ciclopirox olamine); cilostazol; cimetidine; cinacalcet; ciprofloxacin; citalopram; citric acid; Citronella oil; cladribine; clarithromycin; clemastine; clindamycin; clioquinol; clobetasol propionate; clomiphene; clonidine; clopidogrel; Clortrimazole; clotrimazole; Clotrimazole; clozapine; cocaine; Coconut oil; codeine; colistin; colymycin; cromolyn; crotamiton; Crystal violet; cyclizine; cyclobenzaprine; cycloserine; cytarabine; dacarbazine; dalfopristin; dapsone; daptomycin; daunorubicin; deferoxamine; dehydroepiandrosterone; delavirdine; desipramine; desloratadine; desmopressin; desoximetasone; dexamethasone; dexmedetomidine; dexmethylphenidate; dexrazoxane; dextroamphetamine; diazepam; dicyclomine; didanosine; dihydrocodeine; dihydromorphine; diltiazem; diphenhydramine; diphenoxylate; dipyridamole; disopyramide; dobutamine; dofetilide; dolasetron; donepezil; dopa esters; dopamine; dopamnide; dorzolamide; doxepin; doxorubicin; doxycycline; doxylamine; doxypin; duloxetine; dyclonine; echinocandins; econazole; Econazole; eflormthine; eletriptan; emtricitabine; enalapril; ephedrine; epinephrine; epinine; epirubicin; eptifibatide; ergotarnine; erythromycin; escitalopram; esmolol; esomeprazole; estazolam; estradiol; ethacrynic acid; ethinyl estradiol; etidocaine; etomidate; famciclovir; famotidine; felodipine; fentanyl; Fenticonazole; ferulic acid; fexofenadine; flecainide; fluconazole; Fluconazole; flucytosiine; Flucytosine or 5-fluorocytosine; fluocinolone acetonide; fluocinonide; fluoxetine; fluphenazine; flurazepam; fluvoxamine; formoterol; furosemide; galactarolactone; galactonic acid; galactonolactone; galactose; galantamine; gatifloxacin; gefitinib; gemcitabine; gemifloxacin; gluconic acid; glycolic acid; glycolic acid; glycopeptides such as vancomycin and teicoplanin; griseofulvin; Griseofulvin; guaifenesin; guanethidine; haloperidol; haloprogin; Haloprogin; herbal extract, an alkaloid, a flvanoid, Abafungin; hexylresorcinol; homatropine; homosalate; hydralazine; hydrochlorothiazide; hydrocortisone; hydrocortisone 17-butyrate; hydrocortisone 17-valerate; hydrocortisone 21-acetate; hydromorphone; hydroquinone; hydroquinone monoether; hydroxyzine; hyoscyamine; hypoxanthine; ibuprofen; ichthammol; idarubicin; imatinib; imipramine; imiquimod; indinavir; indomethacin; Iodine; irbesartan; irinotecan; Isavuconazole; Isoconazole; isoetharine; isoproterenol; itraconazole; Itraconazole; kanamycin; ketamine; ketanserin; ketoconazole; ketoprofen; ketotifen; kojic acid; labetalol; lactic acid; lactobionic acid; lactobionic acid; lamivudine; lamotrigine; lansoprazole; lemon myrtle; letrozole; leuprolide; levalbuterol; levofloxacin; lidocaine; lincosamides such as lincomycin and clindamycin; linezolid; lobeline; loperamide; losartan; loxapine; lucensomycin; lysergic diethylamide; macrolides or ketolides such as erythromycin, azithromycin, clarithromycin, and telithromycin; mafenide; malic acid; maltobionic acid; mandelic acid; mandelic acid; maprotiline; mebendazole; mecamylamine; meclizine; meclocycline; memantine; menthol; meperidine; mepivacaine; mercaptopurine; mescaline; metanephrine; metaproterenol; metaraminol; metformin; methadone; methamphetamine; methotrexate; methoxamine; methyl nicotinate; methyl salicylate; methyldopa esters; methyldopamide; methyllactic acid; methylphenidate; metiamide; metolazone; metoprolol; metronidazole; mexiletine; Micafungin; miconazole; Miconazole; midazolam; midodrine; miglustat; minocycline; minoxidil; mirtazapine; mitoxantrone; moexiprilat; molindone; monobenzone; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, and astreonam; morphine; moxifloxacin; moxonidine; mupirocin; nadolol; naftifine; Naftifine; nalbuphine; nalmefene; naloxone; naproxen; natamycin; Neem Seed Oil; nefazodone; nelfinavir; neomycin; nevirapine; N-guanylhistamine; nicardipine; nicotine; nifedipine; nikkomycins; nimodipine; nisoldipine; nizatidine; norepinephrine; nystatin; nystatin; octopamine; octreotide; octyl methoxycinnamate; octyl salicylate; ofloxacin; olanzapine; Olive leaf extract; olmesartan medoxomil; olopatadine; omeprazole; Omoconazole; ondansetron; Orange oil; oxazolidinones such as linezolid; oxiconazole; Oxiconazole; oxotremorine; oxybenzone; oxybutynin; oxycodone; oxymetazoline; padimate O; palmarosa oil; palonosetron; pantothenic acid; pantoyl lactone; paroxetine; patchouli; pemoline; penciclovir; penicillamine; penicillins; pentazocine; pentobarbital; pentostatin; pentoxifylline; pergolide; perindopril; permethrin; phencyclidine; phenelzine; pheniramine; phenmetrazine; phenobarbital; phenol; phenoxybenzamine; phenpropimorph; phentolamine; phenylephrine; phenylpropanolamine; phenytoin; phosphonomycin; physostigmine; pilocarpine; pimozide; pindolol; pioglitazone; pipamazine; piperonyl butoxide; pirenzepine; Piroctone; piroctone olamine; podofilox; podophyllin; Polygodial; polyhydroxy acids; polymyxin; Posaconazole; pradimicins; pramoxine; pratipexole; prazosin; prednisone; prenalterol; prilocaine; procainamide; procaine; procarbazine; promazine; promethazine; promethazine propionate; propafenone; propoxyphene; propranolol; propylthiouracil; protriptyline; pseudoephedrine; pyrethrin; pyrilamine; pyrimethamine; quetiapine; quinapril; quinethazone; quinidine; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, besifloxacin, besifloxaxin, clintafloxacin, ganefloxacin, gemifloxacin and pazufloxacin; quinupristin; rabeprazole; Ravuconazole; reserpine; resorcinol; retinal; retinoic acid; retinol; retinyl acetate; retinyl palmitate; ribavirin; ribonic acid; ribonolactone; rifampin; rifamycins such as rifampicin (also called rifampin), rifapentine, rifabutin, bezoxazinorifamycin and rifaximin; rifapentine; rifaximin; riluzole; rimantadine; risedronic acid; risperidone; ritodrine; rivasfigmine; rizatriptan; ropinirole; ropivacaine; salicylamide; salicylic acid; salicylic acid; salmeterol; scopolamine; selegiline; Selenium; selenium sulfide; serotonin; Sertaconazole; sertindole; sertraline; sibutramine; sildenafil; sordarins; sotalol; streptogramins such as quinupristin and daflopristin; streptomycin; strychnine; sulconazole; Sulconazole; sulfabenz; sulfabenzamide; sulfabromomethazine; sulfacetamide; sulfachlorpyridazine; sulfacytine; sulfadiazine; sulfadimethoxine; sulfadoxine; sulfaguanole; sulfalene; sulfamethizole; sulfamethoxazole; sulfanilamide; sulfapyrazine; sulfapyridine; sulfasalazine; sulfasomizole; sulfathiazole; sulfisoxazole; tadalafil; tamsulosin; tartaric acid; tazarotene; Tea tree oil—ISO 4730 (“Oil of Melaleuca, Terpinen-4-ol type”); tegaserol; telithromycin; telmisartan; temozolomide; tenofovir disoproxil; terazosin; terbinafine; Terbinafine; terbutaline; terconazole; Terconazole; terfenadine; tetracaine; tetracycline; tetracyclines such as tetracycline, chlortetracycline, demeclocycline, minocycline, oxytetracycline, methacycline, doxycycline; tetrahydrozoline; theobromine; theophylline; thiabendazole; thioridazine; thiothixene; thymol; tiagabine; timolol; tinidazole; tioconazole; Tioconazole; tirofiban; tizanidine; tobramycin; tocainide; tolazoline; tolbutamide; tolnaftate; Tolnaftate; tolterodine; tramadol; tranylcypromine; trazodone; triamcinolone acetonide; triamcinolone diacetate; triamcinolone hexacetonide; triamterene; triazolam; triclosan; triclosan; Triclosan; triflupromazine; trimethoprim; trimethoprim; trimipramine; tripelennamine; triprolidine; tromethamine; tropic acid; tyramine; undecylenic acid; Undecylenic acid; urea; urocanic acid; ursodiol; vardenafil; venlafaxine; verapamil; vitamin C; vitamin E acetate; voriconazole; Voriconazole; warfarin; xanthine; zafirlukast; zaleplon; zinc pyrithione; Zinc Selenium sulfide; ziprasidone; zolmitriptan; Zolpidem; WS-3; WS-23; menthol; 3-substituted-P-menthanes; N-substituted-P-menthane-3-carboxamides; isopulegol; 3-(1-menthoxy)propane-1,2-diol; 3-(1-menthoxy)-2-methylpropane-1,2-diol; p-menthane-2,3-diol; p-menthane-3,8-diol; 6-isopropyl-9-methyl-1,4-dioxaspiro[4,5]decane-2-methanol; menthyl succinate and its alkaline earth metal salts; trimethylcyclohexanol; N-ethyl-2-isopropyl-5-methylcyclohexanecarboxamide; Japanese mint oil; peppermint oil; menthone; menthone glycerol ketal; menthyl lactate; 3-(1-menthoxy)ethan-1-ol; 3-(1-menthoxy)propan-1-ol; 3-(1-menthoxy)butan-1-ol; 1-menthylacetic acid N-ethylamide; 1-menthyl-4-hydroxypentanoate; 1-menthyl-3-hydroxybutyrate; N,2,3-trimethyl-2-(1-methylethyl)-butanamide; n-ethyl-t-2-c-6 nonadienamide; N,N-dimethyl menthyl succinamide; menthyl pyrrolidone carboxylate; aloe; avocado oil; green tea extract; hops extract; chamomile extract; colloidal oatmeal; calamine; cucumber extract; sodium palmate; sodium palm kernelate; butyrospermum parkii (i.e., shea butter); menthe piperita (i.e.; peppermint) leaf oil; sericin; pyridoxine (a form of vitamin B6); retinyl palmitate and/or other forms of vitamin A; tocopheryl acetate and/or other forms of vitamin E; lauryl laurate; hyaluronic acid; aloe barbadensis leaf juice powder; euterpe oleracea (i.e., acai berry) fruit extract; riboflavin (i.e., vitamin B2); thiamin HCl and/or other forms of vitamin B1; ethylenediaminetetraacetic acid (EDTA); citrate; ethylene glycol tetraacetic acid (EGTA); 1,2-bis(o-aminophenoxy)ethane-N,N,N′,N′-tetraacetic acid (BAPTA); diethylene triamine pentaacetic acid (DTPA); 2,3-dimercapto-1-propanesulfonic acid (DMPS); dimercaptosuccinic acid (DMSA); α-lipoic acid; salicylaldehyde isonicotinoyl hydrazone (SIH); hexyl thioethylamine hydrochloride (HTA); desferrioxamine; ascorbic acid (vitamin C); cysteine; glutathione; dihydrolipoic acid; 2-mercaptoethane sulfonic acid; 2-mercaptobenzimidazole sulfonic acid; 6-hydroxy-2,5,7,8-tetramethylchroman-2-carboxylic acid; sodium metabisulfite; vitamin E isomers such as α-, β-, γ-, and δ-tocopherols and α-, β-, γ-, and δ-tocotrienols; polyphenols such as 2-tert-butyl-4-methyl phenol, 2-tert-butyl-5-methyl phenol, and 2-tert-butyl-6-methyl phenol; butylated hydroxyanisole (BHA) such as 2-tert-butyl-4-hydroxyanisole and 3-tert-butyl-4-hydroxyanisole; butylhydroxytoluene (BHT); tert-butylhydroquinone (TBHQ); ascorbyl palmitate; n-propyl gallate; soy extract; soy isoflavones; retinoids such as retinol; kojic acid; kojic dipalmitate; hydroquinone; arbutin; transexamic acid; vitamins such as niacin and vitamin C; azelaic acid; linolenic acid and linoleic acid; placertia; licorice; and extracts such as chamomile and green tea; hydrogen peroxide; zinc peroxide; sodium peroxide; hydroquinone; 4-isopropylcatechol; hydroquinone monobenzyl ether; kojic acid; lactic acid; ascorbyl acid and derivatives such as magnesium ascorbyl phosphate; arbutin; licorice root; dihydroxyacetone (DHA); glyceryl aldehyde; tyrosine and tyrosine derivatives such as malyltyrosine, tyrosine glucosinate, and ethyl tyrosine; phospho-DOPA; indoles and derivatives; glucosamine; N-acetyl glucosamine; glucosamine sulfate; mannosamine; N-acetyl mannosamine; galactosamine; N-acetyl galactosamine; N-acyl amino acid compounds (e.g., N-undecylenoyl-L-phenylalanine); flavonoids such as quercetin, hesperidin, quercitrin, rutin, tangeritin, and epicatechin; CoQ10; vitamin C; hydroxy acids including C₂-C₃₀ alpha-hydroxy acids such as glycolic acid, lactic acid, 2-hydroxy butanoic acid, malic acid, citric acid tartaric acid, alpha-hydroxyethanoic acid, hydroxycaprylic acid and the like; beta hydroxy acids including salicylic acid and polyhydroxy acids including gluconolactone (G4); retinoic acid; gamma-linolenic acid; ultraviolet absorber of benzoic acid system such as para-aminobenzoic acid (hereinafter, abbreviated as PABA), PABA monoglycerin ester, N,N-dipropoxy PABA ethyl ester, N,N-diethoxy PABA ethyl ester, N,N-dimethyl PABA ethyl ester, N,N-dimethyl PABA butyl ester, and N,N-dimethyl PABA methyl ester and the like; ultraviolet absorber of anthranilic acid system such as homomenthyl-N-acetyl anthranilate and the like; ultraviolet absorber of salicylic acid system such as amyl salicylate, menthyl salicylate, homomenthyl salicylate, octyl salicylate, phenyl salicylate, benzyl salicylate, p-isopropanol phenyl salicylate and the like; ultraviolet absorber of cinnamic acid system such as octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxy cinnamate, isopropyl-p-methoxy cinnamate, isoamyl-p-methoxy cinnamate, octyl-p-methoxy cinnamate(2-ethylhexyl-p-methoxy cinnamate), 2-ethoxyethyl-p-methoxy cinnamate, cyclohexyl-p-methoxy cinnamate, ethyl-α-cyano-β-phenyl cinnamate, 2-ethylhexyl-α-cyano-β-phenyl cinnamate, glyceryl mono-2-ethylhexanoyl-dipara-methoxy cinnamate, methyl bis(trimethylsiloxane)silylisopentyl trimethoxy cinnamate and the like; 3-(4′-methylbenzylidene)-d,l-camphor; 3-benzylidene-d,l-camphor; urocanic acid, urocanic acid ethyl ester; 2-phenyl-5-methylbenzoxazole; 2,2′-hydroxy-5-methylphenylbenzotriazole; 2-(2′-hydroxy-5′-t-octylphenyl)benzotriazole; 2-(2′-hydroxy-5′-methylphenylbenzotriazole; dibenzaladine; dianisoylmethane; 4-methoxy-4′-t-butyldibenzoylmethane; 5-(3,3-dimethyl-2-norbornylidene)-3-pentane-2-one; dimorpholinopyridazinone; titanium oxide; particulate titanium oxide; zinc oxide; particulate zinc oxide; ferric oxide; particulate ferric oxide; ceric oxide; inorganic sunscreens such as titanium dioxide and zinc oxide; organic sunscreens such as octyl-methyl cinnamates and derivatives thereof; retinoids; vitamins such as vitamin E, vitamin A, vitamin C (ascorbic acid), vitamin B, and derivatives thereof such as vitamin E acetate, vitamin C palmitate, and the like; antioxidants including alpha hydroxy acid such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucopehtonic acid, glucopheptono-1,4-lactone, gluconic acid, gluconolactone, glucuronic acid, glucurronolactone, glycolic acid, isopropyl pyruvate, methyl pyruvate, mucic acid, pyruvia acid, saccharic acid, saccaric acid 1,4-lactone, tartaric acid, and tartronic acid; beta hydroxy acids such as beta-hydroxybutyric acid, beta-phenyl-lactic acid, beta-phenylpyruvic acid; botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, rice, and safflower; 21-acetoxypregnenolone; alclometasone; algestone; amcinonide; beclomethasone; betamethasone; budesonide; chloroprednisone; clobetasol; clobetansone; clocortolone; cloprednol; corticosterone; cortisone; cortivazol; deflazacort; desonide; desoximetasone; dexamethasone; diflorasone; diflucortolone; difluprednate; enoxolone; fluazacort; flucloronide; flumethasone flunisolide; fluocinolone acetonide; fluocinonide; fluocortin butyl; fluocortolone; fluorometholone; fluperolone acetate; fluprednidene acetate; fluprednisolone; flurandrenolide; fluticasone propionate; formocortal; halcinonide; halobetasol propionate; halometasone; halopredone acetate; hydrocortamate; hydrocortisone; loteprednol etabonate; mazipredone; medrysone; meprednisone; methylprednisolone; mometasone furcate; paramethosone; prednicarbate; prednisolone; prednisolone 25-diethylamino-acetate; prednisolone sodium phosphate; prednisone; prednival; prednylidene; rimexolone; tixocortol; triamcinolone; triamcinolone acetonide; triamcinolone benetonide; triamcinolone hexacetonide; COX inhibitors such as salicylic acid derivatives (e.g., aspirin, sodium salicylate, choline magnesium trisalicylate, salicylate, diflunisal, sulfasalazine and olsalazine); para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam); alkanones such as nabumetone; diarylsubstituted furanones such as refecoxib; diaryl-substituted pyrazoles such as celecoxib; indole acetic acids such as etodolac; sulfonanilides such as nimesulide; selenium sulfide; sulfur; sulfonated shale oil; salicylic acid; coal tar; povidone-iodine, imidazoles such as ketoconazole, dichlorophenyl imidazolodioxalan, clotrimazole, itraconazoie, miconazole, climbazole, tioconazole, sulconazole, butoconazole, fluconazole, miconazolenitrite; anthralin; piroctone olamine (Octopirox); ciclopirox olamine; anti-psoriasis agents; vitamin A analogs; corticosteroids; and any combinations thereof.
26. The particle of any of paragraphs 1-25, wherein the pyrithione salt is selected from the group consisting of zinc pyrithione, sodium pyrithione, potassium pyrithione, lithium pyrithione, ammonium pyrithione, copper pyrithione, calcium pyrithione, magnesium pyrithione, strontium pyrithione, silver pyrithione, gold pyrithione, manganese pyrithione, and any combinations thereof.
27. The particle of any of paragraphs 1-26, wherein the active agent is besifloxacin.
28. The particle of any of paragraphs 1-27, wherein the particle has a size of from about 5 nm to about 20 μm.
29. The particle of any of paragraphs 1-28, wherein the particle has a size of from about 100 nm to about 10 μm.
30. The particle of any of paragraphs 1-29, wherein the particle has a size of from about 200 nm to about 6 μm.
31. The particle of any of paragraphs 1-30, wherein the particle has a size of from about 1 μm to about 6 μm.
32. The particle of any of paragraphs 1-31, wherein the particle has a size of from about 300 nm to about 700 nm.
33. The particle of any of paragraphs 1-32, wherein the coating layer has a thickness of: (i) from about 1 nm to about 1000 nm.
34. The particle of any of paragraphs 1-33, wherein the coating layer has a thickness of from about 1 nm to about 150 nm.
35. The particle of any of paragraphs 1-34, wherein the active agent is present in an amount from about 1% to about 99% (w/w).
36. The particle of any of paragraphs 1-35, wherein the active agent is present in an amount from about 75% to about 98% (w/w).
37. The particle of any of paragraphs 1-36, wherein the lipid is selected from the group consisting of fatty acids; mono-, di- or tri-esters of fatty acids; salts of fatty acids; fatty alcohols; mono-, di- or tri-esters of fatty alcohols; glycerolipids; phospholipids; glycerophospholipids; sphingolipids; sterol lipids; prenol lipids; saccharolipids; polyketides; and any combination thereof.
38. The particle of any of paragraphs 1-37, wherein the lipid is selected from the group consisting of 1,3-Propanediol Dicaprylate/Dicaprate; 10-undecenoic acid; 1-dotriacontanol; 1-heptacosanol; 1-nonacosanol; 2-ethyl hexanol; Androstanes; Arachidic acid; Arachidonic acid; arachidyl alcohol; Behenic acid; behenyl alcohol; Capmul MCM C10; Capric acid; capric alcohol; capryl alcohol; Caprylic acid; Caprylic/Capric Acid Ester of Saturated Fatty Alcohol C12-C18; Caprylic/Capric Triglyceride; Caprylic/Capric Triglyceride; Ceramide phosphorylcholine (Sphingomyelin, SPH); Ceramide phosphorylethanolamine (Sphingomyelin, Cer-PE); Ceramide phosphorylglycerol; Ceroplastic acid; Cerotic acid; Cerotic acid; ceryl alcohol; Cetearyl alcohol; Ceteth-10; cetyl alcohol; Cholanes; Cholestanes; cholesterol; cis-11-eicosenoic acid; cis-11-octadecenoic acid; cis-13-docosenoic acid; cluytyl alcohol; coenzyme Q10 (CoQ10); Dihomo-γ-linolenic; Docosahexaenoic acid; egg lecithin; Eicosapentaenoic acid; Eicosenoic acid; Elaidic acid; elaidolinolenyl alcohol; elaidolinoleyl alcohol; elaidyl alcohol; Erucic acid; erucyl alcohol; Estranes; Geddic acid; geddyl alcohol; glycerol distearate (type I) EP (Precirol ATO 5); Glycerol Tricaprylate/Caprate; Glycerol Tricaprylate/Caprate (CAPTEX® 355 EP/NF); glyceryl monocaprylate (Capmul MCM C8 EP); Glyceryl Triacetate; Glyceryl Tricaprylate; Glyceryl Tricaprylate/Caprate/Laurate; Glyceryl Tricaprylate/Tricaprate; glyceryl tripalmitate (Tripalmitin); Henatriacontylic acid; Heneicosyl alcohol; Heneicosylic acid; Heptacosylic acid; Heptadecanoic acid; Heptadecyl alcohol; Hexatriacontylic acid; isostearic acid; isostearyl alcohol; Lacceroic acid; Lauric acid; Lauryl alcohol; Lignoceric acid; lignoceryl alcohol; Linoelaidic acid; Linoleic acid; linolenyl alcohol; linoleyl alcohol; Margaric acid; Mead; Melissic acid; melissyl alcohol; Montanic acid; montanyl alcohol; myricyl alcohol; Myristic acid; Myristoleic acid; Myristyl alcohol; neodecanoic acid; neoheptanoic acid; neononanoic acid; Nervonic; Nonacosylic acid; Nonadecyl alcohol; Nonadecylic acid; Nonadecylic acid; Oleic acid; oleyl alcohol; Palmitic acid; Palmitoleic acid; palmitoleyl alcohol; Pelargonic acid; pelargonic alcohol; Pentacosylic acid; Pentadecyl alcohol; Pentadecylic acid; Phosphatidic acid (phosphatidate, PA); Phosphatidylcholine (lecithin, PC); Phosphatidylethanolamine (cephalin, PE); Phosphatidylinositol (PI); Phosphatidylinositol bisphosphate (PIP2); Phosphatidylinositol phosphate (PIP); Phosphatidylinositol triphosphate (PIP3); Phosphatidylserine (PS); polyglyceryl-6-distearate; Pregnanes; Propylene Glycol Dicaprate; Propylene Glycol Dicaprylocaprate; Propylene Glycol Dicaprylocaprate; Psyllic acid; recinoleaic acid; recinoleyl alcohol; Sapienic acid; soy lecithin; Stearic acid; Stearidonic; stearyl alcohol; Tricosylic acid; Tridecyl alcohol; Tridecylic acid; Triolein; Undecyl alcohol; undecylenic acid; Undecylic acid; Vaccenic acid; α-Linolenic acid; γ-Linolenic acid; a fatty acid salt of 10-undecenoic acid, adapalene, arachidic acid, arachidonic acid, behenic acid, butyric acid, capric acid, caprylic acid, cerotic acid, cis-11-eicosenoic acid, cis-11-octadecenoic acid, cis-13-docosenoic acid, docosahexaenoic acid, eicosapentaenoic acid, elaidic acid, erucic acid, heneicosylic acid, heptacosylic acid, heptadecanoic acid, isostearic acid, lauric acid, lignoceric acid, linoelaidic acid, linoleic acid, montanic acid, myristic acid, myristoleic acid, neodecanoic acid, neoheptanoic acid, neononanoic acid, nonadecylic acid, oleic acid, palmitic acid, palmitoleic acid, pelargonic acid, pentacosylic acid, pentadecylic acid, recinoleaic acid, sapienic acid, stearic acid, tricosylic acid, tridecylic acid, undecylenic acid, undecylic acid, vaccenic acid, valeric acid, α-linolenic acid, or γ-linolenic acid; paraffin; and any combinations thereof.
39. The particle any of paragraphs 1-38, wherein the fatty acid salt is selected from the group consisting of zinc, sodium, potassium, lithium, ammonium, copper, calcium, magnesium, strontium, manganese, and combinations thereof.
40. The particle of any of paragraphs 1-39, wherein the lipid comprises 11 or fewer carbon atoms.
41. The particle of any of paragraphs 1-40, wherein the lipid is selected from the group consisting of ethylene glycol distearate (EGDS), caprylic acid, capric acid, lauric acid, myristic acid, palmitic acid, zinc recinoleate, CoQ10, paraffin, triplamitin, polyglyceryl-6-distearate, and any combinations thereof.
42. The particle of any of paragraphs 1-41, wherein the lipid is present in an amount from about 1% to about 99% (w/w).
43. The particle of any of paragraphs 1-42, wherein the lipid is present in an amount from about 2% to about 25% (w/w).
44. The particle of any of paragraphs 1-43, wherein the particle comprises an excess of the active agent relative to the total lipids.
45. The particle of any of paragraphs 1-44, wherein ratio of total lipid to the active agent in the particle is from about 100:1 to about 1:100.
46. The particle of any of paragraphs 1-45, wherein ratio of total lipid to the active agent in the particle is from about 100:1 to about 1:100.
47. The particle of any of paragraphs 1-46, wherein the ratio of total lipid to the active agent in the particle is from about 10:1 to about 1:50.
48. The particle of any of paragraphs 1-47, wherein ratio of total lipid to the active agent in the particle is about 2:1 to about 1:30.
49. The particle of any of paragraphs 1-48, wherein the protein is selected from the group consisting of Actin, Albumin, Amaranth Protein, Ammonium Hydrolyzed Animal Protein, Animal protein, Barley Protein, Brazil Nut Protein, Casein, Collagen, Collagen protein hydrolyzed, Conchiolin Protein, corn protein, Cottonseed Protein, Elastin, Extensin, Fibroin, Fibronectin, Fish Protein, Gadidae Protein, Gelatin, Glutein, Glycoproteins, Hazelnut Protein, Hemoglobin, Hemp Seed Protein, Honey Protein, Hydrolyzed Actin, Hydrolyzed Amaranth Protein, Hydrolyzed animal protein, Hydrolyzed Barley Protein, Hydrolyzed Brazil Nut Protein, Hydrolyzed Conchiolin Protein, Hydrolyzed corn protein, Hydrolyzed Cottonseed Protein, Hydrolyzed Elastin, Hydrolyzed Extensin, Hydrolyzed Fibroin, Hydrolyzed Fibronectin, Hydrolyzed Fish Protein, Hydrolyzed Gadidae Protein, Hydrolyzed Gadidae Protein, Hydrolyzed Gelatin, Hydrolyzed Hair Keratin, Hydrolyzed Hazelnut, Hydrolyzed Hazelnut Protein, Hydrolyzed Hemoglobin, Hydrolyzed Hemp Seed Protein, Hydrolyzed Honey Protein, Hydrolyzed Keratin, Hydrolyzed Lupine Protein, Hydrolyzed Maple Sycamore Protein, Hydrolyzed Milk Protein, Hydrolyzed Oat Protein, Hydrolyzed Pea Protein, Hydrolyzed Potato Protein, Hydrolyzed Reticulin, Hydrolyzed Royal Jelly Protein, Hydrolyzed Sericin, Hydrolyzed Serum Protein, Hydrolyzed Sesame Protein, Hydrolyzed Soy Protein, Hydrolyzed Soymilk Protein, Hydrolyzed Spinal Protein, Hydrolyzed Spongin, Hydrolyzed Sweet Almond Protein, Hydrolyzed Vegetable Protein, Hydrolyzed Wheat Gluten, Hydrolyzed Wheat Protein, Hydrolyzed Whey Protein, Hydrolyzed Yeast Protein, Hydrolyzed Yogurt Protein, Hydrolyzed Zein, Integrin, Jojoba protein HP, Hydrolyzed, keratin, Lupine Protein, Maple Sycamore Protein, MEA-Hydrolyzed Collagen, MEA-Hydrolyzed Silk, Milk Protein, Myosin, Oat Protein, Pea Protein, polylysine, Potato Protein, Reticulin, Rice Quat, Royal Jelly Protein, Sericin, Serum Protein, Sesame Protein, Silk powder, Sodium Hydrolyzed Casein, Soy Protein, Soy Rice Peptides, Soymilk Protein, Spinal Protein, Spongin, Sweet Almond Protein, Vegetable Protein, Wheat Gluten, Whey Protein, Yeast Protein, Yogurt Protein, Zein, and Zinc Hydrolyzed Collagen.
50. The particle of any of paragraphs 1-49, wherein the albumin is bovine serum albumin, egg albumin, Hydrolyzed Lactalbumin, or Lactalbumin.
51. The particle of any of paragraphs 1-50, wherein the protein is present in an amount from about 1% (w/w) to about 99% (w/w).
52. The particle of any of paragraphs 1-51, wherein the protein is present in an amount from about 5% (w/w) to about 50% (w/w).
53. The particle of any of paragraphs 1-52, wherein ratio of the protein to the active agent in the particle is from about 100:1 to about 1:100.
54. The particle of any of paragraphs 1-53, wherein ratio of the protein to the active agent in the particle is about 10:1 to about 1:50.
55. The particle of any of paragraphs 1-54, wherein ratio of the protein to the active agent in the particle is about 2:1 to about 1:30.
56. The particle of any of paragraphs 1-55, wherein the cationic molecule is a polyamine.
57. The particle of any of paragraphs 1-56, wherein the cationic molecule is selected from the group consisting of Putrescine (Butane-1,4-diamine), Cadaverine (Pentane-1,5-diamine), Spermidine, Spermine, Cyclen (1,4,7,10-tetrazacyclododecane), Cyclam (1,4,8,11-Tetraazacyclotetradecane), Linear Polyethyleneimine (Poly(iminoethylene)), Norspermidine, p-Phenylenediamine (1,4-diaminobenzene), Diethylenetriamine (N-(2-aminoethyl)-1,2-ethanediamine), thermospermine, Tris(2-aminoethyl)amine, Hexamethylenediamine, Beta-lysine (3,6-diaminohexanoic acid), m-Phenylenediamine (1,3-diaminobenzene), Diaminopropane (1,2-Diaminopropane), Ethylenediamine dihydroiodide, and polyamine D 400 (Polyoxyalkyleneamine D 400).
58. The particle of any of paragraphs 1-57, wherein the cationic molecule is present in an amount from about 1% (w/w) to about 99% (w/w).
59. The particle of any of paragraphs 1-58, wherein the cationic molecule is present in an amount from about 5% (w/w) to about 50% (w/w).
60. The particle of any of paragraphs 1-59, wherein ratio of the cationic molecule to the active agent in the particle is from about 100:1 to about 1:100.
61. The particle of any of paragraphs 1-60, wherein ratio of the cationic molecule to the active agent in the particle is about 10:1 to about 1:50.
62. The particle of any of paragraphs 1-61, wherein ratio of the cationic molecule to the active agent in the particle is about 2:1 to about 1:30.
63. The particle of any of paragraphs 1-62, wherein the carbohydrate is selected from the group consisting of oligosaccharides, polysaccharides, glycoproteins, glycolipids and any combinations thereof.
64. The particle of any of paragraphs 1-63, wherein the carbohydrate is selected from the group consisting of fructooligosaccharide, galactooligosaccharides, mannanoligosaccharides, glycogen, starch, glycosaminoglycans, cellulose, beta-glucan, maltodextrin, inulin, levan beta (2->6), chitin, chitosan, and any combinations thereof.
65. The particle of any of paragraphs 1-64, wherein the carbohydrate is present in an amount from about 1% (w/w) to about 99% (w/w).
66. The particle of any of paragraphs 1-65, wherein the carbohydrate is present in an amount from about 5% (w/w) to about 50% (w/w).
67. The particle of any of paragraphs 1-66, wherein ratio of the carbohydrate to the active agent in the particle is from about 100:1 to about 1:100.
68. The particle of any of paragraphs 1-67, wherein ratio of the carbohydrate to the active agent in the particle is about 10:1 to about 1:50.
69. The particle of any of paragraphs 1-68, wherein ratio of the carbohydrate to the active agent in the particle is about 2:1 to about 1:30.
70. A composition comprising an effective amount of particle of any of paragraphs 1-69.
71. The composition of paragraph 70, wherein the composition comprises from about 0.01% to about 50% (w/w or w/v) of the particles.
72. The composition of paragraph 70 or 71, wherein the composition comprises from about 10% to about 30% (w/w or w/v) of the particles.
73. The composition of any of paragraphs 70-73, wherein the composition further comprises one or more excipients.
74. The composition of any of paragraphs 70-73, wherein the composition comprises from about 5% to about 99.99% (w/w or w/v) of the one or more excipients.
75. The composition of paragraph 73 or 71, wherein the excipient is a solvent or an additive.
76. The composition of any of paragraphs 73-75, wherein the additive is selected from the group consisting of surfactants, stabilizers, rheology modifiers, conditioning agents, fragrances, potentiating agents, preservatives, opacifiers, pH modifiers, moisturizers, humectants, suspending agents, solubilizers, etc (anything that can go into gels, creams, shampoos, ointments, sprays, solutions, lotions, cream, oil, lotion, serum, gel, shampoo, conditioner, tooth paste, mouth wash, chewing gum, sun screen, nail varnish, ointment, foam, spray, or aerosol, and any combinations thereof.
77. The composition of any of paragraphs 70-76, wherein the additive is selected from the group consisting of 45° Be'glucose syrup, Acrylates/10-30 alkyl acrylate crosspolymer, Acrylates/Acrylamide Copolymer, agar, Allantoin, Aminomethyl propanol, Ammonium lauryl sulfate (ALS), Amodimethicone emulsion, AMP-Acrylates/Allyl Methacrylate Copolymer, Behentrimonium methosulfate, Benzophenone-4, Butylene glycol, Carbapol Aqua SF-1, Carbapol-934, Carbapol-940, Carbopol, Carboxymethyl cellulose, Cassia hydroxy propyltrimoniumchloride, Cetearyl alcohol, Cetearyl alcohol, Cetearyl isononanoate, Cetrimoniumchloride (CTC), Cetyl Alcohol, Cetyl octanoate, Chloromethyl/Methylisothiazolinone, Chloromethyl/Methylisothiazolinone, Citric acid, Citrus Aurantium Dulcis (Orange) Fruit Extract, Cocamidopropyl betain (CAPB), Cocamidopropyl betain (CAPB), Coco monoethanolamide (CMEA), Cocodimonium hydroxypropyl hydrolyzed wheat protein, Coloring agent, CPB, Diisopropyl sebacate, Dimethicone PEG-7 Isostearate, Disodium EDTA, DMDM hydantoin, Dove AD shine, Emulsifying wax, Ethanol, Ethylene glycol distearate (EGDS), Ethylhexyl methoxycinnamate, eucalyptol, Flavors, Forte therapy, Fragrance, Glycerin, Glycerine, Glyceryl monohydroxystearate, Glyceryl monostearate, Glycolic acid, Guar gum, Gum base, Hyaluronic acid, Hydrated silica, Hydrolyzed silk protein, Intense repair, Lactamide MEA acetamide MEA, Lactic acid, Limnanthes Alba (Meadowfoam) Seed Oil, Linalool, Linoleamidopropyl PG-dimonium chloride phosphate, Macrogolcetostearyl ether 20, magnese chloride, Magnesium sulfate, Menthol, Methyl gluceth-20, methyl salicylate, Mineral oil, Mint-type flavor, PEG/PPG-8/3 laurate, PEG-12 dimethicone, PEG-20 Almond Glycerides, PEG-40 hydrogenated castor oil, PEG-80 glyceryl cocoate, peppermint oil, Phenyl trimethicone, Polyacrylate-1 crosspolymer, Polyethylene glycol 1450, Polyoxyethylene/polyoxypropylene block polymer (Poloxamer 407), Polyquaternium-22, Polyquaternium-39, Polysorbate 85, polysorbate-20, PPG-3 myristyl ether, Preservative, Propylene glycol, Propylene glycol monocaprylate, Prunus Armeniaca (Apricot) Kernel Oil, Pyrus Malus (Apple) Fruit Extract, retinyl palmitate, Saccharin sodium, Salicylic acid, Sensomer CT-250, Silica Xerogel, SLES, SLS, sodium chloride, sodium docusate, Sodium hydroxide, Sodium lauryl ether sulfate (SLES), sodium lauryl sulphate, Sodium monofluorophosphate, Sodium saccharin, Sorbitol, Stearamidopropyldimethylamine, Steareth-2, Steareth-21, Stearic acid, Sugar powder, sunflower seed oil, Tea tree oil, Titanium dioxide, Tocopheryl acetate, Triclosan, Triethanolamine, Wheat amino acids, Xanthan gum, zinc carbonate, zinc chloride, zinc recinoleate, zinc stearate, and any combinations thereof.
78. The composition of any of paragraphs 70-77, wherein the composition further comprises: (i) Carbopol, Ammonium lauryl sulfate (ALS), Sodium lauryl ether sulfate (SLES), Sodium hydroxide, Coco monoethanolamide (CMEA), Ethylene glycol distearate (EGDS), Propylene glycol monocaprylate, Menthol, Magnesium sulfate, Amodimethicone emulsion, Propylene glycol, Zinc carbonate, Cocamidopropyl betaine (CAPB), Cassia hydroxyl propyltrimoniumchloride, Chloromethyl/Methylisothiazolinone, Linalool, Fragrance, Citric acid, and Sodium Chloride; or (ii) Carbopol, Ammonium lauryl sulfate (ALS), Sodium lauryl ether sulfate (SLES), Sodium hydroxide, Ethylene glycol distearate (EGDS), Propylene glycol monocaprylate, Menthol, Magnesium sulfate, Amodimethicone emulsion, Propylene glycol, Zinc carbonate, Cocamidopropyl betaine (CAPB), Cassia hydroxyl propyltrimoniumchloride, Chloromethyl/Methylisothiazolinone, Linalool, Fragrance, Citric acid, and Sodium Chloride.
79. The composition of any of paragraphs 70-78, wherein the composition further comprises: (i) Carbopol, Sodium lauryl ether sulphate (SLES), Sodium hydroxide, Steareth-2, Steareth-21, Propylene glycol monocaprylate, Macrogolcetostearyl ether 20, Coco monoethanolamide (CMEA), Cetyl Alcohol, Stearamidopropyldimethylamine, Lactic acid, Cocamidopropyl betain (CAPB), Cetrimoniumchloride (CTC), Polyquaternium-22, Amodimethicone emulsion, Cassia hydroxy propyltrimoniumchloride, Propylene glycol, Glycerine, Zinc carbonate, Titanium dioxide, Linalool, Fragrance, and Chloromethyl/Methylisothiazolinone; or (ii) Carbopol, Sodium lauryl ether sulphate (SLES), Sodium hydroxide, Steareth-2, Steareth-21, Propylene glycol monocaprylate, Macrogolcetostearyl ether 20, Coco monoethanolamide (CMEA), Cetyl Alcohol, Stearamidopropyldimethylamine, Lactic acid, Cocamidopropyl betain (CAPB), Cetrimoniumchloride (CTC), Polyquaternium-22, Amodimethicone emulsion, Cassia hydroxy propyltrimoniumchloride, Propylene glycol, Glycerine, Titanium dioxide, Linalool, Fragrance, and Chloromethyl/Methylisothiazolinone.
80. The composition of any of paragraphs 70-79, wherein the composition further comprises: (i) Stearic acid, Mineral oil, Glyceryl monostearate, Glyceryl monohydroxystearate, Cetearyl alcohol, Cetyl octanoate, Emulsifying wax, Cabopol, Triethanolamine, Water, Glycerin, Propylene glycol, Fragrance, and Preservative; (ii) Stearic acid, Mineral oil, Glyceryl monostearate, Glyceryl monohydroxystearate, Cetearyl alcohol, Cetyl octanoate, Cabopol, Triethanolamine, Glycerin, Salicylic acid, Citrus Aurantium Dulcis (Orange) Fruit Extract, Fragrance, and Preservative; (iii) Emulsifying wax, Behentrimonium methosulfate and Cetearyl alcohol, PPG-3 myristyl ether, Cetearyl isononanoate, Dimethicone PEG-7 Isostearate, Ethyl hexyl methoxycinnamate, Cabopol, Acrylates/Acrylamide Copolymer (and) Mineral Oil, (and) Polysorbate 85, Triethanolamine, Glycerin, Hyaluronic acid, Wheat amino acids, Lactamide MEA and acetamide MEA, Hydrolyzed silk protein, Salicylic acid, Propylene glycol, Pyrus Malus (Apple) Fruit Extract, Citrus Aurantium Dulcis (Orange) Fruit Extract, Tocopheryl acetate, Fragrance, and Preservative; or (iv) Glyceryl monohydroxystearate, Limnanthes Alba (Meadowfoam) Seed Oil, Prunus Armeniaca (Apricot) Kernel Oil, Phenyl trimethicone, Diisopropyl sebacate, Ethyl hexyl methoxycinnamate, Cabopol, Acrylates/Acrylamide Copolymer (and) Mineral Oil, (and) Polysorbate 85, AMP-Acrylates/Allyl Methacrylate Copolymer, Triethanolamine, PEG-20 Almond Glycerides, Titanium dioxide, Propylene glycol, Linoleamidopropyl PG-dimonium chloride phosphate, Cocodimonium hydroxypropyl hydrolyzed wheat protein, Tocopheryl acetate, Fragrance, and Preservative.
81. The composition of any of paragraphs 70-80, wherein the composition further comprises: (i) Glycerin, Methyl gluceth-20, Benzophenone-4, Acrylates/10-30 alkyl acrylate crosspolymer, PEG/PPG-8/3 laurate, Aminomethyl propanol, Polyquaternium-39, PEG-80 glyceryl cocoate, Coloring agent, Preservative, and Fragrance; (ii) Disodium EDTA, Propylene glycol, Carbopol, Aminomethyl propanol, Tea tree oil, PEG-40 hydrogenated castor oil, polysorbate-20, Coloring agent, Preservative, and Fragrance; (iii) Butylene glycol, Glycerin, Methyl gluceth-20, Allantoin, Disodium EDTA, PEG-12 dimethicone, Polyacrylate-1 crosspolymer, Glycolic acid, Triethanolamine, Tocopheryl acetate, retinyl palmitate, sunflower seed oil, agar, Coloring agent, Preservative, and Fragrance; or (iv) Glycerin, Methyl gluceth-20, Carbopol, Triethanolamine, Ethanol, Triclosan, Coloring agent, Preservative, and Fragrance.
82. The composition of any of paragraphs 70-81, wherein the composition further comprises Carboxymethyl cellulose, Polyethylene glycol 1450, Sorbitol, Glycerin, Sodium monofluorophosphate, Sodium saccharin, Preservative, Coloring agent, Silica Xerogel, Hydrated silica, Mint-type flavor, and Sodium lauryl sulfate.
83. The composition of any of paragraphs 70-82, wherein the composition further comprises Ethyl alcohol, menthol, methyl salicylate, peppermint oil, eucalyptol, Glycerin, Polyoxyethylene/polyoxypropylene block polymer (Poloxamer 407), and Saccharin sodium.
84. The composition of any of paragraphs 70-83, wherein the composition further comprises Gum base, 45° Be'glucose syrup, Sugar powder, Flavors, Glycerin, and Preservative.
85. The composition of any of paragraphs 70-84, wherein the composition is a cream, oil, lotion, serum, gel, shampoo, conditioner, tooth paste, mouth wash, chewing gum, sun screen, nail varnish, ointment, foam, spray, or aerosol.
86. The composition of any of paragraphs 70-85, wherein the composition is an anti-dandruff hair care composition selected from the group consisting of a shampoo, a conditioner, a rinse, a lotion, an aerosol, a gel, a mousse, and a hair dye.
87. The composition of any of paragraphs 70-86, wherein the composition is a skin care composition selected from the group consisting of lotions, creams, gels, sticks, sprays, ointments, cleansing liquid washes, cleansing solid bars, pastes, foams, powders, shaving creams, and wipes.
88. The composition of any of paragraphs 70-87, wherein the composition is an oral care composition selected from the group consisting of tooth pastes, mouth washes, and chewing gums.
89. The composition of any of paragraphs 70-88, wherein the composition is an antifungal, antibacterial, anti-inflammatory, anti-aging, anti-wrinkle, or skin whitening or skin bleaching composition.
90. The composition of any of paragraphs 70-89, wherein the composition is an anti-acne composition.
91. The composition of any of paragraphs 70-90, wherein the composition is a shampoo, the active agent is an anti-fungal agent and the coating layer lipid is EGDS, and wherein the excipient is propylene glycol monocaprylate.
92. The composition of any of paragraphs 70-91, wherein the composition is a gel or cream, wherein the active agent is an anti-propionibacterium agent and the coating layer lipid is Lauric acid and/or stearic acid, and wherein the excipient is TPGS.
93. The particle of any of paragraphs 1-69, wherein the lipid is stable in shampoo formulation and acts a food for a pathogen.
94. The particle of any of paragraphs 1-69, wherein the lipid is stable in shampoo formulation and enhances targeting, binding, or retention of the active agent to a desired site of action.
95. The particle of any of paragraphs 1-69, wherein the lipid is stable in shampoo formulation and has a synergistic effect on activity of the active agent.

Exemplary embodiments of the invention can also be described by any one of the following numbered paragraphs.
1. A particle comprising an active agent and a lipid, wherein the particle comprises a coating layer of the lipid on a core comprising the active agent.
2. The particle of paragraph 1, wherein the lipid is selected from the group consisting of fatty acids; mono-, di- or tri-esters of fatty acids; salts of fatty acids; fatty alcohols; mono-, di- or tri-esters of fatty alcohols; glycerolipids; phospholipids; glycerophospholipids; sphingolipids; sterol lipids; prenol lipids; saccharolipids; polyketides; and any combination thereof.
3. The particle of any of paragraphs 1-2, wherein the lipid is selected from the group consisting of 1,3-Propanediol Dicaprylate/Dicaprate; 10-undecenoic acid; 1-dotriacontanol; 1-heptacosanol; 1-nonacosanol; 2-ethyl hexanol; Androstanes; Arachidic acid; Arachidonic acid; arachidyl alcohol; Behenic acid; behenyl alcohol; Capmul MCM C10; Capric acid; capric alcohol; capryl alcohol; Caprylic acid; Caprylic/Capric Acid Ester of Saturated Fatty Alcohol C12-C18; Caprylic/Capric Triglyceride; Caprylic/Capric Triglyceride; Ceramide phosphorylcholine (Sphingomyelin, SPH); Ceramide phosphorylethanolamine (Sphingomyelin, Cer-PE); Ceramide phosphorylglycerol; Ceroplastic acid; Cerotic acid; Cerotic acid; ceryl alcohol; Cetearyl alcohol; Ceteth-10; cetyl alcohol; Cholanes; Cholestanes; cholesterol; cis-11-eicosenoic acid; cis-11-octadecenoic acid; cis-13-docosenoic acid; cluytyl alcohol; coenzyme Q10 (CoQ10); Dihomo-γ-linolenic; Docosahexaenoic acid; egg lecithin; Eicosapentaenoic acid; Eicosenoic acid; Elaidic acid; elaidolinolenyl alcohol; elaidolinoleyl alcohol; elaidyl alcohol; Erucic acid; erucyl alcohol; Estranes; Geddic acid; geddyl alcohol; glycerol distearate (type I) EP (Precirol ATO 5); Glycerol Tricaprylate/Caprate; Glycerol Tricaprylate/Caprate (CAPTEX® 355 EP/NF); glyceryl monocaprylate (Capmul MCM C8 EP); Glyceryl Triacetate; Glyceryl Tricaprylate; Glyceryl Tricaprylate/Caprate/Laurate; Glyceryl Tricaprylate/Tricaprate; glyceryl tripalmitate (Tripalmitin); Henatriacontylic acid; Heneicosyl alcohol; Heneicosylic acid; Heptacosylic acid; Heptadecanoic acid; Heptadecyl alcohol; Henatriacontylic acid; isostearic acid; isostearyl alcohol; Lacceroic acid; Lauric acid; Lauryl alcohol; Lignoceric acid; lignoceryl alcohol; Linoelaidic acid; Linoleic acid; linolenyl alcohol; linoleyl alcohol; Margaric acid; Mead; Melissic acid; melissyl alcohol; Montanic acid; montanyl alcohol; myricyl alcohol; Myristic acid; Myristoleic acid; Myristyl alcohol; neodecanoic acid; neoheptanoic acid; neononanoic acid; Nervonic; Nonacosylic acid; Nonadecyl alcohol; Nonadecylic acid; Nonadecylic acid; Oleic acid; oleyl alcohol; Palmitic acid; Palmitoleic acid; palmitoleyl alcohol; Pelargonic acid; pelargonic alcohol; Pentacosylic acid; Pentadecyl alcohol; Pentadecylic acid; Phosphatidic acid (phosphatidate, PA); Phosphatidylcholine (lecithin, PC); Phosphatidylethanolamine (cephalin, PE); Phosphatidylinositol (PI); Phosphatidylinositol bisphosphate (PIP2); Phosphatidylinositol phosphate (PIP); Phosphatidylinositol triphosphate (PIP3); Phosphatidylserine (PS); polyglyceryl-6-distearate; Pregnanes; Propylene Glycol Dicaprate; Propylene Glycol Dicaprylocaprate; Propylene Glycol Dicaprylocaprate; Psyllic acid; recinoleaic acid; recinoleyl alcohol; Sapienic acid; soy lecithin; Stearic acid; Stearidonic; stearyl alcohol; Tricosylic acid; Tridecyl alcohol; Tridecylic acid; Triolein; Undecyl alcohol; undecylenic acid; Undecylic acid; Vaccenic acid; α-Linolenic acid; γ-Linolenic acid; a fatty acid salt of 10-undecenoic acid, adapalene, arachidic acid, arachidonic acid, behenic acid, butyric acid, capric acid, caprylic acid, cerotic acid, cis-11-eicosenoic acid, cis-11-octadecenoic acid, cis-13-docosenoic acid, docosahexaenoic acid, eicosapentaenoic acid, elaidic acid, erucic acid, heneicosylic acid, heptacosylic acid, heptadecanoic acid, isostearic acid, lauric acid, lignoceric acid, linoelaidic acid, linoleic acid, montanic acid, myristic acid, myristoleic acid, neodecanoic acid, neoheptanoic acid, neononanoic acid, nonadecylic acid, oleic acid, palmitic acid, palmitoleic acid, pelargonic acid, pentacosylic acid, pentadecylic acid, recinoleaic acid, sapienic acid, stearic acid, tricosylic acid, tridecylic acid, undecylenic acid, undecylic acid, vaccenic acid, valeric acid, α-linolenic acid, or γ-linolenic acid; paraffin; and any combinations thereof.
4. The particle of any of paragraphs 1-3, wherein the fatty acid salt is selected from the group consisting of zinc, sodium, potassium, lithium, ammonium, copper, calcium, magnesium, strontium, manganese, and combinations thereof.
5. The particle of any of paragraphs 1-4, wherein the lipid comprises 11 or fewer carbon atoms.
6. The particle of any of paragraphs 1-5, wherein the lipid is selected from the group consisting of ethylene glycol distearate (EGDS), caprylic acid, capric acid, lauric acid, myristic acid, palmitic acid, zinc recinoleate, CoQ10, paraffin, triplamitin, polyglyceryl-6-distearate, and any combinations thereof.
7. The particle of any of paragraphs 1-6, wherein the active agent is selected from the group consisting of small organic or inorganic molecules, saccharines, oligosaccharides, polysaccharides, peptides; proteins, peptide analogs and derivatives, peptidomimetics, nucleic acids, nucleic acid analogs and derivatives, antibodies, antigen binding fragments of antibodies, lipids, extracts made from biological materials, naturally occurring or synthetic compositions, and any combinations thereof.
8. The particle of any of paragraphs 1-7, wherein the active agent is selected from the group consisting of antifungal agents, antibacterial agents, antimicrobial agents, antioxidant agents, cooling agents, soothing agents, wound healing agents, anti-inflammatory-agents, anti-aging agents, anti-wrinkle agents, skin whitening or bleaching agents, ultraviolet (UV) light absorbing or scattering agents, skin depigmentation agents, dyes or coloring agents, deodorizing agents, fragrances, and any combinations thereof.
9. The particle of any of paragraphs 1-8, wherein the active agent is selected from the group consisting of pyrithione salts; ketoconazole; salicylic acid; curcumin or a derivative of curcumin, curcuminoids; tetrahydro curcuminoids; titanium dioxide (TiO₂); zinc oxide (ZnO); chloroxylenol; flvanoids; CoQ10; vitamin C; herbal extracts; alkaloids; 13-cis retinoic acid; 3,4-methylenedioxymethamphetamine; 5-fluorouracil; 6,8-dimercaptooctanoic acid (dihydrolipoic acid); abacavir; acebutolol; acetaminophen; acetaminosalol; acetazolamide; acetohydroxamic acid; acetylsalicylic acid; acitretin; aclovate; acrivastine; actiq; acyclovir; adapalene; adefovir dipivoxil; adenosine; Albaconazole; albuterol; alfuzosin; Allicin; allopurinol; alloxanthine; allylamines; almotriptan; alpha-hydroxy acids; alprazolam; alprenolol; aluminum acetate; aluminum chloride; aluminum chlorohydroxide; aluminum hydroxide; amantadine; amiloride; aminacrine; aminobenzoic acid (PABA); aminocaproic acid; aminoglycosides such as streptomycin, neomycin, kanamycin, paromycin, gentamicin, tobramycin, amikacin, netilmicin, spectinomycin, sisomicin, dibekalin and isepamicin; aminosalicylic acid; amiodarone; amitriptyline; amlodipine; amocarzine; amodiaquin; Amorolfin; amoxapine; amphetamine; amphotericin B; ampicillin; anagrelide; anastrozole; Anidulafungin; anthralin; antibacterial sulfonamides and antibacterial sulphanilamides, including para-aminobenzoic acid, sulfadiazine, sulfisoxazole, sulfamethoxazole and sulfathalidine; antifungal peptide and derivatives and analogs thereof; apomorphine; aprepitant; arbutin; aripiprazole; ascorbic acid; ascorbyl palmitate; atazanavir; atenolol; atomoxetine; atropine; azathioprine; azelaic acid; azelaic acid; azelastine; azithromycin; bacitracin; bacitracin; beanomicins; beclomefhasone dipropionate; bemegride; benazepril; bendroflumethiazide; benzocaine; Benzoic acid with a keratolytic agent; benzonatate; benzophenone; benztropine; bepridil; beta-hydroxy acids; beta-lactams including penicillin, cephalosporin, and carbapenems such as carbapenem, imipenem, and meropenem; betamethasone dipropionate; betamethasone valerate; brimonidine; brompheniramine; bupivacaine; buprenorphine; bupropion; burimamide; butenafine; Butenafine; butoconazole; Butoconazole; cabergoline; caffeic acid; caffeine; calcipotriene; camphor; Cancidas; candesartan cilexetil; capsaicin; carbamazepine; Caspofungin; cefditoren pivoxil; cefepime; cefpodoxime proxetil; celecoxib; cetirizine; cevimeline; chitosan; chlordiazepoxide; chlorhexidine; chloroquine; chlorothiazide; chloroxylenol; chlorpheniramine; chlorpromazine; chlorpropamide; ciclopirox; Ciclopirox (ciclopirox olamine); cilostazol; cimetidine; cinacalcet; ciprofloxacin; citalopram; citric acid; Citronella oil; cladribine; clarithromycin; clemastine; clindamycin; clioquinol; clobetasol propionate; clomiphene; clonidine; clopidogrel; Clortrimazole; clotrimazole; Clotrimazole; clozapine; cocaine; Coconut oil; codeine; colistin; colymycin; cromolyn; crotamiton; Crystal violet; cyclizine; cyclobenzaprine; cycloserine; cytarabine; dacarbazine; dalfopristin; dapsone; daptomycin; daunorubicin; deferoxamine; dehydroepiandrosterone; delavirdine; desipramine; desloratadine; desmopressin; desoximetasone; dexamethasone; dexmedetomidine; dexmethylphenidate; dexrazoxane; dextroamphetamine; diazepam; dicyclomine; didanosine; dihydrocodeine; dihydromorphine; diltiazem; diphenhydramine; diphenoxylate; dipyridamole; disopyramide; dobutamine; dofetilide; dolasetron; donepezil; dopa esters; dopamine; dopamnide; dorzolamide; doxepin; doxorubicin; doxycycline; doxylamine; doxypin; duloxetine; dyclonine; echinocandins; econazole; Econazole; eflormthine; eletriptan; emtricitabine; enalapril; ephedrine; epinephrine; epinine; epirubicin; eptifibatide; ergotamine; erythromycin; escitalopram; esmolol; esomeprazole; estazolam; estradiol; ethacrynic acid; ethinyl estradiol; etidocaine; etomidate; famciclovir; famotidine; felodipine; fentanyl; Fenticonazole; ferulic acid; fexofenadine; flecainide; fluconazole; Fluconazole; flucytosiine; Flucytosine or 5-fluorocytosine; fluocinolone acetonide; fluocinonide; fluoxetine; fluphenazine; flurazepam; fluvoxamine; formoterol; furosemide; galactarolactone; galactonic acid; galactonolactone; galactose; galantamine; gatifloxacin; gefitinib; gemcitabine; gemifloxacin; gluconic acid; glycolic acid; glycolic acid; glycopeptides such as vancomycin and teicoplanin; griseofulvin; Griseofulvin; guaifenesin; guanethidine; haloperidol; haloprogin; Haloprogin; herbal extract, an alkaloid, a flvanoid, Abafungin; hexylresorcinol; homatropine; homosalate; hydralazine; hydrochlorothiazide; hydrocortisone; hydrocortisone 17-butyrate; hydrocortisone 17-valerate; hydrocortisone 21-acetate; hydromorphone; hydroquinone; hydroquinone monoether; hydroxyzine; hyoscyamine; hypoxanthine; ibuprofen; ichthammol; idarubicin; imatinib; imipramine; imiquimod; indinavir; indomethacin; Iodine; irbesartan; irinotecan; Isavuconazole; Isoconazole; isoetharine; isoproterenol; itraconazole; Itraconazole; kanamycin; ketamine; ketanserin; ketoconazole; ketoprofen; ketotifen; kojic acid; labetalol; lactic acid; lactobionic acid; lactobionic acid; lamivudine; lamotrigine; lansoprazole; lemon myrtle; letrozole; leuprolide; levalbuterol; levofloxacin; lidocaine; lincosamides such as lincomycin and clindamycin; linezolid; lobeline; loperamide; losartan; loxapine; lucensomycin; lysergic diethylamide; macrolides or ketolides such as erythromycin, azithromycin, clarithromycin, and telithromycin; mafenide; malic acid; maltobionic acid; mandelic acid; mandelic acid; maprotiline; mebendazole; mecamylamine; meclizine; meclocycline; memantine; menthol; meperidine; mepivacaine; mercaptopurine; mescaline; metanephrine; metaproterenol; metaraminol; metformin; methadone; methamphetamine; methotrexate; methoxamine; methyl nicotinate; methyl salicylate; methyldopa esters; methyldopamide; methyllactic acid; methylphenidate; metiamide; metolazone; metoprolol; metronidazole; mexiletine; Micafungin; miconazole; Miconazole; midazolam; midodrine; miglustat; minocycline; minoxidil; mirtazapine; mitoxantrone; moexiprilat; molindone; monobenzone; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, and astreonam; morphine; moxifloxacin; moxonidine; mupirocin; nadolol; naftifine; Naftifine; nalbuphine; nalmefene; naloxone; naproxen; natamycin; Neem Seed Oil; nefazodone; nelfinavir; neomycin; nevirapine; N-guanylhistamine; nicardipine; nicotine; nifedipine; nikkomycins; nimodipine; nisoldipine; nizatidine; norepinephrine; nystatin; nystatin; octopamine; octreotide; octyl methoxycinnamate; octyl salicylate; ofloxacin; olanzapine; Olive leaf extract; olmesartan medoxomil; olopatadine; omeprazole; Omoconazole; ondansetron; Orange oil; oxazolidinones such as linezolid; oxiconazole; Oxiconazole; oxotremorine; oxybenzone; oxybutynin; oxycodone; oxymetazoline; padimate O; palmarosa oil; palonosetron; pantothenic acid; pantoyl lactone; paroxetine; patchouli; pemoline; penciclovir; penicillamine; penicillins; pentazocine; pentobarbital; pentostatin; pentoxifylline; pergolide; perindopril; permethrin; phencyclidine; phenelzine; pheniramine; phenmetrazine; phenobarbital; phenol; phenoxybenzamine; phenpropimorph; phentolamine; phenylephrine; phenylpropanolamine; phenytoin; phosphonomycin; physostigmine; pilocarpine; pimozide; pindolol; pioglitazone; pipamazine; piperonyl butoxide; pirenzepine; Piroctone; piroctone olamine; podofilox; podophyllin; Polygodial; polyhydroxy acids; polymyxin; Posaconazole; pradimicins; pramoxine; pratipexole; prazosin; prednisone; prenalterol; prilocaine; procainamide; procaine; procarbazine; promazine; promethazine; promethazine propionate; propafenone; propoxyphene; propranolol; propylthiouracil; protriptyline; pseudoephedrine; pyrethrin; pyrilamine; pyrimethamine; quetiapine; quinapril; quinethazone; quinidine; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin and pazufloxacin; quinupristin; rabeprazole; Ravuconazole; reserpine; resorcinol; retinal; retinoic acid; retinol; retinyl acetate; retinyl palmitate; ribavirin; ribonic acid; ribonolactone; rifampin; rifamycins such as rifampicin (also called rifampin), rifapentine, rifabutin, bezoxazinorifamycin and rifaximin; rifapentine; rifaximin; riluzole; rimantadine; risedronic acid; risperidone; ritodrine; rivasfigmine; rizatriptan; ropinirole; ropivacaine; salicylamide; salicylic acid; salicylic acid; salmeterol; scopolamine; selegiline; Selenium; selenium sulfide; serotonin; Sertaconazole; sertindole; sertraline; sibutramine; sildenafil; sordarins; sotalol; streptogramins such as quinupristin and daflopristin; streptomycin; strychnine; sulconazole; Sulconazole; sulfabenz; sulfabenzamide; sulfabromomethazine; sulfacetamide; sulfachlorpyridazine; sulfacytine; sulfadiazine; sulfadimethoxine; sulfadoxine; sulfaguanole; sulfalene; sulfamethizole; sulfamethoxazole; sulfanilamide; sulfapyrazine; sulfapyridine; sulfasalazine; sulfasomizole; sulfathiazole; sulfisoxazole; tadalafil; tamsulosin; tartaric acid; tazarotene; Tea tree oil—ISO 4730 (“Oil of Melaleuca, Terpinen-4-ol type”); tegaserol; telithromycin; telmisartan; temozolomide; tenofovir disoproxil; terazosin; terbinafine; Terbinafine; terbutaline; terconazole; Terconazole; terfenadine; tetracaine; tetracycline; tetracyclines such as tetracycline, chlortetracycline, demeclocycline, minocycline, oxytetracycline, methacycline, doxycycline; tetrahydrozoline; theobromine; theophylline; thiabendazole; thioridazine; thiothixene; thymol; tiagabine; timolol; tinidazole; tioconazole; Tioconazole; tirofiban; tizanidine; tobramycin; tocainide; tolazoline; tolbutamide; tolnaftate; Tolnaftate; tolterodine; tramadol; tranylcypromine; trazodone; triamcinolone acetonide; triamcinolone diacetate; triamcinolone hexacetonide; triamterene; triazolam; triclosan; triclosan; Triclosan; triflupromazine; trimethoprim; trimethoprim; trimipramine; tripelennamine; triprolidine; tromethamine; tropic acid; tyramine; undecylenic acid; Undecylenic acid; urea; urocanic acid; ursodiol; vardenafil; venlafaxine; verapamil; vitamin C; vitamin E acetate; voriconazole; Voriconazole; warfarin; xanthine; zafirlukast; zaleplon; zinc pyrithione; Zinc Selenium sulfide; ziprasidone; zolmitriptan; Zolpidem; WS-3; WS-23; menthol; 3-substituted-P-menthanes; N-substituted-P-menthane-3-carboxamides; isopulegol; 3-(1-menthoxy)propane-1,2-diol; 3-(1-menthoxy)-2-methylpropane-1,2-diol; p-menthane-2,3-diol; p-menthane-3,8-diol; 6-isopropyl-9-methyl-1,4-dioxaspiro[4, 5]decane-2-methanol; menthyl succinate and its alkaline earth metal salts; trimethylcyclohexanol; N-ethyl-2-isopropyl-5-methylcyclohexanecarboxamide; Japanese mint oil; peppermint oil; menthone; menthone glycerol ketal; menthyl lactate; 3-(1-menthoxy)ethan-1-ol; 3-(1-menthoxy)propan-1-ol; 3-(1-menthoxy)butan-1-ol; 1-menthylacetic acid N-ethyl amide; 1-menthyl-4-hydroxypentanoate; 1-menthyl-3-hydroxybutyrate; N,2,3-trimethyl-2-(1-methylethyl)-butanamide; n-ethyl-t-2-c-6 nonadienamide; N,N-dimethyl menthyl succinamide; menthyl pyrrolidone carboxylate; aloe; avocado oil; green tea extract; hops extract; chamomile extract; colloidal oatmeal; calamine; cucumber extract; sodium palmate; sodium palm kernelate; butyrospermum parkii (i.e., shea butter); menthe piperita (i.e.; peppermint) leaf oil; sericin; pyridoxine (a form of vitamin B6); retinyl palmitate and/or other forms of vitamin A; tocopheryl acetate and/or other forms of vitamin E; lauryl laurate; hyaluronic acid; aloe barbadensis leaf juice powder; euterpe oleracea (i.e., acai berry) fruit extract; riboflavin (i.e., vitamin B2); thiamin HCl and/or other forms of vitamin B1; ethylenediaminetetraacetic acid (EDTA); citrate; ethylene glycol tetraacetic acid (EGTA); 1,2-bis(o-aminophenoxy)ethane-N,N,N′,N′-tetraacetic acid (BAPTA); diethylene triamine pentaacetic acid (DTPA); 2,3-dimercapto-1-propanesulfonic acid (DMPS); dimercaptosuccinic acid (DMSA); α-lipoic acid; salicylaldehyde isonicotinoyl hydrazone (SIH); hexyl thioethylamine hydrochloride (HTA); desferrioxamine; ascorbic acid (vitamin C); cysteine; glutathione; dihydrolipoic acid; 2-mercaptoethane sulfonic acid; 2-mercaptobenzimidazole sulfonic acid; 6-hydroxy-2,5,7,8-tetramethylchroman-2-carboxylic acid; sodium metabisulfite; vitamin E isomers such as α-, β-, γ-, and δ-tocopherols and α-, β-, γ-, and δ-tocotrienols; polyphenols such as 2-tert-butyl-4-methyl phenol, 2-tert-butyl-5-methyl phenol, and 2-tert-butyl-6-methyl phenol; butylated hydroxyanisole (BHA) such as 2-tert-butyl-4-hydroxyanisole and 3-tert-butyl-4-hydroxyanisole; butylhydroxytoluene (BHT); tert-butylhydroquinone (TBHQ); ascorbyl palmitate; n-propyl gallate; soy extract; soy isoflavones; retinoids such as retinol; kojic acid; kojic dipalmitate; hydroquinone; arbutin; transexamic acid; vitamins such as niacin and vitamin C; azelaic acid; linolenic acid and linoleic acid; placertia; licorice; and extracts such as chamomile and green tea; hydrogen peroxide; zinc peroxide; sodium peroxide; hydroquinone; 4-isopropylcatechol; hydroquinone monobenzyl ether; kojic acid; lactic acid; ascorbyl acid and derivatives such as magnesium ascorbyl phosphate; arbutin; licorice root; dihydroxyacetone (DHA); glyceryl aldehyde; tyrosine and tyrosine derivatives such as malyltyrosine, tyrosine glucosinate, and ethyl tyrosine; phospho-DOPA; indoles and derivatives; glucosamine; N-acetyl glucosamine; glucosamine sulfate; mannosamine; N-acetyl mannosamine; galactosamine; N-acetyl galactosamine; N-acyl amino acid compounds (e.g., N-undecylenoyl-L-phenylalanine); flavonoids such as quercetin, hesperidin, quercitrin, rutin, tangeritin, and epicatechin; CoQ10; vitamin C; hydroxy acids including C₂-C₃₀ alpha-hydroxy acids such as glycolic acid, lactic acid, 2-hydroxy butanoic acid, malic acid, citric acid tartaric acid, alpha-hydroxyethanoic acid, hydroxycaprylic acid and the like; beta hydroxy acids including salicylic acid and polyhydroxy acids including gluconolactone (G4); retinoic acid; gamma-linolenic acid; ultraviolet absorber of benzoic acid system such as para-aminobenzoic acid (hereinafter, abbreviated as PABA), PABA monoglycerin ester, N,N-dipropoxy PABA ethyl ester, N,N-diethoxy PABA ethyl ester, N,N-dimethyl PABA ethyl ester, N,N-dimethyl PABA butyl ester, and N,N-dimethyl PABA methyl ester and the like; ultraviolet absorber of anthranilic acid system such as homomenthyl-N-acetyl anthranilate and the like; ultraviolet absorber of salicylic acid system such as amyl salicylate, menthyl salicylate, homomenthyl salicylate, octyl salicylate, phenyl salicylate, benzyl salicylate, p-isopropanol phenyl salicylate and the like; ultraviolet absorber of cinnamic acid system such as octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxy cinnamate, isopropyl-p-methoxy cinnamate, isoamyl-p-methoxy cinnamate, octyl-p-methoxy cinnamate(2-ethylhexyl-p-methoxy cinnamate), 2-ethoxyethyl-p-methoxy cinnamate, cyclohexyl-p-methoxy cinnamate, ethyl-α-cyano-β-phenyl cinnamate, 2-ethylhexyl-α-cyano-β-phenyl cinnamate, glyceryl mono-2-ethylhexanoyl-dipara-methoxy cinnamate, methyl bis(trimethylsiloxane)silylisopentyl trimethoxy cinnamate and the like; 3-(4′-methylbenzylidene)-d,l-camphor; 3-benzylidene-d,l-camphor; urocanic acid, urocanic acid ethyl ester; 2-phenyl-5-methylbenzoxazole; 2,2′-hydroxy-5-methylphenylbenzotriazole; 2-(2′-hydroxy-5′-t-octylphenyl)benzotriazole; 2-(2′-hydroxy-5′-methylphenylbenzotriazole; dibenzaladine; dianisoylmethane; 4-methoxy-4′-t-butyldibenzoylmethane; 5-(3,3-dimethyl-2-norbornylidene)-3-pentane-2-one; dimorpholinopyridazinone; titanium oxide; particulate titanium oxide; zinc oxide; particulate zinc oxide; ferric oxide; particulate ferric oxide; ceric oxide; inorganic sunscreens such as titanium dioxide and zinc oxide; organic sunscreens such as octyl-methyl cinnamates and derivatives thereof; retinoids; vitamins such as vitamin E, vitamin A, vitamin C (ascorbic acid), vitamin B, and derivatives thereof such as vitamin E acetate, vitamin C palmitate, and the like; antioxidants including alpha hydroxy acid such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucopehtonic acid, glucopheptono-1,4-lactone, gluconic acid, gluconolactone, glucuronic acid, glucurronolactone, glycolic acid, isopropyl pyruvate, methyl pyruvate, mucic acid, pyruvia acid, saccharic acid, saccaric acid 1,4-lactone, tartaric acid, and tartronic acid; beta hydroxy acids such as beta-hydroxybutyric acid, beta-phenyl-lactic acid, beta-phenylpyruvic acid; botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, rice, and safflower; 21-acetoxypregnenolone; alclometasone; algestone; amcinonide; beclomethasone; betamethasone; budesonide; chloroprednisone; clobetasol; clobetansone; clocortolone; cloprednol; corticosterone; cortisone; cortivazol; deflazacort; desonide; desoximetasone; dexamethasone; diflorasone; diflucortolone; difluprednate; enoxolone; fluazacort; flucloronide; flumethasone flunisolide; fluocinolone acetonide; fluocinonide; fluocortin butyl; fluocortolone; fluorometholone; fluperolone acetate; fluprednidene acetate; fluprednisolone; flurandrenolide; fluticasone propionate; formocortal; halcinonide; halobetasol propionate; halometasone; halopredone acetate; hydrocortamate; hydrocortisone; loteprednol etabonate; mazipredone; medrysone; meprednisone; methylprednisolone; mometasone furcate; paramethosone; prednicarbate; prednisolone; prednisolone 25-diethylamino-acetate; prednisolone sodium phosphate; prednisone; prednival; prednylidene; rimexolone; tixocortol; triamcinolone; triamcinolone acetonide; triamcinolone benetonide; triamcinolone hexacetonide; COX inhibitors such as salicylic acid derivatives (e.g., aspirin, sodium salicylate, choline magnesium trisalicylate, salicylate, diflunisal, sulfasalazine and olsalazine); para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam); alkanones such as nabumetone; diarylsubstituted furanones such as refecoxib; diaryl-substituted pyrazoles such as celecoxib; indole acetic acids such as etodolac; sulfonanilides such as nimesulide; selenium sulfide; sulfur; sulfonated shale oil; salicylic acid; coal tar; povidone-iodine, imidazoles such as ketoconazole, dichlorophenyl imidazolodioxalan, clotrimazole, itraconazoie, miconazole, climbazole, tioconazole, sulconazole, butoconazole, fluconazole, miconazolenitrite; anthralin; piroctone olamine (Octopirox); ciclopirox olamine; anti-psoriasis agents; vitamin A analogs; corticosteroids; and any combinations thereof.
10. The particle of any of paragraphs 1-9, wherein the pyrithione salt is selected from the group consisting of zinc pyrithione, sodium pyrithione, potassium pyrithione, lithium pyrithione, ammonium pyrithione, copper pyrithione, calcium pyrithione, magnesium pyrithione, strontium pyrithione, silver pyrithione, gold pyrithione, manganese pyrithione, and any combinations thereof.
11. The particle of any of paragraphs 1-10, wherein the particle has a size of about 5 nm to about 20 μm.
12. The particle of any of paragraphs 1-11, wherein the particle has a size of about 100 nm to about 10 μm.
13. The particle of any of paragraphs 1-13, wherein the particle has a size of about 200 nm to about 6 μm or wherein the particle has a size of about 1 μm to about 6 μm.
14. The particle of any of paragraphs 1-13, wherein the particle has a size of about 300 nm to about 700 nm.
15. The particle of any of paragraphs 1-14, wherein the coating layer has a thickness of about 1 nm to about 1000 nm.
16. The particle of any of paragraphs 1-15, wherein the coating layer has a thickness of about 1 nm to about 150 nm.
17. The particle of any of paragraphs 1-16, wherein the active agent in the particle is present in an amount from about 1% to about 99% (w/w).
18. The particle of any of paragraphs 1-17, wherein the active agent in the particle is present in an amount from about 75% to about 98% (w/w).
19. The particle of any of paragraphs 1-18, wherein the particle further comprises a second lipid in addition to the first lipid and the active agent.
20. The particle of paragraph 19, wherein at least a portion of the second lipid is present in the coating layer.
21. The particle of any of paragraphs 19-20, wherein the second lipid is a fatty acid or an ester or a salt thereof.
22. The particle of any of paragraphs 19-21, wherein ratio of the first lipid to the second lipid is about 100:1 to 1:100.
23. The particle of any of paragraphs 19-22, wherein ratio of the first lipid to the second lipid is about 10:1 to 1:10.
24. The particle of any of paragraphs 19-23, wherein ratio of the first lipid to the second lipid is about 2:1 to about 1:2.
25. The particle of any of paragraphs 1-24, wherein total lipid in the particle is present in an amount from about 1% to about 99% (w/w).
26. The particle of any of paragraphs 1-25, wherein total lipid in the particle is present in an amount from about 2% to about 25% (w/w).
27. The particle of any of paragraphs 1-26, wherein the particle comprises an excess of the active agent relative to the total lipids.
28. The particle of any of paragraphs 1-27, wherein ratio of total lipids to the active agent in the particle is from about 100:1 to about 1:100.
29. The particle of any of paragraphs 1-28, wherein ratio of total lipid to the active agent in the particle is about 10:1 to about 1:50.
30. The particle of any of paragraphs 1-29, wherein ratio of total lipid to the active agent in the particle is about 2:1 to about 1:30.
31. The particle of any of paragraphs 1-30, wherein the particle further comprises a protein in addition to the first lipid and the active agent.
32. The particle of paragraph 31, wherein at least a portion of the protein is present in the coating layer.
33. The particle of any of paragraphs 31-32, wherein the protein selected from the group consisting of Actin, Albumin, Amaranth Protein, Ammonium Hydrolyzed Animal Protein, Animal protein, Barley Protein, Brazil Nut Protein, Casein, Collagen, Collagen protein hydrolyzed, Conchiolin Protein, corn protein, Cottonseed Protein, Elastin, Extensin, Fibroin, Fibronectin, Fish Protein, Gadidae Protein, Gelatin, Glutein, Glycoproteins, Hazelnut Protein, Hemoglobin, Hemp Seed Protein, Honey Protein, Hydrolyzed Actin, Hydrolyzed Amaranth Protein, Hydrolyzed animal protein, Hydrolyzed Barley Protein, Hydrolyzed Brazil Nut Protein, Hydrolyzed Conchiolin Protein, Hydrolyzed corn protein, Hydrolyzed Cottonseed Protein, Hydrolyzed Elastin, Hydrolyzed Extensin, Hydrolyzed Fibroin, Hydrolyzed Fibronectin, Hydrolyzed Fish Protein, Hydrolyzed Gadidae Protein, Hydrolyzed Gadidae Protein, Hydrolyzed Gelatin, Hydrolyzed Hair Keratin, Hydrolyzed Hazelnut, Hydrolyzed Hazelnut Protein, Hydrolyzed Hemoglobin, Hydrolyzed Hemp Seed Protein, Hydrolyzed Honey Protein, Hydrolyzed Keratin, Hydrolyzed Lupine Protein, Hydrolyzed Maple Sycamore Protein, Hydrolyzed Milk Protein, Hydrolyzed Oat Protein, Hydrolyzed Pea Protein, Hydrolyzed Potato Protein, Hydrolyzed Reticulin, Hydrolyzed Royal Jelly Protein, Hydrolyzed Sericin, Hydrolyzed Serum Protein, Hydrolyzed Sesame Protein, Hydrolyzed Soy Protein, Hydrolyzed Soymilk Protein, Hydrolyzed Spinal Protein, Hydrolyzed Spongin, Hydrolyzed Sweet Almond Protein, Hydrolyzed Vegetable Protein, Hydrolyzed Wheat Gluten, Hydrolyzed Wheat Protein, Hydrolyzed Whey Protein, Hydrolyzed Yeast Protein, Hydrolyzed Yogurt Protein, Hydrolyzed Zein, Integrin, Jojoba protein HP, Hydrolyzed, keratin, Lupine Protein, Maple Sycamore Protein, MEA-Hydrolyzed Collagen, MEA-Hydrolyzed Silk, Milk Protein, Myosin, Oat Protein, Pea Protein, polylysine, Potato Protein, Reticulin, Rice Quat, Royal Jelly Protein, Sericin, Serum Protein, Sesame Protein, Silk powder, Sodium Hydrolyzed Casein, Soy Protein, Soy Rice Peptides, Soymilk Protein, Spinal Protein, Spongin, Sweet Almond Protein, Vegetable Protein, Wheat Gluten, Whey Protein, Yeast Protein, Yogurt Protein, Zein, and Zinc Hydrolyzed Collagen.
34. The particle of any of paragraphs 31-33, wherein the albumin is bovine serum albumin, egg albumin, Hydrolyzed Lactalbumin, or Lactalbumin.
35. The particle of any of paragraphs 31-34, wherein the protein in the particle is present in an amount from about 0.1% to about 10% (w/w).
36. The particle of any of paragraphs 31-35, wherein the protein in the particle is present in an amount from about 0.5% to about 5% (w/w).
37. The particle of any of paragraphs 31-36, wherein ratio of the protein to the active agent in the particle is from about 100:1 to about 1:100.
38. The particle of any of paragraphs 31-37, wherein ratio of the protein to the active agent in the particle is from about 1:25 to about 1:80.
39. The particle of any of paragraphs 31-38, wherein ratio of the protein to the active agent in the particle is from about 1:50 to about 1:75.
40. The particle of any of paragraphs 31-39, wherein ratio of total lipids to the protein in the particle is about 100:1 to about 1:100.
41. The particle of any of paragraphs 31-40, wherein ratio of total lipids to the protein in the particle is about 10:1 to about 1:10.
42. The particle of any of paragraphs 31-41, wherein ratio of total lipids to the protein in the particle is about 5:1 to about 1:5.
43. The particle of any of paragraphs 31-42, wherein ratio of total lipids to the protein in the particle is about 2:1 to about 1:1.
44. The particle of any of paragraphs 1-43, wherein the particle further comprises a cationic molecule in addition to the first lipid and the active agent.
45. The particle of paragraph 43, wherein at least a portion of the cationic molecule is present in the coating layer.
46. The particle of any of paragraphs 44-45, wherein the cationic molecule is a polyamine.
47. The particle of any of paragraphs 44-46, wherein the cationic molecule is selected from the group consisting of Putrescine (Butane-1,4-diamine), Cadaverine (Pentane-1,5-diamine), Spermidine, Spermine, Cyclen (1,4,7,10-tetrazacyclododecane), Cyclam (1,4,8,11-Tetraazacyclotetradecane), Linear Polyethyleneimine (Poly(iminoethylene)), Norspermidine, p-Phenylenediamine (1,4-diaminobenzene), Diethylenetriamine (N-(2-aminoethyl)-1,2-ethanediamine), thermospermine, Tris(2-aminoethyl)amine, Hexamethylenediamine, Beta-lysine (3,6-diaminohexanoic acid), m-Phenylenediamine (1,3-diaminobenzene), Diaminopropane (1,2-Diaminopropane), Ethylenediamine dihydroiodide, and polyamine D 400 (Polyoxyalkyleneamine D 400).
48. The particle of any of paragraphs 44-47, wherein the cationic molecule in the particle is present in an amount from about 0.1% to about 10% (w/w).
49. The particle of any of paragraphs 44-48, wherein the cationic molecule in the particle is present in an amount from about 0.5% to about 5% (w/w).
50. The particle of any of paragraphs 44-49, wherein ratio of the cationic molecule to the active agent in the particle is from about 100:1 to about 1:100.
51. The particle of any of paragraphs 44-50, wherein ratio of the cationic molecule to the active agent in the particle is from about 10:1 to about 1:10.
52. The particle of any of paragraphs 44-51, wherein ratio of total lipids to the cationic molecule in the particle is about 100:1 to about 1:100.
53. The particle of any of paragraphs 44-52, wherein ratio of total lipids to the cationic molecule in the particle is about 5:1 to about 1:5.
54. The particle of any of paragraphs 1-53, wherein the particle further comprises an excipient in addition to the first lipid and the active agent.
55. The particle of any of paragraphs 1-54, wherein the excipient is sodium docusate.
56. The particle of any of paragraphs 1-55, wherein the particle further comprises a second active agent.
57. The particle of paragraph 1, wherein the coating layer comprises myristic acid and EGDS.
58. The particle of paragraph 57, wherein the active agent is zinc pyrithione and the coating layer comprises ethylene glycol distearate, caprylic acid, capric acid, lauric acid, myristic acid, palmitic acid, zinc recinoleate, or CoQ10.
59. The particle of paragraph 58, wherein the active agent is zinc pyrithione and the coating layer comprises myristic acid and EGDS.
60. The particle of paragraph 1, wherein the active agent is zinc pyrithione and the coating layer comprises paraffin and at least one of myristic acid, lauric acid, capric acid, caprylic acid.
61. The particle of paragraph 1, wherein the active agent is zinc pyrithione and the coating layer comprises ethylene glycol distearate and egg albumin.
62. The particle of paragraph 61, wherein ratio of zinc pyrithione to total of the ethylene glycol distearate and the egg albumin is 30:1.
63. The particle of paragraph 62, wherein ratio of the ethylene glycol distearate to the egg albumin is about 3:2.
64. The particle of paragraph 1, wherein the active agent is ketoconazole and the coating layer comprises EGDS, polyglyceryl-6-distearate or Tripalmitin.
65. The particle of paragraph 1, wherein the active agent is zinc pyrithione and the first lipid is ethylene glycol distearate, zinc recinoleate, palmitic acid, or CoQ10, and wherein ratio of zinc pyrithione to the first lipid is about 5:1.
66. The particle of paragraph 1, wherein the active agent is zinc pyrithione and the first lipid is ethylene glycol distearate, and wherein ratio of zinc pyrithione to ethylene glycol distearate is about 25:1.
67. The particle of paragraph 1, wherein the active agent is zinc pyrithione and the coating layer comprises paraffin and one of myristic acid, lauric acid, capric acid, and caprylic acid, and wherein ratio of zinc pyrithione to total lipids is about 25:1.
68. The particle of paragraph 67, wherein ratio of paraffin to one of one of myristic acid, lauric acid, capric acid, and caprylic acid is about 1:1.
69. The particle of paragraph 1, wherein the active agent is zinc pyrithione and the coating layer comprises paraffin and caprylic acid, and wherein ratio of zinc pyrithione to total lipids is about 5:1.
70. The particle of paragraph 69, wherein ratio of paraffin to the caprylic acid is about 1:1.
71. A particle comprising an active agent and a protein, wherein the particle comprises a coating layer of the protein on a core comprising the active agent.
72. The particle of paragraph 71, wherein the protein is selected from the group consisting of Actin, Albumin, Amaranth Protein, Ammonium Hydrolyzed Animal Protein, Animal protein, Barley Protein, Brazil Nut Protein, Casein, Collagen, Collagen protein hydrolyzed, Conchiolin Protein, corn protein, Cottonseed Protein, Elastin, Extensin, Fibroin, Fibronectin, Fish Protein, Gadidae Protein, Gelatin, Glutein, Glycoproteins, Hazelnut Protein, Hemoglobin, Hemp Seed Protein, Honey Protein, Hydrolyzed Actin, Hydrolyzed Amaranth Protein, Hydrolyzed animal protein, Hydrolyzed Barley Protein, Hydrolyzed Brazil Nut Protein, Hydrolyzed Conchiolin Protein, Hydrolyzed corn protein, Hydrolyzed Cottonseed Protein, Hydrolyzed Elastin, Hydrolyzed Extensin, Hydrolyzed Fibroin, Hydrolyzed Fibronectin, Hydrolyzed Fish Protein, Hydrolyzed Gadidae Protein, Hydrolyzed Gadidae Protein, Hydrolyzed Gelatin, Hydrolyzed Hair Keratin, Hydrolyzed Hazelnut, Hydrolyzed Hazelnut Protein, Hydrolyzed Hemoglobin, Hydrolyzed Hemp Seed Protein, Hydrolyzed Honey Protein, Hydrolyzed Keratin, Hydrolyzed Lupine Protein, Hydrolyzed Maple Sycamore Protein, Hydrolyzed Milk Protein, Hydrolyzed Oat Protein, Hydrolyzed Pea Protein, Hydrolyzed Potato Protein, Hydrolyzed Reticulin, Hydrolyzed Royal Jelly Protein, Hydrolyzed Sericin, Hydrolyzed Serum Protein, Hydrolyzed Sesame Protein, Hydrolyzed Soy Protein, Hydrolyzed Soymilk Protein, Hydrolyzed Spinal Protein, Hydrolyzed Spongin, Hydrolyzed Sweet Almond Protein, Hydrolyzed Vegetable Protein, Hydrolyzed Wheat Gluten, Hydrolyzed Wheat Protein, Hydrolyzed Whey Protein, Hydrolyzed Yeast Protein, Hydrolyzed Yogurt Protein, Hydrolyzed Zein, Integrin, Jojoba protein HP, Hydrolyzed, keratin, Lupine Protein, Maple Sycamore Protein, MEA-Hydrolyzed Collagen, MEA-Hydrolyzed Silk, Milk Protein, Myosin, Oat Protein, Pea Protein, polylysine, Potato Protein, Reticulin, Rice Quat, Royal Jelly Protein, Sericin, Serum Protein, Sesame Protein, Silk powder, Sodium Hydrolyzed Casein, Soy Protein, Soy Rice Peptides, Soymilk Protein, Spinal Protein, Spongin, Sweet Almond Protein, Vegetable Protein, Wheat Gluten, Whey Protein, Yeast Protein, Yogurt Protein, Zein, and Zinc Hydrolyzed Collagen.
73. The particle of any of paragraphs 71-72, wherein the albumin is bovine serum albumin, egg albumin, Hydrolyzed Lactalbumin, or Lactalbumin.
74. The particle of any of paragraphs 71-73, wherein the active agent is selected from the group consisting of small organic or inorganic molecules, saccharines, oligosaccharides, polysaccharides, peptides; proteins, peptide analogs and derivatives, peptidomimetics, nucleic acids, nucleic acid analogs and derivatives, antibodies, antigen binding fragments of antibodies, lipids, extracts made from biological materials, naturally occurring or synthetic compositions, and any combinations thereof.
75. The particle of any of paragraphs 71-74, wherein the active agent is selected from the group consisting of antifungal agents, antibacterial agents, anti-inflammatory-agents, anti-aging agents, anti-wrinkle agents, skin whitening or bleaching agents, ultraviolet (UV) light absorbing or scattering agents, skin depigmentation agents, and any combinations thereof.
76. The particle of any of paragraphs 71-75, wherein the active agent is selected from the group consisting of pyrithione salts, ketoconazole, salicylic acid, curcumin or a derivative of curcumin (curcuminoid or tetrahydro curcuminoid), titanium dioxide (TiO₂), zinc oxide (ZnO), ascorbic acid, and any combinations thereof.
77. The particle of any of paragraphs 71-76, wherein the pyrithione salt is selected from the group consisting of zinc pyrithione, sodium pyrithione, potassium pyrithione, lithium pyrithione, ammonium pyrithione, copper pyrithione, calcium pyrithione, magnesium pyrithione, strontium pyrithione, silver pyrithione, gold pyrithione, manganese pyrithione, and any combinations thereof.
78. The particle of any of paragraphs 71-77, wherein the particle has a size of about 5 nm to about 20 μm.
79. The particle of any of paragraphs 71-78, wherein the particle has a size of about 100 nm to about 10 μm.
80. The particle of any of paragraphs 71-79, wherein the particle has a size of about 200 nm to about 6 μm or wherein the particle has a size of about 1 μm to about 6 μm.
81. The particle of any of paragraphs 71-80, wherein the particle has a size of about 300 nm to about 700 nm.
82. The particle of any of paragraphs 71-81, wherein the coating layer has a thickness of about 1 nm to about 5000 nm.
83. The particle of any of paragraphs 71-82, wherein the coating layer has a thickness of about 1 nm to about 150 nm.
84. The particle of any of paragraphs 71-83, wherein the active agent in the particle is present in an amount from about 1% to about 99% (w/w).
85. The particle of any of paragraphs 71-84, wherein the pyrithione salt in the particle is present in an amount from about 5% to about 50% (w/w).
86. The particle of any of paragraphs 71-85, wherein the protein in the particle is present in an amount from about 1% to about 99% (w/w).
87. The particle of any of paragraphs 1-86, wherein the protein in the particle is present in an amount from about 5% to about 50%.
88. The particle of any of paragraphs 71-87, wherein ratio of the protein to the active agent in the particle is from about 100:1 to about 1:100.
89. The particle of any of paragraphs 71-88, wherein ratio of the protein to the active agent in the particle is about 10:1 to about 1:50.
90. The particle of any of paragraphs 71-89, wherein ratio of the protein to the active agent in the particle is about 2:1 to about 1:30.
91. A particle comprising an active agent and a cationic molecule, wherein the particle comprises a coating layer of the cationic molecule on a core comprising the active agent.
92. The particle of paragraph 91, wherein the cationic molecule is a polyamine.
93. The particle of any of paragraphs 91-92, wherein the cationic molecule is selected from the group consisting of Putrescine (Butane-1,4-diamine), Cadaverine (Pentane-1,5-diamine), Spermidine, Spermine, Cyclen (1,4,7,10-tetrazacyclododecane), Cyclam (1,4,8,11-Tetraazacyclotetradecane), Linear Polyethyleneimine (Poly(iminoethylene)), Norspermidine, p-Phenylenediamine (1,4-diaminobenzene), Diethylenetriamine (N-(2-aminoethyl)-1,2-ethanediamine), thermospermine, Tris(2-aminoethyl)amine, Hexamethylenediamine, Beta-lysine (3,6-diaminohexanoic acid), m-Phenylenediamine (1,3-diaminobenzene), Diaminopropane (1,2-Diaminopropane), Ethylenediamine dihydroiodide, and polyamine D 400 (Polyoxyalkyleneamine D 400).
94. The particle of any of paragraphs 91-93, wherein the active agent is selected from the group consisting of small organic or inorganic molecules, saccharines, oligosaccharides, polysaccharides, peptides; proteins, peptide analogs and derivatives, peptidomimetics, nucleic acids, nucleic acid analogs and derivatives, antibodies, antigen binding fragments of antibodies, lipids, extracts made from biological materials, naturally occurring or synthetic compositions, and any combinations thereof.
95. The particle of any of paragraphs 91-94, wherein the active agent is selected from the group consisting of antifungal agents, antibacterial agents, anti-inflammatory-agents, anti-aging agents, anti-wrinkle agents, skin whitening or bleaching agents, ultraviolet (UV) light absorbing or scattering agents, skin depigmentation agents, and any combinations thereof.
96. The particle of any of paragraphs 91-95, wherein the active agent is selected from the group consisting of pyrithione salts, ketoconazole, salicylic acid, curcumin or a derivative of curcumin (curcuminoid or tetrahydro curcuminoid), titanium dioxide (TiO₂), zinc oxide (ZnO), ascorbic acid, and any combinations thereof.
97. The particle of any of paragraphs 91-96, wherein the pyrithione salt is selected from the group consisting of zinc pyrithione, sodium pyrithione, potassium pyrithione, lithium pyrithione, ammonium pyrithione, copper pyrithione, calcium pyrithione, magnesium pyrithione, strontium pyrithione, silver pyrithione, gold pyrithione, manganese pyrithione, and any combinations thereof.
98. The particle of any of paragraphs 91-91, wherein the particle has a size of about 5 nm to about 20 μm.
99. The particle of any of paragraphs 91-98, wherein the particle has a size of about 100 nm to about 10 μm.
100. The particle of any of paragraphs 91-99, wherein the particle has a size of about 200 nm to about 6 μm or wherein the particle has a size of about 1 μm to about 6 μm.
101. The particle of any of paragraphs 91-100, wherein the particle has a size of about 300 nm to about 700 nm.
102. The particle of any of paragraphs 91-101, wherein the coating layer has a thickness of about 1 nm to about 5000 nm.
103. The particle of any of paragraphs 91-102, wherein the coating layer has a thickness of about 1 nm to about 150 nm.
104. The particle of any of paragraphs 91-101, wherein the active agent in the particle is present in an amount from about 1% to about 99% (w/w).
105. The particle of any of paragraphs 91-104, wherein the active agent in the particle is present in an amount from about 5% to about 50% (w/w).
106. The particle of any of paragraphs 91-101, wherein the cationic molecule in the particle is present in an amount from about 1% to about 99% (w/w).
107. The particle of any of paragraphs 91-106, wherein the cationic molecule in the particle is present in an amount from about 5% to about 50%.
108. The particle of any of paragraphs 91-107, wherein ratio of the cationic molecule to the active agent in the particle is from about 100:1 to about 1:100.
109. The particle of any of paragraphs 91-108, wherein ratio of the cationic molecule to the active agent in the particle is about 10:1 to about 1:50.
110. The particle of any of paragraphs 91-109, wherein ratio of the cationic molecule to the active agent in the particle is about 2:1 to about 1:30.
111. A composition comprising an effective amount of particle of any of paragraphs 1-110.
112. The composition of paragraph 111, wherein the composition comprises from about 0.01% to about 50% (w/w or w/v) of the particles.
113. The composition of any of paragraphs 111-112, wherein the composition comprises from about 10% to about 30% (w/w or w/v) of the particles.
114. The composition of any of paragraphs 111-113, wherein the composition further comprises an excipient.
115. The composition of any of paragraphs 111-114, wherein the composition comprises from about 5% to about 99.99% (w/w or w/v) of the excipient.
116. The composition of any of paragraphs 111-115, wherein the excipient is a solvent or an additive.
117. The composition of any of paragraphs 111-116, wherein the additive is selected from the group consisting of surfactants, stabilizers, rheology modifiers, conditioning agents, fragrances, potentiating agents, preservatives, opacifiers, pH modifiers, and any combinations thereof.
118. The composition of any of paragraphs 111-117, wherein the additive is selected from the group consisting of 45° Be'glucose syrup, Acrylates/10-30 alkyl acrylate crosspolymer, Acrylates/Acrylamide Copolymer, agar, Allantoin, Aminomethyl propanol, Ammonium lauryl sulfate (ALS), Amodimethicone emulsion, AMP-Acrylates/Allyl Methacrylate Copolymer, Behentrimonium methosulfate, Benzophenone-4, Butylene glycol, Carbapol Aqua SF-1, Carbapol-934, Carbapol-940, Carbopol, Carboxymethyl cellulose, Cassia hydroxy propyltrimoniumchloride, Cetearyl alcohol, Cetearyl alcohol, Cetearyl isononanoate, Cetrimoniumchloride (CTC), Cetyl Alcohol, Cetyl octanoate, Chloromethyl/Methylisothiazolinone, Chloromethyl/Methylisothiazolinone, Citric acid, Citrus Aurantium Dulcis (Orange) Fruit Extract, Cocamidopropyl betain (CAPB), Cocamidopropyl betain (CAPB), Coco monoethanolamide (CMEA), Cocodimonium hydroxypropyl hydrolyzed wheat protein, Coloring agent, CPB, Diisopropyl sebacate, Dimethicone PEG-7 Isostearate, Disodium EDTA, DMDM hydantoin, Dove AD shine, Emulsifying wax, Ethanol, Ethylene glycol distearate (EGDS), Ethylhexyl methoxycinnamate, eucalyptol, Flavors, Forte therapy, Fragrance, Glycerin, Glycerine, Glyceryl monohydroxystearate, Glyceryl monostearate, Glycolic acid, Guar gum, Gum base, Hyaluronic acid, Hydrated silica, Hydrolyzed silk protein, Intense repair, Lactamide MEA acetamide MEA, Lactic acid, Limnanthes Alba (Meadowfoam) Seed Oil, Linalool, Linoleamidopropyl PG-dimonium chloride phosphate, Macrogolcetostearyl ether 20, magnese chloride, Magnesium sulfate, Menthol, Methyl gluceth-20, methyl salicylate, Mineral oil, Mint-type flavor, PEG/PPG-8/3 laurate, PEG-12 dimethicone, PEG-20 Almond Glycerides, PEG-40 hydrogenated castor oil, PEG-80 glyceryl cocoate, peppermint oil, Phenyl trimethicone, Polyacrylate-1 crosspolymer, Polyethylene glycol 1450, Polyoxyethylene/polyoxypropylene block polymer (Poloxamer 407), Polyquaternium-22, Polyquaternium-39, Polysorbate 85, polysorbate-20, PPG-3 myristyl ether, Preservative, Propylene glycol, Propylene glycol monocaprylate, Prunus Armeniaca (Apricot) Kernel Oil, Pyrus Malus (Apple) Fruit Extract, retinyl palmitate, Saccharin sodium, Salicylic acid, Sensomer CT-250, Silica Xerogel, SLES, SLS, sodium chloride, sodium docusate, Sodium hydroxide, Sodium lauryl ether sulfate (SLES), sodium lauryl sulphate, Sodium monofluorophosphate, Sodium saccharin, Sorbitol, Stearamidopropyldimethylamine, Steareth-2, Steareth-21, Stearic acid, Sugar powder, sunflower seed oil, Tea tree oil, Titanium dioxide, Tocopheryl acetate, Triclosan, Triethanolamine, Wheat amino acids, Xanthan gum, zinc carbonate, zinc chloride, zinc recinoleate, zinc stearate, and any combinations thereof.
119. The composition of any of paragraphs 111-118, wherein the composition further comprises: (i) Carbopol, Ammonium lauryl sulfate (ALS), Sodium lauryl ether sulfate (SLES), Sodium hydroxide, Coco monoethanolamide (CMEA), Ethylene glycol distearate (EGDS), Propylene glycol monocaprylate, Menthol, Magnesium sulfate, Amodimethicone emulsion, Propylene glycol, Zinc carbonate, Cocamidopropyl betaine (CAPB), Cassia hydroxyl propyltrimoniumchloride, Chloromethyl/Methylisothiazolinone, Linalool, Fragrance, Citric acid, and Sodium Chloride; or (ii) Carbopol, Ammonium lauryl sulfate (ALS), Sodium lauryl ether sulfate (SLES), Sodium hydroxide, Ethylene glycol distearate (EGDS), Propylene glycol monocaprylate, Menthol, Magnesium sulfate, Amodimethicone emulsion, Propylene glycol, Zinc carbonate, Cocamidopropyl betaine (CAPB), Cassia hydroxyl propyltrimoniumchloride, Chloromethyl/Methylisothiazolinone, Linalool, Fragrance, Citric acid, and Sodium Chloride.
120. The composition of any of paragraphs 111-119, wherein the composition further comprises: (i) Carbopol, Sodium lauryl ether sulphate (SLES), Sodium hydroxide, Steareth-2, Steareth-21, Propylene glycol monocaprylate, Macrogolcetostearyl ether 20, Coco monoethanolamide (CMEA), Cetyl Alcohol, Stearamidopropyldimethylamine, Lactic acid, Cocamidopropyl betain (CAPB), Cetrimoniumchloride (CTC), Polyquaternium-22, Amodimethicone emulsion, Cassia hydroxy propyltrimoniumchloride, Propylene glycol, Glycerine, Zinc carbonate, Titanium dioxide, Linalool, Fragrance, and Chloromethyl/Methylisothiazolinone; or (ii) Carbopol, Sodium lauryl ether sulphate (SLES), Sodium hydroxide, Steareth-2, Steareth-21, Propylene glycol monocaprylate, Macrogolcetostearyl ether 20, Coco monoethanolamide (CMEA), Cetyl Alcohol, Stearamidopropyldimethylamine, Lactic acid, Cocamidopropyl betain (CAPB), Cetrimoniumchloride (CTC), Polyquaternium-22, Amodimethicone emulsion, Cassia hydroxy propyltrimoniumchloride, Propylene glycol, Glycerine, Titanium dioxide, Linalool, Fragrance, and Chloromethyl/Methylisothiazolinone.
121. The composition of any of paragraphs 111-120, wherein the composition further comprises: (i) Stearic acid, Mineral oil, Glyceryl monostearate, Glyceryl monohydroxystearate, Cetearyl alcohol, Cetyl octanoate, Emulsifying wax, Cabopol, Triethanolamine, Water, Glycerin, Propylene glycol, Fragrance, and Preservative; (ii) Stearic acid, Mineral oil, Glyceryl monostearate, Glyceryl monohydroxystearate, Cetearyl alcohol, Cetyl octanoate, Cabopol, Triethanolamine, Glycerin, Salicylic acid, Citrus Aurantium Dulcis (Orange) Fruit Extract, Fragrance, and Preservative; (iii) Emulsifying wax, Behentrimonium methosulfate and Cetearyl alcohol, PPG-3 myristyl ether, Cetearyl isononanoate, Dimethicone PEG-7 Isostearate, Ethylhexyl methoxycinnamate, Cabopol, Acrylates/Acrylamide Copolymer (and) Mineral Oil, (and) Polysorbate 85, Triethanolamine, Glycerin, Hyaluronic acid, Wheat amino acids, Lactamide MEA and acetamide MEA, Hydrolyzed silk protein, Salicylic acid, Propylene glycol, Pyrus Malus (Apple) Fruit Extract, Citrus Aurantium Dulcis (Orange) Fruit Extract, Tocopheryl acetate, Fragrance, and Preservative; or (iv) Glyceryl monohydroxystearate, Limnanthes Alba (Meadowfoam) Seed Oil, Prunus Armeniaca (Apricot) Kernel Oil, Phenyl trimethicone, Diisopropyl sebacate, Ethylhexyl methoxycinnamate, Cabopol, Acrylates/Acrylamide Copolymer (and) Mineral Oil, (and) Polysorbate 85, AMP-Acrylates/Allyl Methacrylate Copolymer, Triethanolamine, PEG-20 Almond Glycerides, Titanium dioxide, Propylene glycol, Linoleamidopropyl PG-dimonium chloride phosphate, Cocodimonium hydroxypropyl hydrolyzed wheat protein, Tocopheryl acetate, Fragrance, and Preservative.
122. The composition of any of paragraphs 111-121 wherein the composition further comprises: (i) Glycerin, Methyl gluceth-20, Benzophenone-4, Acrylates/10-30 alkyl acrylate crosspolymer, PEG/PPG-8/3 laurate, Aminomethyl propanol, Polyquaternium-39, PEG-80 glyceryl cocoate, Coloring agent, Preservative, and Fragrance; (ii) Disodium EDTA, Propylene glycol, Carbopol, Aminomethyl propanol, Tea tree oil, PEG-40 hydrogenated castor oil, polysorbate-20, Coloring agent, Preservative, and Fragrance; (iii) Butylene glycol, Glycerin, Methyl gluceth-20, Allantoin, Disodium EDTA, PEG-12 dimethicone, Polyacrylate-1 crosspolymer, Glycolic acid, Triethanolamine, Tocopheryl acetate, retinyl palmitate, sunflower seed oil, agar, Coloring agent, Preservative, and Fragrance; or (iv) Glycerin, Methyl gluceth-20, Carbopol, Triethanolamine, Ethanol, Triclosan, Coloring agent, Preservative, and Fragrance.
123. The composition of any of paragraphs 111-122, wherein the composition further comprises Carboxymethyl cellulose, Polyethylene glycol 1450, Sorbitol, Glycerin, Sodium monofluorophosphate, Sodium saccharin, Preservative, Coloring agent, Silica Xerogel, Hydrated silica, Mint-type flavor, and Sodium lauryl sulfate.
124. The composition of any of paragraphs 111-123, wherein the composition further comprises Ethyl alcohol, menthol, methyl salicylate, peppermint oil, eucalyptol, Glycerin, Polyoxyethylene/polyoxypropylene block polymer (Poloxamer 407), and Saccharin sodium.
125. The composition of any of paragraphs 111-124, wherein the composition further comprises Gum base, 45° Be'glucose syrup, Sugar powder, Flavors, Glycerin, and Preservative.
126. The composition of any of paragraphs 111-125, wherein the composition is a cream, oil, lotion, serum, gel, shampoo, conditioner, tooth paste, mouth wash, chewing gum, sun screen, nail varnish, ointment, foam, spray, or aerosol.
127. The composition of any of paragraphs 111-126, wherein the composition is an anti-dandruff hair care composition selected from the group consisting of a shampoo, a conditioner, a rinse, a lotion, an aerosol, a gel, a mousse, and a hair dye.
128. The composition of any of paragraphs 111-126, wherein the composition is a skin care composition selected from the group consisting of lotions, creams, gels, sticks, sprays, ointments, cleansing liquid washes, cleansing solid bars, pastes, foams, powders, shaving creams, and wipes.
129. The composition of any of paragraphs 111-126, wherein the composition is an oral care composition selected from the group consisting of tooth pastes, mouth washes, and chewing gums.
130. The composition of any of paragraphs 111-126, wherein the composition is an antifungal, antibacterial, anti-inflammatory, anti-aging, anti-wrinkle, or skin whitening or skin bleaching composition.
131. The composition of any of paragraphs 111-126, wherein the composition is an anti-acne composition.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

As used herein, the term “herein” is means the whole of the disclosure and as such is not meant to be limited to a particular section or subsection of the disclosure.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages can mean±5%, ±4%, ±3%, ±2.5%, ±2%, ±1.5%, ±1%, or ±0.5% of the value being referred to.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

The disclosure is further illustrated by the following examples which should not be construed as limiting. The examples are illustrative only, and are not intended to limit, in any manner, any of the aspects described herein. The following examples do not in any way limit the invention.

### EXAMPLES

### Example 1

### Lipid Coated Zinc Pyrithione (ZPT) Particle Dispersions (Compositions D1-D11)

Preparation:

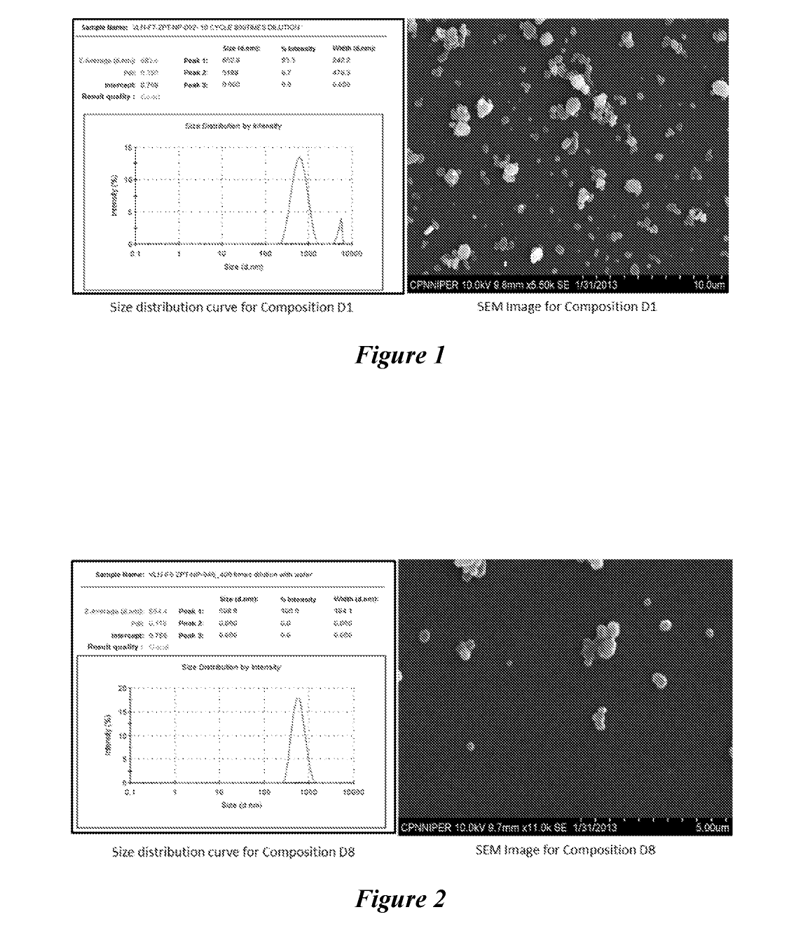

A mixture of lipid and 1% aqueous solution of sodium docusate (and 2% egg albumin, if required) is heated to melt the lipid under continuous vigorous stirring. ZPT powder is added in portions to the stirring hot mixture. The resulting suspension is passed through high pressure homogenizer at about 1200-1500 bar. The output dispersion is collected in a beaker kept in ice bath and recycled about 6-10 times to yield a dispersion of appropriately sized particles (about 200 nm to about 800 nm). The size distribution is determined by ZetaSizer (ZS-90 from Malvern Instruments) and Scanning Electron Microscope (SEM, Hitachi, S-3400 N, Japan) as shown in FIGS. 1 and 2. The resulting dispersions are stabilized by adding carbopol solution followed by neutralization with sodium hydroxide to a pH ranging from about 6.5 to about 7.0. Some of the data for the examples are given in Table 1.

TABLE 1
Dispersion compositions of coated particles
prepared using high pressure homogenizer

| | | | | Core | Avg. |
|---|---|---|---|---|---|
| | | | | Strengthin | Particle |
| Compo- | | | Coat:Core | dispersion | Size |
| sitions | Core | Coat | ratio | (% w/v) | (PDI) |
| D1 | ZPT | EGDS | 1:5 | 10 | 683 (0.350) |
| D2 | ZPT | Zinc | 1:5 | 10 | 559 (0.246) |
| | | Ricinoleate | | | |
| D3 | ZPT | Palmitic acid | 1:5 | 10 | 593 (0.248) |
| D4 | ZPT | Myristic acid: | 1:25 | 50 | 443 (0.719) |
| | | Paraffin (1:1) | | | |
| D5 | ZPT | Lauric acid: | 1:25 | 50 | 292 (0.462) |
| | | Paraffin (1:1) | | | |
| D6 | ZPT | Capric acid: | 1:25 | 50 | 483 (0.779) |
| | | Paraffin (1:1) | | | |
| D7 | ZPT | Caprylic acid: | 1:5 | 10 | 500 (0.223) |
| | | Paraffin (1:1) | | | |
| D8 | ZPT | Caprylic acid: | 1:25 | 10 | 554 (0.118) |
| | | Paraffin (1:1) | | | |
| D9 | ZPT | Coenzyme | 1:5 | 10 | 514 (0.222) |
| | | Q10 | | | |
| D10 | ZPT | EGDS:Egg | 1:30 | 50 | 292 (0.008) |
| | | albumin (3:2) | | | |
| D11 | ZPT | EGDS | 1:25 | 30 | 733 (0.219) |

### Example 2

### Lipid Coated Ketoconazole (KTZ) Particle Dispersions (Compositions D12-D16)

Preparation:

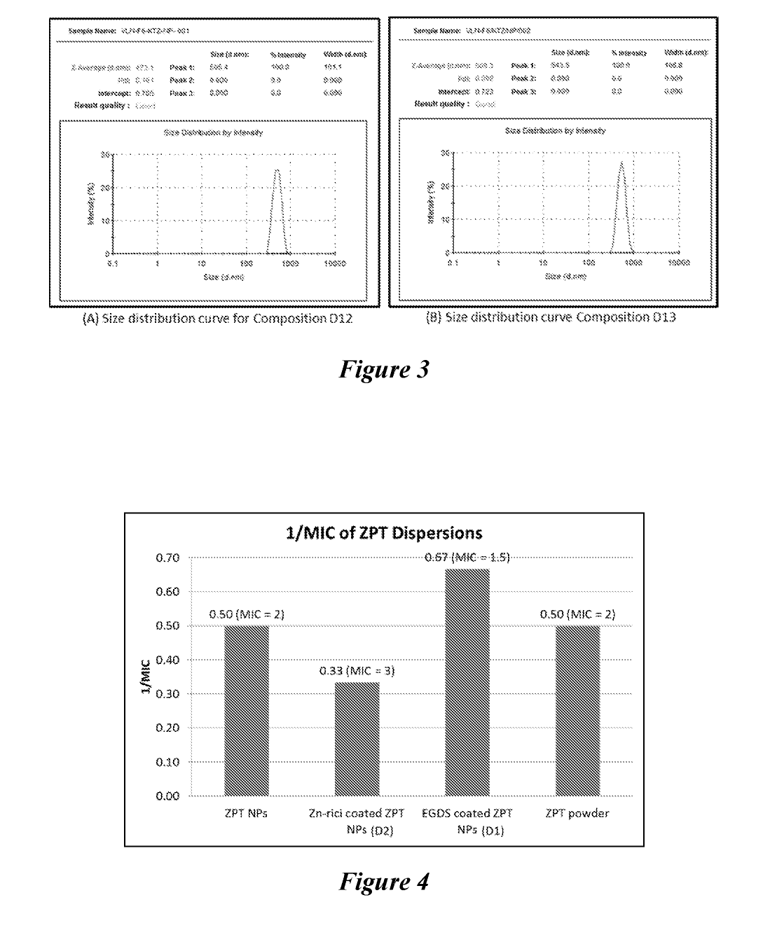

A mixture of lipid and 1% aqueous solution of sodium docusate (or polyvinyl alcohol) is heated to melt the lipid under continuous vigorous stirring. KTZ powder is added in portions to the stirring hot mixture. The resulting suspension is passed through high pressure homogenizer at about 1200-1500 bar. The output dispersion is collected in a beaker kept in ice bath and recycled about 6-10 times to yield a dispersion of appropriately sized particles (about 300 nm to about 700 nm). The size distribution is determined by ZetaSizer (ZS-90 from Malvern Instruments) as shown in FIGS. 3A and 3B. The resulting dispersions are stabilized by adding carbopol solution followed by neutralization with sodium hydroxide to a pH ranging from about 6.5 to about 7.0. Some of the data for the examples are given in Table 2.

TABLE 2
Dispersion compositions of lipid coated ketoconazole
particles prepared using high pressure homogenizer

| | | | | Core | Avg. |
|---|---|---|---|---|---|
| | | | | Strength in | Particle |
| Compo- | | | Coat:Core | dispersion | Size |
| sitions | Core | Coat | ratio | (%) | (PDI) |
| D12 | Ketoconazole | EGDS | 1:5 | 10 | 473 (0.101) |
| D13 | Ketoconazole | Polyglyceryl- | 1:5 | 10 | 509 (0.092) |
| | | 6 distearate | | | |
| D14 | Ketoconazole | Tripalmitin | 1:5 | 10 | 717 (0.189) |

### Example 3

### Lipid Coated Salicylic Acid (SAL) Particle Dispersions (Compositions D17-D18)

Preparation:

A mixture of lipid and 1% aqueous solution of sodium docusate is heated to melt the lipid under continuous vigorous stirring. SAL powder is added in portions to the stirring hot mixture. The resulting suspension is passed through high pressure homogenizer at about 1200-1500 bar. The output dispersion is collected in a beaker kept in ice bath and recycled about 6-10 times to yield a dispersion of appropriately sized particles (about 300 nm to about 700 nm). The size distribution is determined by ZetaSizer (ZS-90 from Malvern Instruments) and Scanning Electron Microscope (SEM, Hitachi, S-3400 N, Japan). The resulting dispersions are stabilized by adding carbopol solution followed by neutralization with sodium hydroxide to a pH ranging from about 6.5 to about 7.0.

### Example 4

### Coenzyme Q10 (CoQ10) Coated Curcuminoids (CMD) or Tetrahydro Curcuminoids (THC) Particle Dispersions (Compositions D19-D20)

Preparation:

A mixture of CoQ10 and 1% aqueous solution of sodium docusate is heated to melt CoQ10 under continuous vigorous stirring. CMD (or THC) powder is added in portions to the stirring hot mixture. The resulting suspension is passed through high pressure homogenizer at about 1200-1500 bar. The output dispersion is collected in a beaker kept in ice bath and recycled about 6-10 times to yield a dispersion of appropriately sized particles (about 300 nm to about 700 nm). The size distribution is determined by ZetaSizer (ZS-90 from Malvern Instruments) and Scanning Electron Microscope (SEM, Hitachi, S-3400 N, Japan). The resulting dispersions are stabilized by adding carbopol solution followed by neutralization with sodium hydroxide to a pH ranging from about 6.5 to about 7.0.

### Example 5

### CoQ10 Coated Titanium Dioxide Particle Dispersions (Compositions D21-D22)

Preparation:

A mixture of CoQ10 and 1% aqueous solution of sodium docusate is heated to melt CoQ-10 under continuous vigorous stirring. Titanium dioxide (TiO₂) powder is added in portions to the stirring hot mixture. The resulting suspension is passed through high pressure homogenizer at about 1200-1500 bar. The output dispersion is collected in a beaker kept in ice bath and recycled about 6-10 times to yield a dispersion of appropriately sized particles (about 300 nm to about 700 nm). The size distribution is determined by ZetaSizer (ZS-90 from Malvern Instruments) and Scanning Electron Microscope (SEM, Hitachi, S-3400 N, Japan). The resulting dispersions are stabilized by adding carbopol solution followed by neutralization with sodium hydroxide to a pH ranging from about 6.5 to about 7.0.

### Example 6

### CoQ10 Coated Zinc Oxide (ZnO) Particle Dispersions (Compositions D23-D24)

Preparation:

A mixture of CoQ10 and 1% aqueous solution of sodium docusate is heated to melt CoQ-10 under continuous vigorous stirring. ZnO powder is added in portions to the stirring hot mixture. The resulting suspension is passed through high pressure homogenizer at about 1200-1500 bar. The output dispersion is collected in a beaker kept in ice bath and recycled about 6-10 times to yield a dispersion of appropriately sized particles (about 300 nm to about 700 nm). The size distribution is determined by ZetaSizer (ZS-90 from Malvern Instruments) and Scanning Electron Microscope (SEM, Hitachi, S-3400 N, Japan). The resulting dispersions are stabilized by adding carbopol solution followed by neutralization with sodium hydroxide to a pH ranging from about 6.5 to about 7.0.

### Example 7

### Lipid Coated Chloroxylenol Particle Dispersions (Compositions D25-D26)

Preparation:

A mixture of lipid and 1% aqueous solution of sodium docusate is heated to melt lipid under continuous vigorous stirring. Chloroxylenol powder is added in portions to the stirring hot mixture. The resulting suspension is passed through high pressure homogenizer at about 1200-1500 bar. The output dispersion is collected in a beaker kept in ice bath and recycled about 6-10 times to yield a dispersion of appropriately sized particles (about 200 nm to about 700 nm). The size distribution is determined by ZetaSizer (ZS-90 from Malvern Instruments) and Scanning Electron Microscope (SEM, Hitachi, S-3400 N, Japan). The resulting dispersions are stabilized by adding carbopol solution followed by neutralization with sodium hydroxide to a pH ranging from about 6.5 to about 7.0.

### Example 8

### CoQ10 Coated Ascorbic Acid Particle Dispersions (Compositions D27-D28)

Preparation:

A mixture of CoQ10 and 1% aqueous solution of sodium docusate is heated to melt CoQ-10 under continuous vigorous stirring. Ascorbic acid powder is added in portions to the stirring hot mixture to supersaturate the mixture. The resulting suspension is passed through high pressure homogenizer at an appropriate pressure. The output dispersion is collected in a beaker kept in ice bath and recycled about 6-10 times to yield a dispersion of appropriately sized particles (about 100 nm to about 900 nm). The size distribution is determined by ZetaSizer (ZS-90 from Malvern Instruments) and Scanning Electron Microscope (SEM, Hitachi, S-3400 N, Japan). The resulting dispersions are stabilized by adding suitable stabilizer followed by neutralization with pH modulator.

### Example 9

### Preparation of Shampoo Formulations with Lipid Coated Nanoparticle Dispersions (Compositions S1-S3)

Shampoo formulations with lipid coated nanoparticle dispersions (of Examples 1 and 2) are designed and formulated as per the compositions shown in Table 3.

TABLE 3
Shampoo Formulations for Compositions S1, S2 and S3

| | | Strength | Composition (%) | | |
|---|---|---|---|---|---|
| Sr. No. | Ingredient | in % | S1 | S2 | S3 |
| | Phase A | | | | |
| 1 | Water | NA | qs. | qs. | qs. |
| 2 | Carbopol | 30 | 2.5 | 2.5 | 2 |
| 3 | Ammonium lauryl sulfate (ALS) | 27 | 10 | 10 | 10 |
| 3 | Sodium lauryl ether sulfate (SLES) | 28 | 30 | 30 | 25 |
| 4 | Sodium hydroxide | 18 | qs. | qs. | qs. |
| | Phase B | | | | |
| 5 | Coco monoethanolamide (CMEA) | NA | 1 | 0 | 0 |
| 6 | Ethylene glycol distearate (EGDS) | NA | 1 | 1 | 1 |
| 7 | Propylene glycol monocaprylate | NA | 4 | 0 | 4 |
| 8 | Menthol | NA | 0.1 | 0.1 | 0.1 |
| | Phase C | | | | |
| 9 | Lipid coated NPs (D1/D2/D12) | 10 | 10 (D1) | 10 (D2) | 10 (D12) |
| 10 | Magnesium sulfate | NA | 0.5 | 0.5 | 0.5 |
| 11 | Amodimethicone emulsion | NA | 1 | 1 | 1 |
| 12 | Propylene glycol | NA | 1 | 1 | 1 |
| 13 | Zinc carbonate | NA | 1 | 1 | 0 |
| 14 | Cocamidopropyl betaine (CAPB) | 30 | 10 | 10 | 10 |
| 15 | Cassia hydroxyl | 1 | 10 | 10 | 10 |
| | propyltrimoniumchloride | | | | |
| 16 | Chloromethyl/ | 1.51 | 0.05 | 0.05 | 0.05 |
| | Methylisothiazolinone | | | | |
| 17 | Linalool | NA | 1 | 1 | 1 |
| 18 | Fragrance | NA | qs. | qs. | qs. |
| 19 | Citric acid | 50 | qs. | qs. | qs. |
| 20 | Sodium Chloride | 30 | qs. | qs. | qs. |

Method of Preparation:

(1) Phase A: A required amount of water is added to a mixing vessel and stirred slowly (50-55 rpm) using an overhead stirrer. Carbopol is added to water followed by the slow addition of a premix of about 30% aqueous solutions of ammonium lauryl sulfate (ALS) and sodium lauryl ether sulfate (SLES). The mixture is neutralized by sodium hydroxide solution. (2) Phase B: A mixture of CMEA, EGDS, menthol and propylene glycol monocaprylate is heated to melt. The resulting melt is immediately poured to Phase A while stirring at about 60° C. After stirring for about 5 min at the same temperature, it is allowed to cool to about 35° C. to about 40° C. (3) Phase C: Lipid coated nanoparticle dispersion (of Examples 1 and 2) is added to the above stirring mixture. Then, magnesium sulfate is added while stirring followed by additions of amodimethicone emulsion and propylene glycol, followed by the addition of zinc carbonate, CAPB (30% aq.), Cassia hydroxypropyltrimonium chloride and preservatives in the same order as mentioned in the Table 3. The continuously stirring mixture (150-160 rpm) is then allowed to cool to room temperature followed by addition of fragrance. Finally, pH is adjusted with citric acid and viscosity by sodium chloride, and mixture is continued to stir to yield a smooth and shiny shampoo (maximum speed of about 150-160 rpm).

### Example 10

### Preparation of Conditioner Formulations with Lipid Coated Nanoparticle Dispersions (Compositions C1-C3

Conditioner formulations with lipid coated nanoparticle dispersions (of Examples 1 and 2) are designed and formulated as per the compositions shown in Table 4.

TABLE 4
Conditioner Formulations for Compositions C1, C2 and C3

| | | Strength | Composition (%) | | |
|---|---|---|---|---|---|
| Sr. No. | Ingredients | (%) | C1 | C2 | C3 |
| | Phase A | | | | |
| 1 | Water | NA | qs. | qs. | qs. |
| 2 | Carbopol | 30 | 3 | 1.5 | 1.5 |
| 3 | Sodium lauryl ether sulphate | 28 | 5 | 5 | 5 |
| | (SLES) | | | | |
| 4 | Sodium hydroxide | 18 | qs. | qs. | qs. |
| | Phase B | | | | |
| 5 | Steareth-2 | NA | 3 | 3 | 3 |
| 6 | Steareth-21 | NA | 2 | 2 | 2 |
| 7 | Propylene glycol monocaprylate | NA | 9 | 9 | 5 |
| 8 | Macrogolcetostearyl ether 20 | NA | 6 | 6 | 6 |
| 9 | Coco monoethanolamide | NA | 1.2 | 1.2 | 1.2 |
| | (CMEA) | | | | |
| 10 | Cetyl Alcohol | NA | 5 | 5 | 5 |
| 11 | Stearamidopropyldimethylamine | NA | 2 | 1 | 1 |
| 12 | Lactic acid | 50 | qs. | qs. | qs. |
| | Phase C | | | | |
| 13 | Cocamidopropyl betain (CAPB) | 30 | 10 | 10 | 10 |
| 14 | Cetrimoniumchloride (CTC) | 30 | 2 | 2 | 2 |
| 15 | Polyquaternium-22 | NA | 0.5 | 0.5 | 0.5 |
| 16 | Amodimethicone emulsion | NA | 0.5 | 0.5 | 0.5 |
| 17 | Cassia hydroxy | 1 | 5 | 5 | 5 |
| | propyltrimoniumchloride | | | | |
| 18 | Propylene glycol | NA | 2 | 2 | 2 |
| 19 | Glycerine | NA | 5 | 5 | 5 |
| 20 | Lipid coated NPs (D3/D11/D13) | 10/30* | 5 (D3) | 3.33* (D11) | 10 (D13) |
| 21 | Zinc carbonate | NA | 1 | 1 | 0 |
| 22 | Titanium dioxide | NA | 0.5 | 0.5 | 0.5 |
| 23 | Linalool | NA | 1 | 1 | 1 |
| 24 | Fragrance | NA | qs. | qs. | qs. |
| 25 | Chloromethyl/ | 1.51 | 0.05 | 0.05 | 0.05 |
| | Methylisothiazolinone | | | | |

Method of Preparation:

(1) Phase A: A required amount of water is added to a mixing vessel and stirred slowly (50-55 rpm) using an overhead stirrer. Carbopol is added to water followed by the slow addition of about 28% aqueous solution of sodium lauryl ether sulfate (SLES). Then mixture is neutralized by adding sodium hydroxide solution. (2) Phase B: Components of Phase B are mixed and heated to melt. Lactic acid is added to the resulting melted mixture to neutralize. The Phase B is added to Phase A while stirring at about 60° C. After uniform mixing, the mixture is allowed to cool to 35° C. to 40° C. (3) Phase C: To the above stirring mixture, cocamidopropylbetaine, cetrimonium chloride, polyquaternium-22, amodimethicone emulsion, Cassia hydroxypropyltrimonium chloride, propylene glycol and glycerin are added slowly in the same order as mentioned in Table 4 and stirred (50-100 rpm) till uniform mixing. Lipid coated nanoparticles dispersion is added to the stirring mixture followed by addition of zinc carbonate and titanium dioxide. The mixture is then allowed to cool to room temperature. Finally, linalool, fragrance and preservatives are added, and the mixture is allowed to stir in order to yield a smooth uniform conditioner cream (about 150-160 rpm).

### Example 11

### Preparation of Cream Formulations with Lipid Coated Nanoparticle Dispersions (Compositions CR1-CR4)

Cream formulations with lipid coated nanoparticle dispersions (of Examples 2, 3, 4 and 6) are designed and formulated as per the compositions shown in Table 5.

TABLE 5
Cream Formulations for Compositions CR1, CR2, CR3 and CR4

| Sr. | | Strength | Composition (%) | | | |
|---|---|---|---|---|---|---|
| No. | Ingredients | (%) | CR1 | CR2 | CR3 | CR4 |
| | Phase A | | | | | |
| 1 | Stearic acid | N/A | 4.0 | 4.0 | — | — |
| 2 | Mineral oil | 70 | 5.0 | 5.0 | — | — |
| 3 | Glyceryl monostearate | N/A | 1.0 | 1.0 | | 2.0 |
| 4 | Glyceryl | N/A | 2.0 | 2.0 | — | — |
| | monohydroxystearate | | | | | |
| 5 | Cetearyl alcohol | N/A | 3.0 | 3.0 | — | — |
| 6 | Cetyl octanoate | N/A | 5.0 | 10.0 | — | — |
| 7 | Emulsifying wax | N/A | 2.0 | — | 5.0 | — |
| 8 | Behentrimonium | N/A | — | — | 1.5 | — |
| | methosulfate and Cetearyl | | | | | |
| | alcohol | | | | | |
| 9 | PPG-3 myristyl ether | N/A | — | — | 3.0 | — |
| 10 | Cetearyl isononanoate | N/A | — | — | 3.0 | — |
| 11 | Dimethicone PEG-7 | N/A | — | — | 2.0 | — |
| | Isostearate | | | | | |
| 12 | Limnanthes Alba | N/A | — | — | — | 3.0 |
| | (Meadowfoam) Seed Oil | | | | | |
| 13 | Prunus Armeniaca (Apricot) | N/A | — | — | — | 3.0 |
| | Kernel Oil | | | | | |
| 14 | Phenyl trimethicone | N/A | — | — | — | 2.0 |
| 15 | Diisopropyl sebacate | N/A | — | — | — | 2.0 |
| 16 | Ethylhexyl | N/A | — | — | 2.0 | 4.0 |
| | methoxycinnamate | | | | | |
| | Phase B | | | | | |
| 17 | Cabopol | N/A | 0.2 | 0.2 | 0.5 | 0.2 |
| 18 | Acrylates/Acrylamide | N/A | — | — | 3.0 | 2.0 |
| | Copolymer (and) Mineral Oil | | | | | |
| | (and) Polysorbate 85 | | | | | |
| 19 | AMP-Acrylates/Allyl | 26 | — | — | — | 1.0 |
| | Methacrylate Copolymer | | | | | |
| 20 | Triethanolamine | N/A | 1.0 | 1.0 | 0.5 | 0.5 |
| 21 | Water | N/A | qs. | qs. | qs. | qs. |
| 22 | Glycerin | N/A | 3.0 | 3.0 | 3.0 | — |
| 23 | Hyaluronic acid | N/A | — | — | 5.0 | — |
| 24 | Wheat amino acids | N/A | — | — | 1.0 | — |
| 25 | Lactamide MEA and | N/A | — | — | 0.5 | — |
| | acetamide MEA | | | | | |
| | Phase C | | | | | |
| 26 | Hydrolyzed silk protein | N/A | — | — | 1.0 | — |
| 27 | PEG-20 Almond Glycerides | N/A | — | — | — | 0.2 |
| 28 | Lipid coated NPs (D14/D17/ | 10 | 20 (D14) | 10 (D17) | 10 (D19) | 5 (D23) |
| | D19/D23) | | | | | |
| 29 | Titanium dioxide | N/A | — | — | 1.0 | 1.0 |
| 30 | Salicylic acid | N/A | — | 0.5 | — | — |
| 31 | Propylene glycol | N/A | 5.0 | — | 2.0 | 2.0 |
| 32 | Pyrus Malus (Apple) Fruit | N/A | — | — | 10.0 | — |
| | Extract | | | | | |
| 33 | Citrus Aurantium Dulcis | N/A | — | 1.0 | 1.0 | — |
| | (Orange) Fruit Extract | | | | | |
| 34 | Linoleamidopropyl PG- | N/A | — | — | — | 0.3 |
| | dimonium chloride phosphate | | | | | |
| 35 | Cocodimonium | N/A | — | — | — | 0.3 |
| | hydroxypropyl hydrolyzed | | | | | |
| | wheat protein | | | | | |
| 36 | Tocopheryl acetate | N/A | — | — | 0.5 | 0.5 |
| 37 | Fragrance | N/A | qs. | qs. | qs. | qs. |
| 38 | Preservative | N/A | qs. | qs. | qs. | qs. |

Method of Preparation:

(1) Phase A: Solid components of Phase A are mixed and heated to melt. Liquid components of Phase A are then added one-by-one over the melt under stirring condition maintaining the temperature between 70-80° C. (2) Phase B: Carbopol is added to water while stirring. Other polymers of Phase B are dispersed and pH is neutralized by triethanolamine. Remaining components are added one-by-one under stirring and heated at 70-80° C. The Phase B is added to Phase A while stirring at about 70-80° C. After uniform mixing, the mixture is allowed to cool to 35-40° C. (3) Phase C: To the above stirring mixture, the components of Phase C (except fragrance and preservatives) are added slowly at 35-40° C. in the same order as mentioned in Table 5 and stirred (50-100 rpm) till uniform mixing. The mixture is then allowed to cool to room temperature. Finally, fragrance and preservatives are added, and the mixture is continued to stir in order to yield a smooth uniform creamy formulation (about 150-160 rpm).

### Example 12

### Preparation of Gel Formulations with Lipid Coated Nanoparticle Dispersions (Compositions G1-G4)

Gel formulations with lipid coated nanoparticle dispersions (of Examples 3, 4, 5 and 7) are designed and formulated as per the compositions shown in Table 6.

TABLE 6
Gel Formulations for Compositions G1, G2, G3 and G4

| Sr. | | Strength | Composition (%) | | | |
|---|---|---|---|---|---|---|
| No. | Ingredients | (%) | G1 | G2 | G3 | G4 |
| | Phase A | | | | | |
| 1 | Butylene glycol | N/A | — | — | 4.0 | — |
| 2 | Glycerin | N/A | 6.0 | — | 3.0 | 2.0 |
| 3 | Methyl gluceth-20 | N/A | 2.0 | — | 1.0 | — |
| 4 | Water | N/A | qs. | qs. | qs. | qs. |
| 5 | Allantoin | N/A | — | — | 0.05 | — |
| 6 | Disodium EDTA | N/A | — | 0.1 | 0.01 | — |
| 7 | PEG-12 dimethicone | N/A | — | — | 3.0 | — |
| 8 | Propylene glycol | N/A | — | 5.0 | — | — |
| 9 | Polyacrylate-1 crosspolymer | 20 | — | — | 20.0 | — |
| 10 | Carbopol | N/A | — | 0.6 | — | 0.5 |
| 11 | Glycolic acid | 71 | — | — | 14.3 | — |
| 12 | Triethanolamine | N/A | — | — | 7.1 | 0.8 |
| 13 | Benzophenone-4 | N/A | 0.05 | — | — | — |
| 14 | Acrylates/10-30 alkyl acrylate | N/A | 0.6 | — | — | — |
| | crosspolymer | | | | | |
| 15 | PEG/PPG-8/3 laurate | N/A | 5.0 | — | — | — |
| 16 | Aminomethyl propanol | 95 | 0.25 | 0.36 | — | — |
| | Phase B | | | | | |
| 17 | Water | N/A | 10 | — | — | — |
| 18 | Ethanol | N/A | — | — | — | 50 |
| 19 | Polyquaternium-39 | 10 | 1.0 | — | — | — |
| 20 | PEG-80 glyceryl cocoate | N/A | 1.0 | — | — | — |
| 21 | Tea tree oil, PEG-40 | N/A | — | 2.7 | — | — |
| | hydrogenated castor oil, | | | | | |
| | polysorbate-20, water | | | | | |
| | (premix) | | | | | |
| 22 | Triclosan | N/A | — | — | — | 0.2 |
| 23 | Lipid coated NPs (D21/D18/ | 10 | 20 (D21) | 10 (D18) | 10 (D20) | 5 (D25) |
| | D20/D25) | | | | | |
| 24 | Tocopheryl acetate, retinyl | N/A | — | — | 0.3 | — |
| | palmitate, sunflower seed oil, | | | | | |
| | agar | | | | | |
| 25 | Coloring agent | N/A | qs. | qs. | qs. | qs. |
| 26 | Preservative | N/A | qs. | qs. | qs. | qs. |
| 27 | Fragrance | N/A | qs. | qs. | qs. | qs. |

Method of Preparation:

(1) Phase A: All components of Phase A, except polymers and pH modulators, are mixed and dissolved in water. To this solution, gelling polymer is added and allowed to swell, followed by pH adjustment using one or more pH modulators. (2) Phase B: To the above stirring mixture, the components of Phase B (except fragrance and preservatives), premixed with water, are added slowly in the same order as mentioned in Table 6 and stirred till uniform mixing. Finally, fragrance and preservatives are added, and the mixture is continued to stir in order to yield a smooth, uniform transparent/translucent gel formulation.

### Example 13

### Preparation of Toothpastes with Lipid Coated Nanoparticle Dispersions (Compositions T1)

Toothpastes with lipid coated nanoparticle dispersions (of Examples 4) are designed and formulated as per the compositions shown in Table 7.

TABLE 7
Toothpastes for Compositions T1

| | | Strength | Composition T1 |
|---|---|---|---|
| Sr. No. | Ingredients | (%) | (%) |
| 1 | Carboxymethyl cellulose | N/A | 0.3 |
| 2 | Polyethylene glycol 1450 | N/A | 5.0 |
| 3 | Sorbitol | 70 | 46.72 |
| 4 | Glycerin | 96 | 20.90 |
| 5 | Sodium monofluorophosphate | N/A | 0.78 |
| 6 | Sodium saccharin | N/A | 0.2 |
| 7 | Preservative | N/A | qs. |
| 8 | | | |
| 8 | Lipid coated NPs (D20) | 10 | 10 (D20) |
| 9 | Coloring agent | N/A | qs. |
| 10 | Silica Xerogel | N/A | 14.0 |
| 11 | Hydrated silica | N/A | 8.0 |
| 12 | Mint-type flavor | 20 | 2.0 |
| 13 | Sodium lauryl sulfate | N/A | 1.50 |

Method of Preparation:

(1) Carboxymethyl cellulose and polyethylene glycol 1450 are dispersed uniformly into sorbitol and glycerin using high-speed stirrer. (2) A blend of Sodium monofluorophosphate, sodium saccharin and preservative is prepared and dispersed in to gum slurry prepared above and stirred for 10 min. (3) Lipid coated particles are dispersed into the above mixture, followed by addition of coloring agent. (4) To the above stirring mixture, silica Xerogel and hydrated silica are dispersed under stirring, followed by addition of flavoring agent. (5) Finally, sodium lauryl sulfate is mixed carefully to avoid air-bubbles entrapment.

### Example 14

### Preparation of Mouthwashes with Lipid Coated Nanoparticle Dispersions (Compositions M1)

Mouthwashes with lipid coated nanoparticle dispersion (of Examples 7) are designed and formulated as per the composition shown in Table 8.

TABLE 8
Mouthwashes for Compositions M1

| | | Strength | Composition M1 |
|---|---|---|---|
| Sr. No. | Ingredients | (%) | (%) |
| 1 | Ethyl alcohol | N/A | 10.0 |
| 2 | Flavor(menthol, 30%; methyl | N/A | 0.25 |
| | salicylate, 30%; peppermint oil, | | |
| | 30%; eucalyptol, 10%) | | |
| 3 | Glycerin | N/A | 10.0 |
| 4 | Polyoxyethylene/polyoxypropylene | N/A | 2.0 |
| | block polymer (Poloxamer 407) | | |
| 5 | Lipid coated NPs (D26) | 10 | 20 (D26) |
| 6 | Saccharin sodium | N/A | 0.05 |
| 7 | Water | N/A | qs. |

Method of Preparation:

(1) Poloxamer 407 is dispersed into water uniformly using high-speed stirrer. (2) Glycerin is added and dispersed in to slurry prepared above and stirred for 10 min. (3) Lipid coated particles are dispersed into the above mixture, followed by addition of saccharin sodium solution in water. (4) Finally flavoring agent (premixed in ethyl alcohol) is added to above stirring mixture. The formulation is allowed to stir until uniformly mixed mouthwash is obtained.

### Example 15

### Preparation of Chewing Gums with Lipid Coated Nanoparticle Dispersions (Compositions CG1)

Chewing gums with lipid coated nanoparticle dispersion (of Example 8) are designed and formulated as per the composition shown in Table 9.

TABLE 9
Chewing Gums for Compositions CG1

| | | Strength | Composition M1 |
|---|---|---|---|
| Sr. No. | Ingredients | (%) | (%) |
| 1 | Gum base | N/A | 23 |
| 2 | 45° Be'glucose syrup | 80% | 15 |
| 3 | Sugar powder | N/A | 50 |
| 4 | Lipid coated NPs (D27) | 10 | 10 (D27) |
| 5 | Flavors | N/A | qs. |
| 6 | Glycerin | N/A | 0.6 |
| 7 | Preservative | N/A | qs. |

Method of Preparation:

(1) Preheated gum base (about 50° C.) is charged into preheated mixer (about 50° C.). To this, preheated glucose syrup is added at a temperature of 45-50° C., followed by addition of about half amounts of sugar powder, lipid coated particle dispersion and flavor. The blend is then mixed for 3-5 min to allow uniform mixing of the blend. (2) The remaining amounts of the sugar powder, the particle dispersion and the flavor are added to the blend, which is then continued to mix for further 3-4 min. (3) Finally glycerin is added into the mixing blend, and the blend is again allowed to mix for further 3-4 min in order to achieve uniform mixing. The material is then discharged and sent for shape forming section.

### Example 16

### Minimum Inhibitory Concentration (MIC) of Modified ZPT Dispersions and their Formulations

Broth and Agar dilution are routinely used methods for antimicrobial susceptibility testing. To study Minimum Inhibitory Concentration of ZPT dispersions and their formulations, agar plate dilution method is employed with Leeming Notman medium. Experiments are always done in triplicates.

Method:

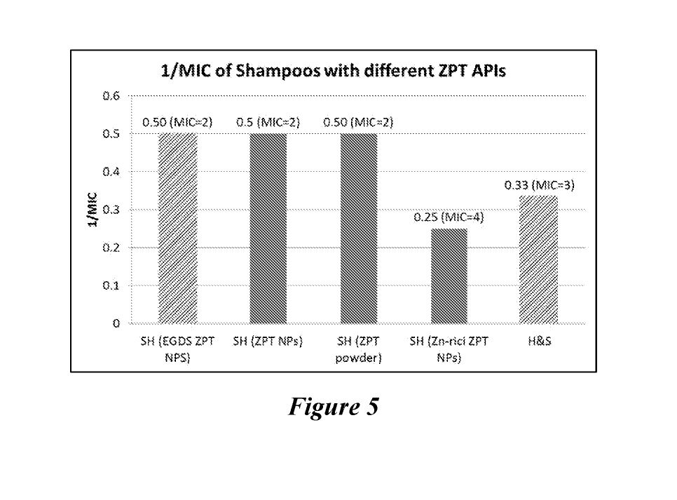

Sterile media are supplemented with chloramphenicol (0.25 mg/ml), cycloheximide (0.04 mg/ml) and olive oil (2%). The media are then supplemented with appropriate concentrations (two-fold serial dilutions) of either unmodified ZPT, modified ZPT or corresponding formulations. For negative controls, no API is added. And in case of MIC studies of formulation, blank formulation (without actives) is used. Appropriately cooled medium is poured into sterile petri plates. Once the agar solidifies, the plates are inoculated with M. furfur, incubated under CO₂ atmosphere at 30±2° C. and the readouts (MIC₁₀₀ and MIC₉₀) are taken after every 24 hr for 6 days. MIC₁₀₀ values for some of the compositions are plotted in the graphs shown in FIG. 4 (API dispersion compositions) and FIG. 5 (shampoo compositions).

### Example 17

### Particle Retention Studies on Goat Skin Model (Ex Vivo Studies) Using Modified ZPT Dispersions

In order to understand retention behavior of coated/uncoated nanoparticles (of different size ranges) and their corresponding formulations on human skin/scalp/hair, suitable models are required to conduct preliminary retention trials. For scalp studies, goat skin (easily available) seems to have close resemblance with human scalp based on hair density [average hair density: 125-200 hair/cm² (human scalp); about 225 hair/cm² (goat skin)].

There is some description in the art of submicron sized particles (320-750 nm) having a greater propensity to retain onto skin, especially into infundibular area of hair follicles (Nanocosmetics and Nanomedicines: New Approaches for Skin Care, Chapter 1, Editors: Ruy Beck, Silvia Guterres, Adriana Pohlmann, 2011, Springer-Verlag Berlin Heidelberg, pg 12). It is believed that hair follicles may act as long-term reservoirs and efficient storage spaces for submicron sized particles (Eur J Pharm Biopharm (2011) 77, 465-468; Eur J Pharm Biopharm (2007) 66, 159-164; Skin Pharmacol Physiol (2008) 21, 150-155; and Skin Pharmacol Physiol (2006) 19, 232-236). Without wishing to be bound by a theory, infundibular spaces of hair follicles can facilitate retention/deposition of lipophilic entities (e.g., the lipid coated particle disclosed in the present disclosure) of appropriate size range since the infundibular spaces are filled with sebum.

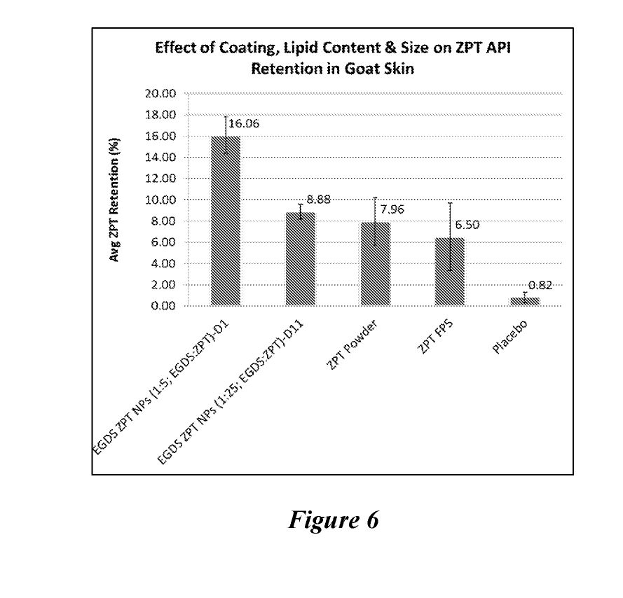

Method: Goat skin, fitted in Franz cell, is incubated for 5 min (including 1 min of gentle messaging) with test sample of 1.2 mg per ml of formulation per 4.9 cm² of exposed goat skin (either dispersion or final formulation). After incubation, each piece of skin is washed with water to remove un-retained particles and excess formulation. The skin is cut in to small pieces and the homogenization in DMSO is effected using high shear homogenizer at 25,000 rpm for 5 min with intervals of 30 sec after every 1 min. After homogenization, the extract is subjected to bath sonication for 10 min at 30° C. followed by centrifugation for 20 minutes at 1700 rpm to collect supernatant. The supernatants after filtration through 0.45 μm filters, are analyzed for zinc content using Atomic Absorption Spectroscopy (AAS). The experiments are always performed at least in triplicates. Percentage of ZPT retained in skin (w.r.t. total applied amount) for some of the API dispersion compositions are plotted in the graphs shown in FIG. 6.

### Example 18

### Dose Response Curves (Using Zones of Inhibition) of In-House Shampoo Formulations

Agar well-diffusion method is employed to run Zone of Inhibition (ZOI) assays. ZOI values may vary for compounds having different diffusion coefficients. ZOI is employed to assess the potency of API and/or formulation to inhibit the growth of microorganisms under study. ZOI values, determined at different API concentrations, can be used to derive dose-response-curves (DRCs) for efficacy comparison of different APIs/formulations.

Method:

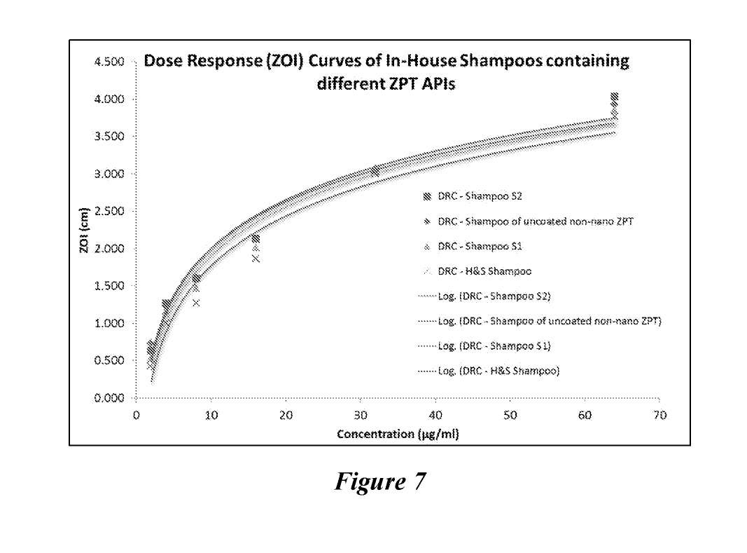

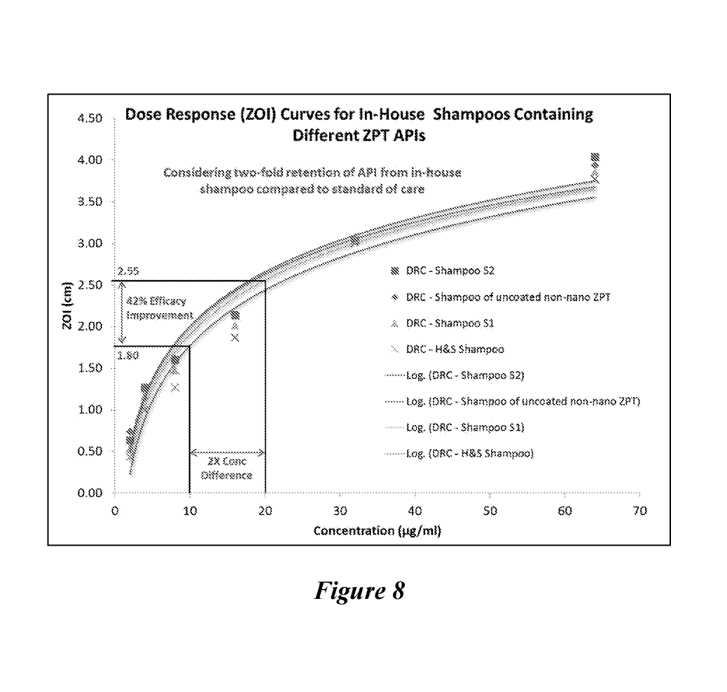

Malassezia furfur culture of specific cfu/ml is used to inoculate Sabaroud's Dextrose agar (SDA) plates [supplemented with chloramphenicol (0.25 mg/ml), cycloheximide (0.04 mg/ml) and olive oil (2%)]. Approximately, 6 mm wells are created in the agar plate using sterile straws. The wells are supplemented with different concentrations of test samples and/or controls (100 μl each). The plates then are incubated at 30±2° C. under CO₂ (5%) atmosphere. Readouts are taken after 42 or 72 hrs. An example of DRC study using in-house shampoos in comparison with standard of care is demonstrated in FIGS. 7 and 8.

### Example 19

### Time-Kill Kinetic Studies of Modified ZPT Dispersions and Corresponding Shampoo Formulations

Time-kill assays are used to evaluate efficacy of antimicrobial agents, either singly or in combination, and results can help in establishing the dose and/or time of application of the active. Time-kill assays can be used to study both concentration-dependent and time-dependent antimicrobial activities.

Method:

M. furfur cells are suspended in Sabouraud Dextrose Broth (SDB) at inoculum concentration of 1-2×10⁶ cells/ml. Cells are taken from a freshly growing (3-7 days old) plate and cell suspension is vortexed to remove the cell clumps as much as possible. Sterile media are supplemented with chloramphenicol (0.25 mg/ml), cycloheximide (0.04 mg/ml) and olive oil (2%). The media are then supplemented with appropriate concentrations (two-fold serial dilutions using SDB) of either unmodified active, modified active or corresponding formulation. For negative controls, no API is added. And in case of studies on formulation, blank formulation (without actives) is added (to get final concentrations of 0.5, 1.0 and 2 μg/ml in case of ZPT). The cultures are incubated on a tube rotator at 34° C. in CO₂ incubator.

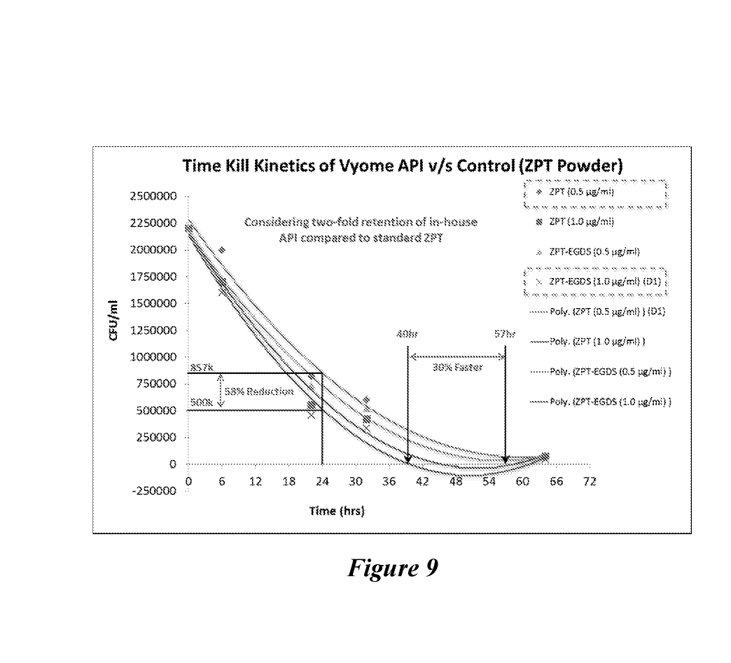

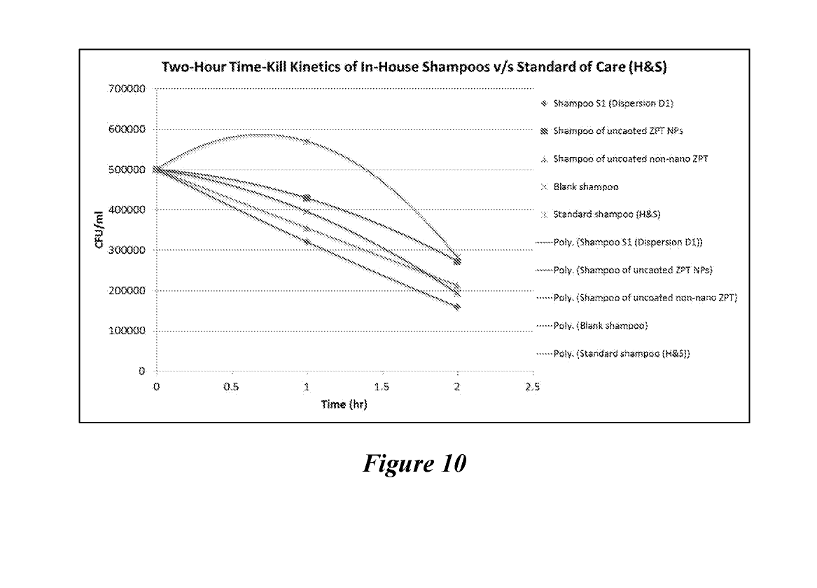

To measure the colony forming units (CFU), at different time points, aliquots (50 μl) of Malassezia cultures are serially diluted with SDBT medium (SDB containing 0.1% Triton X-100) and plated on SDA plates. The plates are incubated at 34° C. in CO₂ incubator for 3 days. The viable colonies are counted and converted to CFU/ml. The results of time kill study using dispersion composition D1 are shown in Table 10 and are plotted in FIG. 9.

TABLE 10
Average CFU counts of composition D1 versus standard
ZPT dispersion at two different concentrations and at
various time points (experiment done in triplicates)

| | | Std ZPT | | |
|---|---|---|---|---|
| | Std ZPT | dispersion | Composition D1 | Composition D1 |
| Time | dispersion | ZPT | ZPT-EGDS | ZPT-EGDS |
| (hrs) | (0.5 μg/ml) | (1.0 μg/ml) | (0.5 μg/ml) | (1.0 μg/ml) |
| 0 | 2200000 | 2200000 | 2200000 | 2200000 |
| 6 | 2000000 | 1700000 | 1720000 | 1600000 |
| 22 | 820000 | 550000 | 730000 | 460000 |
| 32 | 600000 | 420000 | 520000 | 338000 |
| 64 | 80000 | 70000 | 70000 | 40000 |

### Example 20

### Lipid Coated Zinc Pyrithione (ZPT) Particle Dispersions (Compositions D29-D33)

Preparation:

ZPT powder is added in portions to the 1% aqueous solution of sodium docusate while stirring. The resulting suspension is passed through high pressure homogenizer at about 1600 bar. The output dispersion is collected in a beaker kept in ice bath and recycled 12 times to yield a dispersion of appropriately sized particles (about 200 nm to about 800 nm) and heated to 70° C. Weighed quantity of lipid Ethylene glycol distearate [EGDS]/Ethylene glycol dimyristate [EGDM]/Ethylene glycol dipalmitate [EGDP]/Ethylene glycol dilaurate [EGDL]/Lauric acid [LA] is melted and added to hot homogenized dispersion and continued to stir for 15 minutes at same temperature. The size distribution is determined by ZetaSizer (ZS-90 from Malvern Instruments) and Scanning Electron Microscope (SEM, Hitachi, S-3400 N, Japan). The resulting dispersions are stabilized by adding carbopol solution followed by neutralization with sodium hydroxide to a pH ranging from about 6.5 to about 7.0. Some of the examples are given in Table 11.

TABLE 11
Dispersion compositions of coated particles
prepared using high pressure homogenizer

| | | | | Core |
|---|---|---|---|---|
| | | | | Strength in |
| Compo- | | | Coat:Core | dispersion |
| sitions | Core | Coat | ratio | (% w/w) |
| D29 | ZPT | EGDS | 1:5 | 25 |
| D30 | ZPT | EGDM | 1:5 | 25 |
| D31 | ZPT | EGDL | 1:5 | 25 |
| D32 | ZPT | LA | 1:5 | 25 |
| D33 | ZPT | EGDP | 1:5 | 25 |

### Example 21

### Lipid Coated Besifloxacin HCl Particle Dispersions (Compositions D34-D41)

Preparation:

Besifloxacin HCl is dispersed in surfactant solution (Lecithin and Poloxamer 407 dissolved in water). The mixture is homogenized at 30,000 rpm for 5 minutes using High Shear Homogenizer (Fisher Scientific™ PowerGen™ Model 125). With an interval of 1 minute, total 3 cycles of homogenization are run and each cycle is for 5 minutes. The homogenized dispersion is heated to 70° C. Weighed quantity of lipid (lauric acid/myristic acid/palmitic acid/stearic acid/EGDS/EGDL/EGDP/EGDM) is heated to melt. The melted lipid is added to hot homogenized dispersion at same temperature under continuous vigorous stirring for 20 minutes followed by cooling in an ice bath with continuous stirring for 24 hrs. The size distribution is determined by ZetaSizer (ZS-90 from Malvern Instruments) and Scanning Electron Microscope (SEM, Hitachi, S-3400 N, Japan). The resulting dispersions are stabilized by adding suitable stabilizer followed by neutralization with pH modulator. Some of the examples for dispersion preparations are given in Table 12. For dispersion preparation, different types of surfactant solutions such as Lecithin, Poloxamer 407, TPGS (D-α-Tocopherol polyethylene glycol succinate), Poloxamer 188, Sodium docusate may be used alone or in combinations.

TABLE 12
Dispersion compositions of lipid coated Besifloxacin
HCl particles prepared using high shear homogenizer

| | | | | Core |
|---|---|---|---|---|
| | | | | Strengthin |
| Compo- | | | Coat:Core | dispersion |
| sitions | Core | Coat | ratio | (% w/w) |
| D34 | Besifloxacin | Lauric acid | 1:5 | 10 |
| D35 | Besifloxacin | Myristic acid | 1:5 | 10 |
| D36 | Besifloxacin | Palmitic acid | 1:5 | 10 |
| D37 | Besifloxacin | Stearic acid | 1:5 | 10 |
| D38 | Besifloxacin | Ethylene glycol | 1:5 | 10 |
| | | distearate | | |
| D39 | Besifloxacin | Ethylene glycol | 1:5 | 10 |
| | | dilaurate | | |
| D40 | Besifloxacin | Ethylene glycol | 1:5 | 10 |
| | | dipalmitate | | |
| D41 | Besifloxacin | Ethylene glycol | 1:5 | 10 |
| | | dimyristate | | |

### Example 22

### Lipid Coated Prulifloxacin Particle Dispersions (Compositions D42-D49)

Preparation:

Prulifloxacin is dispersed in surfactant solution (Lecithin and Poloxamer 407 dissolved in water). The mixture is homogenized at 30,000 rpm for 5 minutes using High Shear Homogenizer (Fisher Scientific™ PowerGen™ Model 125). With an interval of 1 minute, total 3 cycles of homogenization are run and each cycle is for 5 minutes. The homogenized dispersion is heated to 70° C. Weighed quantity of lipid (lauric acid/myristic acid/palmitic acid/stearic acid/EGDS/EGDL/EGDP/EGDM) is heated to melt. The melted lipid is added to hot homogenized dispersion at same temperature under continuous vigorous stirring for 20 minutes followed by cooling in an ice bath with continuous stirring for 24 hrs. The size distribution is determined by ZetaSizer (ZS-90 from Malvern Instruments) and Scanning Electron Microscope (SEM, Hitachi, S-3400 N, Japan). The resulting dispersions are stabilized by adding suitable stabilizer followed by neutralization with pH modulator. Some of the examples of lipid coated Prulifloxacin dispersions are given in Table 13. For dispersion preparation, different types of surfactant solutions such as Lecithin, Poloxamer 407, TPGS (D-α-Tocopherol polyethylene glycol succinate), Poloxamer 188, Sodium docusate may be used alone or in combinations.

TABLE 13
Dispersion compositions of lipid coated Prulifloxacin
particles prepared using high shear homogenizer

| | | | | Core |
|---|---|---|---|---|
| | | | | Strength in |
| Compo- | | | Coat:Core | dispersion |
| sitions | Core | Coat | ratio | (%w/w) |
| D42 | Prulifloxacin | Lauric acid | 1:5 | 10 |
| D43 | Prulifloxacin | Myristic acid | 1:5 | 10 |
| D44 | Prulifloxacin | Palmitic acid | 1:5 | 10 |
| D45 | Prulifloxacin | Stearic acid | 1:5 | 10 |
| D46 | Prulifloxacin | Ethylene glycol | 1:5 | 10 |
| | | distearate | | |
| D47 | Prulifloxacin | Ethylene glycol | 1:5 | 10 |
| | | dilaurate | | |
| D48 | Prulifloxacin | Ethylene | 1:5 | 10 |
| | | glycoldipalmitate | | |
| D49 | Prulifloxacin | Ethylene glycol | 1:5 | 10 |
| | | dimyristate | | |

### Example 23

### Lipid Coated Ulifloxacin Particle Dispersions (Compositions D50-D57)

Preparation:

Ulifloxacin is dispersed in surfactant solution (Lecithin and Poloxamer 407 dissolved in water). The mixture is homogenized at 30,000 rpm for 5 minutes using High Shear Homogenizer (Fisher Scientific™ PowerGen™ Model 125). With an interval of 1 minute, total 3 cycles of homogenization are run and each cycle is for 5 minutes. The homogenized dispersion is heated to 70° C. Weighed quantity of lipid (lauric acid/myristic acid/palmitic acid/stearic acid/EGDS/EGDL/EGDP/EGDM) is heated to melt. The melted lipid is added to hot homogenized dispersion at same temperature under continuous vigorous stirring for 20 minutes followed by cooling in an ice bath with continuous stirring for 24 hrs. The size distribution is determined by ZetaSizer (ZS-90 from Malvern Instruments) and Scanning Electron Microscope (SEM, Hitachi, S-3400 N, Japan). The resulting dispersions are stabilized by adding suitable stabilizer followed by neutralization with pH modulator. Some of the examples of lipid coated Ulifloxacin dispersions are given in Table 14. For dispersion preparation, different types of surfactant solutions such as Lecithin, Poloxamer 407, TPGS (D-α-Tocopherol polyethylene glycol succinate), Poloxamer 188, Sodium docusate may be used alone or in combinations.

TABLE 14
Dispersion compositions of lipid coated Ulifloxacin
particles prepared using high shear homogenizer

| | | | | Core |
|---|---|---|---|---|
| | | | | Strengthin |
| Compo- | | | Coat:Core | dispersion |
| sitions | Core | Coat | ratio | (%w/w) |
| D50 | Ulifloxacin | Lauric acid | 1:5 | 10 |
| D51 | Ulifloxacin | Myristic acid | 1:5 | 10 |
| D52 | Ulifloxacin | Palmitic acid | 1:5 | 10 |
| D53 | Ulifloxacin | Stearic acid | 1:5 | 10 |
| D54 | Ulifloxacin | Ethylene glycol | 1:5 | 10 |
| | | distearate | | |
| D55 | Ulifloxacin | Ethylene glycol | 1:5 | 10 |
| | | dilaurate | | |
| D56 | Ulifloxacin | Ethylene glycol | 1:5 | 10 |
| | | dipalmitate | | |
| D57 | Ulifloxacin | Ethylene glycol | 1:5 | 10 |
| | | dimyristate | | |

### Example 24

### Lipid Coated Nadifloxacin Particle Dispersions (Compositions D58-D65)

Preparation:

Nadifloxacin is dispersed in surfactant solution (Lecithin and Poloxamer 407 dissolved in water). The mixture is homogenized at 30,000 rpm for 5 minutes using High Shear Homogenizer (Fisher Scientific™ PowerGen™ Model 125). With an interval of 1 minute, total 3 cycles of homogenization are run and each cycle is for 5 minutes. The homogenized dispersion is heated to 70° C. Weighed quantity of lipid (lauric acid/myristic acid/palmitic acid/stearic acid/EGDS/EGDL/EGDP/EGDM) is heated to melt. The melted lipid is added to hot homogenized dispersion at same temperature under continuous vigorous stirring for 20 minutes followed by cooling in an ice bath with continuous stirring for 24 hrs. The size distribution is determined by ZetaSizer (ZS-90 from Malvern Instruments) and Scanning Electron Microscope (SEM, Hitachi, S-3400 N, Japan). The resulting dispersions are stabilized by adding suitable stabilizer followed by neutralization with pH modulator. Some of the examples of lipid coated Nadifloxacin dispersions are given in Table 15. For dispersion preparation, different types of surfactant solutions such as Lecithin, Poloxamer 407, TPGS (D-α-Tocopherol polyethylene glycol succinate), Poloxamer 188, Sodium docusate may be used alone or in combinations.

TABLE 15
Dispersion compositions of lipid coated Nadifloxacin
particles prepared using high shear homogenizer

| | | | | Core |
|---|---|---|---|---|
| | | | | Strength in |
| Compo- | | | Coat:Core | dispersion |
| sitions | Core | Coat | ratio | (% w/w) |
| D58 | Nadifloxacin | Lauric acid | 1:5 | 10 |
| D59 | Nadifloxacin | Myristic acid | 1:5 | 10 |
| D60 | Nadifloxacin | Palmitic acid | 1:5 | 10 |
| D61 | Nadifloxacin | Stearic acid | 1:5 | 10 |
| D62 | Nadifloxacin | Ethylene glycol | 1:5 | 10 |
| | | distearate | | |
| D63 | Nadifloxacin | Ethylene glycol | 1:5 | 10 |
| | | dilaurate | | |
| D64 | Nadifloxacin | Ethylene glycol | 1:5 | 10 |
| | | dipalmitate | | |
| D65 | Nadifloxacin | Ethylene glycol | 1:5 | 10 |
| | | dimyristate | | |

### Example 25

### Lipid Coated Ritapamulin Particle Dispersions (Compositions D66-D73)

Preparation:

Ritapamulin is dispersed in surfactant solution (Lecithin and Poloxamer 407 dissolved in water). The mixture is homogenized at 30,000 rpm for 5 minutes using High Shear Homogenizer (Fisher Scientific™ PowerGen™ Model 125). With an interval of 1 minute, total 3 cycles of homogenization are run and each cycle is for 5 minutes. The homogenized dispersion is heated to 70° C. Weighed quantity of lipid (lauric acid/myristic acid/palmitic acid/stearic acid/EGDS/EGDL/EGDP/EGDM) is heated to melt. The melted lipid is added to hot homogenized dispersion at same temperature under continuous vigorous stirring for 20 minutes followed by cooling in an ice bath with continuous stirring for 24 hrs. The size distribution is determined by ZetaSizer (ZS-90 from Malvern Instruments) and Scanning Electron Microscope (SEM, Hitachi, S-3400 N, Japan). The resulting dispersions are stabilized by adding suitable stabilizer followed by neutralization with pH modulator. Some of the examples of lipid coated Ritapamulin dispersions are given in Table 16. For dispersion preparation, different types of surfactant solutions such as Lecithin, Poloxamer 407, TPGS (D-α-Tocopherol polyethylene glycol succinate), Poloxamer 188, Sodium docusate may be used alone or in combinations.

TABLE 16
Dispersion compositions of lipid coated Ritapamulin
particles prepared using high shear homogenizer

| | | | | Core |
|---|---|---|---|---|
| | | | | Strength in |
| Compo- | | | Coat:Core | dispersion |
| sitions | Core | Coat | ratio | (% w/w) |
| D66 | Ritapamulin | Lauric acid | 1:5 | 10 |
| D67 | Ritapamulin | Myristic acid | 1:5 | 10 |
| D68 | Ritapamulin | Palmitic acid | 1:5 | 10 |
| D69 | Ritapamulin | Stearic acid | 1:5 | 10 |
| D70 | Ritapamulin | Ethylene glycol | 1:5 | 10 |
| | | distearate | | |
| D71 | Ritapamulin | Ethylene glycol | 1:5 | 10 |
| | | dilaurate | | |
| D72 | Ritapamulin | Ethylene glycol | 1:5 | 10 |
| | | dipalmitate | | |
| D73 | Ritapamulin | Ethylene glycol | 1:5 | 10 |
| | | dimyristate | | |

### Example 26

### Lipid Coated Adapalene Particle Dispersions (Compositions D74-D81)

Preparation:

Adapalene is dispersed in surfactant solution (Lecithin and Poloxamer 407 dissolved in water). The mixture is homogenized at 30,000 rpm for 5 minutes using High Shear Homogenizer (Fisher Scientific™ PowerGen™ Model 125). With an interval of 1 minute, total 3 cycles of homogenization are run and each cycle is for 5 minutes. The homogenized dispersion is heated to 70° C. Weighed quantity of lipid (lauric acid/myristic acid/palmitic acid/stearic acid/EGDS/EGDL/EGDP/EGDM) is heated to melt. The melted lipid is added to hot homogenized dispersion at same temperature under continuous vigorous stirring for 20 minutes followed by cooling in an ice bath with continuous stirring for 24 hrs. The size distribution is determined by ZetaSizer (ZS-90 from Malvern Instruments) and Scanning Electron Microscope (SEM, Hitachi, S-3400 N, Japan). The resulting dispersions are stabilized by adding suitable stabilizer followed by neutralization with pH modulator. Some of the examples of lipid coated Adapalene dispersions are given in Table 17. For dispersion preparation, different types of surfactant solutions such as Lecithin, Poloxamer 407, TPGS (D-α-Tocopherol polyethylene glycol succinate), Poloxamer 188, Sodium docusate may be used alone or in combinations.

TABLE 17
Dispersion compositions of lipid coated Adapalene
particles prepared using high shear homogenizer

| | | | | Core |
|---|---|---|---|---|
| | | | | Strength in |
| Compo- | | | Coat:Core | dispersion |
| sitions | Core | Coat | ratio | (% w/w) |
| D74 | Adapalene | Lauric acid | 1:5 | 10 |
| D75 | Adapalene | Myristic acid | 1:5 | 10 |
| D76 | Adapalene | Palmitic acid | 1:5 | 10 |
| D77 | Adapalene | Stearic acid | 1:5 | 10 |
| D78 | Adapalene | Ethylene glycol | 1:5 | 10 |
| | | distearate | | |
| D79 | Adapalene | Ethylene glycol | 1:5 | 10 |
| | | dilaurate | | |
| D80 | Adapalene | Ethylene glycol | 1:5 | 10 |
| | | dipalmitate | | |
| D81 | Adapalene | Ethylene glycol | 1:5 | 10 |
| | | dimyristate | | |

### Example 27

### Lipid Coated Adapalene Microparticle Dispersions (Compositions D82-D89)

Preparation:

Adapalene is dispersed in surfactant solution (Lecithin and Poloxamer 407 dissolved in water). The dispersion is heated to 70° C. Weighed quantity of lipid (lauric acid/myristic acid/palmitic acid/stearic acid/Ethylene glycol distearate) is heated to melt. The melted lipid is added to hot dispersion at same temperature under continuous vigorous stirring for 20 minutes followed by cooling in an ice bath with continuous stirring for 24 hrs. The size distribution is determined by ZetaSizer (ZS-90 from Malvern Instruments) and Scanning Electron Microscope (SEM, Hitachi, S-3400 N, Japan). The resulting dispersions are stabilized by adding suitable stabilizer followed by neutralization with pH modulator. Some of the examples for dispersion preparations are given in Table 18. For dispersion preparation, different types of surfactant solutions such as Lecithin, Poloxamer 407, TPGS (D-α-Tocopherol polyethylene glycol succinate), Poloxamer 188, Sodium docusate may be used alone or in combinations.

TABLE 18
Dispersion compositions of lipid coated Adapalene microparticles

| | | | | Core |
|---|---|---|---|---|
| | | | | Strength in |
| Compo- | | | Coat:Core | dispersion |
| sitions | Core | Coat | ratio | (% w/w) |
| D82 | Adapalene | Lauric acid | 1:5 | 10 |
| D83 | Adapalene | Myristic acid | 1:5 | 10 |
| D84 | Adapalene | Palmitic acid | 1:5 | 10 |
| D85 | Adapalene | Stearic acid | 1:5 | 10 |
| D86 | Adapalene | Ethylene glycol | 1:5 | 10 |
| | | distearate | | |
| D87 | Adapalene | Ethylene glycol | 1:5 | 10 |
| | | dilaurate | | |
| D88 | Adapalene | Ethylene glycol | 1:5 | 10 |
| | | dipalmitate | | |
| D89 | Adapalene | Ethylene glycol | 1:5 | 10 |
| | | dimyristate | | |

### Example 28

### Preparation of Cream Formulations with Lipid Coated Particle Dispersions (Compositions CR5-CR9)

Cream formulations with lipid coated particle dispersions (of Examples 21-25) are designed and formulated as per the compositions shown in Table 19.

TABLE 19
Cream Formulations for Compositions CR5, CR6, CR7, CR8, CR9
and CR10

| Sr. | | Strength | Composition (%) | | | | | |
|---|---|---|---|---|---|---|---|---|
| No. | Ingredients | in % | CR5 | CR6 | CR7 | CR8 | CR9 | CR10 |
| | Phase A | | | | | | | |
| 1 | Water | NA | qs. | qs. | qs. | qs. | qs. | qs. |
| | Phase B | | | | | | | |
| 2 | Cyclopentasiloxane | 96 | 2 | 2 | 2 | 2 | 2 | 2 |
| 3 | Cetyl alcohol | NA | 2 | 2 | 2 | 2 | 2 | 2 |
| 4 | Stearic acid | NA | 15 | 15 | 15 | 15 | 15 | 15 |
| 5 | Cetearyl alcohol | NA | 2 | 2 | 2 | 2 | 2 | 2 |
| 6 | Sorbitanmonooleate | NA | 0.15 | 0.15 | 0.15 | 0.15 | 0.15 | 0.15 |
| 7 | Steareth 21 | NA | 2.70 | 2.70 | 2.70 | 2.70 | 2.70 | 2.70 |
| 8 | Steareth 2 | NA | 0.15 | 0.15 | 0.15 | 0.15 | 0.15 | 0.15 |
| | Phase C | | | | | | | |
| 9 | Glycerol | NA | 12 | 12 | 12 | 12 | 12 | 12 |
| 10 | Propylene Glycol | 10 | 5 | 5 | 5 | 5 | 5 | 5 |
| 11 | Allantoin | NA | 0.2 | 0.2 | 0.2 | 0.2 | 0.2 | 0.2 |
| 12 | Sorbitol | NA | 4 | 4 | 4 | 4 | 4 | 4 |
| 13 | Lipid Coated Particles | 10 | 10 (D34) | 10 | 10 (D44) | 10 (D53) | 10 (D63) | 10 (D70) |
| | (D34/D35/D44/D53/ | | | (D35) | | | | |
| | D63/D70) | | | | | | | |
| 14 | Adapalene* | 10 | — | 1 | — | — | 1 | — |
| 15 | DL-α-Tocopherol acetate | NA | 0.002 | 0.002 | 0.002 | 0.002 | 0.002 | 0.002 |
| 15 | D-Panthenol | NA | 0.25 | 0.25 | 0.25 | 0.25 | 0.25 | 0.25 |
| | (Provitamin B5) | | | | | | | |
| 16 | EDTA | NA | 0.05 | 0.05 | 0.05 | 0.05 | 0.05 | 0.05 |
| 17 | Propyl Paraben | NA | 0.08 | 0.08 | 0.08 | 0.08 | 0.08 | 0.08 |
| 18 | Methyl Paraben | NA | 0.3 | 0.3 | 0.3 | 0.3 | 0.3 | 0.3 |
| 19 | Cetylpyridinium chloride | NA | — | — | 0.2 | — | — | 0.2 |
| 20 | Fragrance | NA | qs. | qs. | qs. | qs. | qs. | qs. |
| 21 | Citric acid | 50 | qs. | qs. | qs. | qs. | qs. | qs. |

*Adapalene in poloxamer 407 (1% aq.)/TPGS (1% aq.)(D-α-Tocopherol polyethylene glycol succinate)/Poloxamer 188 (1% aq.), Sodium docusate(1% aq.)

Method of Preparation:

(1) Phase A: A required amount of water is added to a mixing vessel and stirred slowly (50-55 rpm) using an overhead stirrer and heated to 70° C.
(2) Phase B: A mixture of cetyl alcohol, stearic acid, cetearyl alcohol, sorbitanmonooleate, steareth 21 and steareth 2 is heated to melt. The resulting melt is immediately poured to Phase A while stirring at about 200 rpm followed by stirring at 400 rpm. After stirring for about 5 min at the same temperature, it is allowed to cool to about 60° C. At this temperature, Cyclopentasiloxane is added.
(3) Phase C: The mixture is allowed to cool to about 40° C. followed by additions of glycerol and propylene glycol. Then, suspension of allantoin is added followed by the addition of solution of sorbitol. Lipid coated particle dispersion (of Examples 18, 19, 20, 21, 22 and 23) is added to the above stirring mixture. Then the remaining ingredients of phase C are added in the same order as mentioned in the Table 19. The continuously stirring mixture (700 rpm) is then allowed to cool to room temperature followed by addition of fragrance. Finally, pH is adjusted with citric acid to 5.5 and mixture is continued to stir to yield a smooth cream.

### Example 29

### Preparation of Gel Formulations with Lipid Coated Particle Dispersions (Compositions GL1-GL6)

Gel formulations with lipid coated particle dispersions (of Examples 21, 23-27) are designed and formulated as per the compositions shown in Table 20.

TABLE 20
Gel Formulations for Compositions GL1, GL2, GL3, GL4, GL5 and
GL6

| Sr. | | Strength | Composition (%) | | | | | |
|---|---|---|---|---|---|---|---|---|
| No. | Ingredients | in % | GL1 | GL2 | GL3 | GL4 | GL5 | GL6 |
| 1 | Water | NA | qs. | qs. | qs. | qs. | qs. | qs. |
| 2 | CarbopolUltrez 21 | NA | 0.60 | 0.60 | 0.60 | 0.60 | 0.60 | 0.60 |
| 3 | Sodium hydroxide | 18 | 1.5 | 1.5 | 1.5 | 1.5 | 1.5 | 1.5 |
| 4 | Lipid Coated Particles | 10 | 10 (D39) | 10 (D54) | 10 (D65) | 10 (D67) | 10 (81) | 10 (D88) |
| | (D39/D54/D65/D67/ | | | | | | | |
| | D81/D88) | | | | | | | |
| 5 | Glycerol | NA | 10 | 10 | 10 | 10 | 10 | 10 |
| 6 | Propyl Paraben | NA | 0.03 | 0.03 | 0.03 | 0.03 | 0.03 | 0.03 |
| 7 | Methyl Paraben | NA | 0.3 | 0.3 | 0.3 | 0.3 | 0.3 | 0.3 |
| 8 | Propylene Glycol | NA | 6 | 6 | 6 | 6 | 6 | 6 |
| 9 | Adapalene* | NA | — | 1 | — | 1 | — | — |
| 10 | Allantoin | NA | 0.2 | 0.2 | 0.2 | 0.2 | 0.2 | 0.2 |
| 11 | D-Sorbitol | NA | 3 | 3 | 3 | 3 | 3 | 3 |
| 12 | DL-α-Tocopherol acetate | NA | 0.5 | 0.5 | 0.5 | 0.5 | 0.5 | 0.5 |
| 13 | D-Panthenol (Provitamin | NA | 0.25 | 0.25 | 0.25 | 0.25 | 0.25 | 0.25 |
| | B5 | | | | | | | |
| 14 | EDTA | NA | 0.05 | 0.05 | 0.05 | 0.05 | 0.05 | 0.05 |

*Adapalene in poloxamer 407 (1% aq.)/TPGS (1% aq.)(D-α-Tocopherol polyethylene glycol succinate)/Poloxamer 188 (1% aq.), Sodium docusate (1% aq.)

Method of Preparation:

(1) Carbopol Ultrez 21 is added to the measured quantity of water and allowed to stir at 400 rpm for 10 minutes. Then it is neutralized with aqueous solution of sodium hydroxide.
(2) Dispersion of lipid coated particles is added to the above stirring mixture and stirring speed is increased to 800 rpm. Then glycerol is added followed by the addition of solution of methyl paraben and propyl paraben in propylene glycol to the above stirring mixture.
(3) Then the remaining ingredients are added in the same order as mentioned in the Table 19. The mixture is allowed to stir continuously at 800 rpm for 3 hrs to yield a smooth gel.

### Example 30

### Preparation of Shampoo Formulations with Lipid Coated Particle Dispersions (Compositions S4-S8)

Shampoo formulations with lipid coated particle dispersions (of Example 20) are designed and formulated as per the compositions shown in Table 21.

TABLE 21
Shampoo Formulations for Compositions S4, S5, S6, S7 and S8

| Sr. | | Strength | Composition (%) | | | | |
|---|---|---|---|---|---|---|---|
| No. | Ingredient | in % | S4 | S5 | S6 | S7 | S8 |
| | Phase A | | | | | | |
| 1 | Water | NA | qs. | qs. | qs. | qs. | qs. |
| 2 | Carbopol | 30 | 7 | 7 | 7 | 7 | 7 |
| 3 | Ammonium lauryl sulfate | 27 | 14 | 14 | 14 | 14 | 14 |
| | (ALS) | | | | | | |
| 4 | Sodium lauryl ether sulfate | 28 | 30 | 30 | 30 | 30 | 30 |
| | (SLES) | | | | | | |
| 5 | Sodium hydroxide | 18 | qs. | qs. | qs. | qs. | qs. |
| | Phase B | | | | | | |
| 6 | Coco monoethanolamide | NA | 2 | 2 | 2 | 2 | 2 |
| | (CMEA) | | | | | | |
| 7 | Ethylene glycol distearate | NA | 0 | 0 | 0 | 0 | 0 |
| | (EGDS) | | | | | | |
| 8 | Cetyl alcohol | NA | 0.5 | 0.5 | 1.0 | 0.5 | 0.5 |
| 9 | Laureth-4 | NA | 0.5 | 0.5 | 0.5 | 0.5 | 0.5 |
| 10 | Laureth-23 | NA | 2.5 | 2.5 | 2.5 | 2.5 | 2.5 |
| 11 | Glyceryl monooleate | 60 | 1 | 1 | 1 | 1 | 1 |
| 12 | Propylene glycol | NA | 1 | 3 | 1 | 0 | 0 |
| | monocaprylate | | | | | | |
| | Phase C | | | | | | |
| 13 | N-Hance | NA | 0 | 0.1 | 0 | 0 | 0 |
| 14 | Cocamidopropylbetaine | NA | 1.45 | 1.45 | 1.45 | 2.05 | 2.05 |
| | (CAPB) | | | | | | |
| 15 | Guarhydroxyl | NA | 0 | 0 | 0.1 | 0 | 0 |
| | propyltrimoniumchloride | | | | | | |
| 16 | Cassiahydroxyl | NA | 0.05 | 0 | 0 | 0.05 | 0.05 |
| | propyltrimoniumchloride | | | | | | |
| 17 | Lipid Coated Particles (D29/ | 25 | 4 (D29) | 4 (D30) | 4 (D31) | 4 (D32) | 4 (D33) |
| | D30/D31/D32/D33) | | | | | | |
| 18 | Amodimethicone emulsion | 66.6 | 2 | 2 | 2 | 2 | 2 |
| 19 | Propylene glycol | NA | 3 | 3 | 3 | 3 | 3 |
| 20 | Menthol | NA | 0.1 | 0.1 | 0.1 | 0.1 | 0.1 |
| 21 | D-Panthenol | NA | 0.6 | 0.6 | 0.6 | 0.6 | 0.6 |
| 22 | Magnesium carbonate | NA | 0 | 0.5 | 0.5 | 0.5 | 0.5 |
| 23 | Zinc carbonate | NA | 1 | 1 | 1 | 1 | 1 |
| 24 | Bromelain | NA | 0.5 | 0.5 | 0.5 | 0.5 | 0.5 |
| 25 | Chloromethyl/ | 1.51 | 0.05 | 0.05 | 0.05 | 0.05 | 0.05 |
| | Methylisothiazolinone | | | | | | |
| 26 | Linalool | NA | 0 | 0 | 0 | 0 | 0 |
| 27 | Fragrance | NA | qs. | qs. | qs. | qs. | qs. |
| 26 | Citric acid | 50 | qs. | qs. | qs. | qs. | qs. |
| 27 | Sodium Chloride | 30 | 0 | 0 | 2 | 0 | 0 |

Method of Preparation:

(1) Phase A: A required amount of water is added to a mixing vessel and stirred slowly (50-55 rpm) using an overhead stirrer. Carbopol is added to water followed by the slow addition of a premix of about 30% aqueous solutions of ammonium lauryl sulfate (ALS) and sodium lauryl ether sulfate (SLES). The mixture is neutralized by sodium hydroxide solution.
(2) Phase B: A mixture of CMEA, EGDS, cetyl alcohol, Laureth-4, Laureth-23, glyceryl monooleate and propylene glycol monocaprylate is heated to melt. The resulting melt is immediately poured to Phase A while stirring at about 60° C. After stirring for about 5 min at the same temperature, it is allowed to cool to about 35° C. to 40° C.
(3) Phase C: Cocamidopropylbetaine (CAPB) with Cassia hydroxypropyltrimonium chloride/N-Hance/Guar hydroxypropyltrimonium chloride are added to the above stirring mixture followed by the addition of lipid coated particle dispersion (of Example 17). Then, amodimethicone emulsion is added while stirring followed by additions of solutions of menthol in propylene glycol, and D-Panthenol in propylene glycol. Then the solution of magnesium carbonate is added followed by the addition of zinc carbonate along with bromelain. Then the remaining ingredients of phase C are added in the same order as mentioned in the Table 21. The continuously stirring mixture (300 rpm) is then allowed to cool to room temperature followed by addition of fragrance. Finally, pH is adjusted with citric acid and viscosity by sodium chloride, and mixture is continued to stir to yield a smooth and shiny shampoo.

### Example 31

### Preparation of Conditioner Formulations with Lipid Coated Particle Dispersions (Compositions C4-C6)

Conditioner formulations with lipid coated particle dispersions (of Example 20) are designed and formulated as per the compositions shown in Table 22.

TABLE 22
Conditioner Formulations for Compositions C4-C6

| | | Strength | Composition (%) | | |
|---|---|---|---|---|---|
| Sr. No. | Ingredients | (%) | C4 | C5 | C6 |
| | Phase A | | | | |
| 1 | Water | NA | qs. | qs. | qs. |
| 2 | Carbopol | 30 | 3 | 3 | 3 |
| 3 | Sodium lauryl ether sulphate | 28 | 2 | 2 | 2 |
| | (SLES) | | | | |
| 4 | Sodium hydroxide | 18 | qs. | qs. | qs. |
| | Phase B | | | | |
| 5 | Steareth-2 | NA | 1.85 | 1.85 | 1.85 |
| 6 | Steareth-21 | NA | 3.15 | 3.15 | 3.15 |
| 7 | Propylene glycol monocaprylate | NA | 5 | 3 | 0 |
| 8 | Macrogolcetostearyl ether 20 | NA | 1 | 1 | 1 |
| 9 | Coco monoethanolamide | NA | 0.6 | 0.6 | 0.6 |
| | (CMEA) | | | | |
| 10 | Cetyl Alcohol | NA | 2 | 2 | 2 |
| 11 | Stearamidopropyldimethylamine | NA | 0.5 | 0.5 | 0.5 |
| 12 | Lactic acid | 50 | qs. | qs. | qs. |
| 13 | Tea Tree Oil | NA | 0.1 | 0.1 | 0.1 |
| | Phase C | | | | |
| 13 | Cocamidopropylbetain (CAPB) | 30 | 2 | 2 | 2 |
| 14 | Cetrimoniumchloride (CTC) | 30 | 3 | 3 | 3 |
| 15 | Polyquaternium-22 | NA | 1 | 1 | 1 |
| 16 | Amodimethicone emulsion | 66.6 | 5 | 5 | 5 |
| 17 | Cassia | 1 | 2 | 0 | 0 |
| | hydroxypropyltrimoniumchloride | | | | |
| 18 | Guar | NA | 0 | 0.25 | 0 |
| | hydroxypropyltrimoniumchloride | | | | |
| 19 | N-Hance | NA | 0 | 0 | 0.25 |
| 20 | Propylene glycol | NA | 5.6 | 5.6 | 5.6 |
| 21 | D - Panthenol | NA | 0.6 | 0.6 | 0.6 |
| 22 | Glycerine | NA | 5 | 5 | 5 |
| 23 | Lipid Coated Particles (D29/ | 25 | 4 (D29) | 4 (D31) | 4 (D33) |
| | D31/D33) | | | | |
| 24 | Zinc carbonate | NA | 1 | 1 | 1 |
| 25 | Titanium dioxide | NA | 0.5 | 0.5 | 0.5 |
| 26 | DL-α-Tocopherol acetate | NA | 0.5 | | |
| 27 | Linalool | NA | 0.1 | 0.1 | 0.1 |
| 28 | Fragrance | NA | qs. | qs. | qs. |
| 29 | Chloromethyl/ | 1.51 | 0.05 | 0.05 | 0.05 |
| | Methylisothiazolinone | | | | |

Method of Preparation:
(1) Phase A: A required amount of water is added to a mixing vessel and stirred slowly (50-55 rpm) using an overhead stirrer. Carbopol is added to water followed by the slow addition of about 28% aqueous solution of sodium lauryl ether sulfate (SLES). Then mixture is neutralized by adding sodium hydroxide solution.
(2) Phase B: Components of Phase B are mixed and heated to melt. Lactic acid is added to the resulting melted mixture to neutralize. The Phase B is added to Phase A while stirring at about 60° C. Then tea tree oil is added. After uniform mixing (200 rpm), the mixture is allowed to cool to 35° C. to 40° C.
(3) To the above stirring mixture, cocamidopropylbetaine, cetrimonium chloride, polyquaternium-22, amodimethicone emulsion, Cassia hydroxypropyltrimonium chloride/Guar hydroxypropyltrimoniumchloride/N-Hance, propylene glycol, D-Panthenol and glycerin are added slowly in the same order as mentioned in Table 22 and stirred (300 rpm) till uniform mixing. Lipid coated particle dispersion is added to the stirring mixture followed by addition of zinc carbonate, titanium dioxide and DL-α-Tocopherol acetate. The mixture is then allowed to cool to room temperature. Finally, linalool, fragrance and preservatives are added, and the mixture is allowed to stir in order to yield a smooth uniform conditioner cream.

### Example 32

### Effect of Propylene Glycol Monocaprylate Addition (Below C11 Fatty Acid Ester) on Zinc Pyrithione Activity Towards Malassezia furfur

An activity comparison, by in vitro time-kill kinetics of zinc pyrithione using plain zinc pyrithione (Kopithione, Kumar Organic Products Ltd) versus its combination with different concentrations of propylene glycol monocaprylate (Capmul 908-P, Croda), has been demonstrated.

The time-kill assays are used to evaluate efficacy of antimicrobial agents, either singly or in combination, and the results can help in establishing the dose and/or time of application of the active. The time-kill assays can be used to study both concentration-dependent and time-dependent antimicrobial action.

Method: M. furfur cells were suspended in Sabouraud Dextrose Broth (SDB) at inoculum concentration of 7×107 cells/ml. Cells were taken from a freshly growing (3-7 days old) plate and cell suspension is vortexed to remove the cell clumps as much as possible. Sterile media were supplemented with chloramphenicol (0.25 mg/ml), cycloheximide (0.04 mg/ml) and olive oil (2%). The media were then supplemented with appropriate concentrations (two-fold serial dilutions using SDB) of zinc pyrithione powder (10 μg/ml and 50 μg/ml) with different concentrations of Capmul 908-P (0%, 1%, 3%, 5% & 9%). The cultures were incubated on a tube rotator at 34° C. in CO₂ incubator.

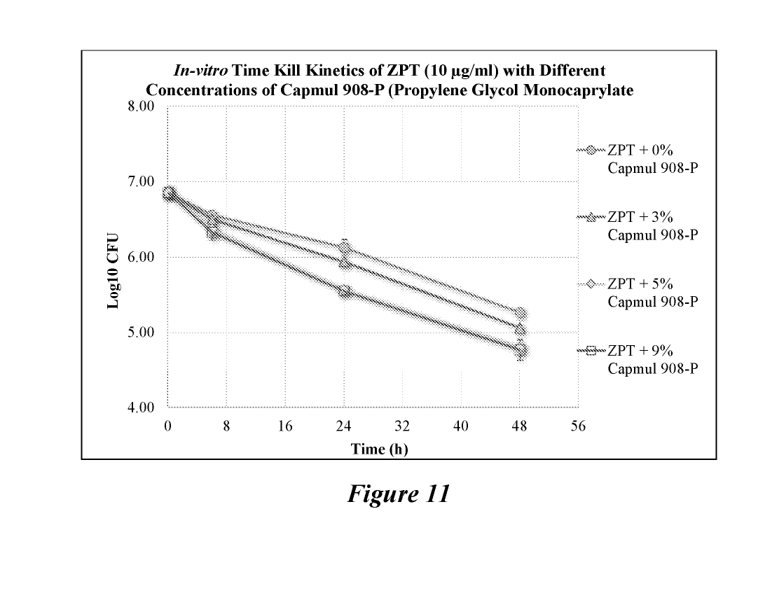

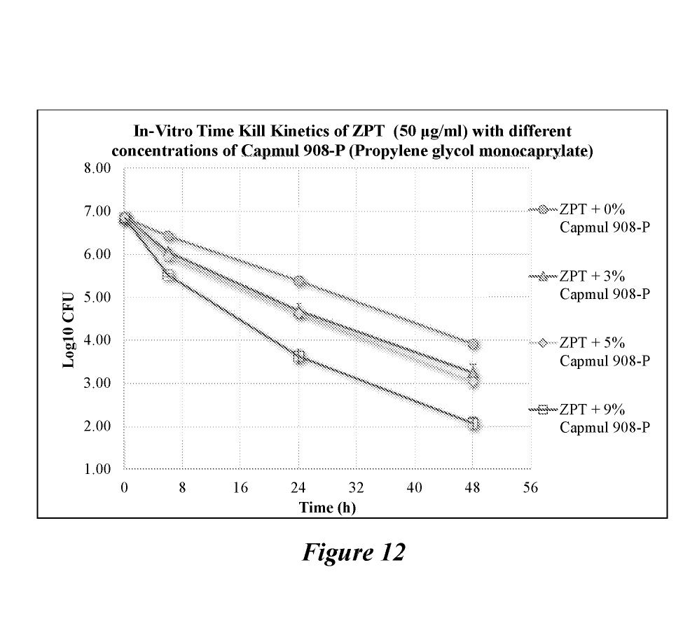

To measure the colony forming units (CFU), at different time points, aliquots (50 μl) of Malassezia cultures were serially diluted with SDBT medium (SDB containing 0.1% Triton X-100) and plated on SDA plates. The plates were incubated at 34° C. in CO₂ incubator for 3 days. The viable colonies were counted and converted to CFU/ml. The results of time kill study using zinc pyrithione powder with different concentration of Capmul 908-P are shown in Tables 23 and 24 and are plotted in FIGS. 11 and 12.

Result: The results of the assays suggest that the anti-microbial activity of zinc pyrithione towards M. furfur, got potentiated with increasing concentration of propylene glycol monocaprylate.

TABLE 23

| | Log CFU Count of M. furfurat ZPT concentration of | | | |
|---|---|---|---|---|
| | 10 μg/ml with different Capmul 908-P concentration | | | |
| Time | Capmul 908-P | Capmul 908-P | Capmul 908-P | Capmul 908-P |
| (hrs) | (nil) | (30 μg/ml) | (50 μg/ml) | (90 μg/ml) |
| 6 | 6.56 | 6.50 | 6.37 | 6.33 |
| 24 | 6.13 | 5.94 | 5.57 | 5.55 |
| 48 | 5.27 | 5.06 | 4.80 | 4.77 |

TABLE 24

| | Log CFU Count of M. furfur at ZPT concentration of | | | |
|---|---|---|---|---|
| | 50 μg/ml with different Capmul 908-P concentration | | | |
| Time | Capmul 908-P | Capmul 908-P | Capmul 908-P | Capmul 908-P |
| (h) | (nil) | (30 μg/ml) | (50 μg/ml) | (90 μg/ml) |
| 6 | 6.43 | 6.06 | 5.94 | 5.52 |
| 24 | 5.39 | 4.69 | 4.62 | 3.63 |
| 48 | 3.92 | 3.25 | 3.04 | 2.08 |

### Example 33

### Effect of Propylene Glycol Monocaprylate on Retention/Deposition of ZPT Particles (Suspended in Shampoo) on Goat Skin Model (Ex-Vivo Studies)

In order to understand the influence of formulation components i.e., propylene glycol monocaprylate, on retention behavior of coated particles and their corresponding formulations on human skin/scalp/hair, suitable models are required to conduct preliminary retention trials. For scalp studies, goat skin (easily available) seems to have close resemblance with human scalp based on hair density [average hair density: 125-200 hair/cm² (human scalp); about 225 hair/cm² (goat skin)].

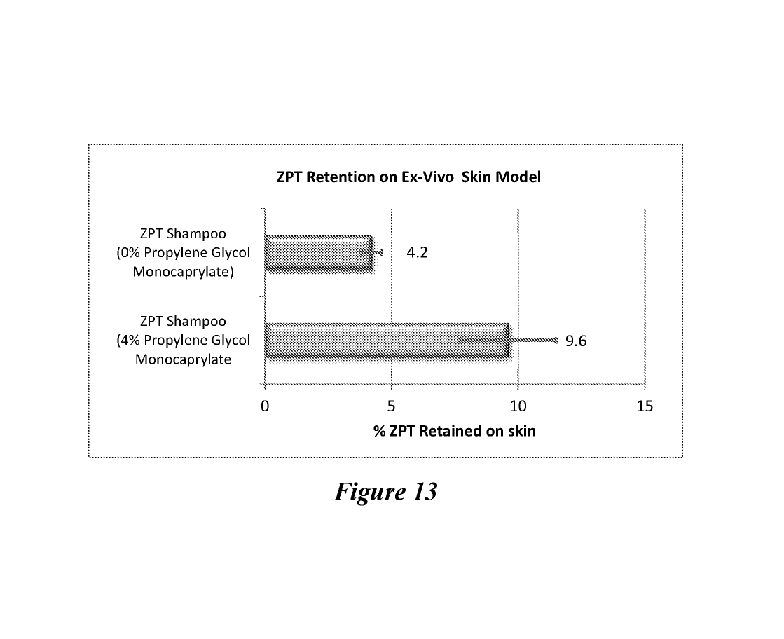

Method: Goat skin, fitted in Franz cell, was incubated for 20 min (including 5 min of gentle messaging) with test sample of 1.77 mg per ml of formulation per 7.07 cm² of exposed goat skin (either dispersion or final formulation). After incubation, each piece of skin was washed with water to remove un-retained particles and excess formulation. The skin was cut into small pieces and the homogenization in DMSO was effected using high shear homogenizer at 25,000 rpm for 5 min with intervals of 30 sec after every 1 min. After homogenization, the extract was subjected to bath sonication for 10 min at 30° C. followed by centrifugation for 20 minutes at 1700 rpm to collect supernatant. The supernatants after filtration through 0.45 μm filters, were analyzed for zinc content using Atomic Absorption Spectroscopy (AAS). The experiments were always performed at least in triplicate. Percentage of ZPT retained in skin (w.r.t. total applied amount) for some of the ZPT formulation compositions are plotted in the graphs shown in FIG. 13.

### Example 34

### Dispersion of Lipid Coated API (One Active in One Coat) Microparticles

### Compositions D90-D104

Preparation:

Besifloxacin hydrochloride is dispersed in surfactant solution (aqueous solution of lecithin: poloxamer 407 {1:1}), and homogenized at 30,000 rpm (three cycles with 1 minute intervals) for 5 minutes using high shear homogenizer. The required quantity of homogenized dispersion is heated to about 70-80° C. with continuous stirring. Weighed quantity of lipid (stearic acid) is melted and added to above hot dispersion. The mixture is allowed to stir vigorously at the same temperature and continued to stir for about 20 minutes followed by stirring in an ice bath for about 10 minutes. Particle size and its distribution of the resulting dispersion is determined by ZetaSizer (ZS-90 from Malvern Instruments), Scanning Electron Microscope (SEM, Hitachi, S-3400 N, Japan) and Mastersizer (Malvern Instruments). To this dispersion is added a suitable stabilizer, and the pH was controlled using a pH modulator.

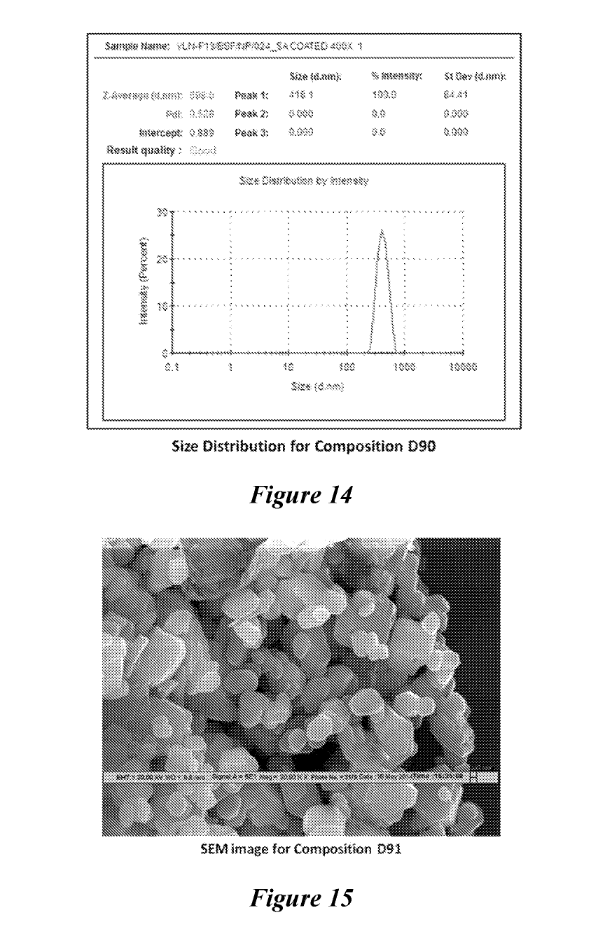

This way several dispersion compositions are prepared, and some of the examples are given in Table 25 and representative images are shown in FIGS. 14, 15 and 16. For dispersion preparation, different types of surfactant solutions such as lecithin, Poloxamer 407, TPGS (D-α-tocopherol polyethylene glycol succinate), poloxamer 188, sodium docusate are used alone or in combination. Similarly, some of the lipids used are lauric acid, myristic acid, palmitic acid, stearic acid, ethylene glycol distearate (EGDS), ethylene glycol dilaurate (EGDL), ethylene glycol dipalmitate (EGDP), ethylene glycol dimyristate (EGDM), propylene glycol distearate (PGDS), propylene glycol dilaurate (PGDL), propylene glycol dipalmitate (PGDP), propylene glycol dimyristate (PGDM), etc. Different stabilizers that are used here are carbopol, xanthan gum, guar gum, hydroxyl propyl methyl cellulose, etc.

TABLE 25
Examples of dispersions of lipid coated API
(one active in one coat) microparticles

| | | | | Average | |
|---|---|---|---|---|---|
| Compo- | Core | Coat^{$} | Coat:Core | Particle Size | |
| sitions | (% w/w) | (% w/w) | ratio | (PDI) | |
| D90 | Besifloxacin | Stearic acid | 1:5 | *0.598 | μm, (0.528) |
| | | (2%) | | | |
| D91 | Zinc pyrithione | EGDS (5%) | 1:5 | **1.537 | μm |
| D92 | Zinc pyrithione | EGDS (5%) | 1:5 | 1.2 | μm(1) |
| D93 | Besifloxacin | Stearic acid | 1:5 | 6.2 | μm |
| | | (2%) | | | |
| D94 | Besifloxacin | Lauric acid | 1:5 | 3.311 | μm (0.291) |
| | | (2%) | | | |

| | | | | |
|---|---|---|---|---|
| D95 | Besifloxacin | Stearic acid | 5:1 | — |
| | | (5%) | | |
| D96 | Salicylic acid | Stearic acid | 3:1 | — |
| | | (3%) | | |
| D97 | Besifloxacin | Palmitic acid | 5:1 | — |
| | | (10%) | | |
| D98 | Adapalene | Lauric acid | 5:1 | — |
| | | (5%) | | |
| D99 | Minoxidil | Lauric acid | 7:1 | — |
| | | (7%) | | |
| D100 | Minoxidil | Stearic acid | 10:1 | — |
| | | (10%) | | |
| D101 | Minoxidil | Palmitic acid | 10:1 | — |
| | | (10%) | | |
| D102 | Ketoconazole | Stearic acid | 5:1 | — |
| | | (5%) | | |
| D103 | Adapalene | Stearic acid | 5:1 | — |
| | | (5%) | | |
| D104 | Zinc pyrithione | EGDS (8%) | 8:1 | — |

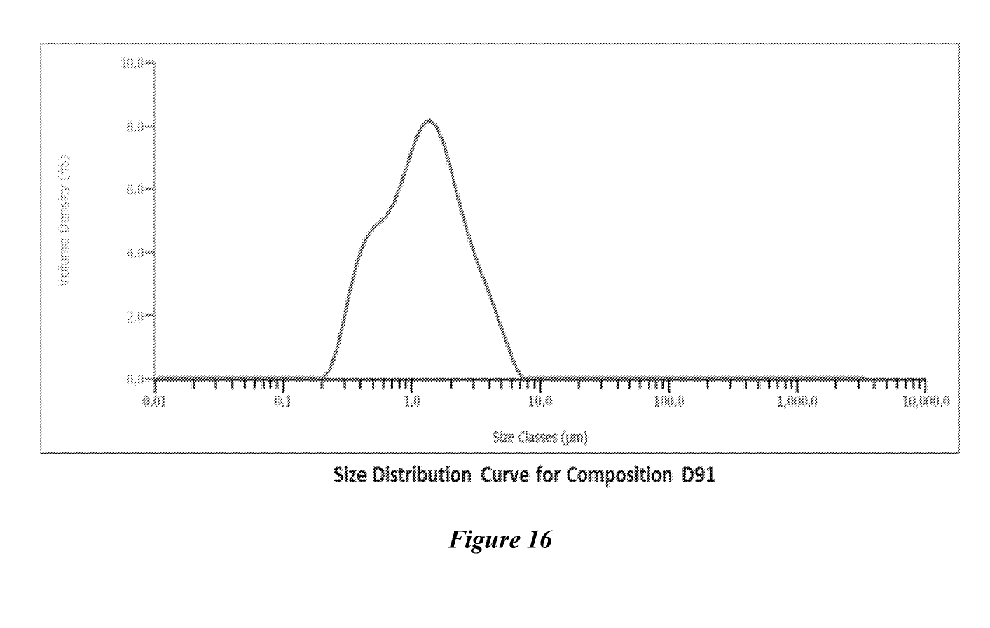

*As reported in FIG. 14.
**As reported in FIG. 16.
^{$}Coat abbreviations are mentioned as EGDS—ethylene glycol distearate

### Example 35

### Dispersion of Lipid Coated API (One Active in One Coat) Particles (Compositions D105-D110)

Preparation:

Zinc pyrithione is dispersed in surfactant solution (2% aqueous solution of poloxamer 407), and heated to about 70-80° C. with continuous stirring. Weighed quantity of lipid (stearic acid) is added to a surfactant solution (2% aqueous solution of poloxamer 407) separately and heated to melt the lipid. The mixture is allowed to stir vigorously at the same temperature. To this stirring mixture, hot (about 70-80° C.) homogenized dispersion of the active is added and allowed to stir continuously at the same temperature for about 20 minutes followed by stirring in an ice bath for about 10 minutes. Size distribution of the resulting dispersion is determined by ZetaSizer (ZS-90 from Malvern Instruments) and Scanning Electron Microscope (SEM, Hitachi, S-3400 N, Japan). To this dispersion is added a suitable stabilizer, and the pH was controlled using a pH modulator.

This way several dispersion compositions are prepared, and some of the examples are given in Table 26. For dispersion preparation, different types of surfactant solutions such as lecithin, Poloxamer 407, TPGS (D-α-tocopherol polyethylene glycol succinate), poloxamer 188, sodium docusate are used alone or in combination. Similarly, some of the lipids used are lauric acid, myristic acid, palmitic acid, stearic acid, EGDS, EGDL, EGDP, EGDM, etc. Different stabilizers that are used here are carbopol, xanthan gum, guar gum, hydroxyl propyl methyl cellulose, etc.

TABLE 26
Examples of dispersions of lipid coated
API (one active in one coat) particles

| Compo- | | Coat | Coat:Core | |
|---|---|---|---|---|
| sitions | Core | (% w/w) | ratio | Appearance |
| D105 | Zinc Pyrithione | Stearic acid | 18:1 | White |
| | | (18%) | | liquid dispersion |
| D106 | Zinc Pyrithione | Palmitic acid | 18:1 | White |
| | | (18%) | | liquid dispersion |
| D107 | Zinc Pyrithione | Ethylene glycol | 10:1 | White |
| | | distearate | | liquid dispersion |
| | | (10%) | | |
| D108 | Zinc Pyrithione | Lauric acid | 7:1 | White |
| | | (7%) | | liquid dispersion |
| D109 | Ketoconazole | Stearic acid | 5:1 | White |
| | | (10%) | | liquid dispersion |
| D110 | Adapalene | Stearic acid | 18:1 | White |
| | | (18%) | | pasty dispersion |

### Example 36

### Dispersion of Lipid Coated API (Two Actives in One Coat) Microparticles (Compositions D111-D121)

Preparation:

Besifloxacin hydrochloride and adapalene, individually, are dispersed in surfactant solution (2% aqueous solution of poloxamer 407), and homogenized at 30,000 rpm (three cycles with 1 minute intervals) for 5 minutes using high shear homogenizer. The required quantities of homogenized dispersions of the two actives are heated individually to about 70-80° C. with continuous stirring. Weighed quantity of lipid (stearic acid) is added to a surfactant solution (2% aqueous solution of poloxamer 407) separately and heated to melt the lipid. The mixture is allowed to stir vigorously at the same temperature. To this stirring mixture, hot (about 70-80° C.) homogenized dispersions of the two actives are added and allowed to stir continuously at the same temperature for about 20 minutes followed by stirring in an ice bath for about 10 minutes. Size distribution of the resulting dispersion is determined by ZetaSizer (ZS-90 from Malvern Instruments), and Scanning Electron Microscope (SEM, Hitachi, S-3400 N, Japan). To this dispersion is added a suitable stabilizer, and the pH was controlled using a pH modulator.

This way several dispersion compositions are prepared, and some of the examples are given in Table 27. For dispersion preparation, different types of surfactant solutions such as lecithin, Poloxamer 407, TPGS (D-α-tocopherol polyethylene glycol succinate), poloxamer 188, sodium docusate are used alone or in combination. Similarly, some of the lipids used are lauric acid, myristic acid, palmitic acid, stearic acid, EGDS, EGDL, EGDP, EGDM, etc. Different stabilizers that are used here are carbopol, xanthan gum, guar gum, hydroxyl propyl methyl cellulose, etc.

TABLE 27
Examples of dispersions of lipid coated API
(two actives in a single coat) microparticles

| Compo- | Core | Coat | Coat:Core | |
|---|---|---|---|---|
| sitions | (API 1:API 2) | (% w/w) | ratio | Appearance |
| D111 | Besifloxacin &Adapalene | Stearic acid | 5:1 | Pale yellowish |
| | (1.0:1.0) | (10%) | | liquid dispersion |
| D112 | Besifloxacin &Ketoconazole | Stearic acid | 5:1 | Pale yellowish |
| | (1.0:1.0) | (10%) | | liquid dispersion |
| D113 | Besifloxacin &Salicylic Acid | Stearic acid | 5:1 | Pale yellowish |
| | (1.0:1.0) | (10%) | | liquid dispersion |
| D114 | Adapalene& Ketoconazole | Palmitic acid | 4:1 | White liquid |
| | (1.0:1.0) | (8%) | | dispersion |
| D115 | Besifloxacin&Adapalene | Stearic acid | 5:1 | Pale yellowish |
| | (1.0:0.1) | (10%) | | liquid dispersion |
| D116 | Clintafloxacin&Adapalene | Stearic acid | 7:1 | White |
| | (1.0:0.1) | (10%) | | liquid dispersion |
| D117 | Besifloxacin &Adapalene | Stearic acid | 5:1 | Pale yellowish |
| | (1.0:0.2) | (10%) | | liquid dispersion |
| D118 | Salicylic Acid & Benzoyl | Lauric acid | 5:1 | White |
| | Peroxide (1.0:1.0) | (10%) | | liquid dispersion |
| D119 | Sitafloxacin& Ketoconazole | EGDS | 5:1 | White |
| | (1.0:1.0) | (10%) | | liquid dispersion |
| D120 | Besifloxacin & Ketoconazole | Palmitic acid | 5:1 | Pale yellowish |
| | (1.0:1.0) | (10%) | | liquid dispersion |
| D121 | Triclosan& Zinc Pyrithione | Lauric acid | 8:1 | White |
| | (1.0:1.0) | (16%) | | liquid dispersion |

### Example 37

### Dispersion of Lipid Coated API (Two Actives in a Single Coat) Particles (Compositions D122-D127)

Preparation:

Besifloxacin stearate and ketoconazole, individually, are dispersed in surfactant solution (2% aqueous solution of poloxamer 407), and heated individually to about 70-80° C. with continuous stirring. Weighed quantity of lipid (stearic acid) is added to a surfactant solution (2% aqueous solution of poloxamer 407) separately and heated to melt the lipid. The mixture is allowed to stir vigorously at the same temperature. To this stirring mixture, hot (about 70-80° C.) homogenized dispersions of the two actives are added and allowed to stir continuously at the same temperature for about 20 minutes followed by stirring in an ice bath for about 10 minutes. Size distribution of the resulting dispersion is determined by ZetaSizer (ZS-90 from Malvern Instruments) and Scanning Electron Microscope (SEM, Hitachi, S-3400 N, Japan). To this dispersion is added a suitable stabilizer, and the pH was controlled using a pH modulator.

This way several dispersion compositions are prepared, and some of the examples are given in Table 28. For dispersion preparation, different types of surfactant solutions such as lecithin, poloxamer 407, TPGS (D-α-tocopherol polyethylene glycol succinate), poloxamer 188, sodium docusate are used alone or in combination. Similarly, some of the lipids used are lauric acid, myristic acid, palmitic acid, stearic acid, EGDS, EGDL, EGDP, EGDM, etc. Different stabilizers that are used here are carbopol, xanthan gum, guar gum, hydroxyl propyl methyl cellulose, etc.

TABLE 28
Examples of dispersions of lipid coated API (two actives in a single coat) particles

| Compo- | Core | Coat | Coat:Core | |
|---|---|---|---|---|
| sitions | (API 1:API 2) | (% w/w) | ratio | Appearance |
| D122 | Besifloxacin &Adapalene | Stearic acid | 9:1 | Pale yellowish |
| | (1.0:1.0) | (18%) | | pasty dispersion |
| D123 | Besifloxacin & | Stearic acid | 9:1 | Pale yellowish |
| | Ketoconazole | (18%) | | pasty dispersion |
| | (1.0:1.0) | | | |
| D124 | Besifloxacin & Zinc | Ethylene glycol | 4:1 | Pale yellowish |
| | Pyrithione | distearate | | liquid dispersion |
| | (1.0:1.0) | (8%) | | |
| D125 | Salicylic acid & | Palmitic acid | 5:1 | White |
| | Ketoconazole | (10%) | | liquid dispersion |
| | (1.0:1.0) | | | |
| D126 | Besifloxacin & | Lauric acid | 2.5:1 | Pale yellowish |
| | Ketoconazole | (5%) | | liquid dispersion |
| | (1.0:1.0) | | | |
| D127 | Adapalene& | Stearic acid | 4:1 | White |
| | Ketoconazole | (8%) | | liquid dispersion |
| | (1.0:1.0) | | | |

### Example 38

### Dispersion of Lipid Coated API (Two Actives in Two Coats Separately) Microparticles (Compositions D128-D136)

Preparation:

Besifloxacin laurate is dispersed in surfactant solution (2% aqueous solution of poloxamer 407), and homogenized at 30,000 rpm (three cycles with 1 minute intervals) for 5 minutes using high shear homogenizer. The required quantity of homogenized dispersion is heated to about 70-80° C. with continuous stirring. Weighed quantity of lipid (stearic acid) is added to a surfactant solution (2% aqueous solution of poloxamer 407) separately and heated to melt the lipid. The mixture is allowed to stir vigorously at the same temperature. To this stirring mixture, hot (about 70-80° C.) homogenized dispersion of besifloxacin is added and allowed to stir continuously at the same temperature for about 20 minutes followed by stirring in an ice bath for about 10 minutes. Similarly, ketoconazole is homogenized and coated with stearic acid. Then, the resulting homogenized and coated dispersions are mixed together followed by stirring for 10-15 minutes. Size distribution of the resulting dispersion is determined by ZetaSizer (ZS-90 from Malvern Instruments) and Scanning Electron Microscope (SEM, Hitachi, S-3400 N, Japan). To this dispersion is added a suitable stabilizer, and the pH was controlled using a pH modulator.

This way several dispersion compositions are prepared, and some of the examples are given in Table 29. For dispersion preparation, different types of surfactant solutions such as lecithin, poloxamer 407, TPGS (D-α-tocopherol polyethylene glycol succinate), poloxamer 188, sodium docusate are used alone or in combination. Similarly, some of the lipids used are lauric acid, myristic acid, palmitic acid, stearic acid, EGDS, EGDL, EGDP, EGDM, etc. Different stabilizers that are used here are carbopol, xanthan gum, guar gum, hydroxyl propyl methyl cellulose, etc.

TABLE 29
Examples of dispersions of lipid coated API (two actives in
two coat separately) microparticles

| | Coated | | | | | | |
|---|---|---|---|---|---|---|---|
| | API 1; | | | | | | |
| | Coated | Coated API 1* | | | Coated API 2* | | |
| | API 2 | | Coat | Coat:Core | | Coat 2 | Coat:Core |
| Compositions | Ratio | Core | (% w/w) | ratio | Core 2 | (% w/w) | ratio |
| D128 | 1.0:2.5 | BSF | Stearic acid | 5:1 | SAL | Stearic acid | 3:1 |
| | | | (5%) | | | (7.5%) | |
| D129 | 1.0:0.1 | BSF | Stearic acid | 5:1 | ADP | Lauric acid | 25:1 |
| | | | (5%) | | | (2.5%) | |
| D130 | 1.0:1.0 | BSF | Stearic acid | 5:1 | KTZ | Stearic acid | 5:1 |
| | | | (5%) | | | (5%) | |
| D131 | 1.0:0.1 | BSF | Stearic acid | 5:1 | ADP | Stearic acid | 5:1 |
| | | | (5%) | | | (5%) | |
| D132 | 1.0:1.0 | TRI | Lauric acid | 6:1 | SAL | Palmitic | 5:1 |
| | | | (6%) | | | acid | |
| | | | | | | (5%) | |
| D133 | 1.0:1.0 | STF | EGDS (5%) | 5:1 | KTZ | Lauric acid | 7:1 |
| | | | | | | (7%) | |
| D134 | 1.0:1.0 | FLZ | Palmitic | 4:1 | CLF | Lauric acid | 7:1 |
| | | | acid (4%) | | | (7%) | |
| D135 | 1.0:1.0 | CLM | Lauric acid | 5:1 | TRI | Lauric acid | 5:1 |
| | | | (5%) | | | (5%) | |
| D136 | 1.0:1.0 | KTZ | Lauric acid | 10:1 | SAL | Stearic acid | 3:1 |
| | | | (10%) | | | (3%) | |

*Core abbreviations are mentioned as-BSF—Besifloxacin; TRI—Triclosan; STF—Sitafloxacin; FLZ—Fluconazole, CLM—Climbazole; KTZ—Ketoconazole; SAL—Salicylic acid, ADP—Adapalene; CLF—Clintafloxacin

### Example 39

### Dispersion of Lipid Coated API (Two Actives in Two Coats Separately) Particles (Compositions D137-D141)

Preparation:

Fluconazole is dispersed in surfactant solution (2% aqueous solution of poloxamer 407), and heated to about 70-80° C. with continuous stirring. Weighed quantity of lipid (palmitic acid) is added to a surfactant solution (2% aqueous solution of poloxamer 407) separately and heated to melt the lipid. The mixture is allowed to stir vigorously at the same temperature. To this stirring mixture, hot (about 70-80° C.) homogenized dispersion of besifloxacin is added and allowed to stir continuously at the same temperature for about 20 minutes followed by stirring in an ice bath for about 10 minutes. Similarly, adapalene is coated with EGDS. Then, the resulting coated dispersions are mixed together followed by stirring for 10-15 minutes. Size distribution of the resulting dispersion is determined by ZetaSizer (ZS-90 from Malvern Instruments) and Scanning Electron Microscope (SEM, Hitachi, S-3400 N, Japan). To this dispersion is added a suitable stabilizer, and the pH was controlled using a pH modulator.

This way several dispersion compositions are prepared, and some of the examples are given in Table 30. For dispersion preparation, different types of surfactant solutions such as lecithin, poloxamer 407, TPGS (D-α-tocopherol polyethylene glycol succinate), poloxamer 188, sodium docusate are used alone or in combination. Similarly, some of the lipids used are lauric acid, myristic acid, palmitic acid, stearic acid, EGDS, EGDL, EGDP, EGDM, etc. Different stabilizers that are used here are carbopol, xanthan gum, guar gum, hydroxyl propyl methyl cellulose, etc.

TABLE 30
Examples of dispersions of lipid coated API (two actives in two coat
separately) particles

| | Coated | | | | | | |
|---|---|---|---|---|---|---|---|
| | API 1; | | | | | | |
| | Coated | Coated API 1* | | | Coated API 2* | | |
| | API 2 | | Coat | Coat:Core | | Coat 2 | Coat:Core |
| Compositions | Ratio | Core | (% w/w) | ratio | Core 2 | (% w/w) | ratio |
| D137 | 1.0:1.0 | FLU | Stearic acid | 5:1 | ADP | EGDS* | 5:1 |
| | | | (5%) | | | (5%) | |
| D138 | 1.0:0.1 | BSF | Stearic acid | 5:1 | ADP | Stearic acid | 5:1 |
| | | | (5%) | | | (0.5%) | |
| D139 | 1.0:1.0 | BSF | Stearic acid | 5:1 | KTZ | Stearic acid | 5:1 |
| | | | (5%) | | | (5%) | |
| D140 | 1.0:1.0 | ADP | Lauric acid | 5:1 | TRI | EGDS | 5:1 |
| | | | (5%) | | | (5%) | |
| D141 | 1.0:1.0 | KTZ | EGDS | 5:1 | ADP | Palmitic | 5:1 |
| | | | (5%) | | | acid | |
| | | | | | | (5%) | |

*Core abbreviations are mentioned as-BSF—Besifloxacin; TRI—Triclosan; FLZ—Fluconazole, KTZ—Ketoconazole; SAL—Salicylic acid, ADP—Adapalene.
**Coat abbreviation is mentioned as-EGDS—Ethylene glycol strearate

### Example 40

### Dispersion of Lipid Coated API (Two Actives in Two Coats Separately) Particles (Compositions D142-D146)

Preparation:

Adapalene is dispersed in surfactant solution (2% aqueous solution of poloxamer 407), and homogenized at 30,000 rpm (three cycles with 1 minute intervals) for 5 minutes using high shear homogenizer. The required quantity of homogenized dispersion is heated to about 50-60° C. with continuous stirring. Weighed quantity of lipid (lauric acid) is added to a surfactant solution (2% aqueous solution of poloxamer 407) separately and heated to melt the lipid. The mixture is allowed to stir vigorously at the same temperature. To this stirring mixture, hot (about 50-60° C.) homogenized dispersion of adapalene is added and allowed to stir continuously at the same temperature for about 20 minutes followed by stirring in an ice bath for about 10 minutes. Similarly, zinc pyrithione is coated with EGDS without homogenization. Then, the resulting coated dispersions are mixed together followed by stirring for 10-15 minutes. Size distribution of the resulting dispersion is determined by ZetaSizer (ZS-90 from Malvern Instruments) and Scanning Electron Microscope (SEM, Hitachi, S-3400 N, Japan). To this dispersion is added a suitable stabilizer, and the pH was controlled using a pH modulator.

This way several dispersion compositions are prepared, and some of the examples are given in Table 31. For dispersion preparation, different types of surfactant solutions such as lecithin, poloxamer 407, TPGS (D-α-tocopherol polyethylene glycol succinate), poloxamer 188, sodium docusate are used alone or in combination. Similarly, some of the lipids used are lauric acid, myristic acid, palmitic acid, stearic acid, EGDS, EGDL, EGDP, EGDM, etc. Different stabilizers that are used here are carbopol, xanthan gum, guar gum, hydroxyl propyl methyl cellulose, etc.

TABLE 31
Examples of dispersions of lipid coated API (two actives in two coats
separately) particles and microparticles

| | Coated | | | | | | |
|---|---|---|---|---|---|---|---|
| | API 1; | | | | | | |
| | Coated | Coated API 1* | | | Coated API 2* | | |
| | API 2 | | Coat | Coat:Core | | Coat 2 | Coat:Core |
| Compositions | Ratio | Core | (% w/w) | ratio | Core 2 | (% w/w) | ratio |
| D142 | 1.0:1.0 | ADP | Lauric acid | 5:1 | ZPT | EGDS** | 10:1 |
| | | | (2.5%) | | | (5%) | |
| D143 | 1.0:0.5 | BSF | Stearic acid | 5:1 | ZPT | Palmitic | 18:1 |
| | | | (5%) | | | acid | |
| | | | | | | (9%) | |
| D144 | 1.0:1.0 | SAL | Palmiticacid | 5:1 | KTZ | Stearic acid | 5:1 |
| | | | (5%) | | | (5%) | |
| D145 | 1.0:0.1 | KTZ | EGDS** | 5:1 | ADP | Stearic acid | 5:1 |
| | | | (5%) | | | (0.5%) | |
| D146 | 1.0:1.0 | TRI | Lauric acid | 5:1 | ADP | EGDP** | 5:1 |
| | | | (5%) | | | (5%) | |

*Core abbreviations are mentioned as-BSF—Besifloxacin; TRI—Triclosan; FLZ—Fluconazole, KTZ—Ketoconazole; SAL—Salicylic acid, ADP—Adapalene.
**Coat abbreviation is mentioned as-EGDS—Ethylene glycol strearate; EGDP—Ethylene glycol dipalmitate.

### Example 41

### Dispersion of Carbohydrate and Lipid Coated API Microparticles (Compositions D147-D150)

Preparation:

Besifloxacin hydrochloride and adapalene, individually, are dispersed in surfactant solution (2% aqueous solution of poloxamer 407), and homogenized at 30,000 rpm (three cycles with 1 minute intervals) for 5 minutes using high shear homogenizer. The required quantities of homogenized dispersions of the two actives are heated individually to about 70-80° C. with continuous stirring. Weighed quantity of lipid (stearic acid) is added to a surfactant solution (2% aqueous solution of poloxamer 407) separately and heated to melt the lipid. The mixture is allowed to stir vigorously at the same temperature. To this stirring mixture, hot (about 70-80° C.) homogenized dispersions of the two actives are added and allowed to stir continuously at the same temperature for about 20 minutes followed by stirring in an ice bath for about 10 minutes. The resulting dispersion is added to chitosan solution (0.3% in 1% aqueous solution of acetic acid) drop wise with continuous stirring for 1 hr. to get homogenized mixture followed by pH adjustment to 5.0-5.5 using aqueous solution of sodium hydroxide (18%).

Size distribution of the resulting dispersion is determined by ZetaSizer (ZS-90 from Malvern Instruments) and Scanning Electron Microscope (SEM, Hitachi, S-3400 N, Japan). To this dispersion is added a suitable stabilizer, and the pH was controlled using a pH modulator.

This way several dispersion compositions are prepared, and some of the examples are given in Table 32. For dispersion preparation, different types of surfactant solutions such as lecithin, Poloxamer 407, TPGS (D-α-tocopherol polyethylene glycol succinate), poloxamer 188, sodium docusate are used alone or in combination. Similarly, some of the lipids used are lauric acid, myristic acid, palmitic acid, stearic acid, EGDS, EGDL, EGDP, EGDM, etc. Different stabilizers that are used here are carbopol, xanthan gum, guar gum, hydroxyl propyl methyl cellulose, etc.

TABLE 32
Examples of dispersions of carbohydrate and lipid coated API
(two active in twocoats-layer on layer coating) particles

| | | Coat | |
|---|---|---|---|
| Compo- | Core | (Coat 1:Coat 2) | Coat1:Coat2:Core |
| sitions | (API 1:API 2) | (% w/w) | ratio |
| D147 | Besifloxacin&Adapalene | Stearic acid:Chitosan | 5:0.3:1 |
| | (0.5:0.5) | (5%:0.3%) | |
| D148 | Besifloxacin& Salicylic acid | Stearic acid:Chitosan | 5:0.3:1 |
| | (0.5:0.5) | (5%:0.3%) | |
| D149 | Adapalene& Ketoconazole | Palmitic acid:Chitosan | 4:0.15:1 |
| | (1.0:1.0) | (8%:0.3%) | |
| D150 | Zinc pyrithione& Salicylic | Palmitic acid | 5:0.3:1 |
| | acid (0.5:0.5) | (5%:0.3%) | |

### Example 42

### Dispersion of Lipid Coated API Particles (Compositions D151-D153)

Preparation:

Besifloxacin hydrochloride and salicylic acid are dispersed in surfactant solution (2% aqueous solution of poloxamer 407). The required quantity of dispersion is heated to about 70-80° C. with continuous stirring. Weighed quantity of lipids (stearic acid and lauric acid) are added to a surfactant solution (2% aqueous solution of poloxamer 407) separately and heated to melt the lipids. The mixture is allowed to stir vigorously at the same temperature. To this stirring mixture, hot (about 70-80° C.) homogenized dispersion of the two actives is added and allowed to stir continuously at the same temperature for about 20 minutes followed by stirring in an ice bath for about 2-3 minutes. Size distribution of the resulting dispersion is determined by ZetaSizer (ZS-90 from Malvern Instruments) and Scanning Electron Microscope (SEM, Hitachi, S-3400 N, Japan). To this dispersion is added a suitable stabilizer, and the pH was controlled using a pH modulator.

This way several dispersion compositions are prepared, and some of the examples are given in Table 33. For dispersion preparation, different types of surfactant solutions such as lecithin, Poloxamer 407, TPGS (D-α-tocopherol polyethylene glycol succinate), poloxamer 188, sodium docusate are used alone or in combination. Similarly, some of the lipids used are lauric acid, myristic acid, palmitic acid, stearic acid, EGDS, EGDL, EGDP, EGDM, etc. Different stabilizers that are used here are carbopol, xanthan gum, guar gum, hydroxyl propyl methyl cellulose, etc.

TABLE 33
Examples of dispersions of lipid coated API (two actives in mixture of two coats) particles

| Compo- | | Coat | Coat:Core | |
|---|---|---|---|---|
| sitions | Core | (% w/w) | ratio | Appearance |
| D151 | Besifloxacin& Salicylic acid | Stearic acid:Lauric acid | 5:1 | Pale yellow, |
| | (1.0:1.0) | (5%:5%) | | pasty dispersion |
| D152 | Ketoconazole & Salicylic acid | Palmitic acid:Lauric acid | 5:1 | White, |
| | (1.0:1.0) | (5%:5%) | | pasty dispersion |
| D153 | Salicylic acid&Adapalene | Palmitic acid:EGDS | 5:1 | White, |
| | (1.0:1.0) | (5%:5%) | | pasty dispersion |

### Example 43

### Dispersion of Carbohydrate Coated API (One Active in One Coat) Particles

### Example 43 (a)

### Dispersion of Chitosan Coated API (One Active in One or Multiple Coats) Particles (Compositions D154-D156)

Preparation:

Zinc pyrithione is dispersed in surfactant solution [2% aqueous solution of tocopherol polyethylene glycol succinate (TPGS):sodium docusate (1:2)], and homogenized for using high pressure homogenizer. This zinc pyrithione suspension is added to chitosan solution (0.3% in 1% aqueous acetic acid) slowly while stirring. After 1 hr of stirring, pH is adjusted to 5.0-5.5 using aqueous solution of sodium hydroxide (18%) and allowed to continue to stir for 4 hrs. Coated particles are characterized for size analysis, zeta potential and drug content. This way several dispersion compositions are prepared, and some of the examples are given in Table 34. For dispersion preparation, different types of surfactant solutions such as lecithin, Poloxamer 407, TPGS (D-α-tocopherol polyethylene glycol succinate), poloxamer 188, sodium docusate are used alone or in combination. Similarly, some of the carbohydrates used are chitosan, chitin, cellulose, starch, hyaluronic acid, dextran, chondroitin sulphate, arabinogalactan, and carrageenan etc. For multiple coating, lipid coated particles are coated further with carbohydrates using above mentioned procedure.

### Example 43(b)

### Dispersion of Chitosan Coated API (One Active in One Coat) Particles (Compositions D157-D160)

Preparation:

A solution of chitosan (0.6%) is prepared in 1% aqueous acetic acid. To the chitosan solution, equal volume of adapalene suspension (2% adapalene suspension in 2% poloxamer solution) is added slowly while stirring. After 1 hr of stirring, pH is adjusted to 5.0-5.5 using aqueous solution of sodium hydroxide (18%) and allowed to continue to stir for 4 hrs. Coated particles are characterized for size analysis, zeta potential and drug content.

This way several dispersion compositions are prepared, and some of the examples are given in Table 34. For dispersion preparation, different types of surfactant solutions such as lecithin, Poloxamer 407, TPGS (D-α-tocopherol polyethylene glycol succinate), poloxamer 188, sodium docusate are used alone or in combination. Similarly, some of the carbohydrates used are chitosan, chitin, cellulose, starch, hyaluronic acid, dextran, chondroitin sulphate, arabinogalactan, and carrageenan etc.

### Example 43 (c)

### Dispersion of Alginate Coated API (One Active in One Coat Particles (Compositions D161-D163)

Preparation:

Adapalene is dispersed in surfactant solution (2% aqueous solution of poloxamer 407). An aqueous solution of sodium alginate (0.5%) is prepared. This solution is added slowly to the dispersion of adapalene while stirring and continued to stir for about 30 minutes to ensure complete mixing. After that; calcium chloride is added slowly and allowed to stir continuously to ensure complete mixing. Size distribution of the resulting dispersion is determined by ZetaSizer (ZS-90 from Malvern Instruments) and Scanning Electron Microscope (SEM, Hitachi, S-3400 N, Japan). To this dispersion is added a suitable stabilizer, and the pH was controlled using a pH modulator.

This way several dispersion compositions are prepared, and some of the examples are given in Table 34. For dispersion preparation, different types of surfactant solutions such as lecithin, TPGS (D-α-tocopherol polyethylene glycol succinate), poloxamer 188, sodium docusate are used alone or in combination. Similarly, some of the carbohydrates used are chitin, cellulose, starch, hyaluronic acid, dextran, chondroitin sulphate, arabinogalactan, and carrageenan etc.

TABLE 34
Examples of dispersions of carbohydrate coated
API (one active in one or more coats) particles

| | | | | Average | |
|---|---|---|---|---|---|
| Compo- | | Coat | Coat:Core | Particle Size | Zeta |
| sitions | Core | (% w/w) | ratio | (PDI) | potential |
| D154 | ZPT (10%) | Chitosan (0.05%) | 1.0:0.03 | 932 nm | −24 |
| | | | | (0.431) | |
| D155 | ZPT (10%) | Chitosan(0.1%) | 1.0:0.01 | 1042 nm | −26.8 |
| | | | | (0.531) | |
| D156 | ZPT (10%) | Chitosan (0.3%) | 1.0:0.02 | 1066 nm | −0.732 |
| | | | | (0.670) | |
| D157 | ZPT (10%) | Lauric acid:Chitosan | 1.0:0.23 | CLSM | — |
| | | (2%:0.3%) | | | |
| D158 | Adapalene | Chitosan | 0.3:1.0 | — | — |
| | | (0.3%) | | | |
| D159 | Salicylic acid | Chitosan | 0.3:1.0 | — | — |
| | | (0.3%) | | | |
| D160 | Zinc | Chitosan | 0.3:1.0 | — | — |
| | pyrithione | (0.3%) | | | |
| D161 | Adapalene | Sodium alginate | 0.05:1 | — | −13.9 |
| | | (0.05%) | | | |
| D162 | Salicylic acid | Sodium alginate | 0.05:1 | — | — |
| | | (0.05%) | | | |
| D163 | Zinc | Sodium alginate | 0.05:1 | — | — |
| | pyrithione | (0.05%) | | | |

### Example 44

### Dispersion of Polypeptide Coated API Microparticles (Compositions D164-D166)

Preparation:

A solution of chitosan (0.6%) is prepared in 1% aqueous acetic acid. To the chitosan solution, equal volume of adapalene suspension (2% adapalene suspension in 2% poloxamer solution) is added slowly while stirring. After 1 hr of stirring, pH is adjusted to 5.0-5.5 using aqueous solution of sodium hydroxide (18%) and allowed to continue to stir for 4 hrs. This dispersion is added to equal volume of 2% aqueous solution of albumin and continued to stir for 3-4 hrs. Particle size distribution of the resulting dispersion is determined by ZetaSizer (ZS-90 from Malvern Instruments) and Scanning Electron Microscope (SEM, Hitachi, S-3400 N, Japan). To this dispersion is added a suitable stabilizer, and the pH was controlled using a pH modulator.

This way several dispersion compositions are prepared, and some of the examples are given in Table 35. For dispersion preparation, different types of surfactant solutions such as lecithin, TPGS (D-α-tocopherol polyethylene glycol succinate), poloxamer 188, sodium docusate are used alone or in combination. Similarly, some of the polypeptide used are collagen, gelatin, fibrin, etc. Different stabilizers that are used here are carbopol, xanthan gum, guar gum, hydroxyl propyl methyl cellulose, etc.

TABLE 35
Examples of dispersions of protein coated API
(two actives in one or more coats) particles

| Compo- | | Coat 1:Coat 2 | Coat:Core | Zeta | |
|---|---|---|---|---|---|
| sitions | Core | (% w/w) | ratio | Potential | Appearance |
| D164 | Adapalene | Chitosan:Albumin | 1.0:1.0 | 5.92 | White |
| | (1%) | (0.15%:1%) | | | flowable |
| | | | | | dispersion |
| D165 | ZPT (1%) | Albumin | 1.0:1.0 | −13.5 | White |
| | | (1%) | | | flowable |
| | | | | | dispersion |
| D166 | ZPT (10%) | Chitosan:Albumin | 1.0:0.13 | — | White |
| | | (0.3%:1%) | | | flowable |
| | | | | | dispersion |

### Example 45

### Minimum Inhibitory Concentration of In-House Besifloxacin Gels

Method:

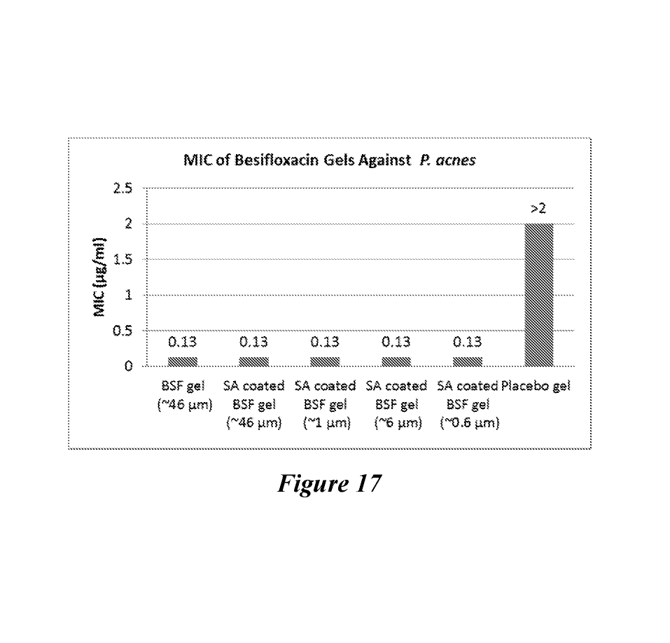

Minimum inhibitory concentration of the tested gel is determined by micro broth dilution method against P. acnes MTCC 1951 (strain susceptible to Clindamycin). BHI broth and BHI agar media are prepared as per the manufacturer's instruction and autoclaved at 121° C. for 15 minutes. P. acnes (MTCC 3297 & MTCC 1951) culture is grown in Brain Heart Infusion agar (BHIA) at 37° C. for 48-72 hrs under anaerobic condition. For MIC determination testing, drug is dissolved in the solvent and further diluted with BHI broth. Then, 96 wells plate are filled with 100 μl of BHI broth containing drug with different concentration to get the final concentrations of 0.0156, 0.03125, 0.0625, 0.125, 0.25, 0.5, 1 and 2 μg/ml in different lanes (lane 1 to lane 10, n=8). Remaining lanes (lane 11 and lane 12) of the 96 well plate are used as growth control and sterility control. Finally, P. acnes culture suspension (approx 1.5×10⁶) is added in all the wells except sterility control wells and plate is incubated at 37° C. for 48-72 hrs under anaerobic condition. At the end of 72 hrs, alamar blue solution (20 μl) is added into the wells and incubated at 37° C. for 2 hrs. Plate is visualized for bacterial inhibitions and MIC value of the tested samples is determined. Gel formulation containing stearic acid coated particles with different sizes are analysed for MIC determinations and results are shown in FIG. 17.

Results:

The MIC results indicate that all the formulations are having MIC value of 0.13 μg/ml and placebo gel did not show any bacterial growth inhibition at tested drug concentrations.

### Example 46

### Dose Response Curves (Using Zones of Inhibition) of In-House Shampoos Versus Marketed Shampoos

Agar well-diffusion method is employed to run Zone of Inhibition (ZOI) assays. ZOI is employed to assess the potency of API and/or formulation to inhibit the growth of microorganisms under study. ZOI values, determined at different API concentrations, can be used to derive dose-response-curves (DRCs) for efficacy comparison of different APIs/formulations.

Method:

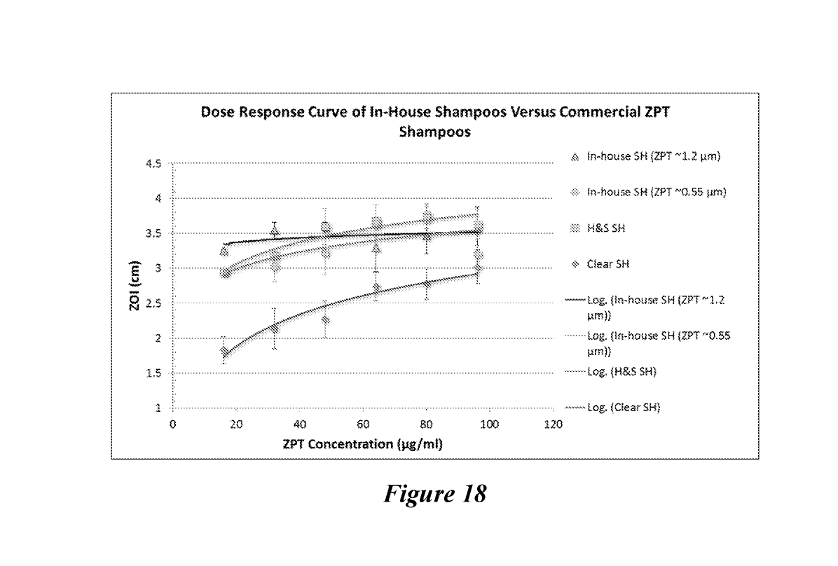

Malassezia furfur culture of specific CFU/ml is used to inoculate Sabaroud's Dextrose agar (SDA) plates [supplemented with chloramphenicol (0.05 mg/ml), cycloheximide (0.04 mg/ml) and olive oil (2%)]. Approximately, 6 mm wells are created in the agar plate using sterile straws. The wells are supplemented with test shampoos (equivalent to different ZPT concentrations, 16 to 96 μg/ml) and/or controls (100 μl each). Then, the plates are incubated at 32° C. under CO₂ (5%) atmosphere. Readouts are taken after 42 hrs or 72 hrs. An example of effect of ZPT particle size on anti-fungal activity, measured using Zone of Inhibition studies has been demonstrated in FIG. 18.

Result:

The results of the Zone of Inhibition assays suggest that the anti-microbial activity of in-house shampoo containing 1.2 μm size particles is higher at initial ZPT concentrations. In-house shampoo is similar in activity to H&S shampoo and 25% better than Clear shampoo.

### Example 47

### Examination of Different Fatty Materials as Substrates or Food for Malassezia furfur

Since most of the common Malassezia spp. lack fatty acids synthase coding genes, they require external supply of fatty acids for cell wall synthesis. Some of the fatty acids and lipids are analyzed as possible food substrates for the growth of Malassezia sp

Procedure:
1. Sabouraud Dextrose Agar (Himedia) is prepared in distilled water as per manufacturer instruction and sterilized by autoclaving at 121° C. for 15 minutes. After autoclaving allow the medium to cool to 50° C. and add antibiotics such as Chloramphenicol & Cycloheximide to get final concentrations of 0.05 mg/ml & 0.04 mg/ml respectively. SDA plates are prepared with various substrates such as fatty acids, lipids and oil.
2. M. furfur cells suspensions is prepared by suspending M. furfur colonies in sterile water and cells density was adjusted to of 5×10³ cells/ml by using cell counting by haemocytometr or by matching with McFarland standards. M. furfur cells suspension 100 μl, is spread on the solidified SDA plates (containing different substrates). Plates are kept at room temperature for 15 minutes to observe the cells suspension and incubates at 32 C for 48-72 hrs.
3. After incubation plates are observed for M. furfur growth. If visual growth is observed it indicates that, M. furfur is able to utilize the substrate as a source of fatty acids.

### Results:

| S. No. | Fatty Material | Concentration | Growth |
|---|---|---|---|
| 1 | No oil | 2% | − |
| 2 | Palmitic acid | 2% | − |
| 3 | Lauric acid | 2% | − |
| 4 | Myristic acid | 2% | − |
| 5 | Coconut oil | 2% | + |
| 6 | Mustard oil | 2% | + |
| 7 | Olive oil | 2% | + |
| 8 | No oil | 2% | − |
| 9 | Oleic acid | 0.5% | + |
| 10 | Linoleic acid | 0.5% | + |
| 11 | Lauric acid | 0.5% | + |
| 12 | Myristic acid | 0.5% | + |
| 13 | Capric acid | 0.5% | − |
| 14 | Caprylic acid | 0.5% | − |
| 15 | Baby oil | 2% | − |
| 16 | Keocarpin oil | 2% | + |
| 17 | Mineral oil | 2% | − |
| 18 | Olive oil | 2% | + |
| 19 | EGDS | 0.03% | + |
| 20 | EGDP | 0.03% | + |

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

1.-93. (canceled)

94. A particle comprising: (i) a core comprising a first active agent or a core comprising lipid, carbohydrate, protein, cationic molecule or any combination thereof; and (ii) a first coating layer comprising at least one molecule selected from a group comprising a lipid, carbohydrate, protein and cationic molecule, or a first active agent, wherein the coating layer at least partially cover the core.

95. The particle of claim 94, wherein the coating acts a food for a pathogen, enhances targeting, binding, or retention of the active agent to a desired site of action, has a synergistic effect on activity of the active agent, and acts as an active agent.

96. The particle of claim 95, wherein the particle further comprises a second active agent, wherein the second active agent is present in the core or in the coating layer and forms a second coating layer on the first coating layer, and wherein the particle comprises third coating layer on the second coating layer, and wherein the third coating layer comprises a lipid, a protein, a polymer and a carbonate, or any combinations thereof; and wherein the particle further comprises at least one second layer on the first coating layer and wherein the second coating layer comprises a lipid, a protein, a polymer and a carbohydrate, or any combinations thereof.

97. The particle of claim 94, wherein the particle further comprises at least two alternating coating layers, wherein one layer in the alternating coating layers comprises an active agent and another layer in the alternating coating layers comprises a lipid, a protein, a polymer, a carbohydrate, or any combinations thereof.

98. The particle of claim 97, wherein outermost layer comprises the active agent.

99. The particle of claim 97, wherein outermost layer comprises the lipid, the protein, the polymer, the carbohydrate, or any combinations thereof.

100. The particle of claim 94, wherein the active agent is selected from the group consisting of small organic or inorganic molecules, saccharines, oligosaccharides, polysaccharides, peptides, proteins, peptide analogs and derivatives, peptidomimetics, nucleic acids, nucleic acid analogs and derivatives, antibodies, antigen binding fragments of antibodies, lipids, extracts made from biological materials, naturally occurring or synthetic compositions, antifungal agents, antibacterial agents, antimicrobial agents, antioxidant agents, cooling agents, soothing agents, wound healing agents, anti-inflammatory-agents, anti-aging agents, anti-wrinkle agents, skin whitening or bleaching agents, ultraviolet (UV) light absorbing or scattering agents, skin depigmentation agents, dyes or coloring agents, deodorizing agents and fragrances, or any combinations thereof; and wherein the said active agent is selected from the group consisting of pyrithione salts; ketoconazole; salicylic acid; curcumin or a derivative of curcumin, curcuminoids; tetrahydro curcuminoids; titanium dioxide (TiO₂); zinc oxide (ZnO); chloroxylenol; flvanoids; CoQ10; vitamin C; herbal extracts; alkaloids; 13-cis retinoic acid; 3,4-methylenedioxymethamphetamine; 5-fluorouracil; 6,8-dimercaptooctanoic acid (dihydrolipoic acid); abacavir; acebutolol; acetaminophen; acetaminosalol; acetazolamide; acetohydroxamic acid; acetylsalicylic acid; acitretin; aclovate; acrivastine; actiq; acyclovir; adapalene; adefovir dipivoxil; adenosine; Albaconazole; albuterol; alfuzosin; Allicin; allopurinol; alloxanthine; allylamines; almotriptan; alpha-hydroxy acids; alprazolam; alprenolol; aluminum acetate; aluminum chloride; aluminum chlorohydroxide; aluminum hydroxide; amantadine; amiloride; aminacrine; aminobenzoic acid (PABA); aminocaproic acid; aminoglycosides such as streptomycin, neomycin, kanamycin, paromycin, gentamicin, tobramycin, amikacin, netilmicin, spectinomycin, sisomicin, dibekalin and isepamicin; aminosalicylic acid; amiodarone; amitriptyline; amlodipine; amocarzine; amodiaquin; Amorolfin; amoxapine; amphetamine; amphotericin B; ampicillin; anagrelide; anastrozole; Anidulafungin; anthralin; antibacterial sulfonamides and antibacterial sulphanilamides, including para-aminobenzoic acid, sulfadiazine, sulfisoxazole, sulfamethoxazole and sulfathalidine; antifungal peptide and derivatives and analogs thereof; apomorphine; aprepitant; arbutin; aripiprazole; ascorbic acid; ascorbyl palmitate; atazanavir; atenolol; atomoxetine; atropine; azathioprine; azelaic acid; azelaic acid; azelastine; azithromycin; bacitracin; bacitracin; beanomicins; beclomefhasone dipropionate; bemegride; benazepril; bendroflumethiazide; benzocaine; Benzoic acid with a keratolytic agent; benzonatate; benzophenone; benztropine; bepridil; beta-hydroxy acids; beta-lactams including penicillin, cephalosporin, and carbapenems such as carbapenem, imipenem, and meropenem; betamethasone dipropionate; betamethasone valerate; brimonidine; brompheniramine; bupivacaine; buprenorphine; bupropion; burimamide; butenafine; Butenafine; butoconazole; Butoconazole; cabergoline; caffeic acid; caffeine; calcipotriene; camphor; Cancidas; candesartan cilexetil; capsaicin; carbamazepine; Caspofungin; cefditoren pivoxil; cefepime; cefpodoxime proxetil; celecoxib; cetirizine; cevimeline; chitosan; chlordiazepoxide; chlorhexidine; chloroquine; chlorothiazide; chloroxylenol; chlorpheniramine; chlorpromazine; chlorpropamide; ciclopirox; Ciclopirox (ciclopirox olamine); cilostazol; cimetidine; cinacalcet; ciprofloxacin; citalopram; citric acid; Citronella oil; cladribine; clarithromycin; clemastine; clindamycin; clioquinol; clobetasol propionate; clomiphene; clonidine; clopidogrel; Clortrimazole; clotrimazole; Clotrimazole; clozapine; cocaine; Coconut oil; codeine; colistin; colymycin; cromolyn; crotamiton; Crystal violet; cyclizine; cyclobenzaprine; cycloserine; cytarabine; dacarbazine; dalfopristin; dapsone; daptomycin; daunorubicin; deferoxamine; dehydroepiandrosterone; delavirdine; desipramine; desloratadine; desmopressin; desoximetasone; dexamethasone; dexmedetomidine; dexmethylphenidate; dexrazoxane; dextroamphetamine; diazepam; dicyclomine; didanosine; dihydrocodeine; dihydromorphine; diltiazem; diphenhydramine; diphenoxylate; dipyridamole; disopyramide; dobutamine; dofetilide; dolasetron; donepezil; dopa esters; dopamine; dopamnide; dorzolamide; doxepin; doxorubicin; doxycycline; doxylamine; doxypin; duloxetine; dyclonine; echinocandins; econazole; Econazole; eflormthine; eletriptan; emtricitabine; enalapril; ephedrine; epinephrine; epinine; epirubicin; eptifibatide; ergotamine; erythromycin; escitalopram; esmolol; esomeprazole; estazolam; estradiol; ethacrynic acid; ethinyl estradiol; etidocaine; etomidate; famciclovir; famotidine; felodipine; fentanyl; Fenticonazole; ferulic acid; fexofenadine; flecainide; fluconazole; Fluconazole; flucytosiine; Flucytosine or 5-fluorocytosine; fluocinolone acetonide; fluocinonide; fluoxetine; fluphenazine; flurazepam; fluvoxamine; formoterol; furosemide; galactarolactone; galactonic acid; galactonolactone; galactose; galantamine; gatifloxacin; gefitinib; gemcitabine; gemifloxacin; gluconic acid; glycolic acid; glycolic acid; glycopeptides such as vancomycin and teicoplanin; griseofulvin; Griseofulvin; guaifenesin; guanethidine; haloperidol; haloprogin; Haloprogin; herbal extract, an alkaloid, a flvanoid, Abafungin; hexylresorcinol; homatropine; homosalate; hydralazine; hydrochlorothiazide; hydrocortisone; hydrocortisone 17-butyrate; hydrocortisone 17-valerate; hydrocortisone 21-acetate; hydromorphone; hydroquinone; hydroquinone monoether; hydroxyzine; hyoscyamine; hypoxanthine; ibuprofen; ichthammol; idarubicin; imatinib; imipramine; imiquimod; indinavir; indomethacin; Iodine; irbesartan; irinotecan; Isavuconazole; Isoconazole; isoetharine; isoproterenol; itraconazole; Itraconazole; kanamycin; ketamine; ketanserin; ketoconazole; ketoprofen; ketotifen; kojic acid; labetalol; lactic acid; lactobionic acid; lactobionic acid; lamivudine; lamotrigine; lansoprazole; lemon myrtle; letrozole; leuprolide; levalbuterol; levofloxacin; lidocaine; lincosamides such as lincomycin and clindamycin; linezolid; lobeline; loperamide; losartan; loxapine; lucensomycin; lysergic diethylamide; macrolides or ketolides such as erythromycin, azithromycin, clarithromycin, and telithromycin; mafenide; malic acid; maltobionic acid; mandelic acid; mandelic acid; maprotiline; mebendazole; mecamylamine; meclizine; meclocycline; memantine; menthol; meperidine; mepivacaine; mercaptopurine; mescaline; metanephrine; metaproterenol; metaraminol; metformin; methadone; methamphetamine; methotrexate; methoxamine; methyl nicotinate; methyl salicylate; methyldopa esters; methyldopamide; methyllactic acid; methylphenidate; metiamide; metolazone; metoprolol; metronidazole; mexiletine; Micafungin; miconazole; Miconazole; midazolam; midodrine; miglustat; minocycline; minoxidil; mirtazapine; mitoxantrone; moexiprilat; molindone; monobenzone; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, and astreonam; morphine; moxifloxacin; moxonidine; mupirocin; nadolol; naftifine; Naftifine; nalbuphine; nalmefene; naloxone; naproxen; natamycin; Neem Seed Oil; nefazodone; nelfinavir; neomycin; nevirapine; N-guanylhistamine; nicardipine; nicotine; nifedipine; nikkomycins; nimodipine; nisoldipine; nizatidine; norepinephrine; nystatin; nystatin; octopamine; octreotide; octyl methoxycinnamate; octyl salicylate; ofloxacin; olanzapine; Olive leaf extract; olmesartan medoxomil; olopatadine; omeprazole; Omoconazole; ondansetron; Orange oil; oxazolidinones such as linezolid; oxiconazole; Oxiconazole; oxotremorine; oxybenzone; oxybutynin; oxycodone; oxymetazoline; padimate O; palmarosa oil; palonosetron; pantothenic acid; pantoyl lactone; paroxetine; patchouli; pemoline; penciclovir; penicillamine; penicillins; pentazocine; pentobarbital; pentostatin; pentoxifylline; pergolide; perindopril; permethrin; phencyclidine; phenelzine; pheniramine; phenmetrazine; phenobarbital; phenol; phenoxybenzamine; phenpropimorph; phentolamine; phenylephrine; phenylpropanolamine; phenytoin; phosphonomycin; physostigmine; pilocarpine; pimozide; pindolol; pioglitazone; pipamazine; piperonyl butoxide; pirenzepine; Piroctone; piroctone olamine; podofilox; podophyllin; Polygodial; polyhydroxy acids; polymyxin; Posaconazole; pradimicins; pramoxine; pratipexole; prazosin; prednisone; prenalterol; prilocaine; procainamide; procaine; procarbazine; promazine; promethazine; promethazine propionate; propafenone; propoxyphene; propranolol; propylthiouracil; protriptyline; pseudoephedrine; pyrethrin; pyrilamine; pyrimethamine; quetiapine; quinapril; quinethazone; quinidine; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, besifloxacin, besifloxaxin, clintafloxacin, ganefloxacin, gemifloxacin and pazufloxacin; quinupristin; rabeprazole; Ravuconazole; reserpine; resorcinol; retinal; retinoic acid; retinol; retinyl acetate; retinyl palmitate; ribavirin; ribonic acid; ribonolactone; rifampin; rifamycins such as rifampicin (also called rifampin), rifapentine, rifabutin, bezoxazinorifamycin and rifaximin; rifapentine; rifaximin; riluzole; rimantadine; risedronic acid; risperidone; ritodrine; rivasfigmine; rizatriptan; ropinirole; ropivacaine; salicylamide; salicylic acid; salicylic acid; salmeterol; scopolamine; selegiline; Selenium; selenium sulfide; serotonin; Sertaconazole; sertindole; sertraline; sibutramine; sildenafil; sordarins; sotalol; streptogramins such as quinupristin and daflopristin; streptomycin; strychnine; sulconazole; Sulconazole; sulfabenz; sulfabenzamide; sulfabromomethazine; sulfacetamide; sulfachlorpyridazine; sulfacytine; sulfadiazine; sulfadimethoxine; sulfadoxine; sulfaguanole; sulfalene; sulfamethizole; sulfamethoxazole; sulfanilamide; sulfapyrazine; sulfapyridine; sulfasalazine; sulfasomizole; sulfathiazole; sulfisoxazole; tadalafil; tamsulosin; tartaric acid; tazarotene; Tea tree oil—ISO 4730 (“Oil of Melaleuca, Terpinen-4-ol type”); tegaserol; telithromycin; telmisartan; temozolomide; tenofovir disoproxil; terazosin; terbinafine; Terbinafine; terbutaline; terconazole; Terconazole; terfenadine; tetracaine; tetracycline; tetracyclines such as tetracycline, chlortetracycline, demeclocycline, minocycline, oxytetracycline, methacycline, doxycycline; tetrahydrozoline; theobromine; theophylline; thiabendazole; thioridazine; thiothixene; thymol; tiagabine; timolol; tinidazole; tioconazole; Tioconazole; tirofiban; tizanidine; tobramycin; tocainide; tolazoline; tolbutamide; tolnaftate; Tolnaftate; tolterodine; tramadol; tranylcypromine; trazodone; triamcinolone acetonide; triamcinolone diacetate; triamcinolone hexacetonide; triamterene; triazolam; triclosan; triclosan; Triclosan; triflupromazine; trimethoprim; trimethoprim; trimipramine; tripelennamine; triprolidine; tromethamine; tropic acid; tyramine; undecylenic acid; Undecylenic acid; urea; urocanic acid; ursodiol; vardenafil; venlafaxine; verapamil; vitamin C; vitamin E acetate; voriconazole; Voriconazole; warfarin; xanthine; zafirlukast; zaleplon; zinc pyrithione; Zinc Selenium sulfide; ziprasidone; zolmitriptan; Zolpidem; WS-3; WS-23; menthol; 3-substituted-P-menthanes; N-substituted-P-menthane-3-carboxamides; isopulegol; 3-(1-menthoxy)propane-1,2-diol; 3-(1-menthoxy)-2-methylpropane-1,2-diol; p-menthane-2,3-diol; p-menthane-3,8-diol; 6-isopropyl-9-methyl-1,4-dioxaspiro[4,5]decane-2-methanol; menthyl succinate and its alkaline earth metal salts; trimethylcyclohexanol; N-ethyl-2-isopropyl-5-methylcyclohexanecarboxamide; Japanese mint oil; peppermint oil; menthone; menthone glycerol ketal; menthyl lactate; 3-(1-menthoxy)ethan-1-ol; 3-(1-menthoxy)propan-1-ol; 3-(1-menthoxy)butan-1-ol; 1-menthylacetic acid N-ethylamide; 1-menthyl-4-hydroxypentanoate; 1-menthyl-3-hydroxybutyrate; N,2,3-trimethyl-2-(1-methylethyl)-butanamide; n-ethyl-t-2-c-6 nonadienamide; N,N-dimethyl menthyl succinamide; menthyl pyrrolidone carboxylate; aloe; avocado oil; green tea extract; hops extract; chamomile extract; colloidal oatmeal; calamine; cucumber extract; sodium palmate; sodium palm kernelate; butyrospermum parkii (i.e., shea butter); menthe piperita (i.e.; peppermint) leaf oil; sericin; pyridoxine (a form of vitamin B6); retinyl palmitate and/or other forms of vitamin A; tocopheryl acetate and/or other forms of vitamin E; lauryl laurate; hyaluronic acid; aloe barbadensis leaf juice powder; euterpe oleracea (i.e., acai berry) fruit extract; riboflavin (i.e., vitamin B2); thiamin HCl and/or other forms of vitamin B1; ethylenediaminetetraacetic acid (EDTA); citrate; ethylene glycol tetraacetic acid (EGTA); 1,2-bis(o-aminophenoxy)ethane-N,N,N′,N′-tetraacetic acid (BAPTA); diethylene triamine pentaacetic acid (DTPA); 2,3-dimercapto-1-propanesulfonic acid (DMPS); dimercaptosuccinic acid (DMSA); α-lipoic acid; salicylaldehyde isonicotinoyl hydrazone (SIH); hexyl thioethylamine hydrochloride (HTA); desferrioxamine; ascorbic acid (vitamin C); cysteine; glutathione; dihydrolipoic acid; 2-mercaptoethane sulfonic acid; 2-mercaptobenzimidazole sulfonic acid; 6-hydroxy-2,5,7,8-tetramethylchroman-2-carboxylic acid; sodium metabisulfite; vitamin E isomers such as α-, β-, γ-, and δ-tocopherols and α-, β-, γ-, and δ-tocotrienols; polyphenols such as 2-tert-butyl-4-methyl phenol, 2-tert-butyl-5-methyl phenol, and 2-tert-butyl-6-methyl phenol; butylated hydroxyanisole (BHA) such as 2-tert-butyl-4-hydroxyanisole and 3-tert-butyl-4-hydroxyanisole; butylhydroxytoluene (BHT); tert-butylhydroquinone (TBHQ); ascorbyl palmitate; n-propyl gallate; soy extract; soy isoflavones; retinoids such as retinol; kojic acid; kojic dipalmitate; hydroquinone; arbutin; transexamic acid; vitamins such as niacin and vitamin C; azelaic acid; linolenic acid and linoleic acid; placertia; licorice; and extracts such as chamomile and green tea; hydrogen peroxide; zinc peroxide; sodium peroxide; hydroquinone; 4-isopropylcatechol; hydroquinone monobenzyl ether; kojic acid; lactic acid; ascorbyl acid and derivatives such as magnesium ascorbyl phosphate; arbutin; licorice root; dihydroxyacetone (DHA); glyceryl aldehyde; tyrosine and tyrosine derivatives such as malyltyrosine, tyrosine glucosinate, and ethyl tyrosine; phospho-DOPA; indoles and derivatives; glucosamine; N-acetyl glucosamine; glucosamine sulfate; mannosamine; N-acetyl mannosamine; galactosamine; N-acetyl galactosamine; N-acyl amino acid compounds (e.g., N-undecylenoyl-L-phenylalanine); flavonoids such as quercetin, hesperidin, quercitrin, rutin, tangeritin, and epicatechin; CoQ10; vitamin C; hydroxy acids including C₂-C₃₀ alpha-hydroxy acids such as glycolic acid, lactic acid, 2-hydroxy butanoic acid, malic acid, citric acid tartaric acid, alpha-hydroxyethanoic acid, hydroxycaprylic acid and the like; beta hydroxy acids including salicylic acid and polyhydroxy acids including gluconolactone (G4); retinoic acid; gamma-linolenic acid; ultraviolet absorber of benzoic acid system such as para-aminobenzoic acid (hereinafter, abbreviated as PABA), PABA monoglycerin ester, N,N-dipropoxy PABA ethyl ester, N,N-diethoxy PABA ethyl ester, N,N-dimethyl PABA ethyl ester, N,N-dimethyl PABA butyl ester, and N,N-dimethyl PABA methyl ester and the like; ultraviolet absorber of anthranilic acid system such as homomenthyl-N-acetyl anthranilate and the like; ultraviolet absorber of salicylic acid system such as amyl salicylate, menthyl salicylate, homomenthyl salicylate, octyl salicylate, phenyl salicylate, benzyl salicylate, p-isopropanol phenyl salicylate and the like; ultraviolet absorber of cinnamic acid system such as octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxy cinnamate, isopropyl-p-methoxy cinnamate, isoamyl-p-methoxy cinnamate, octyl-p-methoxy cinnamate(2-ethylhexyl-p-methoxy cinnamate), 2-ethoxyethyl-p-methoxy cinnamate, cyclohexyl-p-methoxy cinnamate, ethyl-α-cyano-β-phenyl cinnamate, 2-ethylhexyl-α-cyano-β-phenyl cinnamate, glyceryl mono-2-ethylhexanoyl-dipara-methoxy cinnamate, methyl bis(trimethylsiloxane)silylisopentyl trimethoxy cinnamate and the like; 3-(4′-methylbenzylidene)-d,l-camphor; 3-benzylidene-d,l-camphor; urocanic acid, urocanic acid ethyl ester; 2-phenyl-5-methylbenzoxazole; 2,2′-hydroxy-5-methylphenylbenzotriazole; 2-(2′-hydroxy-5′-t-octylphenyl)benzotriazole; 2-(2′-hydroxy-5′-methylphenylbenzotriazole; dibenzaladine; dianisoylmethane; 4-methoxy-4′-t-butyldibenzoylmethane; 5-(3,3-dimethyl-2-norbornylidene)-3-pentane-2-one; dimorpholinopyridazinone; titanium oxide; particulate titanium oxide; zinc oxide; particulate zinc oxide; ferric oxide; particulate ferric oxide; ceric oxide; inorganic sunscreens such as titanium dioxide and zinc oxide; organic sunscreens such as octyl-methyl cinnamates and derivatives thereof; retinoids; vitamins such as vitamin E, vitamin A, vitamin C (ascorbic acid), vitamin B, and derivatives thereof such as vitamin E acetate, vitamin C palmitate, and the like; antioxidants including alpha hydroxy acid such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucopehtonic acid, glucopheptono-1,4-lactone, gluconic acid, gluconolactone, glucuronic acid, glucurronolactone, glycolic acid, isopropyl pyruvate, methyl pyruvate, mucic acid, pyruvia acid, saccharic acid, saccaric acid 1,4-lactone, tartaric acid, and tartronic acid; beta hydroxy acids such as beta-hydroxybutyric acid, beta-phenyl-lactic acid, beta-phenylpyruvic acid; botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, rice, and safflower; 21-acetoxypregnenolone; alclometasone; algestone; amcinonide; beclomethasone; betamethasone; budesonide; chloroprednisone; clobetasol; clobetansone; clocortolone; cloprednol; corticosterone; cortisone; cortivazol; deflazacort; desonide; desoximetasone; dexamethasone; diflorasone; diflucortolone; difluprednate; enoxolone; fluazacort; flucloronide; flumethasone flunisolide; fluocinolone acetonide; fluocinonide; fluocortin butyl; fluocortolone; fluorometholone; fluperolone acetate; fluprednidene acetate; fluprednisolone; flurandrenolide; fluticasone propionate; formocortal; halcinonide; halobetasol propionate; halometasone; halopredone acetate; hydrocortamate; hydrocortisone; loteprednol etabonate; mazipredone; medrysone; meprednisone; methylprednisolone; mometasone furcate; paramethosone; prednicarbate; prednisolone; prednisolone 25-diethylamino-acetate; prednisolone sodium phosphate; prednisone; prednival; prednylidene; rimexolone; tixocortol; triamcinolone; triamcinolone acetonide; triamcinolone benetonide; triamcinolone hexacetonide; COX inhibitors such as salicylic acid derivatives (e.g., aspirin, sodium salicylate, choline magnesium trisalicylate, salicylate, diflunisal, sulfasalazine and olsalazine); para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam); alkanones such as nabumetone; diarylsubstituted furanones such as refecoxib; diaryl-substituted pyrazoles such as celecoxib; indole acetic acids such as etodolac; sulfonanilides such as nimesulide; selenium sulfide; sulfur; sulfonated shale oil; salicylic acid; coal tar; povidone-iodine, imidazoles such as ketoconazole, dichlorophenyl imidazolodioxalan, clotrimazole, itraconazoie, miconazole, climbazole, tioconazole, sulconazole, butoconazole, fluconazole, miconazolenitrite; anthralin; piroctone olamine (Octopirox); ciclopirox olamine; anti-psoriasis agents; vitamin A analogs; corticosteroids; and any combinations thereof; and wherein the pyrithione salt is selected from the group consisting of zinc pyrithione, sodium pyrithione, potassium pyrithione, lithium pyrithione, ammonium pyrithione, copper pyrithione, calcium pyrithione, magnesium pyrithione, strontium pyrithione, silver pyrithione, gold pyrithione, manganese pyrithione, and any combinations thereof.

101. The particle of claim 94, wherein the particle has a size from about 5 nm to about 20 μm or from about 100 nm to about 10 μm or from about 200 nm to about 6 μm from about 1 μm to about 6 μm or from about 300 nm to about 700 nm; and wherein the coating layer has a thickness from about 1 nm to about 1000 nm or from about 1 nm to about 150 nm; and wherein the active agent is present in an amount from about 1% to about 99% (w/w) or from about 75% to about 98% (w/w).

102. The particle of claim 94, wherein the lipid is selected from the group consisting of fatty acids; mono-, di- or tri-esters of fatty acids; salts of fatty acids; fatty alcohols; mono-, di- or tri-esters of fatty alcohols; glycerolipids; phospholipids; glycerophospholipids; sphingolipids; sterol lipids; prenol lipids; saccharolipids; polyketides; and wherein the said lipid is selected from the group consisting of 1,3-Propanediol Dicaprylate/Dicaprate; 10-undecenoic acid; 1-dotriacontanol; 1-heptacosanol; 1-nonacosanol; 2-ethyl hexanol; Androstanes; Arachidic acid; Arachidonic acid; arachidyl alcohol; Behenic acid; behenyl alcohol; Capmul MCM C10; Capric acid; capric alcohol; capryl alcohol; Caprylic acid; Caprylic/Capric Acid Ester of Saturated Fatty Alcohol C12-C18; Caprylic/Capric Triglyceride; Caprylic/Capric Triglyceride; Ceramide phosphorylcholine (Sphingomyelin, SPH); Ceramide phosphorylethanolamine (Sphingomyelin, Cer-PE); Ceramide phosphorylglycerol; Ceroplastic acid; Cerotic acid; Cerotic acid; ceryl alcohol; Cetearyl alcohol; Ceteth-10; cetyl alcohol; Cholanes; Cholestanes; cholesterol; cis-11-eicosenoic acid; cis-11-octadecenoic acid; cis-13-docosenoic acid; cluytyl alcohol; coenzyme Q10 (CoQ10); Dihomo-γ-linolenic; Docosahexaenoic acid; egg lecithin; Eicosapentaenoic acid; Eicosenoic acid; Elaidic acid; elaidolinolenyl alcohol; elaidolinoleyl alcohol; elaidyl alcohol; Erucic acid; erucyl alcohol; Estranes; Geddic acid; geddyl alcohol; glycerol distearate (type I) EP (Precirol ATO 5); Glycerol Tricaprylate/Caprate; Glycerol Tricaprylate/Caprate (CAPTEX® 355 EP/NF); glyceryl monocaprylate (Capmul MCM C8 EP); Glyceryl Triacetate; Glyceryl Tricaprylate; Glyceryl Tricaprylate/Caprate/Laurate; Glyceryl Tricaprylate/Tricaprate; glyceryl tripalmitate (Tripalmitin); Henatriacontylic acid; Heneicosyl alcohol; Heneicosylic acid; Heptacosylic acid; Heptadecanoic acid; Heptadecyl alcohol; Hexatriacontylic acid; isostearic acid; isostearyl alcohol; Lacceroic acid; Lauric acid; Lauryl alcohol; Lignoceric acid; lignoceryl alcohol; Linoelaidic acid; Linoleic acid; linolenyl alcohol; linoleyl alcohol; Margaric acid; Mead; Melissic acid; melissyl alcohol; Montanic acid; montanyl alcohol; myricyl alcohol; Myristic acid; Myristoleic acid; Myristyl alcohol; neodecanoic acid; neoheptanoic acid; neononanoic acid; Nervonic; Nonacosylic acid; Nonadecyl alcohol; Nonadecylic acid; Nonadecylic acid; Oleic acid; oleyl alcohol; Palmitic acid; Palmitoleic acid; palmitoleyl alcohol; Pelargonic acid; pelargonic alcohol; Pentacosylic acid; Pentadecyl alcohol; Pentadecylic acid; Phosphatidic acid (phosphatidate, PA); Phosphatidylcholine (lecithin, PC); Phosphatidylethanolamine (cephalin, PE); Phosphatidylinositol (PI); Phosphatidylinositol bisphosphate (PIP2); Phosphatidylinositol phosphate (PIP); Phosphatidylinositol triphosphate (PIP3); Phosphatidylserine (PS); polyglyceryl-6-distearate; Pregnanes; Propylene Glycol Dicaprate; Propylene Glycol Dicaprylocaprate; Propylene Glycol Dicaprylocaprate; Psyllic acid; recinoleaic acid; recinoleyl alcohol; Sapienic acid; soy lecithin; Stearic acid; Stearidonic; stearyl alcohol; Tricosylic acid; Tridecyl alcohol; Tridecylic acid; Triolein; Undecyl alcohol; undecylenic acid; Undecylic acid; Vaccenic acid; α-Linolenic acid; γ-Linolenic acid; a fatty acid salt of 10-undecenoic acid, adapalene, arachidic acid, arachidonic acid, behenic acid, butyric acid, capric acid, caprylic acid, cerotic acid, cis-11-eicosenoic acid, cis-11-octadecenoic acid, cis-13-docosenoic acid, docosahexaenoic acid, eicosapentaenoic acid, elaidic acid, erucic acid, heneicosylic acid, heptacosylic acid, heptadecanoic acid, isostearic acid, lauric acid, lignoceric acid, linoelaidic acid, linoleic acid, montanic acid, myristic acid, myristoleic acid, neodecanoic acid, neoheptanoic acid, neononanoic acid, nonadecylic acid, oleic acid, palmitic acid, palmitoleic acid, pelargonic acid, pentacosylic acid, pentadecylic acid, recinoleaic acid, sapienic acid, stearic acid, tricosylic acid, tridecylic acid, undecylenic acid, undecylic acid, vaccenic acid, valeric acid, α-linolenic acid, or γ-linolenic acid; paraffin; zinc recinoleate; ethylene glycol distearate (EGDS) and triplamitin or any combinations thereof and wherein the fatty acid salt is selected from the group consisting of zinc, sodium, potassium, lithium, ammonium, copper, calcium, magnesium, strontium, manganese, and combinations thereof.

103. The particle of claim 94, wherein the lipid comprises 11 or fewer carbon atoms; and wherein the lipid is present in an amount from about 1% to about 99% (w/w) or from about 2% to about 25 (w/w); and wherein the particle comprises an excess of the active agent relative to the total lipids, and wherein ratio of the total lipid to the active agent in the particle is from about 100:1 to about 1:100 or from about 10:1 to about 1:50 or from about 2:1 to about 1:30.

104. The particle of claim 94, wherein the protein is selected from the group consisting of Actin, Albumin, Amaranth Protein, Ammonium Hydrolyzed Animal Protein, Animal protein, Barley Protein, Brazil Nut Protein, Casein, Collagen, Collagen protein hydrolyzed, Conchiolin Protein, corn protein, Cottonseed Protein, Elastin, Extensin, Fibroin, Fibronectin, Fish Protein, Gadidae Protein, Gelatin, Glutein, Glycoproteins, Hazelnut Protein, Hemoglobin, Hemp Seed Protein, Honey Protein, Hydrolyzed Actin, Hydrolyzed Amaranth Protein, Hydrolyzed animal protein, Hydrolyzed Barley Protein, Hydrolyzed Brazil Nut Protein, Hydrolyzed Conchiolin Protein, Hydrolyzed corn protein, Hydrolyzed Cottonseed Protein, Hydrolyzed Elastin, Hydrolyzed Extensin, Hydrolyzed Fibroin, Hydrolyzed Fibronectin, Hydrolyzed Fish Protein, Hydrolyzed Gadidae Protein, Hydrolyzed Gadidae Protein, Hydrolyzed Gelatin, Hydrolyzed Hair Keratin, Hydrolyzed Hazelnut, Hydrolyzed Hazelnut Protein, Hydrolyzed Hemoglobin, Hydrolyzed Hemp Seed Protein, Hydrolyzed Honey Protein, Hydrolyzed Keratin, Hydrolyzed Lupine Protein, Hydrolyzed Maple Sycamore Protein, Hydrolyzed Milk Protein, Hydrolyzed Oat Protein, Hydrolyzed Pea Protein, Hydrolyzed Potato Protein, Hydrolyzed Reticulin, Hydrolyzed Royal Jelly Protein, Hydrolyzed Sericin, Hydrolyzed Serum Protein, Hydrolyzed Sesame Protein, Hydrolyzed Soy Protein, Hydrolyzed Soymilk Protein, Hydrolyzed Spinal Protein, Hydrolyzed Spongin, Hydrolyzed Sweet Almond Protein, Hydrolyzed Vegetable Protein, Hydrolyzed Wheat Gluten, Hydrolyzed Wheat Protein, Hydrolyzed Whey Protein, Hydrolyzed Yeast Protein, Hydrolyzed Yogurt Protein, Hydrolyzed Zein, Integrin, Jojoba protein HP, Hydrolyzed, keratin, Lupine Protein, Maple Sycamore Protein, MEA-Hydrolyzed Collagen, MEA-Hydrolyzed Silk, Milk Protein, Myosin, Oat Protein, Pea Protein, polylysine, Potato Protein, Reticulin, Rice Quat, Royal Jelly Protein, Sericin, Serum Protein, Sesame Protein, Silk powder, Sodium Hydrolyzed Casein, Soy Protein, Soy Rice Peptides, Soymilk Protein, Spinal Protein, Spongin, Sweet Almond Protein, Vegetable Protein, Wheat Gluten, Whey Protein, Yeast Protein, Yogurt Protein, Zein, and Zinc Hydrolyzed Collagen; and wherein the albumin is bovine serum albumin, egg albumin, Hydrolyzed Lactalbumin, or Lactalbumin.

105. The particle of claim 94, wherein the protein is present in an amount from about 1% (w/w) to about 99% (w/w) or from about 5% (w/w) to about 50% (w/w); and wherein ratio of the protein to the active agent in the particle is from about 100:1 to about 1:100 or from about 10:1 to about 1:50 or from about 2:1 to about 1:30.

106. The particle of claim 94, wherein the cationic molecule is a polyamine; and wherein the said cationic molecule is selected from the group consisting of Putrescine (Butane-1,4-diamine), Cadaverine (Pentane-1,5-diamine), Spermidine, Spermine, Cyclen (1,4,7,10-tetrazacyclododecane), Cyclam (1,4,8,11-Tetraazacyclotetradecane), Linear Polyethyleneimine (Poly(iminoethylene)), Norspermidine, p-Phenylenediamine (1,4-diaminobenzene), Diethylenetriamine (N-(2-aminoethyl)-1,2-ethanediamine), thermospermine, Tris(2-aminoethyl)amine, Hexamethylenediamine, Beta-lysine (3,6-diaminohexanoic acid), m-Phenylenediamine (1,3-diaminobenzene), Diaminopropane (1,2-Diaminopropane), Ethylenediamine dihydroiodide, and polyamine D 400 (Polyoxyalkyleneamine D 400).

107. The particle of claim 94, wherein the cationic molecule is present in an amount from about 1% (w/w) to about 99% (w/w) or from about 5% (w/w) to about 50% (w/w); and wherein ratio of the cationic molecule to the active agent in the particle is from about 100:1 to about 1:100 or from about 10:1 to about 1:50 or from about 2:1 to about 1:30.

108. The particle of claim 94, wherein the carbohydrate is selected from the group consisting of oligosaccharides, polysaccharides, glycoproteins, glycolipids and any combinations thereof; and wherein the said carbohydrate is selected from the group consisting of fructooligosaccharide, galactooligosaccharides, mannanoligosaccharides, glycogen, starch, glycosaminoglycans, cellulose, beta-glucan, maltodextrin, inulin, levan beta (2->6), chitin, chitosan, and any combinations thereof.

109. The particle of claim 94, wherein the carbohydrate is present in an amount from about 1% (w/w) to about 99% (w/w) or from about 5% (w/w) to about 50% (w/w); and wherein ratio of the carbohydrate to the active agent in the particle is from about 100:1 to about 1:100 or from about 10:1 to about 1:50 or from about 2:1 to about 1:30.

110. A composition comprising an effective amount of the particle of claim 94.

111. The composition of claim 110, wherein the composition comprises from about 0.01% to about 50% (w/w or w/v) of the particles or from about 10% to about 30% (w/w or w/v) of the particles.

112. The composition of any of claim 110, wherein the composition further comprises one or more excipients from about 5% to about 99.99% (w/w or w/v) and wherein the excipient is a solvent or an additive; and wherein the additive is selected from the group consisting of surfactants, stabilizers, rheology modifiers, conditioning agents, fragrances, potentiating agents, preservatives, opacifiers, pH modifiers, moisturizers, humectants, suspending agents, solubilizers, and any combinations thereof; and wherein said additive is selected from the group consisting of 45° Be'glucose syrup, Acrylates/10-30 alkyl acrylate crosspolymer, Acrylates/Acrylamide Copolymer, agar, Allantoin, Aminomethyl propanol, Ammonium lauryl sulfate (ALS), Amodimethicone emulsion, AMP-Acrylates/Allyl Methacrylate Copolymer, Behentrimonium methosulfate, Benzophenone-4, Butylene glycol, Carbapol Aqua SF-1, Carbapol-934, Carbapol-940, Carbopol, Carboxymethyl cellulose, Cassia hydroxy propyltrimoniumchloride, Cetearyl alcohol, Cetearyl alcohol, Cetearyl isononanoate, Cetrimoniumchloride (CTC), Cetyl Alcohol, Cetyl octanoate, Chloromethyl/Methylisothiazolinone, Chloromethyl/Methylisothiazolinone, Citric acid, Citrus Aurantium Dulcis (Orange) Fruit Extract, Cocamidopropyl betain (CAPB), Cocamidopropyl betain (CAPB), Coco monoethanolamide (CMEA), Cocodimonium hydroxypropyl hydrolyzed wheat protein, Coloring agent, CPB, Diisopropyl sebacate, Dimethicone PEG-7 Isostearate, Disodium EDTA, DMDM hydantoin, Dove AD shine, Emulsifying wax, Ethanol, Ethylene glycol distearate (EGDS), Ethylhexyl methoxycinnamate, eucalyptol, Flavors, Forte therapy, Fragrance, Glycerin, Glycerine, Glyceryl monohydroxystearate, Glyceryl monostearate, Glycolic acid, Guar gum, Gum base, Hyaluronic acid, Hydrated silica, Hydrolyzed silk protein, Intense repair, Lactamide MEA acetamide MEA, Lactic acid, Limnanthes Alba (Meadowfoam) Seed Oil, Linalool, Linoleamidopropyl PG-dimonium chloride phosphate, Macrogolcetostearyl ether 20, magnese chloride, Magnesium sulfate, Menthol, Methyl gluceth-20, methyl salicylate, Mineral oil, Mint-type flavor, PEG/PPG-8/3 laurate, PEG-12 dimethicone, PEG-20 Almond Glycerides, PEG-40 hydrogenated castor oil, PEG-80 glyceryl cocoate, peppermint oil, Phenyl trimethicone, Polyacrylate-1 crosspolymer, Polyethylene glycol 1450, Polyoxyethylene/polyoxypropylene block polymer (Poloxamer 407), Polyquaternium-22, Polyquaternium-39, Polysorbate 85, polysorbate-20, PPG-3 myristyl ether, Preservative, Propylene glycol, Propylene glycol monocaprylate, Prunus Armeniaca (Apricot) Kernel Oil, Pyrus Malus (Apple) Fruit Extract, retinyl palmitate, Saccharin sodium, Salicylic acid, Sensomer CT-250, Silica Xerogel, SLES, SLS, sodium chloride, sodium docusate, Sodium hydroxide, Sodium lauryl ether sulfate (SLES), sodium lauryl sulphate, Sodium monofluorophosphate, Sodium saccharin, Sorbitol, Stearamidopropyldimethylamine, Steareth-2, Steareth-21, Stearic acid, Sugar powder, sunflower seed oil, Tea tree oil, Titanium dioxide, Tocopheryl acetate, Triclosan, Triethanolamine, Wheat amino acids, Xanthan gum, zinc carbonate, zinc chloride, zinc recinoleate, zinc stearate, and any combinations thereof.

113. The composition of claim 110, wherein the composition further comprises: (i) Carbopol, Ammonium lauryl sulfate (ALS), Sodium lauryl ether sulfate (SLES), Sodium hydroxide, Coco monoethanolamide (CMEA), Ethylene glycol distearate (EGDS), Propylene glycol monocaprylate, Menthol, Magnesium sulfate, Amodimethicone emulsion, Propylene glycol, Zinc carbonate, Cocamidopropyl betaine (CAPB), Cassia hydroxyl propyltrimoniumchloride, Chloromethyl/Methylisothiazolinone, Linalool, Fragrance, Citric acid, and Sodium Chloride; or (ii) Carbopol, Ammonium lauryl sulfate (ALS), Sodium lauryl ether sulfate (SLES), Sodium hydroxide, Ethylene glycol distearate (EGDS), Propylene glycol monocaprylate, Menthol, Magnesium sulfate, Amodimethicone emulsion, Propylene glycol, Zinc carbonate, Cocamidopropyl betaine (CAPB), Cassia hydroxyl propyltrimoniumchloride, Chloromethyl/Methylisothiazolinone, Linalool, Fragrance, Citric acid, and Sodium Chloride, or (iii) Carbopol, Sodium lauryl ether sulphate (SLES), Sodium hydroxide, Steareth-2, Steareth-21, Propylene glycol monocaprylate, Macrogolcetostearyl ether 20, Coco monoethanolamide (CMEA), Cetyl Alcohol, Stearamidopropyldimethylamine, Lactic acid, Cocamidopropyl betain (CAPB), Cetrimoniumchloride (CTC), Polyquaternium-22, Amodimethicone emulsion, Cassia hydroxy propyltrimoniumchloride, Propylene glycol, Glycerine, Zinc carbonate, Titanium dioxide, Linalool, Fragrance, and Chloromethyl/Methylisothiazolinone; or (iv) Carbopol, Sodium lauryl ether sulphate (SLES), Sodium hydroxide, Steareth-2, Steareth-21, Propylene glycol monocaprylate, Macrogolcetostearyl ether 20, Coco monoethanolamide (CMEA), Cetyl Alcohol, Stearamidopropyldimethylamine, Lactic acid, Cocamidopropyl betain (CAPB), Cetrimoniumchloride (CTC), Polyquaternium-22, Amodimethicone emulsion, Cassia hydroxy propyltrimoniumchloride, Propylene glycol, Glycerine, Titanium dioxide, Linalool, Fragrance, and Chloromethyl/Methylisothiazolinone or (v) Stearic acid, Mineral oil, Glyceryl monostearate, Glyceryl monohydroxystearate, Cetearyl alcohol, Cetyl octanoate, Emulsifying wax, Cabopol, Triethanolamine, Water, Glycerin, Propylene glycol, Fragrance, and Preservative; or (vi) Stearic acid, Mineral oil, Glyceryl monostearate, Glyceryl monohydroxystearate, Cetearyl alcohol, Cetyl octanoate, Cabopol, Triethanolamine, Glycerin, Salicylic acid, Citrus Aurantium Dulcis (Orange) Fruit Extract, Fragrance, and Preservative; or (vii) Emulsifying wax, Behentrimonium methosulfate and Cetearyl alcohol, PPG-3 myristyl ether, Cetearyl isononanoate, Dimethicone PEG-7 Isostearate, Ethylhexyl methoxycinnamate, Cabopol, Acrylates/Acrylamide Copolymer (and) Mineral Oil, (and) Polysorbate 85, Triethanolamine, Glycerin, Hyaluronic acid, Wheat amino acids, Lactamide MEA and acetamide MEA, Hydrolyzed silk protein, Salicylic acid, Propylene glycol, Pyrus Malus (Apple) Fruit Extract, Citrus Aurantium Dulcis (Orange) Fruit Extract, Tocopheryl acetate, Fragrance, and Preservative; or (viii) Glyceryl monohydroxystearate, Limnanthes Alba (Meadowfoam) Seed Oil, Prunus Armeniaca (Apricot) Kernel Oil, Phenyl trimethicone, Diisopropyl sebacate, Ethylhexyl methoxycinnamate, Cabopol, Acrylates/Acrylamide Copolymer (and) Mineral Oil, (and) Polysorbate 85, AMP-Acrylates/Allyl Methacrylate Copolymer, Triethanolamine, PEG-20 Almond Glycerides, Titanium dioxide, Propylene glycol, Linoleamidopropyl PG-dimonium chloride phosphate, Cocodimonium hydroxypropyl hydrolyzed wheat protein, Tocopheryl acetate, Fragrance, and Preservative; or (ix) Glycerin, Methyl gluceth-20, Benzophenone-4, Acrylates/10-30 alkyl acrylate crosspolymer, PEG/PPG-8/3 laurate, Aminomethyl propanol, Polyquaternium-39, PEG-80 glyceryl cocoate, Coloring agent, Preservative, and Fragrance; or (x) Disodium EDTA, Propylene glycol, Carbopol, Aminomethyl propanol, Tea tree oil, PEG-40 hydrogenated castor oil, polysorbate-20, Coloring agent, Preservative, and Fragrance; or (xi) Butylene glycol, Glycerin, Methyl gluceth-20, Allantoin, Disodium EDTA, PEG-12 dimethicone, Polyacrylate-1 crosspolymer, Glycolic acid, Triethanolamine, Tocopheryl acetate, retinyl palmitate, sunflower seed oil, agar, Coloring agent, Preservative, and Fragrance; or (xii) Glycerin, Methyl gluceth-20, Carbopol, Triethanolamine, Ethanol, Triclosan, Coloring agent, Preservative, and Fragrance; or (xiii) Carboxymethyl cellulose, Polyethylene glycol 1450, Sorbitol, Glycerin, Sodium monofluorophosphate, Sodium saccharin, Preservative, Coloring agent, Silica Xerogel, Hydrated silica, Mint-type flavor, and Sodium lauryl sulfate; or (xiv) Ethyl alcohol, menthol, methyl salicylate, peppermint oil, eucalyptol, Glycerin, Polyoxyethylene/polyoxypropylene block polymer (Poloxamer 407), and Saccharin sodium; or (xv) Gum base, 45° Be'glucose syrup, Sugar powder, Flavors, Glycerin, and Preservative

114. The composition of claim 110, wherein the composition is a cream, oil, lotion, serum, gel, shampoo, conditioner, tooth paste, mouth wash, chewing gum, sun screen, nail varnish, ointment, foam, spray, or aerosol or wherein the composition is an anti-dandruff hair care composition selected from the group consisting of a shampoo, a conditioner, a rinse, a lotion, an aerosol, a gel, a mousse, and a hair dye or wherein the composition is a skin care composition selected from the group consisting of lotions, creams, gels, sticks, sprays, ointments, cleansing liquid washes, cleansing solid bars, pastes, foams, powders, shaving creams, and wipes or wherein the composition is an oral care composition selected from the group consisting of tooth pastes, mouth washes, and chewing gums.

115. The composition of claim 110, wherein the composition is an antifungal, antibacterial, anti-inflammatory, anti-aging, anti-wrinkle, or skin whitening or skin bleaching composition or an anti-acne composition.

116. The composition of claim 110, wherein the composition is a shampoo, the active agent is an anti-fungal agent and the coating layer lipid is EGDS, and wherein the excipient is propylene glycol monocaprylate or the composition is a gel or cream, wherein the active agent is an anti-propionibacterium agent and the coating layer lipid is Lauric acid and/or stearic acid, and wherein the excipient is TPGS.

117. The particle of claim 94, wherein the lipid is stable in shampoo formulation and acts a food for a pathogen, enhances targeting, binding, or retention of the active agent to a desired site of action, and has a synergistic effect on activity of the active agent.
